(19) United States
(12) Patent Application Publication
(10) Pub. No.: US 2022/0073911 A1
(43) Pub. Date: Mar. 10, 2022

(54) METHODS FOR APTAMER SELECTION

(71) Applicant: DOTS Technology Corp., Natick, MA (US)

(72) Inventors: Adi Gilboa-Geffen, Wayland, MA (US); Sarah Elizabeth Stidham, Brighton, MA (US); Olivia Jean Alley, Cambridge, MA (US)

(21) Appl. No.: 17/265,550

(22) PCT Filed: Aug. 2, 2019
(86) PCT No.: PCT/US2019/044772

§ 371 Date: Feb. 3, 2021

Related U.S. Application Data
(60) Provisional application No. 62/714,102, filed on Aug. 3, 2018.

(51) Int. Cl. C12N 15/11; C12N 15/10; C12Q 1/6806; C12Q 1/686; G01N 33/02

(52) U.S. Cl. CPC C12N 15/111; C12N 15/1048; C12Q 1/6806; C12N 2310/3517; G01N 33/02; C12N 2310/16; C12Q 1/686

(57) ABSTRACT

The present disclosure relates to methods for identifying aptamers against allergen proteins and signaling polynucleotides (SPNs) for allergen detection. The screening method of the present disclosure combines several positive SELEX selections, and on-chip positive and counter selections to identify aptamer sequences that are preferentially bind to target proteins when competing with short complementary sequences.

### CROSS REFERENCE TO RELATED APPLICATIONS

The present application claims priority to U.S. Provisional Application No. 62/714,102 filed Aug. 3, 2018, entitled with “Methods for Aptamer Selection”; the contents of which are incorporated herein by reference in their entirety.

### REFERENCE TO THE SEQUENCE LISTING

The instant application is being filed along with a Sequence Listing text file in electronic format. The Sequence Listing is provided as a file entitled 2066_1011PCT_SL.txt, created on Aug. 1, 2019, which is 14,790,207 bytes in size. The subject matter of the Sequence Listing is incorporated herein by reference in its entirety.

### FIELD OF THE DISCLOSURE

The present disclosure relates to methods for identification of aptamers against a target of interest (e.g., an allergen). The disclosure also provides aptamers, signaling polynucleotides (SPNs), DNA chips, detection sensors and kits, and assays for detecting a target in a sample.

### BACKGROUND OF THE DISCLOSURE

Nucleic acid aptamers are single-stranded oligonucleotides (DNAs, RNAs or DNA/RNA hybrids) that can bind to target molecules with high affinity and specificity. Nucleic acid aptamers are generally selected from a library of oligonucleotides with randomized sequences by an iterative process of adsorption, recovery and reamplification, for example, by conventional SELEX (Systematic Evolution of Ligands by Exponential Enrichment) and other closely related methods (See, e.g., U.S. Pat. Nos. 5,270,163; 5,567,588; 5,637,459; 5,670,637; 5,705,337; and 5,723,592). Aptamers can adapt unique secondary and tertiary structures and recognize targets with high affinity and specificity.

Aptamers provide a cost-effective alternative to antibodies as there is no need for aptamer selection in animals or cell lines, they have shelf-lives of years, and they can be easily modified to reduce cross-reactivity with undesired molecules. Aptamers have significant advantages over antibodies, such as better specificity and affinity, wider varieties of targets, easier synthesis and modification, higher stability and lower cost. These properties favor aptamers as new detection agents for wide applications in biosensor development, among other fields, for detecting the presence, absence and/or amount of target molecules in a sample. For example, aptamers and aptamer-based assays have been shown, among many other useful applications (e.g., diagnostic tests and therapy) as a promising alternative in food safety control, e.g., detection and control of pathogens, toxins, allergens and other forbidden contaminants in food matrices (Amaya-Gonzalez, et al., Sensors, 2013, 13:16292-16311; and Amaya-Gonzalez, et al., Anal. Chem. 2014, 86(5), 2733-2739). Aptamers based assays replace many immunoblotting methods using antibodies (e.g., ELISA).

Allergy (e.g., food allergy) is a common medical condition. It has been estimated that in the United States, up to 2 percent of adults and up to 8 percent of children, particularly those under three years of age, suffer from food allergies (about 15 million people), and this prevalence is believed to be increasing. Allergen detection, either in clinical settings or consumer based, is important to a person who is allergic to certain types of food, e.g., gluten and peanuts. Sensitive and specific detection agents against allergens are keys in developing detection assays that can efficiently and quickly test a suspect food product before consuming it. Aptamers that selectively bind to an allergen have been employed in many allergen detection sensors and assays (Weng and Neethirajan, Biosens Bioelectron, 2016, 85: 649-656; Svobodova et al., Food Chem., 2014, 165: 419-423; Tran et al., Biosens. Bioelectron, 2013, 43, 245-251; and Nadal et al., Plos One, 2012, 7(4): e35253). Studies have shown that an aptamer-based assay has significant advantages as compared to antibody-based immunoassay (e.g., ELISA).

The present disclosure developed a modified selection method for identifying aptamer sequences against a specific allergen target; the aptamers and/or signal polynucleotides derived from the aptamers can be directly used in detection assays with increased specificity and sensitivity. Specifically, the modified selection method combines several positive, negative and counter selection processes to identify aptamers that can specifically recognize a target molecule (e.g., an allergen protein) but the features (e.g., the primary and secondary structures) of the aptamers block the same aptamers bound to the target to hybridize to short oligonucleotides comprising sequences complementary to the same aptamers. Therefore, the target and the short complementary sequence do not bind to the same aptamer simultaneously.

### SUMMARY OF THE DISCLOSURE

The present disclosure provides screening methods tailored for selection of aptamers against target molecules that can be directly employed in competition-based target detection assays, such as allergen detection assays; the methods comprising several positive, negative (counter) selection processes to identify aptamers having particular primary and secondary structural features that when the aptamers are bound to target molecules to form aptamer.target complexes, they do not simultaneously hybridize to short oligonucleotides complementary to the aptamer sequences. The identified aptamers are suitable to develop chip sensors for target detection in which the aptamers or signal polynucleotides derived from the aptamers compete binding to their target molecule in the presence of oligonucleotides (i.e., anchor sequences) that are complementary to the sequences of the aptamers.

In some embodiments, the screening method comprises (a) preparing an input DNA library comprising a plurality of single stranded DNA (ssDNA) molecules, each of which comprises a central randomized nucleic acid sequence flanked by a constant sequence at the 5′ end and a constant sequence at the 3′ end, the constant 5′ end and the constant 3′ end functioning as primers; (b) selecting a pool of ssDNA molecules, from the input DNA library of (a), that substantially bind to a target material; (c) selecting a pool of ssDNA molecules, from the target binding pool of ssDNA molecules obtained in (b), that do not bind to the complementary sequences in the presence of the target material (i.e., do not simultaneously bind to the target and complementary sequences); (d) counter-selecting ssDNA molecules, from the positive binding pool of ssDNA molecules obtained in (c), that do not bind to the complementary sequences in the absence of the target material, or that substantially bind to counter target materials; and (e) subtracting the pool of ssDNA molecules obtained in (d) from the positive binding pool of ssDNA molecules in (c), and identifying candidate ssDNA molecules that specifically bind to the target of interest. Each sub-pool of ssDNA molecules can be identified through positive SELEX and/or on-chip selection processes and each process can be repeated several rounds at the same condition.

Accordingly, the aptamers identified via the present screening methods and signaling polynucleotides (SPNs) derived from the aptamers bind to their target molecule with high affinity and specificity. In some embodiments, the aptamers and SPNs may not hybridize to short complementary sequences in the presence of the target molecule, while they can bind to the short complementary sequences in the absence of the target molecule.

In some embodiments, the present screening method further comprises amplifying the ssDNA molecules in each pool after each selection process. The ssDNA molecules may be amplified by PCR using a pair of primers labeled with a fluorophore probe. The amplified and regenerated ssDNA molecules are therefore labeled with the fluorophore probe.

In some embodiments, the pool of ssDNA molecules that substantially bind to a target may be selected by a modified Graphene Oxide (GO)-SELEX process using an input ssDNA library and a target material. This positive selection may comprise the steps of (i) contacting the input ssDNA library with the target material wherein complexes are formed between the target and a plurality of ssDNA molecules present in the input library; (ii) partitioning the complexes formed in step (i) using a Graphene Oxide (GO) solution, and isolating the ssDNA molecules in the complexes to produce a subset of ssDNA molecules for the target material; (iii) contacting the subset of ssDNA molecules in (ii) with the same target material wherein complexes are formed between the target and a second plurality of ssDNA molecules present in the subset of ssDNA molecules to generate a second subset group of ssDNA molecules; and (iv) optionally repeating steps (ii) to (iii), one, two, three or more times to produce a respective third, fourth, fifth or more subset group of ssDNA molecules, thereby producing the enriched pool of ssDNA molecules that substantially bind to the target material.

In some embodiments, the positive pool of ssDNA molecules that do not bind to the complementary sequences in the presence of the target material may be selected through on-chip positive binding selection process using the target binding pool of ssDNA molecules (e.g., the pool of ssDNA molecules selected by the GO-SELEX process), the same target material and a solid support that is coated with a plurality of short oligonucleotides comprising sequences complementary to the sequences of the ssDNA molecules, e.g., the constant sequence at the 5′ end of the ssDNA molecules.

In some embodiments, the positive binding pool of ssDNA molecules selected by the on-chip positive selection process may be further refined to subtract non-specific ssDNA molecules. The counter selection may comprise: (i) counter selecting a pool of ssDNA molecules, from the positive pool of ssDNA molecules (e.g., the pool from the on-chip positive selection), that do not bind to the complementary sequences even in the absence of the target material (i.e. the non-binding ssDNA molecules); this selection including an on-chip non-binding counter process that uses the positive binding pool of ssDNA molecules as the input and a chip that is coated with short oligonucleotides comprising complementary sequences of the ssDNA molecules; and (ii) counter selecting a pool of ssDNA molecules, from the positive binding pool of ssDNA molecules, that substantially bind to counter target molecules; this selection including an on-chip counter binding process using the positive binding pool of ssDNA molecules as the input, one or more counter target materials and a chip that is coated with short oligonucleotides comprising sequences complementary to the sequences of the ssDNA molecules.

In some embodiments, the target material may be a common allergen such as a common food allergen. In one embodiment, the target material is peanut, almond, brazil nut, cashew, hazelnut, pecan, pistachio, walnut, gluten, whey and/or casein.

In another aspect, the present disclosure provides aptamers, signaling polynucleotides (SPNs), DNA chips, aptamer-based detection sensors and kits for detecting the presence, absence, and/or amount of a target (e.g., an allergen) in a sample.

In some embodiments, aptamer sequences that specifically bind to an allergen are selected by the present selection processes, wherein the allergen is a common food allergen, e.g., peanut, almond, brazil nut, cashew, hazelnut, pecan, pistachio, walnut, gluten, whey and casein. Aptamers that can bind to all nuts including peanut, almond, brazil nut, cashew, hazelnut, pecan, pistachio and walnut, may also be selected, for example, by multiple SELEX methods.

In some embodiments, aptamer sequences that specifically bind to peanut are selected, which may comprise a unique nucleic acid sequence selected from the group consisting of SEQ ID NOs.3 to 1002. In some examples, the aptamer against peanut may comprise a nucleic acid sequence selected from the group consisting of SEQ ID NOs. 1003 to 4002.

In some embodiments, aptamer sequences that specifically bind to almond are selected, which may comprise a unique nucleic acid sequence selected from the group consisting of SEQ ID Nos. 4003 to 5002. In some examples, the aptamer against almond may comprise a nucleic acid sequence selected from the group consisting of SEQ ID NOs. 5003 to 8002.

In some embodiments, aptamer sequences that specifically bind to brazil nut are selected, which may comprise a unique nucleic acid sequence selected from the group consisting of SEQ ID NOs. 8003 to 9002. In some examples, the aptamer against brazil nut may comprise a nucleic acid sequence selected from the group consisting of SEQ ID NOs. 9003 to 12002.

In some embodiments, aptamer sequences that specifically bind to cashew are selected, which may comprise a unique nucleic acid sequence selected from the group consisting of SEQ ID NOs. 12003 to 13002. In some examples, the aptamer against cashew may comprise a nucleic acid sequence selected from the group consisting of SEQ ID NOs. 13003 to 16002.

In some embodiments, aptamer sequences that specifically bind to hazelnut are selected, which may comprise a unique nucleic acid sequence selected from the group consisting of SEQ ID NOs. 16003 to 17002. In some examples, the aptamer against hazelnut may comprise a nucleic acid sequence selected from the group consisting of SEQ ID NOs. 17003 to 20002.

In some embodiments, aptamer sequences that specifically bind to pecan are selected, which may comprise a unique nucleic acid sequence selected from the group consisting of SEQ ID NOs. 20003 to 21002. In some examples, the aptamer against pecan may comprise a nucleic acid sequence selected from the group consisting of SEQ ID NOs. 21003 to 24002.

In some embodiments, aptamer sequences that specifically bind to pistachio are selected which may comprise a unique nucleic acid sequence selected from the group consisting of SEQ ID NOs. 24003 to 25002. In some examples, the aptamer against pistachio may comprise a nucleic acid sequence selected from the group consisting of SEQ ID NOs. 25003 to 28002.

In some embodiments, aptamer sequences that specifically bind to walnut are selected, which may comprise a unique nucleic acid sequence selected from the group consisting of SEQ ID NOs. 28003 to 29002. In some examples, the aptamer against walnut may comprise a nucleic acid sequence selected from the group consisting of SEQ ID NOs. 29003 to 32002.

In some embodiments, aptamer sequences that can bind to all nuts are selected, which may comprise a unique nucleic acid sequence selected from the group consisting of SEQ ID NOs. 32003 to 33002. In some examples, the aptamer against all nuts may comprise a nucleic acid sequence selected from the group consisting of SEQ ID NOs. 33003 to 36002.

In some embodiments, aptamer sequences that specifically bind to gluten are selected, which may comprise a unique nucleic acid sequence selected from the group consisting of SEQ ID NOs. 40003 to 41002. In some examples, the aptamer against gluten may comprise a nucleic acid sequence selected from the group consisting of SEQ ID NOs. 41003 to 44002.

In some embodiments, aptamer sequences that specifically bind to whey are selected, which may comprise a unique nucleic acid sequence selected from the group consisting of SEQ ID NOs. 44003 to 45002. In some examples, the aptamer against whey may comprise a nucleic acid sequence selected from the group consisting of SEQ ID NOs. 45003 to 48002.

In some embodiments, aptamer sequences that specifically bind to casein are selected, which may comprise a unique nucleic acid sequence selected from the group consisting of SEQ ID NOs. 48003 to 49002. In some examples, the aptamer against casein may comprise a nucleic acid sequence selected from the group consisting of SEQ ID NOs. 49003 to 52002.

In some embodiments, aptamer sequences that specifically bind to a target control material may be selected. Such control sequences can be used together with the aptamer sequences that bind to the target in a detection assay. The control aptamer sequences have similar response to the sample (e.g., the food matrix) as the target specific aptamers. However, the control aptamers will not respond to the target (e.g., a target allergen) and have no binding affinity to the target specific aptamers or to the short anchor sequences complementary to the target specific aptamers. For example, aptamer sequences that bind to peanut control material may be used together with the aptamer sequences against peanut for detecting the presence/absence of peanut in a food sample. The peanut control sequences and the aptamer specific to peanut may demonstrate a similar response to the food type to be tested. Therefore, the signal from the peanut control sequences can be used as internal sample control.

In some examples, aptamer sequences that bind to peanut control material are selected which may comprise a unique nucleic acid sequence selected from the group consisting of SEQ ID NOs. 36003 to 37002. In some examples, the aptamer sequence for peanut control may comprise a nucleic acid sequence selected from the group consisting of SEQ ID NOs. 37003 to 40002.

In accordance with the present disclosure, a SPN may comprise an aptamer selected by the present method that specifically binds to a target of interest and a short nucleic acid sequence that is complementary to the aptamer sequence. The short complementary sequences may be printed on a solid surface for a detection assay. In some embodiments, the short complementary sequence may comprise a nucleic acid sequence selected from the group consisting of SEQ ID NOs. 52003 to 52042.

In further another aspect, the present disclosure provides methods for detecting the presence, absence and/or amount of a target in a sample using aptamers and SPNs identified by the present screening methods. In some embodiments, the target is a food allergen and the sample to be tested is a food sample. The food allergen may be peanut, almond, brazil nut, cashew, hazelnut, pecan, pistachio, walnut, gluten, whey and casein.

### BRIEF DESCRIPTION OF THE DRAWINGS

FIG. 1 is a flow chart demonstrating an embodiment of the aptamer screening methods of the present disclosure.

### DETAILED DESCRIPTION OF THE DISCLOSURE

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Unless defined otherwise, all technical and scientific terms used herein have the same meaning as commonly understood by one of ordinary skill in the art to which this disclosure belongs. In the case of conflict, the present description will control.

The present screening methods modify conventional aptamer selection methods, combining several positive and negative (counter) selections to identify aptamers that specifically bind to a target of interest. These selections mimic the conditions of competition-based detection assays in which aptamers (or SPNs derived from the aptamers) are used to capture their target and short oligonucleotides comprising sequences complementary to the aptamers are used to detect the presence or absence of the aptamer:target complexes. The competition particularly is between the target to which an aptamer can bind with high level of specificity and affinity, and complementary sequences of the aptamer. The selected aptamer sequences can specifically bind to their target, but only hybridize to short complementary sequences in the absence of the target. The selected aptamer sequences cannot bind to the short complementary sequences in the presence of their target. The present screening methods also select control aptamer sequences for a specific target material. The control aptamer sequences can be used in parallel with target specific aptamers and serve as internal control. The detailed description of the screening methods is included.

### Definitions

In order for the present disclosure to be more readily understood, certain terms and phrases are defined below. Additional terms and phrases are also defined and set forth through the specification.

As used herein, the term “aptamer” refers to a nucleic acid molecule or a peptide that can bind to a specific target molecule. A nucleic acid aptamer is a nucleic acid molecule having at least one binding site for a target molecule, such as another nucleic acid sequence, protein, peptide, antibody, small organic molecule, mineral, cell and tissue. A nucleic acid aptamer can be a single stranded or double stranded deoxyribonucleic acid (ssDNA or dsDNA), or ribonucleic acid (RNA), or a hybrid of DNA/RNA. Nucleic acid aptamers typically range from 10-150 nucleotides in length, for example, from 15-120 nucleotides in length, or from 20-100 nucleotides in length, or from 20-80 nucleotides in length, or from 30-90 nucleotides in length, or from 50-90 nucleotides in length. The nucleic acid sequence of an aptamer may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In the context of the present disclosure, the term “aptamer” refers to a nucleic acid aptamer. The terms “a single stranded DNA(ssDNA) molecule,” and “aptamer” are used interchangeably.

An aptamer can fold into specific and stable secondary, tertiary, or quaternary conformational structures that enable it to bind to a target with high specificity and affinity. The structures may include, but are not limited to, hairpin loop, bulge loop, internal loop, multi-branch loop, pseudoknot, or combinations thereof. For example, the binding site of an aptamer may comprise a stem loop conformation or G-quartets.

Aptamers against a target may be naturally occurring or made by synthetic or recombinant means. An aptamer can be selected from a random oligonucleotide library through repeated rounds of in vitro partition, selection and amplification of nucleic acid molecules, e.g., conventional SELEX. As used herein, the term “SELEX” refers to a methodology known in the art as “Systematic Evolution of Ligands by Exponential Enrichment (SELEX)”. SELEX, or equivalently In vitro selection, is a powerful and widely used method to select nucleic acid sequences (i.e., aptamers) that bind to a target (e.g., a protein) with specificity and affinity (Ellington A D, et al., Nature, 1990, 346: 818-822; Tuerk C, et al., Science, 1990, 249: 505-510; and Gold L, et al., Anmi Rev Biochem, 1995, 64: 763-797). The SELEX process and various modifications are described in the art, e.g., U.S. Pat. Nos. 5,270,163; 5,567,588; 5,696,249; 5,853,984; 6,083,696; 6,376190; 6, 262, 774; 6,569,620; 6,706,482; 6,730,482; 6,933,116; 8,975,388; 8,975026; and 9,382,533; the contents of each of which are incorporated herein by reference in their entirety. The SELEX process is based on the unique insight that nucleic acids have sufficient capacity for forming a variety of two- and three-dimensional structures and sufficient chemical versatility available within their monomers to act as ligands (i.e., form specific binding complexes) with virtually any chemical compound, whether monomeric or polymeric. Molecules of any size or composition can serve as targets. SELEX relies as a starting point upon a large library of single stranded oligonucleotides comprising randomized sequences. The oligonucleotides can be modified or unmodified DNAs, RNAs, or DNA/RNA hybrids. In some examples, the library comprises 100% randomized or partially randomized oligonucleotides.

Nucleic acid aptamers show robust binding affinities to their target, preferably binding to the target with an equilibrium (K_{d}) less than 10⁻⁶, 10⁻⁸, 10⁻¹⁰, or 10⁻¹². Aptamers also bind to the target molecule with a very high degree of specificity. It is preferred that aptamers have a K_{d} with the target molecule at least 10, 100, 1000, 10,000, or 100,000-fold lower than the K_{d} of other non-targeted molecules. In some examples, the aptamer selection process may be tailored to select aptamers with pre-defined parameters such as equilibrium (K_{d}), rate constants (K_{off} and Kₒₙ) and thermodynamic parameters (ΔH and ΔS) of aptamer-target interaction.

Aptamers may comprise naturally occurring nucleotides, and/or modified nucleotides including but not limited to chemically modified nucleobases, unnatural bases (e.g., 2-aminopurine), nucleotide analogs, addition of a label (e.g., a fluorophore), addition of a conjugate, or mixtures of any of the above. The nucleic acid sequence of an aptamer can be modified as desired so long as the functional aspects are still maintained (e.g., binding to the target).

As used herein, the terms “nucleic acid”, “oligonucleotide” and “polynucleotide” are used interchangeably to refer to a polymer of nucleotides of any length, and such nucleotides may include deoxyribonucleotides (DNAs), ribonucleotides (RNAs), and/or analogs or chemically modified deoxyribonucleotides or ribonucleotides and RNA/DNA hybrids. The terms “nucleic acid”, “oligonucleotide” and “polynucleotide” include double- or single-stranded molecules as well as triple-helical molecules. A nucleic acid molecule may comprise at least one chemical modification.

As used herein, the term “primary structure” of a nucleic acid molecule refers to its nucleotide sequence. The “secondary structure” of a nucleic acid molecule include, but is not limited to, a hairpin loop, a bulge loop, an internal loop, a multi-branch loop, a pseudoknot or combinations thereof. “Pre-selected secondary structures” refer to those secondary structures that are selected and engineered into an aptamer by design.

As used herein, the term “complementary” refer to the natural binding of polynucleotides by base pairing such as A-T(U) and C-G pairs. Two single-stranded molecules may be partially complementary such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. As used herein, the term “hybridization” or “hybridize to” refers to the process by which a polynucleotide strand anneals with a complementary strand through base pairing under defined hybridization conditions. Specific hybridization is an indication that two nucleic acid sequences share a high degree of identity. Specific hybridization complexes form under permissive annealing conditions.

As used herein, the term “high affinity” refers to the binding of a candidate aptamer to a target with binding dissociation constant Kₐ less than 100 nM. The “specific binding affinity” of an aptamer for its target means that the aptamer binds to its target generally with a much higher degree of affinity than it binds to other components in a test sample. In similar, the term “specifically binds” means that an aptamer reacts or associates more frequently, more rapidly, with greater duration and with greater affinity with a particular target molecule, than it does with non-target molecules. For example, an aptamer against a target allergen binds to that allergen or a structural part or fragment thereof with greater affinity, avidity, more readily, and/or with greater duration than it binds to unrelated allergen proteins and/or parts or fragments thereof. It is also understood by reading this definition that, for example, an aptamer that specifically binds to a first target may or may not specifically bind to a second target. As such, “specific binding” does not necessarily require exclusive binding or non-detectable binding of another molecule, this is encompassed by the term “selective binding”. The specificity of binding is defined in terms of the comparative dissociation constants (K_{d}) of the aptamer for its target as compared to the dissociation constant with respect to the aptamer and other materials in the environment or unrelated molecules in general. Typically, the K_{d} for the aptamer with respect to the target will be 2-fold, 5-fold, 6-fold, 7-fold, 8-fold, 9-fold, or 10-fold less than the Kₐ with respect to the target and the unrelated molecule or accompanying molecule in the environment. Even more preferably, the Kₐ will be 50-fold, 100-fold, 150-fold or 200-fold less.

As used herein, the term “amplification” or “amplifying” means any process or combination of steps that increases the amount or number of copies of a molecule or class of molecules. The amplification of a nucleic acid molecule is generally carried out but not limiting to using polymerase chain reaction (PCR) (e.g., U.S. Pat. Nos. 4,683,195 and 4,683,202; the contents of each of which are herein incorporated by reference in their entirety).

As used herein, the term “library,” or “pool,” or “subset” refers to a plurality of compounds, e.g. single stranded DNA (ssDNA) molecules.

As used herein, the terms “target molecule,” “target material” and “target” are used interchangeably to refer to any molecule to which an aptamer can bind. “Target molecules” or “targets” can be, for example, proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, small molecules, dyes, nutrients, pollutants, growth factors, cells, tissues, or microorganisms and any fragment or portion of any of the foregoing. In one embodiment, a target may be an allergenic protein.

As used herein, the term “counter target” refers to a molecule belonging to a family which has a similar structure, a similar active site, or similar activity to a target or a target material. In the context of the present disclosure, a counter target can be any molecules to which a selected aptamer against a target of interest has no cross-specificity. Counter targets may be used in counter selection processes to refine aptamer candidates for separating sequences that cross-recognize other closely related molecules.

As used herein, the term “allergen” means a compound, substance or composition that causes, elicits or triggers an immune reaction in a subject. As such, allergens are typically referred to as antigens. An allergen is typically a protein or a polypeptide.

As used herein, the terms “polypeptide,” “peptide,” and “protein” are used interchangeably herein to refer to polymers of amino acids of any length.

As used herein, the term “sample” means a composition that contains or is assumed to contain one or more targets to be tested. A sample may be, but is not limited to, a biological sample obtained from a subject (including human and animal), a sample obtained from the environment (e.g., soil sample, water sample, agriculture sample such as a plant and a crop sample), a chemical sample, and a food sample.

### Combined Selection Processes

In accordance with the present disclosure, the selection method is modified to identify aptamer candidates that can recognize a target molecule with high specificity and affinity and lower cross-reactivity with counter targets, and that do not hybridize to oligonucleotides that are complementary to the aptamer sequences in the presence of the target. The selected aptamers and SPNs derived from these aptamers can be used as detection agents in competition-based detection assays in which target molecules in a test sample and the complementary oligonucleotides compete binding to the aptamers (or SPNs).

In accordance with the present disclosure, the aptamer screening method may comprise (a) preparing an input DNA library comprising a plurality of single stranded DNA (ssDNA) molecules, each of which comprises a central randomized nucleic acid sequence flanked by a constant sequence at the 5′ end and a constant sequence at the 3′ end, the constant 5′ end and the constant 3′ end functioning as primers; (b) selecting a pool of ssDNA molecules, from the input DNA library of (a), that substantially bind to a target material; (c) selecting a pool of ssDNA molecules, from the target binding pool of ssDNA molecules obtained in (b), that do not bind to the complementary sequences in the presence of the target material (i.e., do not simultaneously bind to the target and complementary sequences); (d) counter-selecting ssDNA molecules, from the positive binding pool of ssDNA molecules obtained in (c), that do not bind to the complementary sequences in the absence of the target material (referred to as non-binding ssDNA molecules), or that substantially bind to counter target materials (cross-specificity); and (e) subtracting the pool of ssDNA molecules obtained in (d) from the positive binding pool of ssDNA molecules in (c), and identifying candidate ssDNA molecules that specifically bind to the target of interest.

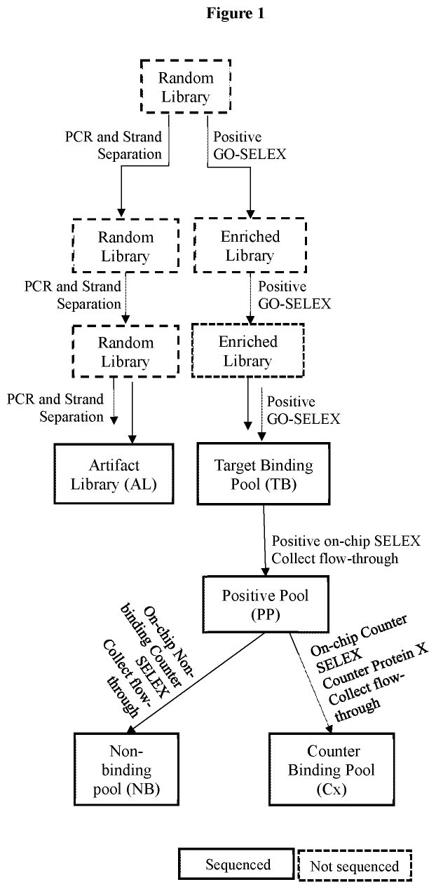

The present screening methods combine several positive target binding selections (e.g., positive SELEX and on-ship SELEX), non-binding counter selections, complementary hybridization selections and counter target binding selections. Candidate aptamers are identified through repeated positive and negative selections, sequence amplification and sequencing analysis. The modified screening method affords improved efficiency in aptamer selection as compared to conventional SELEX and other known methods in the art and ensures selection of aptamers that preferably bind to a target molecule to short complementary nucleic acid sequences. The flow chart in FIG. 1 demonstrates an exemplary embodiment of the present screening methods for identification of aptamer sequences specific to a target that can be used in competition-based detection assays.

### Target Binding Selections

In some embodiments, a pool of ssDNA molecules that substantially bind to a target molecule may be selected by a positive target-binding selection process comprising repeated target binding, partition, isolation and amplification of nucleic acid sequences using an input library comprising randomized ssDNA (single stranded DNA) molecules and a target material. Conventional aptamer selection processes may be used such as systematic evolution of ligands by exponential enrichment (SELEX), selected and amplified binding site (SAAB), cyclic amplification and selection of targets (CASTing), or the like. As a non-limiting example, a plurality of sequences that form ssDNA:target complexes may be identified by performing several rounds of positive Graphene Oxide (GO)-SELEX selection using an input ssDNA library comprising randomized single stranded DNA sequences and a target material (FIG. 1).

SELEX procedure generally involves a progressive selection, from a large library of double-stranded or single-stranded nucleic acids (DNAs, RNAs or DNA/RNA hybrids), of variable nucleic acid sequences that bind to a target of interest with high affinities and specificities by repeated rounds of target partition and amplification.

Each round of SELEX process consists of several steps including preparation of nucleic acid libraries, formation of nucleic acid-target complexes, separation between bound and unbound sequences, elution of aptamers, PCR amplification, and identification of aptamers specific to the target. Each round of selection enriches aptamer candidates from the nucleic acid library.

The input nucleic acid library may comprise a plurality of single-stranded DNA (ssDNA) molecules with randomized sequences. The ssDNA may be 50-150 nucleotides in length, for example, the ssDNA in the library is about 50 to 140 nucleotides in length, or about 50 to 130 nucleotides in length, or about 50 to 120 nucleotides in length, or about 50 to 100 nucleotides in length, or about 60 to 80 nucleotides in length, or about 70 to 90 nucleotides in length, or about 70-80 nucleotides in length. In some embodiments, the ssDNA in the library may be 60 nucleotides in length, or 61 nucleotides in length, or 62 nucleotides in length, or 63 nucleotides in length, or 64 nucleotides in length, or 65 nucleotides in length, or 66 nucleotides in length, or 67 nucleotides in length, or 68 nucleotides in length, or 69 nucleotides in length, or 70 nucleotides in length, or 71 nucleotides in length, or 72 nucleotides in length, or 73 nucleotides in length, or 74 nucleotides in length, or 75 nucleotides in length, or 76 nucleotides in length, or 77 nucleotides in length, or 78 nucleotides in length, or 79 nucleotides in length, or 80 nucleotides in length, or 81 nucleotides in length, or 82 nucleotides in length, or 83 nucleotides in length, or 84 nucleotides in length, or 85 nucleotides in length, or 86 nucleotides in length, or 87 nucleotides in length, or 88 nucleotides in length, or 89 nucleotides in length, or 90 nucleotides in length, or 91 nucleotides in length, or 92 nucleotides in length, or 93 nucleotides in length, or 94 nucleotides in length, or 95 nucleotides in length, or 96 nucleotides in length, or 97 nucleotides in length, or 98 nucleotides in length, or 99 nucleotides in length, or 100 nucleotides in length. Each ssDNA molecule in the library comprises a randomized nucleic acid sequence at the center flanked by a constant sequence at the 5′ end and a constant sequence at the 3′ end that serve as PCR primers, where the sequences of the primers are known, and the central randomized sequence may be 30 to 50 nucleotides in length. The randomized sequences can be produced in a number of ways including chemical synthesis and size selection from randomly cleaved cellular nucleic acids. Sequence variation in test nucleic acids can also be introduced or increased by mutagenesis before or during the selection/amplification iterations.

As a non-limiting example, the input ssDNA molecule library may be generated by automated chemical synthesis on a DNA synthesizer.

As used herein, the “central randomized nucleic acid sequence” within an ss DNA may also be referred to as the “inner sequence” of the ssDNA.

In one preferred embodiment, the ssDNA molecules in the input library are 76 nucleotides in length, wherein a central randomized nucleic acid sequence with 30 nucleotides in length is flanked by two 23 nucleotides primers at the 5′ end and 3′-end of each ssDNA. As a non-limiting example, the 5′ end primer may comprise a nucleic acid sequence of 5′ TAGGGAAGAGAAGGACATATGAT3′ (SEQ ID NO. 1) and the 3′ end primer may comprise a nucleic acid sequence of 5′ TTGACTAGTACATGACCACTTGA 3′ (SEQ ID NO. 2).

As used herein, the term “primer” refers to a short nucleic acid which is capable of acting as a point of initiation of synthesis (e.g., PCR) when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification but may alternatively be double stranded.

The input DNA library may be mixed with a target wherein the complexes are formed between the target and a plurality of ssDNA molecules present in the library. The target may be any molecule (e.g., nucleic acids, proteins, small molecules, sugars, toxins, biomarkers, cells and pathogens). In some embodiments, the target is a protein, such as an allergen protein or mixed allergen components of an allergen. The allergen may include, but is not limited to, a food allergen, an allergen from the environment such as plants, animals, microorganisms, air or water, and a medical allergen (i.e., any allergen found in a medicine or medical device).

Food allergens include, but are not limited to proteins in legumes such as peanuts, peas, lentils and beans, as well as the legume-related plant lupin, tree nuts such as almond, cashew, walnut, Brazil nut, filbert/hazelnut, pecan, pistachio, walnut, beechnut, butternut, chestnut, chinquapin nut, coconut, ginkgo nut, lychee nut, macadamia nut, nangai nut and pine nut, egg, fish, shellfish such as crab, crawfish, lobster, shrimp and prawns, mollusks such as clams, oysters, mussels and scallops, milk, soy, wheat, gluten, corn, meat such as beef, pork, lamb, mutton and chicken, gelatin, sulphite, seeds such as sesame, sunflower and poppy seeds, and spices such as coriander, garlic and mustard, fruits, vegetables such as celery, and rice. Some exemplary allergenic proteins from food allergens may include the parvalbumins in codfish, tropomyosin in crustaceans, arginine kinase and myosin light chain, casein, α-lactalbumin and 3 lactoglobulin in milk, and globulin or vicilin seed storage protein.

Other target molecules include, but are not limited to, pathogens from a pathogenic microorganism in a sample, such as bacteria, yeasts, fungi, spores, viruses and prions; disease proteins (e.g., biomarkers for diseases diagnosis and prognosis); pesticides and fertilizers remained in the environment; and toxins. Targets may include non-protein compounds such as minerals and small molecules (e.g., antibiotics).

In some embodiments, the steps for selecting an enriched pool of ssDNA molecules that substantially bind to the target material may comprise (i) contacting the input ssDNA library with the target material wherein complexes are formed between the target and a plurality of ssDNA molecules present in the input library; (ii) partitioning the complexes formed in step (i) from the unbound ssDNA molecules and isolating the ssDNA molecules in the complexes to produce a subset of ssDNA molecules for the target material and amplifying the isolated subset of ssDNA molecules; (iii) contacting the enriched subset of ssDNA molecules from step (ii) with the same target material wherein complexes are formed between the target and a second plurality of ssDNA molecules present in the enriched library to generate a second enriched subset group of ssDNA molecules; and (iv) optionally repeating steps of binding, partition, isolation and amplification (steps (i) to (iii)), one, two, three, four or more times as desired to yield highly specific, high affinity ssDNA molecules to the target molecule, thereby producing the enriched pool of ssDNA molecules that substantially bind to the target material (e.g., Pool 1 in Table 1).

In one embodiment, a graphene oxide (GO)-SELEX process modified from the general SELEX method is performed to select the target binding pool. As used herein, the terms “graphene,” “graphene oxide (GO),” “graphene oxide nanosheet” and “graphene nanosheet” mean two-dimensional carbon structures and are used interchangeably throughout the present specification. The exposed nucleobases in the ssDNA molecules can be absorbed to the surface of graphene oxide (Chen et al., J. Agric. Food Chem. 2014; 62, 10368-10374). Accordingly, when a graphene oxide (GO) solution is added to the mixture of a ssDNA library and a target material, GO can adsorb the ssDNA sequences that are not bound to a specific target, to its surface, and let the sequences bound to the target free. The unbound sequences and GO can then be removed, e.g., by centrifugation, while the ssDNA molecules that bind to a specific target are not absorbed to the surface of GO and then recovered and employed in the following selection process. This process can avoid the need to immobilize the target material as used in conventional SELEX.

The GO-SELEX process is inexpensive, fast, and simple. In a conventional SELEX, many expensive, less efficient and time-consuming methods such as chromatography, an affinity column, and the like, are used to separate nucleic acid molecules which are bound to a target material from nucleic acid molecules which are not bound to the target material. The GO-SELEX process is characterized in that the separation of binding ssDNA molecules from non-binding ssDNA molecules can be carried out simply by centrifugation even if the target material or the counter-target material is not specifically immobilized to a specific carrier (Nguyen et al., Chem. Commun. 2014, 50, 10513-10516; the contents of which are incorporated by reference herein in their entirety.)

After removing GO absorbed ssDNA molecules (e.g., by centrifugation), the target material may be removed from the collected DNA:target complexes. Methods for participating proteins in a solution well known in the art, for example, ethanol precipitation and strataclean resin may be used. As a non-limiting example, a strataclean resin may be added to the supernatant recovered after centrifugation. The target material bound to the strataclean resin can be removed by centrifugation. The target removal step may be repeated for two, three, four or more times. A supernatant containing the enriched ssDNA molecules that bind to the target material may be used for next target binding selection round (FIG. 1).

In some embodiments, the final concentration of ssDNA molecules that bind to the target material may be measured and compared to the initial concentration of the input ssDNA library. The ratio of the concentrations will be used to determine if another round of the GO-SELEX selection is needed. If the ratio is below 50%, another round of positive GO-SELEX process is carried out with the same condition. The same process is repeated until the recovery of ssDNA molecules that bind to the target material reaches to a satisfactory ratio, e.g., above 50% recovery. Rounds of partition and isolation are repeated until a desired goal is achieved, for example, two, three, four, five, six, seven, eight or more times with the same condition. In the most general case, selection is continued until no significant improvement in binding strength is achieved on repetition of the selection round.

The target binding pool of ssDNA molecules selected from the positive GO-SELEX process may be further amplified by performing a PCR using labeled primers, e.g., a biotinylated reverse primer and a fluorophore-labeled forward primer. In other aspects, the ssDNA molecules can be amplified by any other known method, such as sequencing the selected sequences and synthesizing them synthetically using an oligonucleotide synthesizer for the next round of binding and selection.

The fluorophore that is conjugated to the forward primer may be, but is not limited to, Cy5, Alexa Fluor 350, Alexa Fluor 430, Alexa Fluor 488, Alexa Fluor 532, Alexa Fluor 546, Alexa Fluor 594, Alexa Fluor 647, Alexa Fluor 658, Cyanine-3, Cyanine-5, fluorescein, Texas red, FITC (Fluorescein Isothiocyanate), rhodamine, or the like. In one preferred embodiment, the forward primer is conjugated with Cy5. In another embodiment, the forward primer is conjugated with Alexa Fluor 647.

After PCR amplification, the resulted double stranded DNA (dsDNA) molecules may be cleaned and further denatured to regenerate single stranded DNA (ssDNA) molecules. The biotinylated reverse primer allows for removal of the complementary strands to regenerate ssDNA molecules from the dsDNA molecules created during PCR amplification. As a non-limiting example, streptavidin coated magnetic beads may be added to the PCR product. The biotinylated complementary strands bind to the streptavidin coated magnetic beads. After denaturation of ssDNA molecules (e.g., addition of a base), the bound biotinylated complementary strands are separated and removed using a magnetic force. The desired ssDNA molecules with the fluorophore tags are collected. The fluorophore (e.g., Cy5 and Alexa Fluor 647) tagged ssDNA molecules that substantially bind to the target are used for next selection process.

The fluorophore (e.g., Cy5) tagged ssDNA molecules give several advantages in developing detection agents used in competition-based detection assays. The addition of Cy5 or other fluorescence markers to the ssDNA sequences at the beginning of aptamer selection can ensure that all aptamer candidates have proper secondary and tertiary structures when they are further developed as signaling polynucleotides (SPNs) used in detection assays. The addition of Cy5 or another fluorescence marker at the later stage may influence the secondary and tertiary structures of aptamer candidates. This modification can significantly reduce false hits during the selection.

As a non-limiting example, the GO-SELEX process to identify a pool of sequences that substantially bind to a target may comprise the steps of (i) mixing the input ssDNA library (e.g., Pool 0 in Table 1) with an allergen composition in a buffer solution; and these are induced to be bound to each other at normal temperature; (ii) adding a graphene oxide solution to the mixture of step (i) to remove ssDNA molecules which are not bound to the target; (iii) removing the target from the collected ssDNA molecules and amplifying the ssDNA molecules by performing a PCR using the PCR primers at the ends of the ssDNA molecules; and (iv) denaturing the double stranded PCT products and collecting ssDNA molecules labeled with fluorophore. Optionally, the positive GO-SELEX selection may be repeated for 2, 3, 4, 5, 6, 7, 8, 9, 10, or more rounds. The ssDNA molecules in the input library comprise approximately 76 nucleotides in length, including a primer for PCR amplification at each end and about 30 nucleotides (the binding site) at its center (i.e., the inner sequence of an aptamer). The target material is an allergen material, particularly a food allergen, comprising one allergenic component, or a mixture of allergenic components from a single allergen. Food allergens may include but are not limited to proteins in legumes such as peanuts, peas, lentils and beans, tree nuts (e.g., almond, brazil nut, cashew, hazelnut, pecan, pistachio and walnut), wheat, milk, fish, egg white and sea food.

In some embodiments, the 5′ constant sequence (i.e., the 5′ primer) comprises a nucleic acid sequence of SEQ ID NO. 1 and the 3′ constant sequence (i.e., the 3′ primer) comprises a nucleic acid sequence of SEQ ID NO. 2.

### On-Chip Target Binding and Competition Selections

The target binding pool of ssDNA molecules (e.g., Pool 1 in Table 1) may be further partitioned to select a subset of sequences that substantially bind to the target and compete with short oligonucleotides having sequences complementary to the ssDNA molecules. In some embodiments, this positive target binding selection may be performed using solid supports (e.g., glass or plastic chips) that are coated with short oligonucleotides comprising sequences complementary to the ssDNA molecules. By this process, families of nucleic acid sequences which can simultaneously bind to a target molecule and their complementary sequences may be subtracted from the pool. This additional positive selection process is tailored to differentiate ssDNA molecules that bind to the target and to the complementary sequences attached to a solid support (e.g., a glass or plastic chip).

The short oligonucleotide anchors may comprise sequences complementary to the constant sequences at the ends of the ssDNA molecules. The oligonucleotide anchors may comprise sequences complementary to either the 5′ end or 3′ end sequence of the aptamers. The complementary sequence contains about 5-25 nucleotides, or 5-18 nucleotides, or 6-20 nucleotides, or 8-20 nucleotides. For example, it may comprise 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides. In one preferred example, the complementary anchor sequence contains 5-15 nucleotides. The oligonucleotide anchor may be 100%, or 99%, or 98%, or 97%, or 96%, or 95%, or 94%, or 93%, or 92%, or 91%, or 90% complementary to the sequence of an ssDNA. The short complementary sequences are covalently linked to a solid support such as a glass chip, directly or through a linker.

The solid support on which the oligonucleotides are covalently attached may include, but is not limited to, a glass, a polymer support (e.g., see, U.S. Pat. No. 5,919,525), polyacrylamide gel, or plastic (e.g., a microwell plate), or a nylon membrane. The glass may be a polymer glass (e.g., acrylic glass, polycarbonate and polyethylene terephthalate), or a silicate glass (e.g., Pyrex glass, quartz and germanium-oxide glass), or a porous glass, etc., Polymers may include, but are not limited to, polyimide, photoresist, SU-8 negative photoresist, polydimethylsiloxane (PDMS), silicone elastomer PDMS and COC. In one preferred embodiment, the solid support is a glass chip.

Different technologies may be used for attaching the short complementary sequences to the solid support at determined sites. These methods are well-known in the pertinent art. For example, the oligonucleotides can be deposited on specific sites on the solid support as microdroplets by ink jet, or piezoelectric, or other similar methods. The solid support may be pre-treated to provide active attaching surfaces for oligonucleotides. In addition, the density of the attached oligonucleotides may be measured and controlled on the solid support.

In one embodiment, The on-chip target binding selection may comprise the steps: (i) mixing the target binding pool of ssDNA molecules (i.e., Pool 1) with the same target material in a buffer solution; and they are induced to be bound to each other at normal temperature; (ii) contacting the mixture of step (i) with a solid support of which the surface is covalently coated with short oligonucleotides comprising sequences complementary to the sequences of ssDNA molecules; (iii) collecting the ssDNA:target complexes that are not bound to the solid support (e.g., the flow-through) (FIG. 1); and (iv) removing the target material from the collected ssDNA:target complexes and collecting an enriched subset of ssDNA molecules. Optionally, the collected mixture in (iii) is again contacted with the solid support coated with the complementary oligonucleotides for two, three, four, five, six, seven, eight, or more times and the flow-through after the final incubation is processed to recover the ssDNA molecules from the complexes.

By this selection, a subset of ssDNA molecules in Pool 1 that are not bound to the target material will hybridize to the complimentary sequences covalently attached to the solid support and be removed from the pool. In addition, a subset of ssDNA molecules bound to the target material may also hybridize to the complimentary sequences attached to the solid support. These ssDNA molecules stay on the solid support and are subtracted from the collected ssDNA pool.

The collected ssDNA molecules from the final incubation are cleaned and separated from the target material as described herein. Similar to the positive GO-SELEX selection, the concentration of the recovered ssDNA molecules is measured and compared to the input pool (Pool 1). In some embodiments, the on-chip positive selection may be repeated for two, three, four, five, six, seven, eight or more rounds until the ratio of the recovered ssDNA molecules reaches to a desired recovery ratio (e.g., more than 50% from the input pool).

The ssDNA molecules are then amplified by performing PCR and single stranded DNA molecules are recovered as described in the positive GO-SELEX process. By this selection process, a pool of ssDNA molecules that bind to the target material but do not hybridize to the complementary sequences in the presence of the target material is selected (i.e. positive binding pool (Pool 2) in Table 1). The selection process mimics a condition used in a competition-based detection assay. The combination of regular SELEX (e.g., GO-SELEX) and on-chip positive target binding processes increases the specificity and affinity of aptamers.

### On-Chip Negative (Counter) Selections

The positive pool of ssDNA molecules (Pool 2) containing aptamer candidates that bind to the target material in the presence of the complementary sequences may be further screened to isolate ssDNA molecules that do not bind to the complementary sequences even when the sequences are free, and sequences that substantially bind to counter target materials in addition to the target of interest. In some embodiments, these non-specific ssDNA sequences may be isolated by counter selection processes using solid supports (e.g., glass chips) precoated with short oligonucleotides comprising sequences complementary to the ssDNA molecules.

In some embodiments, an on-chip non-binding counter selection is performed to identify ssDNA molecules that do not hybridize to their complementary sequences in the pool even when they are free. A DNA solution comprising the positive pool of ssDNA molecules (Pool 2), without addition of the target material, is directly incubated with a solid support (e.g., a glass chip) that is precoated with short oligonucleotides comprising sequences complementary to the aptamers in the pool. After incubation, the DNA solution including the unbound ssDNA molecules is collected (i.e. the flow-through) (FIG. 1). The flow-through DNA solution is incubated again with the solid support (e.g., a glass chip) that is precoated with short complementary oligonucleotides and a second flow-through is collected. The incubation step may be repeated two, three, four, five, six, seven, eight or more times, preferably eight times.

In one preferred embodiment, the on-chip non-binding counter process may comprise the steps: (i) preparing a DNA solution comprising the positive binding pool of ssDNA molecules (Pool 2); (ii) contacting the DNA solution with a solid support coated with short oligonucleotides comprising sequences complementary to the ssDNA molecules; (iii) collecting the ssDNA solution after incubation; and (iv) contacting the collected solution again with a new solid support coated with the complementary sequences. These steps may be repeated for two, three, four, five, six, seven or eight rounds and the collected ssDNA solution from the final incubation will be cleaned and amplified for sequencing. In one embodiment, these steps are repeated for eight rounds and the collected ssDNA solution from the final incubation are cleaned and amplified for sequencing.

This on-chip non-binding counter selection creates a non-binding pool of ssDNA molecules (i.e., Pool 3 in Table 1) including ssDNA molecules that cannot hybridize to the complementary sequences even in the absence of the target material.

In other embodiments, an on-chip counter binding selection is performed to isolate any sequences that can bind to non-specific counter targets from the positive binding pool of ssDNA molecules (Pool 2 in Table 1). This counter selection process improves the target specificity of selected ssDNA molecules by eliminating nucleic acid sequences with cross-reactivity to one or more non-target molecules (e.g., counter targets).

In one preferred embodiment, the on-chip counter selection process may comprise the steps of (i) preparing a ssDNA solution comprising the positive binding pool of ssDNA molecules (Pool 2) and incubating the ssDNA solution with a counter target or a mixture of counter targets; (ii) contacting the mixture of step (i) with a solid support that is coated with short oligonucleotides comprising sequences complementary to the ssDNA molecules; (iii) collecting the ssDNA/counter target complexes after step (ii) (i.e. the flow-through) (FIG. 1); and (iv) contacting the collected solution in step (iii) to a solid support that is coated with complementary oligonucleotides. The incubation and collection steps may be repeated for two, three, four, five, six, seven, eight or more rounds, preferably eight rounds. The collected solution after the final incubation step will be cleaned and/or amplified for sequencing.

In some embodiments, the on-chip counter binding selection may be repeated for as many counter targets as desired, beginning each time with the same pool of ssDNA molecules from the positive binding pool (i.e., Pool 2 in Table 1). In some alternative embodiments, multiple counter targets can be run within the same round in parallel.

By this on-chip counter selection process, ssDNA sequences with cross-specificity towards undesirable related proteins (counter target material) are removed from the positive binding pool. This counter selection process creates a pool of ssDNA molecules (i.e., Pool 4 in Table 1) including the ssDNA molecules that can cross react with a counter target or several counter targets.

As non-limiting examples, the counter targets may be allergen proteins in the same family, including allergen proteins from different sources that can be attributed to these structurally related allergen families, e.g., prolamins family including seed storage proteins (e.g., Sec c 20 in Rye; Tri a 19 in wheat and Tri a 36 in wheat), non-specific lipid transfer proteins family (e.g., Act d 10 in Kiwi, Api g 2 in celery, Ara h 9 in peanut, Cas s 8 in chestnut, Cor a 8 in hazelnut, Jug r 3 in walnut, Lyc e 3 in tomato, Mus a 3 in banana, and Pru du 3 in almond), 2S albumins family including seed storage proteins (e.g., Ana o 3 in cashew nut, Ara h 2 in peanut, Ber e 1 in Brazil nut, Fag e 2 in buckwheat, Gly m 8 in soybean, Jug r 1 in walnut, Ses i 1 in sesame, and Sin a 1 in mustard), Bet VI family including pathogenesis related proteins (e.g., Api g 1/celery, Ara h 8/peanut, Cor a 1/hazelnut, Dau c 1/carrot, Gly m 4/soybean, Mal d 1/apple, and Pru p 1/peach), 7S (vicilin-like) globulins family (e.g., Ana o 1/cashew nut; Ara h 1/peanut; Gly m 5/soybean; Jug r 2/walnut; Pis v 3/pistachio), 11S (legumin-like) globulins family (e.g., Ana o 2/cashew nut; Ara h 3/peanut; Ber e 2/Brazil nut; Cor a 9/hazelnut; Gly m 6/soybean; Jug r 4/walnut; Pru du 6/almond), Cysteine protease C1 family (e.g., Act d 1/kiwi; Gly m Bd 30K/soybean), Profilins family including actin binding proteins (e.g., Act d 9/kiwi; Api g 4/celery; Ara h 5/peanut; Cuc m 2/melon; Dau c 4/carrot; Gly m 3/soybean; Lyc e 1/tomato; Mus a 1/banana; Ory s 12/rice; Pru av 4/cherry; Pru du 4/almond; Pru p 4/peach and Tri a 12/wheat), tropomyosin family including actin binding proteins in muscle (e.g., Pen m 1/shrimp), parvalbumin family including muscle proteins (e.g., Cyp c 1/carp; Gad c 1/cod; Ran e 2/frog; Sal s 1/salmon; Seb m 1/redfish; Xip g 1/swordfish), caseins family including mammalian milk proteins (e.g., Bos d 8-Bos d 12/cow's milk), transferrin family including sulfur-rich ion-binding glycoproteins from milk and hen's egg white (e.g., Bos d Lactoferrin/cow's milk; Gal d 3/hen's egg), serpins family including Serine protease inhibitors (e.g., Gal d 2/hen's egg), Arginine kinases family including Adenosine triphosphate:guanido phosphotransferases (e.g., Pen m 2/shrimp), Lipocalins family including carrier proteins (e.g., Bos d 5/cow's milk), Ovomucoids family including Kazal inhibitors (e.g., Gal d 1/hen's egg), Lysozyme family (e.g., Bos d 4/cow's milk; Gal d 4/hen's egg), and Albumins family including Serum albumins (e.g., Bos d 6/cow's milk; Gal d 5/hen's egg).

### Deep Sequencing

In accordance with the present screening method, the ssDNA pools (e.g., Pool 1, Pool 2, Pool 3 and Pool 4 in Table 1) from each selection may be cleaned, amplified and sequenced. In one embodiment, the method comprises an amplification of the individual ssDNA molecules using a polymerase chain reaction (PCR). The sequences within each pool are identified using deep sequencing. In some embodiments, the ssDNA molecules in the target binding pool (i.e., Pool 1) are amplified and sequenced. In parallel, an artifact library may be made by amplifying the input ssDNA library and the sequences in this artifact library are sequenced (See, e.g., the flow-chart of FIG. 1). The artifact library may be made from repeating the PCR amplification and strand separation steps for the same number of rounds for the positive GO-SELEX selection (FIG. 1). These sequences, resulted from over amplification by PCR, are removed from the target binding pool (Pool 2 in Table 1).

The ssDNA molecules in the positive binding pool from the final round of the on-chip target binding selection (e.g., Pool 2 in Table 1) may be sequenced. The ssDNA molecules within this pool contain ssDNA sequences that preferentially bind to their target in the presence of their complementary sequences.

The non-specific ssDNA molecules from the final round of the on-chip non-binding counter selection and from the final round of the on-chip counter selection (e.g., Pools 3 and 4 in Table 1) may be sequenced. The ssDNA molecules within these pools contain ssDNA sequences that fail to hybridize to the complementary sequences even in the absence of the target material and sequences with cross-specificity to other counter targets.

The ssDNA sequences from each pool may be barcoded for identity. Following barcoding, ssDNA molecules from each pool may be pooled together and run deep sequencing in a single lane on the Illumina MiSeq System.

TABLE 1
Summary of Selection rounds

| DNA pool | Description | |
|---|---|---|
| Input library | A library of random synthesized single | Input |
| (Pool 0) | stranded DNA molecules comprising a central | sequences |
| | randomized region and two primer regions | |
| | at both ends. | |
| Target | A sub-pool of ssDNA molecules that positively | Sequenced |
| binding pool | bind to the target of interest selected by | |
| (Pool 1) | the GO-SELEX process | |
| Positive pool | A sub-pool of ssDNA molecules that | Sequenced |
| (Pool 2) | preferentially bind to the target of interest | |
| | to various short complementary sequences, | |
| | selected by the on-chip positive target | |
| | binding process | |
| Non-binder | A sub-pool of ssDNA molecules that do not | Sequenced |
| pool | bind to various short complementary sequences | |
| (Pool 3) | in the absence of the target selected from the | |
| | on-chip non-binding counter selection | |
| Counter | A sub-pool of ssDNA molecules that bind to | Sequenced |
| binding pool | various counter targets in addition to binding | |
| (Pool 4) | to the target of interest, selected by the on- | |
| | chip counter selection | |
| Aptamer | a final subset of ssDNA molecules after | selected |
| candidate | extracting the sequences in Pool 3 and Pool | |
| pool | 4 from Pool 2 | |
| (Pool 5) | | |

### Data Analysis and Bioinformatics

After sequencing and barcoding of the ssDNA sequences in each pool, and running the deep sequencing, the data are analyzed using any available bioinformatics tools. In some embodiments, heat maps are generated for each individual pool, which represent the frequencies of ssDNA sequences in each pool, by using a local occurrence of the open-source bioinformatics tool Galaxy (Thiel and Giangrande, Methods 2016, 97, 3-10; the contents of which are incorporated herein by reference in their entirety).

Potential aptamer hits are selected by analyzing the over-expressed sequences in each pool using the heat maps for sequences in each pool. Essentially, the heat maps of the ssDNA molecules from the non-binding pool (Pool 3) and the counter binding pool (Pool 4) are subtracted from the heat maps of the ssDNA molecules from the positive binding pool (Pool 2). The final data represent a pool of potential aptamer hits with characteristics including: (i) binding to the target protein with high specificity and affinity, (ii) hybridizing to their short complementary sequences only in the absence of the target but not binding to the short complementary sequences in the presence of the target; and (iii) no cross-reactivity to non-specific counter targets. These characteristics of the aptamer candidates make them suitable for target detection in a sample, e.g., in competition-based assays.

From the final pool of potential aptamer hits (e.g., Pool 5 in Table 1), a sequence family tree may be constructed to show the similarities between different aptamer sequences. Multiple sequences from various branches of the family tree structure can be selected and folded using expected assay conditions. The secondary and tertiary structures will be assessed, and those sequences that show multiple well-defined structures are selected for synthesis and further evaluation. The structures or motifs may include hairpin loops, symmetric and asymmetric bulges, pseudoknots and myriad combinations of the same. The equilibrium dissociation constant (K_{d}), and other parameters of the selected aptamers will be measured.

In accordance with the present disclosure, the selection method may further comprise the steps of (i) amplifying all the sequences in the first, second, third and fourth sub-pools, and barcoding each sequence from each pool; (ii) pooling the sequences from each sub-pool together and running sequencing together; (iii) analyzing the data from (ii) and separating each sequence data into the original sub-pool according to the barcode information; (iv) generating heat maps for each individual sub-pool that represent the frequencies of each sequence in the pool; and (v) subtracting the sequences in the heat maps of the third sub-pool and the fourth sub-pool from the heat maps of the second sub-pool, wherein the final pool of sequences are candidate aptamers that specifically bind to the target of interest and preferentially bind to the target of the interest in competing the binding of various short complementary sequences.

In accordance with the present disclosure, sequences that specifically bind to peanut, tree nuts including almond, brazil nut, cashew, hazelnut, pecan, pistachio and walnut, gluten, milk allergens whey and casein are selected. Aptamer sequences that bind to all nuts are also selected. As used here, the term “all nuts” refers to peanut and the tree nuts including almond, brazil nut, cashew, hazelnut, pecan, pistachio and walnut. A selected aptamer sequence that is specific to “all nuts” can bind to any of the nuts (i.e., peanut, almond, brazil nut, cashew, hazelnut, pecan, pistachio and walnut), e.g., one, two, three, four, five, six, seven or eight nuts present in samples.

In some embodiments, the sequences that specifically bind to peanut comprise an inner sequence selected from the group consisting of the nucleic acid sequences of SEQ ID NO. 3 to 1002.

In some embodiments, the sequences that specifically bind to almond comprise an inner sequence selected from the group consisting of the nucleic acid sequences of SEQ ID NO. 4003 to 5002.

In some embodiments, the sequences that specifically bind to brazil nut comprise an inner sequence selected from the group consisting of the nucleic acid sequences of SEQ ID NO. 8003 to 9002.

In some embodiments, the sequences that specifically bind to cashew comprise an inner sequence selected from the group consisting of the nucleic acid sequences of SEQ ID NO. 12003 to 13002.

In some embodiments, the sequences that specifically bind to hazelnut comprise an inner sequence selected from the group consisting of the nucleic acid sequences of SEQ ID NO. 16003 to 17002.

In some embodiments, the sequences that specifically bind to pecan comprise an inner sequence selected from the group consisting of the nucleic acid sequences of SEQ ID NO. 20003 to 21002.

In some embodiments, the sequences that specifically bind to pistachio comprise an inner sequence selected from the group consisting of the nucleic acid sequences of SEQ ID NO. 24003 to 25002.

In some embodiments, the sequences that specifically bind to walnut comprise an inner sequence selected from the group consisting of the nucleic acid sequences of SEQ ID NO. 28003 to 29002.

In some embodiments, the sequences that specifically bind to all nuts comprise an inner sequence selected from the group consisting of the nucleic acid sequences of SEQ ID NO. 32003 to 33002.

In some embodiments, the sequences that specifically bind to gluten comprise an inner sequence selected from the group consisting of the nucleic acid sequences of SEQ ID NO. 40003 to 41002.

In some embodiments, the sequences that specifically bind to whey comprise an inner sequence selected from the group consisting of the nucleic acid sequences of SEQ ID NO. 44003 to 45002.

In some embodiments, the sequences that specifically bind to casein comprise an inner sequence selected from the group consisting of the nucleic acid sequences of SEQ ID NO. 48003 to 49002.

### Multiple SELEX Selections

In some embodiments, the present selection method may be modified to identify aptamer sequences that bind to multiple targets. The multiple target selection process provides an efficient method for identifying the best binding aptamers to a group of targets.

Many allergens, particularly food allergens, are composed of multiple allergenic components. These components may induce component specific IgE in a person's body. Some people are allergic to only one specific component but not the other components of the same allergen. Some people are allergic to all the components of an allergen. For example, milk includes two primary allergenic components: the whey proteins (alpha-lactalbumin and beta-lactoglobulin) and caseins. A person who is allergic to milk, may be allergic to only whey or casein, or to both whey and casein. In this context, an aptamer ligand that binds to the whey proteins only, or caseins only, or both the whey proteins and caseins, may be desirable to milk allergy.

In some embodiments, the present screening methods may be modified for selecting aptamers that can bind to the multiple components of an allergen.

As a non-limiting example, two, three, four or more rounds of the positive GO-SELEX selection are performed using the whole allergen material as the target material (e.g., the whole milk including casein and the whey proteins). The ssDNA molecules selected from this process (Pool 1) include a mixture of ssDNA sequences that bind to any of the various components of milk (e.g., caseins and the whey proteins). These milk binding sequences are used to run two parallel selection processes: a selection process for casein only and a selection process for the whey protein only. The two selection processes are performed as previously described for a single target. Importantly, during the counter selection process, a separate selection will be performed using only the other component as the counter target. That is, in the selection for the aptamer sequences that specifically bind to casein, a whey protein is used as the counter target in the counter selection process, while casein is used as the counter target for selecting aptamer sequences that specifically bind to a whey protein.

Following similar procedures, the various sub-pools of ssDNA molecules will be barcoded and submitted for deep sequencing. The casein and whey samples will be pooled separately from one another and run in separate lanes during deep sequencing. The bioinformatic analysis of the sequencing data will reveal the pool of aptamer hits for the target alone. In order to find an aptamer sequence that binds both casein and whey, overlaps between the special counter rounds may be collected.

In another example, a mixture of all nuts may be used as target materials, sequences that can recognize all nuts may be selected by the present methods. The selected sequences may bind to any nut, and the combinations of any nuts present in a test sample.

### Aptamers, Signaling Polynucleotides (SPNs) and Detection Sensors

In another aspect of the disclosure, aptamers that specifically bind to allergen targets, signaling polynucleotides (SPNs) derived from the selected aptamers, and detection sensors comprising these aptamers and SPNs are provided. An aptamer that binds to a target allergen with high specificity and affinity may not hybridize to the short complementary sequence in the presence of the target allergen, and demonstrates little or no cross-specificity to any counter target.

A SPN may be derived from an aptamer sequence selected by the present method. The SPN may further comprise additional nucleotides at one end or both ends of the aptamer sequence. The sequence may be further modified to change its secondary and/or tertiary structures to make it more stable, to increase the binding affinity and/or specificity, or to add a fluorescence marker, or to be modified to comprise one or more conjugates.

Detection sensors comprising selected aptamers and SPNs are provided. In some embodiments, the detection sensor may include a SPN, a solid support and a short oligonucleotide comprising a nucleic acid sequence complementary to the SPN, wherein the oligonucleotide is covalently anchored to the solid support by one of the ends, directly or through a linker (e.g., a 6 carbon atom arm). The SPN comprises an inner sequence that specifically binds to a target of interest and it hybridizes to the complementary oligonucleotide when it is not bound to the target of interest. In one example, the short complementary sequences and the target of interest will compete binding to the SPN. In this competitive assay, for example, the SPN can either bind to the short complementary sequences attached on the solid support or a target of interest in a sample. Under conditions sufficient to allow the target of interest in the sample to compete with the short complementary sequences attached on the solid support, the SPN:target complexes can be detected and measured.

In accordance with the present disclosure, aptamer sequences that specifically bind to peanut, tree nuts including almond, brazil nut, cashew, hazelnut, pecan, pistachio and walnut, gluten, milk allergens whey and casein are selected. Aptamer sequences that bind to all nuts are also selected.

In some embodiments, the sequences that specifically bind to peanut comprise an inner sequence selected from the group consisting of the nucleic acid sequences of SEQ ID NOs.3 to 1002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO.1. Accordingly, the aptamer that specifically binds to peanut may comprise a nucleic acid sequence selected from the group consisting of SEQ ID NOs.1003 to 2002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO.2. Accordingly, the aptamer that specifically binds to peanut may comprise a nucleic acid sequence selected from the group consisting of SEQ ID NOs. 2003 to 3002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to peanut may comprise a nucleic acid sequence selected from the group consisting of SEQ ID NOs. 3003 to 4002. In one embodiment, the aptamer of the present disclosure that specifically binds to peanut may comprise a nucleic acid sequence selected from the group consisting of SEQ ID NOs. 3 to 4002 listed in Table 2, or variant thereof.

TABLE 2
Aptamer sequences against peanut
Aptamer sequence

| | | | 5′ sequence |
|---|---|---|---|
| | 5′-sequence | Inner | and inner |
| Inner | and inner | sequence and | sequence and |
| sequence | sequence | 3′ sequence | 3′ sequence |
| (SEQ ID NO.) | (SEQ ID NO.) | (SEQ ID NO.) | (SEQ ID NO.) |
| 3 | 1003 | 2003 | 3003 |
| 4 | 1004 | 2004 | 3004 |
| 5 | 1005 | 2005 | 3005 |
| 6 | 1006 | 2006 | 3006 |
| 7 | 1007 | 2007 | 3007 |
| 8 | 1008 | 2008 | 3008 |
| 9 | 1009 | 2009 | 3009 |
| 10 | 1010 | 2010 | 3010 |
| 11 | 1011 | 2011 | 3011 |
| 12 | 1012 | 2012 | 3012 |
| 13 | 1013 | 2013 | 3013 |
| 14 | 1014 | 2014 | 3014 |
| 15 | 1015 | 2015 | 3015 |
| 16 | 1016 | 2016 | 3016 |
| 17 | 1017 | 2017 | 3017 |
| 18 | 1018 | 2018 | 3018 |
| 19 | 1019 | 2019 | 3019 |
| 20 | 1020 | 2020 | 3020 |
| 21 | 1021 | 2021 | 3021 |
| 22 | 1022 | 2022 | 3022 |
| 23 | 1023 | 2023 | 3023 |
| 24 | 1024 | 2024 | 3024 |
| 25 | 1025 | 2025 | 3025 |
| 26 | 1026 | 2026 | 3026 |
| 27 | 1027 | 2027 | 3027 |
| 28 | 1028 | 2028 | 3028 |
| 29 | 1029 | 2029 | 3029 |
| 30 | 1030 | 2030 | 3030 |
| 31 | 1031 | 2031 | 3031 |
| 32 | 1032 | 2032 | 3032 |
| 33 | 1033 | 2033 | 3033 |
| 34 | 1034 | 2034 | 3034 |
| 35 | 1035 | 2035 | 3035 |
| 36 | 1036 | 2036 | 3036 |
| 37 | 1037 | 2037 | 3037 |
| 38 | 1038 | 2038 | 3038 |
| 39 | 1039 | 2039 | 3039 |
| 40 | 1040 | 2040 | 3040 |
| 41 | 1041 | 2041 | 3041 |
| 42 | 1042 | 2042 | 3042 |
| 43 | 1043 | 2043 | 3043 |
| 44 | 1044 | 2044 | 3044 |
| 45 | 1045 | 2045 | 3045 |
| 46 | 1046 | 2046 | 3046 |
| 47 | 1047 | 2047 | 3047 |
| 48 | 1048 | 2048 | 3048 |
| 49 | 1049 | 2049 | 3049 |
| 50 | 1050 | 2050 | 3050 |
| 51 | 1051 | 2051 | 3051 |
| 52 | 1052 | 2052 | 3052 |
| 53 | 1053 | 2053 | 3053 |
| 54 | 1054 | 2054 | 3054 |
| 55 | 1055 | 2055 | 3055 |
| 56 | 1056 | 2056 | 3056 |
| 57 | 1057 | 2057 | 3057 |
| 58 | 1058 | 2058 | 3058 |
| 59 | 1059 | 2059 | 3059 |
| 60 | 1060 | 2060 | 3060 |
| 61 | 1061 | 2061 | 3061 |
| 62 | 1062 | 2062 | 3062 |
| 63 | 1063 | 2063 | 3063 |
| 64 | 1064 | 2064 | 3064 |
| 65 | 1065 | 2065 | 3065 |
| 66 | 1066 | 2066 | 3066 |
| 67 | 1067 | 2067 | 3067 |
| 68 | 1068 | 2068 | 3068 |
| 69 | 1069 | 2069 | 3069 |
| 70 | 1070 | 2070 | 3070 |
| 71 | 1071 | 2071 | 3071 |
| 72 | 1072 | 2072 | 3072 |
| 73 | 1073 | 2073 | 3073 |
| 74 | 1074 | 2074 | 3074 |
| 75 | 1075 | 2075 | 3075 |
| 76 | 1076 | 2076 | 3076 |
| 77 | 1077 | 2077 | 3077 |
| 78 | 1078 | 2078 | 3078 |
| 79 | 1079 | 2079 | 3079 |
| 80 | 1080 | 2080 | 3080 |
| 81 | 1081 | 2081 | 3081 |
| 82 | 1082 | 2082 | 3082 |
| 83 | 1083 | 2083 | 3083 |
| 84 | 1084 | 2084 | 3084 |
| 85 | 1085 | 2085 | 3085 |
| 86 | 1086 | 2086 | 3086 |
| 87 | 1087 | 2087 | 3087 |
| 88 | 1088 | 2088 | 3088 |
| 89 | 1089 | 2089 | 3089 |
| 90 | 1090 | 2090 | 3090 |
| 91 | 1091 | 2091 | 3091 |
| 92 | 1092 | 2092 | 3092 |
| 93 | 1093 | 2093 | 3093 |
| 94 | 1094 | 2094 | 3094 |
| 95 | 1095 | 2095 | 3095 |
| 96 | 1096 | 2096 | 3096 |
| 97 | 1097 | 2097 | 3097 |
| 98 | 1098 | 2098 | 3098 |
| 99 | 1099 | 2099 | 3099 |
| 100 | 1100 | 2100 | 3100 |
| 101 | 1101 | 2101 | 3101 |
| 102 | 1102 | 2102 | 3102 |
| 103 | 1103 | 2103 | 3103 |
| 104 | 1104 | 2104 | 3104 |
| 105 | 1105 | 2105 | 3105 |
| 106 | 1106 | 2106 | 3106 |
| 107 | 1107 | 2107 | 3107 |
| 108 | 1108 | 2108 | 3108 |
| 109 | 1109 | 2109 | 3109 |
| 110 | 1110 | 2110 | 3110 |
| 111 | 1111 | 2111 | 3111 |
| 112 | 1112 | 2112 | 3112 |
| 113 | 1113 | 2113 | 3113 |
| 114 | 1114 | 2114 | 3114 |
| 115 | 1115 | 2115 | 3115 |
| 116 | 1116 | 2116 | 3116 |
| 117 | 1117 | 2117 | 3117 |
| 118 | 1118 | 2118 | 3118 |
| 119 | 1119 | 2119 | 3119 |
| 120 | 1120 | 2120 | 3120 |
| 121 | 1121 | 2121 | 3121 |
| 122 | 1122 | 2122 | 3122 |
| 123 | 1123 | 2123 | 3123 |
| 124 | 1124 | 2124 | 3124 |
| 125 | 1125 | 2125 | 3125 |
| 126 | 1126 | 2126 | 3126 |
| 127 | 1127 | 2127 | 3127 |
| 128 | 1128 | 2128 | 3128 |
| 129 | 1129 | 2129 | 3129 |
| 130 | 1130 | 2130 | 3130 |
| 131 | 1131 | 2131 | 3131 |
| 132 | 1132 | 2132 | 3132 |
| 133 | 1133 | 2133 | 3133 |
| 134 | 1134 | 2134 | 3134 |
| 135 | 1135 | 2135 | 3135 |
| 136 | 1136 | 2136 | 3136 |
| 137 | 1137 | 2137 | 3137 |
| 138 | 1138 | 2138 | 3138 |
| 139 | 1139 | 2139 | 3139 |
| 140 | 1140 | 2140 | 3140 |
| 141 | 1141 | 2141 | 3141 |
| 142 | 1142 | 2142 | 3142 |
| 143 | 1143 | 2143 | 3143 |
| 144 | 1144 | 2144 | 3144 |
| 145 | 1145 | 2145 | 3145 |
| 146 | 1146 | 2146 | 3146 |
| 147 | 1147 | 2147 | 3147 |
| 148 | 1148 | 2148 | 3148 |
| 149 | 1149 | 2149 | 3149 |
| 150 | 1150 | 2150 | 3150 |
| 151 | 1151 | 2151 | 3151 |
| 152 | 1152 | 2152 | 3152 |
| 153 | 1153 | 2153 | 3153 |
| 154 | 1154 | 2154 | 3154 |
| 155 | 1155 | 2155 | 3155 |
| 156 | 1156 | 2156 | 3156 |
| 157 | 1157 | 2157 | 3157 |
| 158 | 1158 | 2158 | 3158 |
| 159 | 1159 | 2159 | 3159 |
| 160 | 1160 | 2160 | 3160 |
| 161 | 1161 | 2161 | 3161 |
| 162 | 1162 | 2162 | 3162 |
| 163 | 1163 | 2163 | 3163 |
| 164 | 1164 | 2164 | 3164 |
| 165 | 1165 | 2165 | 3165 |
| 166 | 1166 | 2166 | 3166 |
| 167 | 1167 | 2167 | 3167 |
| 168 | 1168 | 2168 | 3168 |
| 169 | 1169 | 2169 | 3169 |
| 170 | 1170 | 2170 | 3170 |
| 171 | 1171 | 2171 | 3171 |
| 172 | 1172 | 2172 | 3172 |
| 173 | 1173 | 2173 | 3173 |
| 174 | 1174 | 2174 | 3174 |
| 175 | 1175 | 2175 | 3175 |
| 176 | 1176 | 2176 | 3176 |
| 177 | 1177 | 2177 | 3177 |
| 178 | 1178 | 2178 | 3178 |
| 179 | 1179 | 2179 | 3179 |
| 180 | 1180 | 2180 | 3180 |
| 181 | 1181 | 2181 | 3181 |
| 182 | 1182 | 2182 | 3182 |
| 183 | 1183 | 2183 | 3183 |
| 184 | 1184 | 2184 | 3184 |
| 185 | 1185 | 2185 | 3185 |
| 186 | 1186 | 2186 | 3186 |
| 187 | 1187 | 2187 | 3187 |
| 188 | 1188 | 2188 | 3188 |
| 189 | 1189 | 2189 | 3189 |
| 190 | 1190 | 2190 | 3190 |
| 191 | 1191 | 2191 | 3191 |
| 192 | 1192 | 2192 | 3192 |
| 193 | 1193 | 2193 | 3193 |
| 194 | 1194 | 2194 | 3194 |
| 195 | 1195 | 2195 | 3195 |
| 196 | 1196 | 2196 | 3196 |
| 197 | 1197 | 2197 | 3197 |
| 198 | 1198 | 2198 | 3198 |
| 199 | 1199 | 2199 | 3199 |
| 200 | 1200 | 2200 | 3200 |
| 201 | 1201 | 2201 | 3201 |
| 202 | 1202 | 2202 | 3202 |
| 203 | 1203 | 2203 | 3203 |
| 204 | 1204 | 2204 | 3204 |
| 205 | 1205 | 2205 | 3205 |
| 206 | 1206 | 2206 | 3206 |
| 207 | 1207 | 2207 | 3207 |
| 208 | 1208 | 2208 | 3208 |
| 209 | 1209 | 2209 | 3209 |
| 210 | 1210 | 2210 | 3210 |
| 211 | 1211 | 2211 | 3211 |
| 212 | 1212 | 2212 | 3212 |
| 213 | 1213 | 2213 | 3213 |
| 214 | 1214 | 2214 | 3214 |
| 215 | 1215 | 2215 | 3215 |
| 216 | 1216 | 2216 | 3216 |
| 217 | 1217 | 2217 | 3217 |
| 218 | 1218 | 2218 | 3218 |
| 219 | 1219 | 2219 | 3219 |
| 220 | 1220 | 2220 | 3220 |
| 221 | 1221 | 2221 | 3221 |
| 222 | 1222 | 2222 | 3222 |
| 223 | 1223 | 2223 | 3223 |
| 224 | 1224 | 2224 | 3224 |
| 225 | 1225 | 2225 | 3225 |
| 226 | 1226 | 2226 | 3226 |
| 227 | 1227 | 2227 | 3227 |
| 228 | 1228 | 2228 | 3228 |
| 229 | 1229 | 2229 | 3229 |
| 230 | 1230 | 2230 | 3230 |
| 231 | 1231 | 2231 | 3231 |
| 232 | 1232 | 2232 | 3232 |
| 233 | 1233 | 2233 | 3233 |
| 234 | 1234 | 2234 | 3234 |
| 235 | 1235 | 2235 | 3235 |
| 236 | 1236 | 2236 | 3236 |
| 237 | 1237 | 2237 | 3237 |
| 238 | 1238 | 2238 | 3238 |
| 239 | 1239 | 2239 | 3239 |
| 240 | 1240 | 2240 | 3240 |
| 241 | 1241 | 2241 | 3241 |
| 242 | 1242 | 2242 | 3242 |
| 243 | 1243 | 2243 | 3243 |
| 244 | 1244 | 2244 | 3244 |
| 245 | 1245 | 2245 | 3245 |
| 246 | 1246 | 2246 | 3246 |
| 247 | 1247 | 2247 | 3247 |
| 248 | 1248 | 2248 | 3248 |
| 249 | 1249 | 2249 | 3249 |
| 250 | 1250 | 2250 | 3250 |
| 251 | 1251 | 2251 | 3251 |
| 252 | 1252 | 2252 | 3252 |
| 253 | 1253 | 2253 | 3253 |
| 254 | 1254 | 2254 | 3254 |
| 255 | 1255 | 2255 | 3255 |
| 256 | 1256 | 2256 | 3256 |
| 257 | 1257 | 2257 | 3257 |
| 258 | 1258 | 2258 | 3258 |
| 259 | 1259 | 2259 | 3259 |
| 260 | 1260 | 2260 | 3260 |
| 261 | 1261 | 2261 | 3261 |
| 262 | 1262 | 2262 | 3262 |
| 263 | 1263 | 2263 | 3263 |
| 264 | 1264 | 2264 | 3264 |
| 265 | 1265 | 2265 | 3265 |
| 266 | 1266 | 2266 | 3266 |
| 267 | 1267 | 2267 | 3267 |
| 268 | 1268 | 2268 | 3268 |
| 269 | 1269 | 2269 | 3269 |
| 270 | 1270 | 2270 | 3270 |
| 271 | 1271 | 2271 | 3271 |
| 272 | 1272 | 2272 | 3272 |
| 273 | 1273 | 2273 | 3273 |
| 274 | 1274 | 2274 | 3274 |
| 275 | 1275 | 2275 | 3275 |
| 276 | 1276 | 2276 | 3276 |
| 277 | 1277 | 2277 | 3277 |
| 278 | 1278 | 2278 | 3278 |
| 279 | 1279 | 2279 | 3279 |
| 280 | 1280 | 2280 | 3280 |
| 281 | 1281 | 2281 | 3281 |
| 282 | 1282 | 2282 | 3282 |
| 283 | 1283 | 2283 | 3283 |
| 284 | 1284 | 2284 | 3284 |
| 285 | 1285 | 2285 | 3285 |
| 286 | 1286 | 2286 | 3286 |
| 287 | 1287 | 2287 | 3287 |
| 288 | 1288 | 2288 | 3288 |
| 289 | 1289 | 2289 | 3289 |
| 290 | 1290 | 2290 | 3290 |
| 291 | 1291 | 2291 | 3291 |
| 292 | 1292 | 2292 | 3292 |
| 293 | 1293 | 2293 | 3293 |
| 294 | 1294 | 2294 | 3294 |
| 295 | 1295 | 2295 | 3295 |
| 296 | 1296 | 2296 | 3296 |
| 297 | 1297 | 2297 | 3297 |
| 298 | 1298 | 2298 | 3298 |
| 299 | 1299 | 2299 | 3299 |
| 300 | 1300 | 2300 | 3300 |
| 301 | 1301 | 2301 | 3301 |
| 302 | 1302 | 2302 | 3302 |
| 303 | 1303 | 2303 | 3303 |
| 304 | 1304 | 2304 | 3304 |
| 305 | 1305 | 2305 | 3305 |
| 306 | 1306 | 2306 | 3306 |
| 307 | 1307 | 2307 | 3307 |
| 308 | 1308 | 2308 | 3308 |
| 309 | 1309 | 2309 | 3309 |
| 310 | 1310 | 2310 | 3310 |
| 311 | 1311 | 2311 | 3311 |
| 312 | 1312 | 2312 | 3312 |
| 313 | 1313 | 2313 | 3313 |
| 314 | 1314 | 2314 | 3314 |
| 315 | 1315 | 2315 | 3315 |
| 316 | 1316 | 2316 | 3316 |
| 317 | 1317 | 2317 | 3317 |
| 318 | 1318 | 2318 | 3318 |
| 319 | 1319 | 2319 | 3319 |
| 320 | 1320 | 2320 | 3320 |
| 321 | 1321 | 2321 | 3321 |
| 322 | 1322 | 2322 | 3322 |
| 323 | 1323 | 2323 | 3323 |
| 324 | 1324 | 2324 | 3324 |
| 325 | 1325 | 2325 | 3325 |
| 326 | 1326 | 2326 | 3326 |
| 327 | 1327 | 2327 | 3327 |
| 328 | 1328 | 2328 | 3328 |
| 329 | 1329 | 2329 | 3329 |
| 330 | 1330 | 2330 | 3330 |
| 331 | 1331 | 2331 | 3331 |
| 332 | 1332 | 2332 | 3332 |
| 333 | 1333 | 2333 | 3333 |
| 334 | 1334 | 2334 | 3334 |
| 335 | 1335 | 2335 | 3335 |
| 336 | 1336 | 2336 | 3336 |
| 337 | 1337 | 2337 | 3337 |
| 338 | 1338 | 2338 | 3338 |
| 339 | 1339 | 2339 | 3339 |
| 340 | 1340 | 2340 | 3340 |
| 341 | 1341 | 2341 | 3341 |
| 342 | 1342 | 2342 | 3342 |
| 343 | 1343 | 2343 | 3343 |
| 344 | 1344 | 2344 | 3344 |
| 345 | 1345 | 2345 | 3345 |
| 346 | 1346 | 2346 | 3346 |
| 347 | 1347 | 2347 | 3347 |
| 348 | 1348 | 2348 | 3348 |
| 349 | 1349 | 2349 | 3349 |
| 350 | 1350 | 2350 | 3350 |
| 351 | 1351 | 2351 | 3351 |
| 352 | 1352 | 2352 | 3352 |
| 353 | 1353 | 2353 | 3353 |
| 354 | 1354 | 2354 | 3354 |
| 355 | 1355 | 2355 | 3355 |
| 356 | 1356 | 2356 | 3356 |
| 357 | 1357 | 2357 | 3357 |
| 358 | 1358 | 2358 | 3358 |
| 359 | 1359 | 2359 | 3359 |
| 360 | 1360 | 2360 | 3360 |
| 361 | 1361 | 2361 | 3361 |
| 362 | 1362 | 2362 | 3362 |
| 363 | 1363 | 2363 | 3363 |
| 364 | 1364 | 2364 | 3364 |
| 365 | 1365 | 2365 | 3365 |
| 366 | 1366 | 2366 | 3366 |
| 367 | 1367 | 2367 | 3367 |
| 368 | 1368 | 2368 | 3368 |
| 369 | 1369 | 2369 | 3369 |
| 370 | 1370 | 2370 | 3370 |
| 371 | 1371 | 2371 | 3371 |
| 372 | 1372 | 2372 | 3372 |
| 373 | 1373 | 2373 | 3373 |
| 374 | 1374 | 2374 | 3374 |
| 375 | 1375 | 2375 | 3375 |
| 376 | 1376 | 2376 | 3376 |
| 377 | 1377 | 2377 | 3377 |
| 378 | 1378 | 2378 | 3378 |
| 379 | 1379 | 2379 | 3379 |
| 380 | 1380 | 2380 | 3380 |
| 381 | 1381 | 2381 | 3381 |
| 382 | 1382 | 2382 | 3382 |
| 383 | 1383 | 2383 | 3383 |
| 384 | 1384 | 2384 | 3384 |
| 385 | 1385 | 2385 | 3385 |
| 386 | 1386 | 2386 | 3386 |
| 387 | 1387 | 2387 | 3387 |
| 388 | 1388 | 2388 | 3388 |
| 389 | 1389 | 2389 | 3389 |
| 390 | 1390 | 2390 | 3390 |
| 391 | 1391 | 2391 | 3391 |
| 392 | 1392 | 2392 | 3392 |
| 393 | 1393 | 2393 | 3393 |
| 394 | 1394 | 2394 | 3394 |
| 395 | 1395 | 2395 | 3395 |
| 396 | 1396 | 2396 | 3396 |
| 397 | 1397 | 2397 | 3397 |
| 398 | 1398 | 2398 | 3398 |
| 399 | 1399 | 2399 | 3399 |
| 400 | 1400 | 2400 | 3400 |
| 401 | 1401 | 2401 | 3401 |
| 402 | 1402 | 2402 | 3402 |
| 403 | 1403 | 2403 | 3403 |
| 404 | 1404 | 2404 | 3404 |
| 405 | 1405 | 2405 | 3405 |
| 406 | 1406 | 2406 | 3406 |
| 407 | 1407 | 2407 | 3407 |
| 408 | 1408 | 2408 | 3408 |
| 409 | 1409 | 2409 | 3409 |
| 410 | 1410 | 2410 | 3410 |
| 411 | 1411 | 2411 | 3411 |
| 412 | 1412 | 2412 | 3412 |
| 413 | 1413 | 2413 | 3413 |
| 414 | 1414 | 2414 | 3414 |
| 415 | 1415 | 2415 | 3415 |
| 416 | 1416 | 2416 | 3416 |
| 417 | 1417 | 2417 | 3417 |
| 418 | 1418 | 2418 | 3418 |
| 419 | 1419 | 2419 | 3419 |
| 420 | 1420 | 2420 | 3420 |
| 421 | 1421 | 2421 | 3421 |
| 422 | 1422 | 2422 | 3422 |
| 423 | 1423 | 2423 | 3423 |
| 424 | 1424 | 2424 | 3424 |
| 425 | 1425 | 2425 | 3425 |
| 426 | 1426 | 2426 | 3426 |
| 427 | 1427 | 2427 | 3427 |
| 428 | 1428 | 2428 | 3428 |
| 429 | 1429 | 2429 | 3429 |
| 430 | 1430 | 2430 | 3430 |
| 431 | 1431 | 2431 | 3431 |
| 432 | 1432 | 2432 | 3432 |
| 433 | 1433 | 2433 | 3433 |
| 434 | 1434 | 2434 | 3434 |
| 435 | 1435 | 2435 | 3435 |
| 436 | 1436 | 2436 | 3436 |
| 437 | 1437 | 2437 | 3437 |
| 438 | 1438 | 2438 | 3438 |
| 439 | 1439 | 2439 | 3439 |
| 440 | 1440 | 2440 | 3440 |
| 441 | 1441 | 2441 | 3441 |
| 442 | 1442 | 2442 | 3442 |
| 443 | 1443 | 2443 | 3443 |
| 444 | 1444 | 2444 | 3444 |
| 445 | 1445 | 2445 | 3445 |
| 446 | 1446 | 2446 | 3446 |
| 447 | 1447 | 2447 | 3447 |
| 448 | 1448 | 2448 | 3448 |
| 449 | 1449 | 2449 | 3449 |
| 450 | 1450 | 2450 | 3450 |
| 451 | 1451 | 2451 | 3451 |
| 452 | 1452 | 2452 | 3452 |
| 453 | 1453 | 2453 | 3453 |
| 454 | 1454 | 2454 | 3454 |
| 455 | 1455 | 2455 | 3455 |
| 456 | 1456 | 2456 | 3456 |
| 457 | 1457 | 2457 | 3457 |
| 458 | 1458 | 2458 | 3458 |
| 459 | 1459 | 2459 | 3459 |
| 460 | 1460 | 2460 | 3460 |
| 461 | 1461 | 2461 | 3461 |
| 462 | 1462 | 2462 | 3462 |
| 463 | 1463 | 2463 | 3463 |
| 464 | 1464 | 2464 | 3464 |
| 465 | 1465 | 2465 | 3465 |
| 466 | 1466 | 2466 | 3466 |
| 467 | 1467 | 2467 | 3467 |
| 468 | 1468 | 2468 | 3468 |
| 469 | 1469 | 2469 | 3469 |
| 470 | 1470 | 2470 | 3470 |
| 471 | 1471 | 2471 | 3471 |
| 472 | 1472 | 2472 | 3472 |
| 473 | 1473 | 2473 | 3473 |
| 474 | 1474 | 2474 | 3474 |
| 475 | 1475 | 2475 | 3475 |
| 476 | 1476 | 2476 | 3476 |
| 477 | 1477 | 2477 | 3477 |
| 478 | 1478 | 2478 | 3478 |
| 479 | 1479 | 2479 | 3479 |
| 480 | 1480 | 2480 | 3480 |
| 481 | 1481 | 2481 | 3481 |
| 482 | 1482 | 2482 | 3482 |
| 483 | 1483 | 2483 | 3483 |
| 484 | 1484 | 2484 | 3484 |
| 485 | 1485 | 2485 | 3485 |
| 486 | 1486 | 2486 | 3486 |
| 487 | 1487 | 2487 | 3487 |
| 488 | 1488 | 2488 | 3488 |
| 489 | 1489 | 2489 | 3489 |
| 490 | 1490 | 2490 | 3490 |
| 491 | 1491 | 2491 | 3491 |
| 492 | 1492 | 2492 | 3492 |
| 493 | 1493 | 2493 | 3493 |
| 494 | 1494 | 2494 | 3494 |
| 495 | 1495 | 2495 | 3495 |
| 496 | 1496 | 2496 | 3496 |
| 497 | 1497 | 2497 | 3497 |
| 498 | 1498 | 2498 | 3498 |
| 499 | 1499 | 2499 | 3499 |
| 500 | 1500 | 2500 | 3500 |
| 501 | 1501 | 2501 | 3501 |
| 502 | 1502 | 2502 | 3502 |
| 503 | 1503 | 2503 | 3503 |
| 504 | 1504 | 2504 | 3504 |
| 505 | 1505 | 2505 | 3505 |
| 506 | 1506 | 2506 | 3506 |
| 507 | 1507 | 2507 | 3507 |
| 508 | 1508 | 2508 | 3508 |
| 509 | 1509 | 2509 | 3509 |
| 510 | 1510 | 2510 | 3510 |
| 511 | 1511 | 2511 | 3511 |
| 512 | 1512 | 2512 | 3512 |
| 513 | 1513 | 2513 | 3513 |
| 514 | 1514 | 2514 | 3514 |
| 515 | 1515 | 2515 | 3515 |
| 516 | 1516 | 2516 | 3516 |
| 517 | 1517 | 2517 | 3517 |
| 518 | 1518 | 2518 | 3518 |
| 519 | 1519 | 2519 | 3519 |
| 520 | 1520 | 2520 | 3520 |
| 521 | 1521 | 2521 | 3521 |
| 522 | 1522 | 2522 | 3522 |
| 523 | 1523 | 2523 | 3523 |
| 524 | 1524 | 2524 | 3524 |
| 525 | 1525 | 2525 | 3525 |
| 526 | 1526 | 2526 | 3526 |
| 527 | 1527 | 2527 | 3527 |
| 528 | 1528 | 2528 | 3528 |
| 529 | 1529 | 2529 | 3529 |
| 530 | 1530 | 2530 | 3530 |
| 531 | 1531 | 2531 | 3531 |
| 532 | 1532 | 2532 | 3532 |
| 533 | 1533 | 2533 | 3533 |
| 534 | 1534 | 2534 | 3534 |
| 535 | 1535 | 2535 | 3535 |
| 536 | 1536 | 2536 | 3536 |
| 537 | 1537 | 2537 | 3537 |
| 538 | 1538 | 2538 | 3538 |
| 539 | 1539 | 2539 | 3539 |
| 540 | 1540 | 2540 | 3540 |
| 541 | 1541 | 2541 | 3541 |
| 542 | 1542 | 2542 | 3542 |
| 543 | 1543 | 2543 | 3543 |
| 544 | 1544 | 2544 | 3544 |
| 545 | 1545 | 2545 | 3545 |
| 546 | 1546 | 2546 | 3546 |
| 547 | 1547 | 2547 | 3547 |
| 548 | 1548 | 2548 | 3548 |
| 549 | 1549 | 2549 | 3549 |
| 550 | 1550 | 2550 | 3550 |
| 551 | 1551 | 2551 | 3551 |
| 552 | 1552 | 2552 | 3552 |
| 553 | 1553 | 2553 | 3553 |
| 554 | 1554 | 2554 | 3554 |
| 555 | 1555 | 2555 | 3555 |
| 556 | 1556 | 2556 | 3556 |
| 557 | 1557 | 2557 | 3557 |
| 558 | 1558 | 2558 | 3558 |
| 559 | 1559 | 2559 | 3559 |
| 560 | 1560 | 2560 | 3560 |
| 561 | 1561 | 2561 | 3561 |
| 562 | 1562 | 2562 | 3562 |
| 563 | 1563 | 2563 | 3563 |
| 564 | 1564 | 2564 | 3564 |
| 565 | 1565 | 2565 | 3565 |
| 566 | 1566 | 2566 | 3566 |
| 567 | 1567 | 2567 | 3567 |
| 568 | 1568 | 2568 | 3568 |
| 569 | 1569 | 2569 | 3569 |
| 570 | 1570 | 2570 | 3570 |
| 571 | 1571 | 2571 | 3571 |
| 572 | 1572 | 2572 | 3572 |
| 573 | 1573 | 2573 | 3573 |
| 574 | 1574 | 2574 | 3574 |
| 575 | 1575 | 2575 | 3575 |
| 576 | 1576 | 2576 | 3576 |
| 577 | 1577 | 2577 | 3577 |
| 578 | 1578 | 2578 | 3578 |
| 579 | 1579 | 2579 | 3579 |
| 580 | 1580 | 2580 | 3580 |
| 581 | 1581 | 2581 | 3581 |
| 582 | 1582 | 2582 | 3582 |
| 583 | 1583 | 2583 | 3583 |
| 584 | 1584 | 2584 | 3584 |
| 585 | 1585 | 2585 | 3585 |
| 586 | 1586 | 2586 | 3586 |
| 587 | 1587 | 2587 | 3587 |
| 588 | 1588 | 2588 | 3588 |
| 589 | 1589 | 2589 | 3589 |
| 590 | 1590 | 2590 | 3590 |
| 591 | 1591 | 2591 | 3591 |
| 592 | 1592 | 2592 | 3592 |
| 593 | 1593 | 2593 | 3593 |
| 594 | 1594 | 2594 | 3594 |
| 595 | 1595 | 2595 | 3595 |
| 596 | 1596 | 2596 | 3596 |
| 597 | 1597 | 2597 | 3597 |
| 598 | 1598 | 2598 | 3598 |
| 599 | 1599 | 2599 | 3599 |
| 600 | 1600 | 2600 | 3600 |
| 601 | 1601 | 2601 | 3601 |
| 602 | 1602 | 2602 | 3602 |
| 603 | 1603 | 2603 | 3603 |
| 604 | 1604 | 2604 | 3604 |
| 605 | 1605 | 2605 | 3605 |
| 606 | 1606 | 2606 | 3606 |
| 607 | 1607 | 2607 | 3607 |
| 608 | 1608 | 2608 | 3608 |
| 609 | 1609 | 2609 | 3609 |
| 610 | 1610 | 2610 | 3610 |
| 611 | 1611 | 2611 | 3611 |
| 612 | 1612 | 2612 | 3612 |
| 613 | 1613 | 2613 | 3613 |
| 614 | 1614 | 2614 | 3614 |
| 615 | 1615 | 2615 | 3615 |
| 616 | 1616 | 2616 | 3616 |
| 617 | 1617 | 2617 | 3617 |
| 618 | 1618 | 2618 | 3618 |
| 619 | 1619 | 2619 | 3619 |
| 620 | 1620 | 2620 | 3620 |
| 621 | 1621 | 2621 | 3621 |
| 622 | 1622 | 2622 | 3622 |
| 623 | 1623 | 2623 | 3623 |
| 624 | 1624 | 2624 | 3624 |
| 625 | 1625 | 2625 | 3625 |
| 626 | 1626 | 2626 | 3626 |
| 627 | 1627 | 2627 | 3627 |
| 628 | 1628 | 2628 | 3628 |
| 629 | 1629 | 2629 | 3629 |
| 630 | 1630 | 2630 | 3630 |
| 631 | 1631 | 2631 | 3631 |
| 632 | 1632 | 2632 | 3632 |
| 633 | 1633 | 2633 | 3633 |
| 634 | 1634 | 2634 | 3634 |
| 635 | 1635 | 2635 | 3635 |
| 636 | 1636 | 2636 | 3636 |
| 637 | 1637 | 2637 | 3637 |
| 638 | 1638 | 2638 | 3638 |
| 639 | 1639 | 2639 | 3639 |
| 640 | 1640 | 2640 | 3640 |
| 641 | 1641 | 2641 | 3641 |
| 642 | 1642 | 2642 | 3642 |
| 643 | 1643 | 2643 | 3643 |
| 644 | 1644 | 2644 | 3644 |
| 645 | 1645 | 2645 | 3645 |
| 646 | 1646 | 2646 | 3646 |
| 647 | 1647 | 2647 | 3647 |
| 648 | 1648 | 2648 | 3648 |
| 649 | 1649 | 2649 | 3649 |
| 650 | 1650 | 2650 | 3650 |
| 651 | 1651 | 2651 | 3651 |
| 652 | 1652 | 2652 | 3652 |
| 653 | 1653 | 2653 | 3653 |
| 654 | 1654 | 2654 | 3654 |
| 655 | 1655 | 2655 | 3655 |
| 656 | 1656 | 2656 | 3656 |
| 657 | 1657 | 2657 | 3657 |
| 658 | 1658 | 2658 | 3658 |
| 659 | 1659 | 2659 | 3659 |
| 660 | 1660 | 2660 | 3660 |
| 661 | 1661 | 2661 | 3661 |
| 662 | 1662 | 2662 | 3662 |
| 663 | 1663 | 2663 | 3663 |
| 664 | 1664 | 2664 | 3664 |
| 665 | 1665 | 2665 | 3665 |
| 666 | 1666 | 2666 | 3666 |
| 667 | 1667 | 2667 | 3667 |
| 668 | 1668 | 2668 | 3668 |
| 669 | 1669 | 2669 | 3669 |
| 670 | 1670 | 2670 | 3670 |
| 671 | 1671 | 2671 | 3671 |
| 672 | 1672 | 2672 | 3672 |
| 673 | 1673 | 2673 | 3673 |
| 674 | 1674 | 2674 | 3674 |
| 675 | 1675 | 2675 | 3675 |
| 676 | 1676 | 2676 | 3676 |
| 677 | 1677 | 2677 | 3677 |
| 678 | 1678 | 2678 | 3678 |
| 679 | 1679 | 2679 | 3679 |
| 680 | 1680 | 2680 | 3680 |
| 681 | 1681 | 2681 | 3681 |
| 682 | 1682 | 2682 | 3682 |
| 683 | 1683 | 2683 | 3683 |
| 684 | 1684 | 2684 | 3684 |
| 685 | 1685 | 2685 | 3685 |
| 686 | 1686 | 2686 | 3686 |
| 687 | 1687 | 2687 | 3687 |
| 688 | 1688 | 2688 | 3688 |
| 689 | 1689 | 2689 | 3689 |
| 690 | 1690 | 2690 | 3690 |
| 691 | 1691 | 2691 | 3691 |
| 692 | 1692 | 2692 | 3692 |
| 693 | 1693 | 2693 | 3693 |
| 694 | 1694 | 2694 | 3694 |
| 695 | 1695 | 2695 | 3695 |
| 696 | 1696 | 2696 | 3696 |
| 697 | 1697 | 2697 | 3697 |
| 698 | 1698 | 2698 | 3698 |
| 699 | 1699 | 2699 | 3699 |
| 700 | 1700 | 2700 | 3700 |
| 701 | 1701 | 2701 | 3701 |
| 702 | 1702 | 2702 | 3702 |
| 703 | 1703 | 2703 | 3703 |
| 704 | 1704 | 2704 | 3704 |
| 705 | 1705 | 2705 | 3705 |
| 706 | 1706 | 2706 | 3706 |
| 707 | 1707 | 2707 | 3707 |
| 708 | 1708 | 2708 | 3708 |
| 709 | 1709 | 2709 | 3709 |
| 710 | 1710 | 2710 | 3710 |
| 711 | 1711 | 2711 | 3711 |
| 712 | 1712 | 2712 | 3712 |
| 713 | 1713 | 2713 | 3713 |
| 714 | 1714 | 2714 | 3714 |
| 715 | 1715 | 2715 | 3715 |
| 716 | 1716 | 2716 | 3716 |
| 717 | 1717 | 2717 | 3717 |
| 718 | 1718 | 2718 | 3718 |
| 719 | 1719 | 2719 | 3719 |
| 720 | 1720 | 2720 | 3720 |
| 721 | 1721 | 2721 | 3721 |
| 722 | 1722 | 2722 | 3722 |
| 723 | 1723 | 2723 | 3723 |
| 724 | 1724 | 2724 | 3724 |
| 725 | 1725 | 2725 | 3725 |
| 726 | 1726 | 2726 | 3726 |
| 727 | 1727 | 2727 | 3727 |
| 728 | 1728 | 2728 | 3728 |
| 729 | 1729 | 2729 | 3729 |
| 730 | 1730 | 2730 | 3730 |
| 731 | 1731 | 2731 | 3731 |
| 732 | 1732 | 2732 | 3732 |
| 733 | 1733 | 2733 | 3733 |
| 734 | 1734 | 2734 | 3734 |
| 735 | 1735 | 2735 | 3735 |
| 736 | 1736 | 2736 | 3736 |
| 737 | 1737 | 2737 | 3737 |
| 738 | 1738 | 2738 | 3738 |
| 739 | 1739 | 2739 | 3739 |
| 740 | 1740 | 2740 | 3740 |
| 741 | 1741 | 2741 | 3741 |
| 742 | 1742 | 2742 | 3742 |
| 743 | 1743 | 2743 | 3743 |
| 744 | 1744 | 2744 | 3744 |
| 745 | 1745 | 2745 | 3745 |
| 746 | 1746 | 2746 | 3746 |
| 747 | 1747 | 2747 | 3747 |
| 748 | 1748 | 2748 | 3748 |
| 749 | 1749 | 2749 | 3749 |
| 750 | 1750 | 2750 | 3750 |
| 751 | 1751 | 2751 | 3751 |
| 752 | 1752 | 2752 | 3752 |
| 753 | 1753 | 2753 | 3753 |
| 754 | 1754 | 2754 | 3754 |
| 755 | 1755 | 2755 | 3755 |
| 756 | 1756 | 2756 | 3756 |
| 757 | 1757 | 2757 | 3757 |
| 758 | 1758 | 2758 | 3758 |
| 759 | 1759 | 2759 | 3759 |
| 760 | 1760 | 2760 | 3760 |
| 761 | 1761 | 2761 | 3761 |
| 762 | 1762 | 2762 | 3762 |
| 763 | 1763 | 2763 | 3763 |
| 764 | 1764 | 2764 | 3764 |
| 765 | 1765 | 2765 | 3765 |
| 766 | 1766 | 2766 | 3766 |
| 767 | 1767 | 2767 | 3767 |
| 768 | 1768 | 2768 | 3768 |
| 769 | 1769 | 2769 | 3769 |
| 770 | 1770 | 2770 | 3770 |
| 771 | 1771 | 2771 | 3771 |
| 772 | 1772 | 2772 | 3772 |
| 773 | 1773 | 2773 | 3773 |
| 774 | 1774 | 2774 | 3774 |
| 775 | 1775 | 2775 | 3775 |
| 776 | 1776 | 2776 | 3776 |
| 777 | 1777 | 2777 | 3777 |
| 778 | 1778 | 2778 | 3778 |
| 779 | 1779 | 2779 | 3779 |
| 780 | 1780 | 2780 | 3780 |
| 781 | 1781 | 2781 | 3781 |
| 782 | 1782 | 2782 | 3782 |
| 783 | 1783 | 2783 | 3783 |
| 784 | 1784 | 2784 | 3784 |
| 785 | 1785 | 2785 | 3785 |
| 786 | 1786 | 2786 | 3786 |
| 787 | 1787 | 2787 | 3787 |
| 788 | 1788 | 2788 | 3788 |
| 789 | 1789 | 2789 | 3789 |
| 790 | 1790 | 2790 | 3790 |
| 791 | 1791 | 2791 | 3791 |
| 792 | 1792 | 2792 | 3792 |
| 793 | 1793 | 2793 | 3793 |
| 794 | 1794 | 2794 | 3794 |
| 795 | 1795 | 2795 | 3795 |
| 796 | 1796 | 2796 | 3796 |
| 797 | 1797 | 2797 | 3797 |
| 798 | 1798 | 2798 | 3798 |
| 799 | 1799 | 2799 | 3799 |
| 800 | 1800 | 2800 | 3800 |
| 801 | 1801 | 2801 | 3801 |
| 802 | 1802 | 2802 | 3802 |
| 803 | 1803 | 2803 | 3803 |
| 804 | 1804 | 2804 | 3804 |
| 805 | 1805 | 2805 | 3805 |
| 806 | 1806 | 2806 | 3806 |
| 807 | 1807 | 2807 | 3807 |
| 808 | 1808 | 2808 | 3808 |
| 809 | 1809 | 2809 | 3809 |
| 810 | 1810 | 2810 | 3810 |
| 811 | 1811 | 2811 | 3811 |
| 812 | 1812 | 2812 | 3812 |
| 813 | 1813 | 2813 | 3813 |
| 814 | 1814 | 2814 | 3814 |
| 815 | 1815 | 2815 | 3815 |
| 816 | 1816 | 2816 | 3816 |
| 817 | 1817 | 2817 | 3817 |
| 818 | 1818 | 2818 | 3818 |
| 819 | 1819 | 2819 | 3819 |
| 820 | 1820 | 2820 | 3820 |
| 821 | 1821 | 2821 | 3821 |
| 822 | 1822 | 2822 | 3822 |
| 823 | 1823 | 2823 | 3823 |
| 824 | 1824 | 2824 | 3824 |
| 825 | 1825 | 2825 | 3825 |
| 826 | 1826 | 2826 | 3826 |
| 827 | 1827 | 2827 | 3827 |
| 828 | 1828 | 2828 | 3828 |
| 829 | 1829 | 2829 | 3829 |
| 830 | 1830 | 2830 | 3830 |
| 831 | 1831 | 2831 | 3831 |
| 832 | 1832 | 2832 | 3832 |
| 833 | 1833 | 2833 | 3833 |
| 834 | 1834 | 2834 | 3834 |
| 835 | 1835 | 2835 | 3835 |
| 836 | 1836 | 2836 | 3836 |
| 837 | 1837 | 2837 | 3837 |
| 838 | 1838 | 2838 | 3838 |
| 839 | 1839 | 2839 | 3839 |
| 840 | 1840 | 2840 | 3840 |
| 841 | 1841 | 2841 | 3841 |
| 842 | 1842 | 2842 | 3842 |
| 843 | 1843 | 2843 | 3843 |
| 844 | 1844 | 2844 | 3844 |
| 845 | 1845 | 2845 | 3845 |
| 846 | 1846 | 2846 | 3846 |
| 847 | 1847 | 2847 | 3847 |
| 848 | 1848 | 2848 | 3848 |
| 849 | 1849 | 2849 | 3849 |
| 850 | 1850 | 2850 | 3850 |
| 851 | 1851 | 2851 | 3851 |
| 852 | 1852 | 2852 | 3852 |
| 853 | 1853 | 2853 | 3853 |
| 854 | 1854 | 2854 | 3854 |
| 855 | 1855 | 2855 | 3855 |
| 856 | 1856 | 2856 | 3856 |
| 857 | 1857 | 2857 | 3857 |
| 858 | 1858 | 2858 | 3858 |
| 859 | 1859 | 2859 | 3859 |
| 860 | 1860 | 2860 | 3860 |
| 861 | 1861 | 2861 | 3861 |
| 862 | 1862 | 2862 | 3862 |
| 863 | 1863 | 2863 | 3863 |
| 864 | 1864 | 2864 | 3864 |
| 865 | 1865 | 2865 | 3865 |
| 866 | 1866 | 2866 | 3866 |
| 867 | 1867 | 2867 | 3867 |
| 868 | 1868 | 2868 | 3868 |
| 869 | 1869 | 2869 | 3869 |
| 870 | 1870 | 2870 | 3870 |
| 871 | 1871 | 2871 | 3871 |
| 872 | 1872 | 2872 | 3872 |
| 873 | 1873 | 2873 | 3873 |
| 874 | 1874 | 2874 | 3874 |
| 875 | 1875 | 2875 | 3875 |
| 876 | 1876 | 2876 | 3876 |
| 877 | 1877 | 2877 | 3877 |
| 878 | 1878 | 2878 | 3878 |
| 879 | 1879 | 2879 | 3879 |
| 880 | 1880 | 2880 | 3880 |
| 881 | 1881 | 2881 | 3881 |
| 882 | 1882 | 2882 | 3882 |
| 883 | 1883 | 2883 | 3883 |
| 884 | 1884 | 2884 | 3884 |
| 885 | 1885 | 2885 | 3885 |
| 886 | 1886 | 2886 | 3886 |
| 887 | 1887 | 2887 | 3887 |
| 888 | 1888 | 2888 | 3888 |
| 889 | 1889 | 2889 | 3889 |
| 890 | 1890 | 2890 | 3890 |
| 891 | 1891 | 2891 | 3891 |
| 892 | 1892 | 2892 | 3892 |
| 893 | 1893 | 2893 | 3893 |
| 894 | 1894 | 2894 | 3894 |
| 895 | 1895 | 2895 | 3895 |
| 896 | 1896 | 2896 | 3896 |
| 897 | 1897 | 2897 | 3897 |
| 898 | 1898 | 2898 | 3898 |
| 899 | 1899 | 2899 | 3899 |
| 900 | 1900 | 2900 | 3900 |
| 901 | 1901 | 2901 | 3901 |
| 902 | 1902 | 2902 | 3902 |
| 903 | 1903 | 2903 | 3903 |
| 904 | 1904 | 2904 | 3904 |
| 905 | 1905 | 2905 | 3905 |
| 906 | 1906 | 2906 | 3906 |
| 907 | 1907 | 2907 | 3907 |
| 908 | 1908 | 2908 | 3908 |
| 909 | 1909 | 2909 | 3909 |
| 910 | 1910 | 2910 | 3910 |
| 911 | 1911 | 2911 | 3911 |
| 912 | 1912 | 2912 | 3912 |
| 913 | 1913 | 2913 | 3913 |
| 914 | 1914 | 2914 | 3914 |
| 915 | 1915 | 2915 | 3915 |
| 916 | 1916 | 2916 | 3916 |
| 917 | 1917 | 2917 | 3917 |
| 918 | 1918 | 2918 | 3918 |
| 919 | 1919 | 2919 | 3919 |
| 920 | 1920 | 2920 | 3920 |
| 921 | 1921 | 2921 | 3921 |
| 922 | 1922 | 2922 | 3922 |
| 923 | 1923 | 2923 | 3923 |
| 924 | 1924 | 2924 | 3924 |
| 925 | 1925 | 2925 | 3925 |
| 926 | 1926 | 2926 | 3926 |
| 927 | 1927 | 2927 | 3927 |
| 928 | 1928 | 2928 | 3928 |
| 929 | 1929 | 2929 | 3929 |
| 930 | 1930 | 2930 | 3930 |
| 931 | 1931 | 2931 | 3931 |
| 932 | 1932 | 2932 | 3932 |
| 933 | 1933 | 2933 | 3933 |
| 934 | 1934 | 2934 | 3934 |
| 935 | 1935 | 2935 | 3935 |
| 936 | 1936 | 2936 | 3936 |
| 937 | 1937 | 2937 | 3937 |
| 938 | 1938 | 2938 | 3938 |
| 939 | 1939 | 2939 | 3939 |
| 940 | 1940 | 2940 | 3940 |
| 941 | 1941 | 2941 | 3941 |
| 942 | 1942 | 2942 | 3942 |
| 943 | 1943 | 2943 | 3943 |
| 944 | 1944 | 2944 | 3944 |
| 945 | 1945 | 2945 | 3945 |
| 946 | 1946 | 2946 | 3946 |
| 947 | 1947 | 2947 | 3947 |
| 948 | 1948 | 2948 | 3948 |
| 949 | 1949 | 2949 | 3949 |
| 950 | 1950 | 2950 | 3950 |
| 951 | 1951 | 2951 | 3951 |
| 952 | 1952 | 2952 | 3952 |
| 953 | 1953 | 2953 | 3953 |
| 954 | 1954 | 2954 | 3954 |
| 955 | 1955 | 2955 | 3955 |
| 956 | 1956 | 2956 | 3956 |
| 957 | 1957 | 2957 | 3957 |
| 958 | 1958 | 2958 | 3958 |
| 959 | 1959 | 2959 | 3959 |
| 960 | 1960 | 2960 | 3960 |
| 961 | 1961 | 2961 | 3961 |
| 962 | 1962 | 2962 | 3962 |
| 963 | 1963 | 2963 | 3963 |
| 964 | 1964 | 2964 | 3964 |
| 965 | 1965 | 2965 | 3965 |
| 966 | 1966 | 2966 | 3966 |
| 967 | 1967 | 2967 | 3967 |
| 968 | 1968 | 2968 | 3968 |
| 969 | 1969 | 2969 | 3969 |
| 970 | 1970 | 2970 | 3970 |
| 971 | 1971 | 2971 | 3971 |
| 972 | 1972 | 2972 | 3972 |
| 973 | 1973 | 2973 | 3973 |
| 974 | 1974 | 2974 | 3974 |
| 975 | 1975 | 2975 | 3975 |
| 976 | 1976 | 2976 | 3976 |
| 977 | 1977 | 2977 | 3977 |
| 978 | 1978 | 2978 | 3978 |
| 979 | 1979 | 2979 | 3979 |
| 980 | 1980 | 2980 | 3980 |
| 981 | 1981 | 2981 | 3981 |
| 982 | 1982 | 2982 | 3982 |
| 983 | 1983 | 2983 | 3983 |
| 984 | 1984 | 2984 | 3984 |
| 985 | 1985 | 2985 | 3985 |
| 986 | 1986 | 2986 | 3986 |
| 987 | 1987 | 2987 | 3987 |
| 988 | 1988 | 2988 | 3988 |
| 989 | 1989 | 2989 | 3989 |
| 990 | 1990 | 2990 | 3990 |
| 991 | 1991 | 2991 | 3991 |
| 992 | 1992 | 2992 | 3992 |
| 993 | 1993 | 2993 | 3993 |
| 994 | 1994 | 2994 | 3994 |
| 995 | 1995 | 2995 | 3995 |
| 996 | 1996 | 2996 | 3996 |
| 997 | 1997 | 2997 | 3997 |
| 998 | 1998 | 2998 | 3998 |
| 999 | 1999 | 2999 | 3999 |
| 1000 | 2000 | 3000 | 4000 |
| 1001 | 2001 | 3001 | 4001 |
| 1002 | 2002 | 3002 | 4002 |

In some embodiments, the sequences that specifically bind to almond comprise an inner sequence selected from the group consisting of the nucleic acid sequences of SEQ ID NOs. 4003 to 5002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to almond may comprise a nucleic acid sequence selected from the group consisting of SEQ ID NOs. 5003 to 6002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to almond may comprise a nucleic acid sequence selected from the group consisting of SEQ ID NOs. 6003 to 7002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to almond may comprise a nucleic acid sequence selected from the group consisting of SEQ ID NOs. 7003 to 8002. In one embodiment, the aptamer of the present disclosure that specifically binds to almond may comprise a nucleic acid sequence selected from the group consisting of SEQ ID NOs. 4003 to 8002 listed in Table 3, or variant thereof.

TABLE 3
Aptamer sequences against almond
Aptamer Sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 4003 | 5003 | 6003 | 7003 |
| 4004 | 5004 | 6004 | 7004 |
| 4005 | 5005 | 6005 | 7005 |
| 4006 | 5006 | 6006 | 7006 |
| 4007 | 5007 | 6007 | 7007 |
| 4008 | 5008 | 6008 | 7008 |
| 4009 | 5009 | 6009 | 7009 |
| 4010 | 5010 | 6010 | 7010 |
| 4011 | 5011 | 6011 | 7011 |
| 4012 | 5012 | 6012 | 7012 |
| 4013 | 5013 | 6013 | 7013 |
| 4014 | 5014 | 6014 | 7014 |
| 4015 | 5015 | 6015 | 7015 |
| 4016 | 5016 | 6016 | 7016 |
| 4017 | 5017 | 6017 | 7017 |
| 4018 | 5018 | 6018 | 7018 |
| 4019 | 5019 | 6019 | 7019 |
| 4020 | 5020 | 6020 | 7020 |
| 4021 | 5021 | 6021 | 7021 |
| 4022 | 5022 | 6022 | 7022 |
| 4023 | 5023 | 6023 | 7023 |
| 4024 | 5024 | 6024 | 7024 |
| 4025 | 5025 | 6025 | 7025 |
| 4026 | 5026 | 6026 | 7026 |
| 4027 | 5027 | 6027 | 7027 |
| 4028 | 5028 | 6028 | 7028 |
| 4029 | 5029 | 6029 | 7029 |
| 4030 | 5030 | 6030 | 7030 |
| 4031 | 5031 | 6031 | 7031 |
| 4032 | 5032 | 6032 | 7032 |
| 4033 | 5033 | 6033 | 7033 |
| 4034 | 5034 | 6034 | 7034 |
| 4035 | 5035 | 6035 | 7035 |
| 4036 | 5036 | 6036 | 7036 |
| 4037 | 5037 | 6037 | 7037 |
| 4038 | 5038 | 6038 | 7038 |
| 4039 | 5039 | 6039 | 7039 |
| 4040 | 5040 | 6040 | 7040 |
| 4041 | 5041 | 6041 | 7041 |
| 4042 | 5042 | 6042 | 7042 |
| 4043 | 5043 | 6043 | 7043 |
| 4044 | 5044 | 6044 | 7044 |
| 4045 | 5045 | 6045 | 7045 |
| 4046 | 5046 | 6046 | 7046 |
| 4047 | 5047 | 6047 | 7047 |
| 4048 | 5048 | 6048 | 7048 |
| 4049 | 5049 | 6049 | 7049 |
| 4050 | 5050 | 6050 | 7050 |
| 4051 | 5051 | 6051 | 7051 |
| 4052 | 5052 | 6052 | 7052 |
| 4053 | 5053 | 6053 | 7053 |
| 4054 | 5054 | 6054 | 7054 |
| 4055 | 5055 | 6055 | 7055 |
| 4056 | 5056 | 6056 | 7056 |
| 4057 | 5057 | 6057 | 7057 |
| 4058 | 5058 | 6058 | 7058 |
| 4059 | 5059 | 6059 | 7059 |
| 4060 | 5060 | 6060 | 7060 |
| 4061 | 5061 | 6061 | 7061 |
| 4062 | 5062 | 6062 | 7062 |
| 4063 | 5063 | 6063 | 7063 |
| 4064 | 5064 | 6064 | 7064 |
| 4065 | 5065 | 6065 | 7065 |
| 4066 | 5066 | 6066 | 7066 |
| 4067 | 5067 | 6067 | 7067 |
| 4068 | 5068 | 6068 | 7068 |
| 4069 | 5069 | 6069 | 7069 |
| 4070 | 5070 | 6070 | 7070 |
| 4071 | 5071 | 6071 | 7071 |
| 4072 | 5072 | 6072 | 7072 |
| 4073 | 5073 | 6073 | 7073 |
| 4074 | 5074 | 6074 | 7074 |
| 4075 | 5075 | 6075 | 7075 |
| 4076 | 5076 | 6076 | 7076 |
| 4077 | 5077 | 6077 | 7077 |
| 4078 | 5078 | 6078 | 7078 |
| 4079 | 5079 | 6079 | 7079 |
| 4080 | 5080 | 6080 | 7080 |
| 4081 | 5081 | 6081 | 7081 |
| 4082 | 5082 | 6082 | 7082 |
| 4083 | 5083 | 6083 | 7083 |
| 4084 | 5084 | 6084 | 7084 |
| 4085 | 5085 | 6085 | 7085 |
| 4086 | 5086 | 6086 | 7086 |
| 4087 | 5087 | 6087 | 7087 |
| 4088 | 5088 | 6088 | 7088 |
| 4089 | 5089 | 6089 | 7089 |
| 4090 | 5090 | 6090 | 7090 |
| 4091 | 5091 | 6091 | 7091 |
| 4092 | 5092 | 6092 | 7092 |
| 4093 | 5093 | 6093 | 7093 |
| 4094 | 5094 | 6094 | 7094 |
| 4095 | 5095 | 6095 | 7095 |
| 4096 | 5096 | 6096 | 7096 |
| 4097 | 5097 | 6097 | 7097 |
| 4098 | 5098 | 6098 | 7098 |
| 4099 | 5099 | 6099 | 7099 |
| 4100 | 5100 | 6100 | 7100 |
| 4101 | 5101 | 6101 | 7101 |
| 4102 | 5102 | 6102 | 7102 |
| 4103 | 5103 | 6103 | 7103 |
| 4104 | 5104 | 6104 | 7104 |
| 4105 | 5105 | 6105 | 7105 |
| 4106 | 5106 | 6106 | 7106 |
| 4107 | 5107 | 6107 | 7107 |
| 4108 | 5108 | 6108 | 7108 |
| 4109 | 5109 | 6109 | 7109 |
| 4110 | 5110 | 6110 | 7110 |
| 4111 | 5111 | 6111 | 7111 |
| 4112 | 5112 | 6112 | 7112 |
| 4113 | 5113 | 6113 | 7113 |
| 4114 | 5114 | 6114 | 7114 |
| 4115 | 5115 | 6115 | 7115 |
| 4116 | 5116 | 6116 | 7116 |
| 4117 | 5117 | 6117 | 7117 |
| 4118 | 5118 | 6118 | 7118 |
| 4119 | 5119 | 6119 | 7119 |
| 4120 | 5120 | 6120 | 7120 |
| 4121 | 5121 | 6121 | 7121 |
| 4122 | 5122 | 6122 | 7122 |
| 4123 | 5123 | 6123 | 7123 |
| 4124 | 5124 | 6124 | 7124 |
| 4125 | 5125 | 6125 | 7125 |
| 4126 | 5126 | 6126 | 7126 |
| 4127 | 5127 | 6127 | 7127 |
| 4128 | 5128 | 6128 | 7128 |
| 4129 | 5129 | 6129 | 7129 |
| 4130 | 5130 | 6130 | 7130 |
| 4131 | 5131 | 6131 | 7131 |
| 4132 | 5132 | 6132 | 7132 |
| 4133 | 5133 | 6133 | 7133 |
| 4134 | 5134 | 6134 | 7134 |
| 4135 | 5135 | 6135 | 7135 |
| 4136 | 5136 | 6136 | 7136 |
| 4137 | 5137 | 6137 | 7137 |
| 4138 | 5138 | 6138 | 7138 |
| 4139 | 5139 | 6139 | 7139 |
| 4140 | 5140 | 6140 | 7140 |
| 4141 | 5141 | 6141 | 7141 |
| 4142 | 5142 | 6142 | 7142 |
| 4143 | 5143 | 6143 | 7143 |
| 4144 | 5144 | 6144 | 7144 |
| 4145 | 5145 | 6145 | 7145 |
| 4146 | 5146 | 6146 | 7146 |
| 4147 | 5147 | 6147 | 7147 |
| 4148 | 5148 | 6148 | 7148 |
| 4149 | 5149 | 6149 | 7149 |
| 4150 | 5150 | 6150 | 7150 |
| 4151 | 5151 | 6151 | 7151 |
| 4152 | 5152 | 6152 | 7152 |
| 4153 | 5153 | 6153 | 7153 |
| 4154 | 5154 | 6154 | 7154 |
| 4155 | 5155 | 6155 | 7155 |
| 4156 | 5156 | 6156 | 7156 |
| 4157 | 5157 | 6157 | 7157 |
| 4158 | 5158 | 6158 | 7158 |
| 4159 | 5159 | 6159 | 7159 |
| 4160 | 5160 | 6160 | 7160 |
| 4161 | 5161 | 6161 | 7161 |
| 4162 | 5162 | 6162 | 7162 |
| 4163 | 5163 | 6163 | 7163 |
| 4164 | 5164 | 6164 | 7164 |
| 4165 | 5165 | 6165 | 7165 |
| 4166 | 5166 | 6166 | 7166 |
| 4167 | 5167 | 6167 | 7167 |
| 4168 | 5168 | 6168 | 7168 |
| 4169 | 5169 | 6169 | 7169 |
| 4170 | 5170 | 6170 | 7170 |
| 4171 | 5171 | 6171 | 7171 |
| 4172 | 5172 | 6172 | 7172 |
| 4173 | 5173 | 6173 | 7173 |
| 4174 | 5174 | 6174 | 7174 |
| 4175 | 5175 | 6175 | 7175 |
| 4176 | 5176 | 6176 | 7176 |
| 4177 | 5177 | 6177 | 7177 |
| 4178 | 5178 | 6178 | 7178 |
| 4179 | 5179 | 6179 | 7179 |
| 4180 | 5180 | 6180 | 7180 |
| 4181 | 5181 | 6181 | 7181 |
| 4182 | 5182 | 6182 | 7182 |
| 4183 | 5183 | 6183 | 7183 |
| 4184 | 5184 | 6184 | 7184 |
| 4185 | 5185 | 6185 | 7185 |
| 4186 | 5186 | 6186 | 7186 |
| 4187 | 5187 | 6187 | 7187 |
| 4188 | 5188 | 6188 | 7188 |
| 4189 | 5189 | 6189 | 7189 |
| 4190 | 5190 | 6190 | 7190 |
| 4191 | 5191 | 6191 | 7191 |
| 4192 | 5192 | 6192 | 7192 |
| 4193 | 5193 | 6193 | 7193 |
| 4194 | 5194 | 6194 | 7194 |
| 4195 | 5195 | 6195 | 7195 |
| 4196 | 5196 | 6196 | 7196 |
| 4197 | 5197 | 6197 | 7197 |
| 4198 | 5198 | 6198 | 7198 |
| 4199 | 5199 | 6199 | 7199 |
| 4200 | 5200 | 6200 | 7200 |
| 4201 | 5201 | 6201 | 7201 |
| 4202 | 5202 | 6202 | 7202 |
| 4203 | 5203 | 6203 | 7203 |
| 4204 | 5204 | 6204 | 7204 |
| 4205 | 5205 | 6205 | 7205 |
| 4206 | 5206 | 6206 | 7206 |
| 4207 | 5207 | 6207 | 7207 |
| 4208 | 5208 | 6208 | 7208 |
| 4209 | 5209 | 6209 | 7209 |
| 4210 | 5210 | 6210 | 7210 |
| 4211 | 5211 | 6211 | 7211 |
| 4212 | 5212 | 6212 | 7212 |
| 4213 | 5213 | 6213 | 7213 |
| 4214 | 5214 | 6214 | 7214 |
| 4215 | 5215 | 6215 | 7215 |
| 4216 | 5216 | 6216 | 7216 |
| 4217 | 5217 | 6217 | 7217 |
| 4218 | 5218 | 6218 | 7218 |
| 4219 | 5219 | 6219 | 7219 |
| 4220 | 5220 | 6220 | 7220 |
| 4221 | 5221 | 6221 | 7221 |
| 4222 | 5222 | 6222 | 7222 |
| 4223 | 5223 | 6223 | 7223 |
| 4224 | 5224 | 6224 | 7224 |
| 4225 | 5225 | 6225 | 7225 |
| 4226 | 5226 | 6226 | 7226 |
| 4227 | 5227 | 6227 | 7227 |
| 4228 | 5228 | 6228 | 7228 |
| 4229 | 5229 | 6229 | 7229 |
| 4230 | 5230 | 6230 | 7230 |
| 4231 | 5231 | 6231 | 7231 |
| 4232 | 5232 | 6232 | 7232 |
| 4233 | 5233 | 6233 | 7233 |
| 4234 | 5234 | 6234 | 7234 |
| 4235 | 5235 | 6235 | 7235 |
| 4236 | 5236 | 6236 | 7236 |
| 4237 | 5237 | 6237 | 7237 |
| 4238 | 5238 | 6238 | 7238 |
| 4239 | 5239 | 6239 | 7239 |
| 4240 | 5240 | 6240 | 7240 |
| 4241 | 5241 | 6241 | 7241 |
| 4242 | 5242 | 6242 | 7242 |
| 4243 | 5243 | 6243 | 7243 |
| 4244 | 5244 | 6244 | 7244 |
| 4245 | 5245 | 6245 | 7245 |
| 4246 | 5246 | 6246 | 7246 |
| 4247 | 5247 | 6247 | 7247 |
| 4248 | 5248 | 6248 | 7248 |
| 4249 | 5249 | 6249 | 7249 |
| 4250 | 5250 | 6250 | 7250 |
| 4251 | 5251 | 6251 | 7251 |
| 4252 | 5252 | 6252 | 7252 |
| 4253 | 5253 | 6253 | 7253 |
| 4254 | 5254 | 6254 | 7254 |
| 4255 | 5255 | 6255 | 7255 |
| 4256 | 5256 | 6256 | 7256 |
| 4257 | 5257 | 6257 | 7257 |
| 4258 | 5258 | 6258 | 7258 |
| 4259 | 5259 | 6259 | 7259 |
| 4260 | 5260 | 6260 | 7260 |
| 4261 | 5261 | 6261 | 7261 |
| 4262 | 5262 | 6262 | 7262 |
| 4263 | 5263 | 6263 | 7263 |
| 4264 | 5264 | 6264 | 7264 |
| 4265 | 5265 | 6265 | 7265 |
| 4266 | 5266 | 6266 | 7266 |
| 4267 | 5267 | 6267 | 7267 |
| 4268 | 5268 | 6268 | 7268 |
| 4269 | 5269 | 6269 | 7269 |
| 4270 | 5270 | 6270 | 7270 |
| 4271 | 5271 | 6271 | 7271 |
| 4272 | 5272 | 6272 | 7272 |
| 4273 | 5273 | 6273 | 7273 |
| 4274 | 5274 | 6274 | 7274 |
| 4275 | 5275 | 6275 | 7275 |
| 4276 | 5276 | 6276 | 7276 |
| 4277 | 5277 | 6277 | 7277 |
| 4278 | 5278 | 6278 | 7278 |
| 4279 | 5279 | 6279 | 7279 |
| 4280 | 5280 | 6280 | 7280 |
| 4281 | 5281 | 6281 | 7281 |
| 4282 | 5282 | 6282 | 7282 |
| 4283 | 5283 | 6283 | 7283 |
| 4284 | 5284 | 6284 | 7284 |
| 4285 | 5285 | 6285 | 7285 |
| 4286 | 5286 | 6286 | 7286 |
| 4287 | 5287 | 6287 | 7287 |
| 4288 | 5288 | 6288 | 7288 |
| 4289 | 5289 | 6289 | 7289 |
| 4290 | 5290 | 6290 | 7290 |
| 4291 | 5291 | 6291 | 7291 |
| 4292 | 5292 | 6292 | 7292 |
| 4293 | 5293 | 6293 | 7293 |
| 4294 | 5294 | 6294 | 7294 |
| 4295 | 5295 | 6295 | 7295 |
| 4296 | 5296 | 6296 | 7296 |
| 4297 | 5297 | 6297 | 7297 |
| 4298 | 5298 | 6298 | 7298 |
| 4299 | 5299 | 6299 | 7299 |
| 4300 | 5300 | 6300 | 7300 |
| 4301 | 5301 | 6301 | 7301 |
| 4302 | 5302 | 6302 | 7302 |
| 4303 | 5303 | 6303 | 7303 |
| 4304 | 5304 | 6304 | 7304 |
| 4305 | 5305 | 6305 | 7305 |
| 4306 | 5306 | 6306 | 7306 |
| 4307 | 5307 | 6307 | 7307 |
| 4308 | 5308 | 6308 | 7308 |
| 4309 | 5309 | 6309 | 7309 |
| 4310 | 5310 | 6310 | 7310 |
| 4311 | 5311 | 6311 | 7311 |
| 4312 | 5312 | 6312 | 7312 |
| 4313 | 5313 | 6313 | 7313 |
| 4314 | 5314 | 6314 | 7314 |
| 4315 | 5315 | 6315 | 7315 |
| 4316 | 5316 | 6316 | 7316 |
| 4317 | 5317 | 6317 | 7317 |
| 4318 | 5318 | 6318 | 7318 |
| 4319 | 5319 | 6319 | 7319 |
| 4320 | 5320 | 6320 | 7320 |
| 4321 | 5321 | 6321 | 7321 |
| 4322 | 5322 | 6322 | 7322 |
| 4323 | 5323 | 6323 | 7323 |
| 4324 | 5324 | 6324 | 7324 |
| 4325 | 5325 | 6325 | 7325 |
| 4326 | 5326 | 6326 | 7326 |
| 4327 | 5327 | 6327 | 7327 |
| 4328 | 5328 | 6328 | 7328 |
| 4329 | 5329 | 6329 | 7329 |
| 4330 | 5330 | 6330 | 7330 |
| 4331 | 5331 | 6331 | 7331 |
| 4332 | 5332 | 6332 | 7332 |
| 4333 | 5333 | 6333 | 7333 |
| 4334 | 5334 | 6334 | 7334 |
| 4335 | 5335 | 6335 | 7335 |
| 4336 | 5336 | 6336 | 7336 |
| 4337 | 5337 | 6337 | 7337 |
| 4338 | 5338 | 6338 | 7338 |
| 4339 | 5339 | 6339 | 7339 |
| 4340 | 5340 | 6340 | 7340 |
| 4341 | 5341 | 6341 | 7341 |
| 4342 | 5342 | 6342 | 7342 |
| 4343 | 5343 | 6343 | 7343 |
| 4344 | 5344 | 6344 | 7344 |
| 4345 | 5345 | 6345 | 7345 |
| 4346 | 5346 | 6346 | 7346 |
| 4347 | 5347 | 6347 | 7347 |
| 4348 | 5348 | 6348 | 7348 |
| 4349 | 5349 | 6349 | 7349 |
| 4350 | 5350 | 6350 | 7350 |
| 4351 | 5351 | 6351 | 7351 |
| 4352 | 5352 | 6352 | 7352 |
| 4353 | 5353 | 6353 | 7353 |
| 4354 | 5354 | 6354 | 7354 |
| 4355 | 5355 | 6355 | 7355 |
| 4356 | 5356 | 6356 | 7356 |
| 4357 | 5357 | 6357 | 7357 |
| 4358 | 5358 | 6358 | 7358 |
| 4359 | 5359 | 6359 | 7359 |
| 4360 | 5360 | 6360 | 7360 |
| 4361 | 5361 | 6361 | 7361 |
| 4362 | 5362 | 6362 | 7362 |
| 4363 | 5363 | 6363 | 7363 |
| 4364 | 5364 | 6364 | 7364 |
| 4365 | 5365 | 6365 | 7365 |
| 4366 | 5366 | 6366 | 7366 |
| 4367 | 5367 | 6367 | 7367 |
| 4368 | 5368 | 6368 | 7368 |
| 4369 | 5369 | 6369 | 7369 |
| 4370 | 5370 | 6370 | 7370 |
| 4371 | 5371 | 6371 | 7371 |
| 4372 | 5372 | 6372 | 7372 |
| 4373 | 5373 | 6373 | 7373 |
| 4374 | 5374 | 6374 | 7374 |
| 4375 | 5375 | 6375 | 7375 |
| 4376 | 5376 | 6376 | 7376 |
| 4377 | 5377 | 6377 | 7377 |
| 4378 | 5378 | 6378 | 7378 |
| 4379 | 5379 | 6379 | 7379 |
| 4380 | 5380 | 6380 | 7380 |
| 4381 | 5381 | 6381 | 7381 |
| 4382 | 5382 | 6382 | 7382 |
| 4383 | 5383 | 6383 | 7383 |
| 4384 | 5384 | 6384 | 7384 |
| 4385 | 5385 | 6385 | 7385 |
| 4386 | 5386 | 6386 | 7386 |
| 4387 | 5387 | 6387 | 7387 |
| 4388 | 5388 | 6388 | 7388 |
| 4389 | 5389 | 6389 | 7389 |
| 4390 | 5390 | 6390 | 7390 |
| 4391 | 5391 | 6391 | 7391 |
| 4392 | 5392 | 6392 | 7392 |
| 4393 | 5393 | 6393 | 7393 |
| 4394 | 5394 | 6394 | 7394 |
| 4395 | 5395 | 6395 | 7395 |
| 4396 | 5396 | 6396 | 7396 |
| 4397 | 5397 | 6397 | 7397 |
| 4398 | 5398 | 6398 | 7398 |
| 4399 | 5399 | 6399 | 7399 |
| 4400 | 5400 | 6400 | 7400 |
| 4401 | 5401 | 6401 | 7401 |
| 4402 | 5402 | 6402 | 7402 |
| 4403 | 5403 | 6403 | 7403 |
| 4404 | 5404 | 6404 | 7404 |
| 4405 | 5405 | 6405 | 7405 |
| 4406 | 5406 | 6406 | 7406 |
| 4407 | 5407 | 6407 | 7407 |
| 4408 | 5408 | 6408 | 7408 |
| 4409 | 5409 | 6409 | 7409 |
| 4410 | 5410 | 6410 | 7410 |
| 4411 | 5411 | 6411 | 7411 |
| 4412 | 5412 | 6412 | 7412 |
| 4413 | 5413 | 6413 | 7413 |
| 4414 | 5414 | 6414 | 7414 |
| 4415 | 5415 | 6415 | 7415 |
| 4416 | 5416 | 6416 | 7416 |
| 4417 | 5417 | 6417 | 7417 |
| 4418 | 5418 | 6418 | 7418 |
| 4419 | 5419 | 6419 | 7419 |
| 4420 | 5420 | 6420 | 7420 |
| 4421 | 5421 | 6421 | 7421 |
| 4422 | 5422 | 6422 | 7422 |
| 4423 | 5423 | 6423 | 7423 |
| 4424 | 5424 | 6424 | 7424 |
| 4425 | 5425 | 6425 | 7425 |
| 4426 | 5426 | 6426 | 7426 |
| 4427 | 5427 | 6427 | 7427 |
| 4428 | 5428 | 6428 | 7428 |
| 4429 | 5429 | 6429 | 7429 |
| 4430 | 5430 | 6430 | 7430 |
| 4431 | 5431 | 6431 | 7431 |
| 4432 | 5432 | 6432 | 7432 |
| 4433 | 5433 | 6433 | 7433 |
| 4434 | 5434 | 6434 | 7434 |
| 4435 | 5435 | 6435 | 7435 |
| 4436 | 5436 | 6436 | 7436 |
| 4437 | 5437 | 6437 | 7437 |
| 4438 | 5438 | 6438 | 7438 |
| 4439 | 5439 | 6439 | 7439 |
| 4440 | 5440 | 6440 | 7440 |
| 4441 | 5441 | 6441 | 7441 |
| 4442 | 5442 | 6442 | 7442 |
| 4443 | 5443 | 6443 | 7443 |
| 4444 | 5444 | 6444 | 7444 |
| 4445 | 5445 | 6445 | 7445 |
| 4446 | 5446 | 6446 | 7446 |
| 4447 | 5447 | 6447 | 7447 |
| 4448 | 5448 | 6448 | 7448 |
| 4449 | 5449 | 6449 | 7449 |
| 4450 | 5450 | 6450 | 7450 |
| 4451 | 5451 | 6451 | 7451 |
| 4452 | 5452 | 6452 | 7452 |
| 4453 | 5453 | 6453 | 7453 |
| 4454 | 5454 | 6454 | 7454 |
| 4455 | 5455 | 6455 | 7455 |
| 4456 | 5456 | 6456 | 7456 |
| 4457 | 5457 | 6457 | 7457 |
| 4458 | 5458 | 6458 | 7458 |
| 4459 | 5459 | 6459 | 7459 |
| 4460 | 5460 | 6460 | 7460 |
| 4461 | 5461 | 6461 | 7461 |
| 4462 | 5462 | 6462 | 7462 |
| 4463 | 5463 | 6463 | 7463 |
| 4464 | 5464 | 6464 | 7464 |
| 4465 | 5465 | 6465 | 7465 |
| 4466 | 5466 | 6466 | 7466 |
| 4467 | 5467 | 6467 | 7467 |
| 4468 | 5468 | 6468 | 7468 |
| 4469 | 5469 | 6469 | 7469 |
| 4470 | 5470 | 6470 | 7470 |
| 4471 | 5471 | 6471 | 7471 |
| 4472 | 5472 | 6472 | 7472 |
| 4473 | 5473 | 6473 | 7473 |
| 4474 | 5474 | 6474 | 7474 |
| 4475 | 5475 | 6475 | 7475 |
| 4476 | 5476 | 6476 | 7476 |
| 4477 | 5477 | 6477 | 7477 |
| 4478 | 5478 | 6478 | 7478 |
| 4479 | 5479 | 6479 | 7479 |
| 4480 | 5480 | 6480 | 7480 |
| 4481 | 5481 | 6481 | 7481 |
| 4482 | 5482 | 6482 | 7482 |
| 4483 | 5483 | 6483 | 7483 |
| 4484 | 5484 | 6484 | 7484 |
| 4485 | 5485 | 6485 | 7485 |
| 4486 | 5486 | 6486 | 7486 |
| 4487 | 5487 | 6487 | 7487 |
| 4488 | 5488 | 6488 | 7488 |
| 4489 | 5489 | 6489 | 7489 |
| 4490 | 5490 | 6490 | 7490 |
| 4491 | 5491 | 6491 | 7491 |
| 4492 | 5492 | 6492 | 7492 |
| 4493 | 5493 | 6493 | 7493 |
| 4494 | 5494 | 6494 | 7494 |
| 4495 | 5495 | 6495 | 7495 |
| 4496 | 5496 | 6496 | 7496 |
| 4497 | 5497 | 6497 | 7497 |
| 4498 | 5498 | 6498 | 7498 |
| 4499 | 5499 | 6499 | 7499 |
| 4500 | 5500 | 6500 | 7500 |
| 4501 | 5501 | 6501 | 7501 |
| 4502 | 5502 | 6502 | 7502 |
| 4503 | 5503 | 6503 | 7503 |
| 4504 | 5504 | 6504 | 7504 |
| 4505 | 5505 | 6505 | 7505 |
| 4506 | 5506 | 6506 | 7506 |
| 4507 | 5507 | 6507 | 7507 |
| 4508 | 5508 | 6508 | 7508 |
| 4509 | 5509 | 6509 | 7509 |
| 4510 | 5510 | 6510 | 7510 |
| 4511 | 5511 | 6511 | 7511 |
| 4512 | 5512 | 6512 | 7512 |
| 4513 | 5513 | 6513 | 7513 |
| 4514 | 5514 | 6514 | 7514 |
| 4515 | 5515 | 6515 | 7515 |
| 4516 | 5516 | 6516 | 7516 |
| 4517 | 5517 | 6517 | 7517 |
| 4518 | 5518 | 6518 | 7518 |
| 4519 | 5519 | 6519 | 7519 |
| 4520 | 5520 | 6520 | 7520 |
| 4521 | 5521 | 6521 | 7521 |
| 4522 | 5522 | 6522 | 7522 |
| 4523 | 5523 | 6523 | 7523 |
| 4524 | 5524 | 6524 | 7524 |
| 4525 | 5525 | 6525 | 7525 |
| 4526 | 5526 | 6526 | 7526 |
| 4527 | 5527 | 6527 | 7527 |
| 4528 | 5528 | 6528 | 7528 |
| 4529 | 5529 | 6529 | 7529 |
| 4530 | 5530 | 6530 | 7530 |
| 4531 | 5531 | 6531 | 7531 |
| 4532 | 5532 | 6532 | 7532 |
| 4533 | 5533 | 6533 | 7533 |
| 4534 | 5534 | 6534 | 7534 |
| 4535 | 5535 | 6535 | 7535 |
| 4536 | 5536 | 6536 | 7536 |
| 4537 | 5537 | 6537 | 7537 |
| 4538 | 5538 | 6538 | 7538 |
| 4539 | 5539 | 6539 | 7539 |
| 4540 | 5540 | 6540 | 7540 |
| 4541 | 5541 | 6541 | 7541 |
| 4542 | 5542 | 6542 | 7542 |
| 4543 | 5543 | 6543 | 7543 |
| 4544 | 5544 | 6544 | 7544 |
| 4545 | 5545 | 6545 | 7545 |
| 4546 | 5546 | 6546 | 7546 |
| 4547 | 5547 | 6547 | 7547 |
| 4548 | 5548 | 6548 | 7548 |
| 4549 | 5549 | 6549 | 7549 |
| 4550 | 5550 | 6550 | 7550 |
| 4551 | 5551 | 6551 | 7551 |
| 4552 | 5552 | 6552 | 7552 |
| 4553 | 5553 | 6553 | 7553 |
| 4554 | 5554 | 6554 | 7554 |
| 4555 | 5555 | 6555 | 7555 |
| 4556 | 5556 | 6556 | 7556 |
| 4557 | 5557 | 6557 | 7557 |
| 4558 | 5558 | 6558 | 7558 |
| 4559 | 5559 | 6559 | 7559 |
| 4560 | 5560 | 6560 | 7560 |
| 4561 | 5561 | 6561 | 7561 |
| 4562 | 5562 | 6562 | 7562 |
| 4563 | 5563 | 6563 | 7563 |
| 4564 | 5564 | 6564 | 7564 |
| 4565 | 5565 | 6565 | 7565 |
| 4566 | 5566 | 6566 | 7566 |
| 4567 | 5567 | 6567 | 7567 |
| 4568 | 5568 | 6568 | 7568 |
| 4569 | 5569 | 6569 | 7569 |
| 4570 | 5570 | 6570 | 7570 |
| 4571 | 5571 | 6571 | 7571 |
| 4572 | 5572 | 6572 | 7572 |
| 4573 | 5573 | 6573 | 7573 |
| 4574 | 5574 | 6574 | 7574 |
| 4575 | 5575 | 6575 | 7575 |
| 4576 | 5576 | 6576 | 7576 |
| 4577 | 5577 | 6577 | 7577 |
| 4578 | 5578 | 6578 | 7578 |
| 4579 | 5579 | 6579 | 7579 |
| 4580 | 5580 | 6580 | 7580 |
| 4581 | 5581 | 6581 | 7581 |
| 4582 | 5582 | 6582 | 7582 |
| 4583 | 5583 | 6583 | 7583 |
| 4584 | 5584 | 6584 | 7584 |
| 4585 | 5585 | 6585 | 7585 |
| 4586 | 5586 | 6586 | 7586 |
| 4587 | 5587 | 6587 | 7587 |
| 4588 | 5588 | 6588 | 7588 |
| 4589 | 5589 | 6589 | 7589 |
| 4590 | 5590 | 6590 | 7590 |
| 4591 | 5591 | 6591 | 7591 |
| 4592 | 5592 | 6592 | 7592 |
| 4593 | 5593 | 6593 | 7593 |
| 4594 | 5594 | 6594 | 7594 |
| 4595 | 5595 | 6595 | 7595 |
| 4596 | 5596 | 6596 | 7596 |
| 4597 | 5597 | 6597 | 7597 |
| 4598 | 5598 | 6598 | 7598 |
| 4599 | 5599 | 6599 | 7599 |
| 4600 | 5600 | 6600 | 7600 |
| 4601 | 5601 | 6601 | 7601 |
| 4602 | 5602 | 6602 | 7602 |
| 4603 | 5603 | 6603 | 7603 |
| 4604 | 5604 | 6604 | 7604 |
| 4605 | 5605 | 6605 | 7605 |
| 4606 | 5606 | 6606 | 7606 |
| 4607 | 5607 | 6607 | 7607 |
| 4608 | 5608 | 6608 | 7608 |
| 4609 | 5609 | 6609 | 7609 |
| 4610 | 5610 | 6610 | 7610 |
| 4611 | 5611 | 6611 | 7611 |
| 4612 | 5612 | 6612 | 7612 |
| 4613 | 5613 | 6613 | 7613 |
| 4614 | 5614 | 6614 | 7614 |
| 4615 | 5615 | 6615 | 7615 |
| 4616 | 5616 | 6616 | 7616 |
| 4617 | 5617 | 6617 | 7617 |
| 4618 | 5618 | 6618 | 7618 |
| 4619 | 5619 | 6619 | 7619 |
| 4620 | 5620 | 6620 | 7620 |
| 4621 | 5621 | 6621 | 7621 |
| 4622 | 5622 | 6622 | 7622 |
| 4623 | 5623 | 6623 | 7623 |
| 4624 | 5624 | 6624 | 7624 |
| 4625 | 5625 | 6625 | 7625 |
| 4626 | 5626 | 6626 | 7626 |
| 4627 | 5627 | 6627 | 7627 |
| 4628 | 5628 | 6628 | 7628 |
| 4629 | 5629 | 6629 | 7629 |
| 4630 | 5630 | 6630 | 7630 |
| 4631 | 5631 | 6631 | 7631 |
| 4632 | 5632 | 6632 | 7632 |
| 4633 | 5633 | 6633 | 7633 |
| 4634 | 5634 | 6634 | 7634 |
| 4635 | 5635 | 6635 | 7635 |
| 4636 | 5636 | 6636 | 7636 |
| 4637 | 5637 | 6637 | 7637 |
| 4638 | 5638 | 6638 | 7638 |
| 4639 | 5639 | 6639 | 7639 |
| 4640 | 5640 | 6640 | 7640 |
| 4641 | 5641 | 6641 | 7641 |
| 4642 | 5642 | 6642 | 7642 |
| 4643 | 5643 | 6643 | 7643 |
| 4644 | 5644 | 6644 | 7644 |
| 4645 | 5645 | 6645 | 7645 |
| 4646 | 5646 | 6646 | 7646 |
| 4647 | 5647 | 6647 | 7647 |
| 4648 | 5648 | 6648 | 7648 |
| 4649 | 5649 | 6649 | 7649 |
| 4650 | 5650 | 6650 | 7650 |
| 4651 | 5651 | 6651 | 7651 |
| 4652 | 5652 | 6652 | 7652 |
| 4653 | 5653 | 6653 | 7653 |
| 4654 | 5654 | 6654 | 7654 |
| 4655 | 5655 | 6655 | 7655 |
| 4656 | 5656 | 6656 | 7656 |
| 4657 | 5657 | 6657 | 7657 |
| 4658 | 5658 | 6658 | 7658 |
| 4659 | 5659 | 6659 | 7659 |
| 4660 | 5660 | 6660 | 7660 |
| 4661 | 5661 | 6661 | 7661 |
| 4662 | 5662 | 6662 | 7662 |
| 4663 | 5663 | 6663 | 7663 |
| 4664 | 5664 | 6664 | 7664 |
| 4665 | 5665 | 6665 | 7665 |
| 4666 | 5666 | 6666 | 7666 |
| 4667 | 5667 | 6667 | 7667 |
| 4668 | 5668 | 6668 | 7668 |
| 4669 | 5669 | 6669 | 7669 |
| 4670 | 5670 | 6670 | 7670 |
| 4671 | 5671 | 6671 | 7671 |
| 4672 | 5672 | 6672 | 7672 |
| 4673 | 5673 | 6673 | 7673 |
| 4674 | 5674 | 6674 | 7674 |
| 4675 | 5675 | 6675 | 7675 |
| 4676 | 5676 | 6676 | 7676 |
| 4677 | 5677 | 6677 | 7677 |
| 4678 | 5678 | 6678 | 7678 |
| 4679 | 5679 | 6679 | 7679 |
| 4680 | 5680 | 6680 | 7680 |
| 4681 | 5681 | 6681 | 7681 |
| 4682 | 5682 | 6682 | 7682 |
| 4683 | 5683 | 6683 | 7683 |
| 4684 | 5684 | 6684 | 7684 |
| 4685 | 5685 | 6685 | 7685 |
| 4686 | 5686 | 6686 | 7686 |
| 4687 | 5687 | 6687 | 7687 |
| 4688 | 5688 | 6688 | 7688 |
| 4689 | 5689 | 6689 | 7689 |
| 4690 | 5690 | 6690 | 7690 |
| 4691 | 5691 | 6691 | 7691 |
| 4692 | 5692 | 6692 | 7692 |
| 4693 | 5693 | 6693 | 7693 |
| 4694 | 5694 | 6694 | 7694 |
| 4695 | 5695 | 6695 | 7695 |
| 4696 | 5696 | 6696 | 7696 |
| 4697 | 5697 | 6697 | 7697 |
| 4698 | 5698 | 6698 | 7698 |
| 4699 | 5699 | 6699 | 7699 |
| 4700 | 5700 | 6700 | 7700 |
| 4701 | 5701 | 6701 | 7701 |
| 4702 | 5702 | 6702 | 7702 |
| 4703 | 5703 | 6703 | 7703 |
| 4704 | 5704 | 6704 | 7704 |
| 4705 | 5705 | 6705 | 7705 |
| 4706 | 5706 | 6706 | 7706 |
| 4707 | 5707 | 6707 | 7707 |
| 4708 | 5708 | 6708 | 7708 |
| 4709 | 5709 | 6709 | 7709 |
| 4710 | 5710 | 6710 | 7710 |
| 4711 | 5711 | 6711 | 7711 |
| 4712 | 5712 | 6712 | 7712 |
| 4713 | 5713 | 6713 | 7713 |
| 4714 | 5714 | 6714 | 7714 |
| 4715 | 5715 | 6715 | 7715 |
| 4716 | 5716 | 6716 | 7716 |
| 4717 | 5717 | 6717 | 7717 |
| 4718 | 5718 | 6718 | 7718 |
| 4719 | 5719 | 6719 | 7719 |
| 4720 | 5720 | 6720 | 7720 |
| 4721 | 5721 | 6721 | 7721 |
| 4722 | 5722 | 6722 | 7722 |
| 4723 | 5723 | 6723 | 7723 |
| 4724 | 5724 | 6724 | 7724 |
| 4725 | 5725 | 6725 | 7725 |
| 4726 | 5726 | 6726 | 7726 |
| 4727 | 5727 | 6727 | 7727 |
| 4728 | 5728 | 6728 | 7728 |
| 4729 | 5729 | 6729 | 7729 |
| 4730 | 5730 | 6730 | 7730 |
| 4731 | 5731 | 6731 | 7731 |
| 4732 | 5732 | 6732 | 7732 |
| 4733 | 5733 | 6733 | 7733 |
| 4734 | 5734 | 6734 | 7734 |
| 4735 | 5735 | 6735 | 7735 |
| 4736 | 5736 | 6736 | 7736 |
| 4737 | 5737 | 6737 | 7737 |
| 4738 | 5738 | 6738 | 7738 |
| 4739 | 5739 | 6739 | 7739 |
| 4740 | 5740 | 6740 | 7740 |
| 4741 | 5741 | 6741 | 7741 |
| 4742 | 5742 | 6742 | 7742 |
| 4743 | 5743 | 6743 | 7743 |
| 4744 | 5744 | 6744 | 7744 |
| 4745 | 5745 | 6745 | 7745 |
| 4746 | 5746 | 6746 | 7746 |
| 4747 | 5747 | 6747 | 7747 |
| 4748 | 5748 | 6748 | 7748 |
| 4749 | 5749 | 6749 | 7749 |
| 4750 | 5750 | 6750 | 7750 |
| 4751 | 5751 | 6751 | 7751 |
| 4752 | 5752 | 6752 | 7752 |
| 4753 | 5753 | 6753 | 7753 |
| 4754 | 5754 | 6754 | 7754 |
| 4755 | 5755 | 6755 | 7755 |
| 4756 | 5756 | 6756 | 7756 |
| 4757 | 5757 | 6757 | 7757 |
| 4758 | 5758 | 6758 | 7758 |
| 4759 | 5759 | 6759 | 7759 |
| 4760 | 5760 | 6760 | 7760 |
| 4761 | 5761 | 6761 | 7761 |
| 4762 | 5762 | 6762 | 7762 |
| 4763 | 5763 | 6763 | 7763 |
| 4764 | 5764 | 6764 | 7764 |
| 4765 | 5765 | 6765 | 7765 |
| 4766 | 5766 | 6766 | 7766 |
| 4767 | 5767 | 6767 | 7767 |
| 4768 | 5768 | 6768 | 7768 |
| 4769 | 5769 | 6769 | 7769 |
| 4770 | 5770 | 6770 | 7770 |
| 4771 | 5771 | 6771 | 7771 |
| 4772 | 5772 | 6772 | 7772 |
| 4773 | 5773 | 6773 | 7773 |
| 4774 | 5774 | 6774 | 7774 |
| 4775 | 5775 | 6775 | 7775 |
| 4776 | 5776 | 6776 | 7776 |
| 4777 | 5777 | 6777 | 7777 |
| 4778 | 5778 | 6778 | 7778 |
| 4779 | 5779 | 6779 | 7779 |
| 4780 | 5780 | 6780 | 7780 |
| 4781 | 5781 | 6781 | 7781 |
| 4782 | 5782 | 6782 | 7782 |
| 4783 | 5783 | 6783 | 7783 |
| 4784 | 5784 | 6784 | 7784 |
| 4785 | 5785 | 6785 | 7785 |
| 4786 | 5786 | 6786 | 7786 |
| 4787 | 5787 | 6787 | 7787 |
| 4788 | 5788 | 6788 | 7788 |
| 4789 | 5789 | 6789 | 7789 |
| 4790 | 5790 | 6790 | 7790 |
| 4791 | 5791 | 6791 | 7791 |
| 4792 | 5792 | 6792 | 7792 |
| 4793 | 5793 | 6793 | 7793 |
| 4794 | 5794 | 6794 | 7794 |
| 4795 | 5795 | 6795 | 7795 |
| 4796 | 5796 | 6796 | 7796 |
| 4797 | 5797 | 6797 | 7797 |
| 4798 | 5798 | 6798 | 7798 |
| 4799 | 5799 | 6799 | 7799 |
| 4800 | 5800 | 6800 | 7800 |
| 4801 | 5801 | 6801 | 7801 |
| 4802 | 5802 | 6802 | 7802 |
| 4803 | 5803 | 6803 | 7803 |
| 4804 | 5804 | 6804 | 7804 |
| 4805 | 5805 | 6805 | 7805 |
| 4806 | 5806 | 6806 | 7806 |
| 4807 | 5807 | 6807 | 7807 |
| 4808 | 5808 | 6808 | 7808 |
| 4809 | 5809 | 6809 | 7809 |
| 4810 | 5810 | 6810 | 7810 |
| 4811 | 5811 | 6811 | 7811 |
| 4812 | 5812 | 6812 | 7812 |
| 4813 | 5813 | 6813 | 7813 |
| 4814 | 5814 | 6814 | 7814 |
| 4815 | 5815 | 6815 | 7815 |
| 4816 | 5816 | 6816 | 7816 |
| 4817 | 5817 | 6817 | 7817 |
| 4818 | 5818 | 6818 | 7818 |
| 4819 | 5819 | 6819 | 7819 |
| 4820 | 5820 | 6820 | 7820 |
| 4821 | 5821 | 6821 | 7821 |
| 4822 | 5822 | 6822 | 7822 |
| 4823 | 5823 | 6823 | 7823 |
| 4824 | 5824 | 6824 | 7824 |
| 4825 | 5825 | 6825 | 7825 |
| 4826 | 5826 | 6826 | 7826 |
| 4827 | 5827 | 6827 | 7827 |
| 4828 | 5828 | 6828 | 7828 |
| 4829 | 5829 | 6829 | 7829 |
| 4830 | 5830 | 6830 | 7830 |
| 4831 | 5831 | 6831 | 7831 |
| 4832 | 5832 | 6832 | 7832 |
| 4833 | 5833 | 6833 | 7833 |
| 4834 | 5834 | 6834 | 7834 |
| 4835 | 5835 | 6835 | 7835 |
| 4836 | 5836 | 6836 | 7836 |
| 4837 | 5837 | 6837 | 7837 |
| 4838 | 5838 | 6838 | 7838 |
| 4839 | 5839 | 6839 | 7839 |
| 4840 | 5840 | 6840 | 7840 |
| 4841 | 5841 | 6841 | 7841 |
| 4842 | 5842 | 6842 | 7842 |
| 4843 | 5843 | 6843 | 7843 |
| 4844 | 5844 | 6844 | 7844 |
| 4845 | 5845 | 6845 | 7845 |
| 4846 | 5846 | 6846 | 7846 |
| 4847 | 5847 | 6847 | 7847 |
| 4848 | 5848 | 6848 | 7848 |
| 4849 | 5849 | 6849 | 7849 |
| 4850 | 5850 | 6850 | 7850 |
| 4851 | 5851 | 6851 | 7851 |
| 4852 | 5852 | 6852 | 7852 |
| 4853 | 5853 | 6853 | 7853 |
| 4854 | 5854 | 6854 | 7854 |
| 4855 | 5855 | 6855 | 7855 |
| 4856 | 5856 | 6856 | 7856 |
| 4857 | 5857 | 6857 | 7857 |
| 4858 | 5858 | 6858 | 7858 |
| 4859 | 5859 | 6859 | 7859 |
| 4860 | 5860 | 6860 | 7860 |
| 4861 | 5861 | 6861 | 7861 |
| 4862 | 5862 | 6862 | 7862 |
| 4863 | 5863 | 6863 | 7863 |
| 4864 | 5864 | 6864 | 7864 |
| 4865 | 5865 | 6865 | 7865 |
| 4866 | 5866 | 6866 | 7866 |
| 4867 | 5867 | 6867 | 7867 |
| 4868 | 5868 | 6868 | 7868 |
| 4869 | 5869 | 6869 | 7869 |
| 4870 | 5870 | 6870 | 7870 |
| 4871 | 5871 | 6871 | 7871 |
| 4872 | 5872 | 6872 | 7872 |
| 4873 | 5873 | 6873 | 7873 |
| 4874 | 5874 | 6874 | 7874 |
| 4875 | 5875 | 6875 | 7875 |
| 4876 | 5876 | 6876 | 7876 |
| 4877 | 5877 | 6877 | 7877 |
| 4878 | 5878 | 6878 | 7878 |
| 4879 | 5879 | 6879 | 7879 |
| 4880 | 5880 | 6880 | 7880 |
| 4881 | 5881 | 6881 | 7881 |
| 4882 | 5882 | 6882 | 7882 |
| 4883 | 5883 | 6883 | 7883 |
| 4884 | 5884 | 6884 | 7884 |
| 4885 | 5885 | 6885 | 7885 |
| 4886 | 5886 | 6886 | 7886 |
| 4887 | 5887 | 6887 | 7887 |
| 4888 | 5888 | 6888 | 7888 |
| 4889 | 5889 | 6889 | 7889 |
| 4890 | 5890 | 6890 | 7890 |
| 4891 | 5891 | 6891 | 7891 |
| 4892 | 5892 | 6892 | 7892 |
| 4893 | 5893 | 6893 | 7893 |
| 4894 | 5894 | 6894 | 7894 |
| 4895 | 5895 | 6895 | 7895 |
| 4896 | 5896 | 6896 | 7896 |
| 4897 | 5897 | 6897 | 7897 |
| 4898 | 5898 | 6898 | 7898 |
| 4899 | 5899 | 6899 | 7899 |
| 4900 | 5900 | 6900 | 7900 |
| 4901 | 5901 | 6901 | 7901 |
| 4902 | 5902 | 6902 | 7902 |
| 4903 | 5903 | 6903 | 7903 |
| 4904 | 5904 | 6904 | 7904 |
| 4905 | 5905 | 6905 | 7905 |
| 4906 | 5906 | 6906 | 7906 |
| 4907 | 5907 | 6907 | 7907 |
| 4908 | 5908 | 6908 | 7908 |
| 4909 | 5909 | 6909 | 7909 |
| 4910 | 5910 | 6910 | 7910 |
| 4911 | 5911 | 6911 | 7911 |
| 4912 | 5912 | 6912 | 7912 |
| 4913 | 5913 | 6913 | 7913 |
| 4914 | 5914 | 6914 | 7914 |
| 4915 | 5915 | 6915 | 7915 |
| 4916 | 5916 | 6916 | 7916 |
| 4917 | 5917 | 6917 | 7917 |
| 4918 | 5918 | 6918 | 7918 |
| 4919 | 5919 | 6919 | 7919 |
| 4920 | 5920 | 6920 | 7920 |
| 4921 | 5921 | 6921 | 7921 |
| 4922 | 5922 | 6922 | 7922 |
| 4923 | 5923 | 6923 | 7923 |
| 4924 | 5924 | 6924 | 7924 |
| 4925 | 5925 | 6925 | 7925 |
| 4926 | 5926 | 6926 | 7926 |
| 4927 | 5927 | 6927 | 7927 |
| 4928 | 5928 | 6928 | 7928 |
| 4929 | 5929 | 6929 | 7929 |
| 4930 | 5930 | 6930 | 7930 |
| 4931 | 5931 | 6931 | 7931 |
| 4932 | 5932 | 6932 | 7932 |
| 4933 | 5933 | 6933 | 7933 |
| 4934 | 5934 | 6934 | 7934 |
| 4935 | 5935 | 6935 | 7935 |
| 4936 | 5936 | 6936 | 7936 |
| 4937 | 5937 | 6937 | 7937 |
| 4938 | 5938 | 6938 | 7938 |
| 4939 | 5939 | 6939 | 7939 |
| 4940 | 5940 | 6940 | 7940 |
| 4941 | 5941 | 6941 | 7941 |
| 4942 | 5942 | 6942 | 7942 |
| 4943 | 5943 | 6943 | 7943 |
| 4944 | 5944 | 6944 | 7944 |
| 4945 | 5945 | 6945 | 7945 |
| 4946 | 5946 | 6946 | 7946 |
| 4947 | 5947 | 6947 | 7947 |
| 4948 | 5948 | 6948 | 7948 |
| 4949 | 5949 | 6949 | 7949 |
| 4950 | 5950 | 6950 | 7950 |
| 4951 | 5951 | 6951 | 7951 |
| 4952 | 5952 | 6952 | 7952 |
| 4953 | 5953 | 6953 | 7953 |
| 4954 | 5954 | 6954 | 7954 |
| 4955 | 5955 | 6955 | 7955 |
| 4956 | 5956 | 6956 | 7956 |
| 4957 | 5957 | 6957 | 7957 |
| 4958 | 5958 | 6958 | 7958 |
| 4959 | 5959 | 6959 | 7959 |
| 4960 | 5960 | 6960 | 7960 |
| 4961 | 5961 | 6961 | 7961 |
| 4962 | 5962 | 6962 | 7962 |
| 4963 | 5963 | 6963 | 7963 |
| 4964 | 5964 | 6964 | 7964 |
| 4965 | 5965 | 6965 | 7965 |
| 4966 | 5966 | 6966 | 7966 |
| 4967 | 5967 | 6967 | 7967 |
| 4968 | 5968 | 6968 | 7968 |
| 4969 | 5969 | 6969 | 7969 |
| 4970 | 5970 | 6970 | 7970 |
| 4971 | 5971 | 6971 | 7971 |
| 4972 | 5972 | 6972 | 7972 |
| 4973 | 5973 | 6973 | 7973 |
| 4974 | 5974 | 6974 | 7974 |
| 4975 | 5975 | 6975 | 7975 |
| 4976 | 5976 | 6976 | 7976 |
| 4977 | 5977 | 6977 | 7977 |
| 4978 | 5978 | 6978 | 7978 |
| 4979 | 5979 | 6979 | 7979 |
| 4980 | 5980 | 6980 | 7980 |
| 4981 | 5981 | 6981 | 7981 |
| 4982 | 5982 | 6982 | 7982 |
| 4983 | 5983 | 6983 | 7983 |
| 4984 | 5984 | 6984 | 7984 |
| 4985 | 5985 | 6985 | 7985 |
| 4986 | 5986 | 6986 | 7986 |
| 4987 | 5987 | 6987 | 7987 |
| 4988 | 5988 | 6988 | 7988 |
| 4989 | 5989 | 6989 | 7989 |
| 4990 | 5990 | 6990 | 7990 |
| 4991 | 5991 | 6991 | 7991 |
| 4992 | 5992 | 6992 | 7992 |
| 4993 | 5993 | 6993 | 7993 |
| 4994 | 5994 | 6994 | 7994 |
| 4995 | 5995 | 6995 | 7995 |
| 4996 | 5996 | 6996 | 7996 |
| 4997 | 5997 | 6997 | 7997 |
| 4998 | 5998 | 6998 | 7998 |
| 4999 | 5999 | 6999 | 7999 |
| 5000 | 6000 | 7000 | 8000 |
| 5001 | 6001 | 7001 | 8001 |
| 5002 | 6002 | 7002 | 8002 |

In some embodiments, the sequences that specifically bind to brazil nut comprise an inner sequence selected from the group consisting of the nucleic acid sequences of SEQ ID NOs. 8003 to 9002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to brazil nut may comprise a nucleic acid sequence selected from the group consisting of SEQ ID NOs. 9003 to 10002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to brazil nut may comprise a nucleic acid sequence selected from the group consisting of SEQ ID NOs. 10003 to 11002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to brazil nut may comprise a nucleic acid sequence selected from the group consisting of SEQ ID NOs. 11003 to 12002. In one embodiment, the aptamer of the present disclosure that specifically binds to brazil nut may comprise a nucleic acid sequence selected from the group consisting of SEQ ID NOs. 8003 to 12002 listed in Table 4, or variant thereof.

TABLE 4
Aptamer sequences against brazil nut
Aptamer sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 8003 | 9003 | 10003 | 11003 |
| 8004 | 9004 | 10004 | 11004 |
| 8005 | 9005 | 10005 | 11005 |
| 8006 | 9006 | 10006 | 11006 |
| 8007 | 9007 | 10007 | 11007 |
| 8008 | 9008 | 10008 | 11008 |
| 8009 | 9009 | 10009 | 11009 |
| 8010 | 9010 | 10010 | 11010 |
| 8011 | 9011 | 10011 | 11011 |
| 8012 | 9012 | 10012 | 11012 |
| 8013 | 9013 | 10013 | 11013 |
| 8014 | 9014 | 10014 | 11014 |
| 8015 | 9015 | 10015 | 11015 |
| 8016 | 9016 | 10016 | 11016 |
| 8017 | 9017 | 10017 | 11017 |
| 8018 | 9018 | 10018 | 11018 |
| 8019 | 9019 | 10019 | 11019 |
| 8020 | 9020 | 10020 | 11020 |
| 8021 | 9021 | 10021 | 11021 |
| 8022 | 9022 | 10022 | 11022 |
| 8023 | 9023 | 10023 | 11023 |
| 8024 | 9024 | 10024 | 11024 |
| 8025 | 9025 | 10025 | 11025 |
| 8026 | 9026 | 10026 | 11026 |
| 8027 | 9027 | 10027 | 11027 |
| 8028 | 9028 | 10028 | 11028 |
| 8029 | 9029 | 10029 | 11029 |
| 8030 | 9030 | 10030 | 11030 |
| 8031 | 9031 | 10031 | 11031 |
| 8032 | 9032 | 10032 | 11032 |
| 8033 | 9033 | 10033 | 11033 |
| 8034 | 9034 | 10034 | 11034 |
| 8035 | 9035 | 10035 | 11035 |
| 8036 | 9036 | 10036 | 11036 |
| 8037 | 9037 | 10037 | 11037 |
| 8038 | 9038 | 10038 | 11038 |
| 8039 | 9039 | 10039 | 11039 |
| 8040 | 9040 | 10040 | 11040 |
| 8041 | 9041 | 10041 | 11041 |
| 8042 | 9042 | 10042 | 11042 |
| 8043 | 9043 | 10043 | 11043 |
| 8044 | 9044 | 10044 | 11044 |
| 8045 | 9045 | 10045 | 11045 |
| 8046 | 9046 | 10046 | 11046 |
| 8047 | 9047 | 10047 | 11047 |
| 8048 | 9048 | 10048 | 11048 |
| 8049 | 9049 | 10049 | 11049 |
| 8050 | 9050 | 10050 | 11050 |
| 8051 | 9051 | 10051 | 11051 |
| 8052 | 9052 | 10052 | 11052 |
| 8053 | 9053 | 10053 | 11053 |
| 8054 | 9054 | 10054 | 11054 |
| 8055 | 9055 | 10055 | 11055 |
| 8056 | 9056 | 10056 | 11056 |
| 8057 | 9057 | 10057 | 11057 |
| 8058 | 9058 | 10058 | 11058 |
| 8059 | 9059 | 10059 | 11059 |
| 8060 | 9060 | 10060 | 11060 |
| 8061 | 9061 | 10061 | 11061 |
| 8062 | 9062 | 10062 | 11062 |
| 8063 | 9063 | 10063 | 11063 |
| 8064 | 9064 | 10064 | 11064 |
| 8065 | 9065 | 10065 | 11065 |
| 8066 | 9066 | 10066 | 11066 |
| 8067 | 9067 | 10067 | 11067 |
| 8068 | 9068 | 10068 | 11068 |
| 8069 | 9069 | 10069 | 11069 |
| 8070 | 9070 | 10070 | 11070 |
| 8071 | 9071 | 10071 | 11071 |
| 8072 | 9072 | 10072 | 11072 |
| 8073 | 9073 | 10073 | 11073 |
| 8074 | 9074 | 10074 | 11074 |
| 8075 | 9075 | 10075 | 11075 |
| 8076 | 9076 | 10076 | 11076 |
| 8077 | 9077 | 10077 | 11077 |
| 8078 | 9078 | 10078 | 11078 |
| 8079 | 9079 | 10079 | 11079 |
| 8080 | 9080 | 10080 | 11080 |
| 8081 | 9081 | 10081 | 11081 |
| 8082 | 9082 | 10082 | 11082 |
| 8083 | 9083 | 10083 | 11083 |
| 8084 | 9084 | 10084 | 11084 |
| 8085 | 9085 | 10085 | 11085 |
| 8086 | 9086 | 10086 | 11086 |
| 8087 | 9087 | 10087 | 11087 |
| 8088 | 9088 | 10088 | 11088 |
| 8089 | 9089 | 10089 | 11089 |
| 8090 | 9090 | 10090 | 11090 |
| 8091 | 9091 | 10091 | 11091 |
| 8092 | 9092 | 10092 | 11092 |
| 8093 | 9093 | 10093 | 11093 |
| 8094 | 9094 | 10094 | 11094 |
| 8095 | 9095 | 10095 | 11095 |
| 8096 | 9096 | 10096 | 11096 |
| 8097 | 9097 | 10097 | 11097 |
| 8098 | 9098 | 10098 | 11098 |
| 8099 | 9099 | 10099 | 11099 |
| 8100 | 9100 | 10100 | 11100 |
| 8101 | 9101 | 10101 | 11101 |
| 8102 | 9102 | 10102 | 11102 |
| 8103 | 9103 | 10103 | 11103 |
| 8104 | 9104 | 10104 | 11104 |
| 8105 | 9105 | 10105 | 11105 |
| 8106 | 9106 | 10106 | 11106 |
| 8107 | 9107 | 10107 | 11107 |
| 8108 | 9108 | 10108 | 11108 |
| 8109 | 9109 | 10109 | 11109 |
| 8110 | 9110 | 10110 | 11110 |
| 8111 | 9111 | 10111 | 11111 |
| 8112 | 9112 | 10112 | 11112 |
| 8113 | 9113 | 10113 | 11113 |
| 8114 | 9114 | 10114 | 11114 |
| 8115 | 9115 | 10115 | 11115 |
| 8116 | 9116 | 10116 | 11116 |
| 8117 | 9117 | 10117 | 11117 |
| 8118 | 9118 | 10118 | 11118 |
| 8119 | 9119 | 10119 | 11119 |
| 8120 | 9120 | 10120 | 11120 |
| 8121 | 9121 | 10121 | 11121 |
| 8122 | 9122 | 10122 | 11122 |
| 8123 | 9123 | 10123 | 11123 |
| 8124 | 9124 | 10124 | 11124 |
| 8125 | 9125 | 10125 | 11125 |
| 8126 | 9126 | 10126 | 11126 |
| 8127 | 9127 | 10127 | 11127 |
| 8128 | 9128 | 10128 | 11128 |
| 8129 | 9129 | 10129 | 11129 |
| 8130 | 9130 | 10130 | 11130 |
| 8131 | 9131 | 10131 | 11131 |
| 8132 | 9132 | 10132 | 11132 |
| 8133 | 9133 | 10133 | 11133 |
| 8134 | 9134 | 10134 | 11134 |
| 8135 | 9135 | 10135 | 11135 |
| 8136 | 9136 | 10136 | 11136 |
| 8137 | 9137 | 10137 | 11137 |
| 8138 | 9138 | 10138 | 11138 |
| 8139 | 9139 | 10139 | 11139 |
| 8140 | 9140 | 10140 | 11140 |
| 8141 | 9141 | 10141 | 11141 |
| 8142 | 9142 | 10142 | 11142 |
| 8143 | 9143 | 10143 | 11143 |
| 8144 | 9144 | 10144 | 11144 |
| 8145 | 9145 | 10145 | 11145 |
| 8146 | 9146 | 10146 | 11146 |
| 8147 | 9147 | 10147 | 11147 |
| 8148 | 9148 | 10148 | 11148 |
| 8149 | 9149 | 10149 | 11149 |
| 8150 | 9150 | 10150 | 11150 |
| 8151 | 9151 | 10151 | 11151 |
| 8152 | 9152 | 10152 | 11152 |
| 8153 | 9153 | 10153 | 11153 |
| 8154 | 9154 | 10154 | 11154 |
| 8155 | 9155 | 10155 | 11155 |
| 8156 | 9156 | 10156 | 11156 |
| 8157 | 9157 | 10157 | 11157 |
| 8158 | 9158 | 10158 | 11158 |
| 8159 | 9159 | 10159 | 11159 |
| 8160 | 9160 | 10160 | 11160 |
| 8161 | 9161 | 10161 | 11161 |
| 8162 | 9162 | 10162 | 11162 |
| 8163 | 9163 | 10163 | 11163 |
| 8164 | 9164 | 10164 | 11164 |
| 8165 | 9165 | 10165 | 11165 |
| 8166 | 9166 | 10166 | 11166 |
| 8167 | 9167 | 10167 | 11167 |
| 8168 | 9168 | 10168 | 11168 |
| 8169 | 9169 | 10169 | 11169 |
| 8170 | 9170 | 10170 | 11170 |
| 8171 | 9171 | 10171 | 11171 |
| 8172 | 9172 | 10172 | 11172 |
| 8173 | 9173 | 10173 | 11173 |
| 8174 | 9174 | 10174 | 11174 |
| 8175 | 9175 | 10175 | 11175 |
| 8176 | 9176 | 10176 | 11176 |
| 8177 | 9177 | 10177 | 11177 |
| 8178 | 9178 | 10178 | 11178 |
| 8179 | 9179 | 10179 | 11179 |
| 8180 | 9180 | 10180 | 11180 |
| 8181 | 9181 | 10181 | 11181 |
| 8182 | 9182 | 10182 | 11182 |
| 8183 | 9183 | 10183 | 11183 |
| 8184 | 9184 | 10184 | 11184 |
| 8185 | 9185 | 10185 | 11185 |
| 8186 | 9186 | 10186 | 11186 |
| 8187 | 9187 | 10187 | 11187 |
| 8188 | 9188 | 10188 | 11188 |
| 8189 | 9189 | 10189 | 11189 |
| 8190 | 9190 | 10190 | 11190 |
| 8191 | 9191 | 10191 | 11191 |
| 8192 | 9192 | 10192 | 11192 |
| 8193 | 9193 | 10193 | 11193 |
| 8194 | 9194 | 10194 | 11194 |
| 8195 | 9195 | 10195 | 11195 |
| 8196 | 9196 | 10196 | 11196 |
| 8197 | 9197 | 10197 | 11197 |
| 8198 | 9198 | 10198 | 11198 |
| 8199 | 9199 | 10199 | 11199 |
| 8200 | 9200 | 10200 | 11200 |
| 8201 | 9201 | 10201 | 11201 |
| 8202 | 9202 | 10202 | 11202 |
| 8203 | 9203 | 10203 | 11203 |
| 8204 | 9204 | 10204 | 11204 |
| 8205 | 9205 | 10205 | 11205 |
| 8206 | 9206 | 10206 | 11206 |
| 8207 | 9207 | 10207 | 11207 |
| 8208 | 9208 | 10208 | 11208 |
| 8209 | 9209 | 10209 | 11209 |
| 8210 | 9210 | 10210 | 11210 |
| 8211 | 9211 | 10211 | 11211 |
| 8212 | 9212 | 10212 | 11212 |
| 8213 | 9213 | 10213 | 11213 |
| 8214 | 9214 | 10214 | 11214 |
| 8215 | 9215 | 10215 | 11215 |
| 8216 | 9216 | 10216 | 11216 |
| 8217 | 9217 | 10217 | 11217 |
| 8218 | 9218 | 10218 | 11218 |
| 8219 | 9219 | 10219 | 11219 |
| 8220 | 9220 | 10220 | 11220 |
| 8221 | 9221 | 10221 | 11221 |
| 8222 | 9222 | 10222 | 11222 |
| 8223 | 9223 | 10223 | 11223 |
| 8224 | 9224 | 10224 | 11224 |
| 8225 | 9225 | 10225 | 11225 |
| 8226 | 9226 | 10226 | 11226 |
| 8227 | 9227 | 10227 | 11227 |
| 8228 | 9228 | 10228 | 11228 |
| 8229 | 9229 | 10229 | 11229 |
| 8230 | 9230 | 10230 | 11230 |
| 8231 | 9231 | 10231 | 11231 |
| 8232 | 9232 | 10232 | 11232 |
| 8233 | 9233 | 10233 | 11233 |
| 8234 | 9234 | 10234 | 11234 |
| 8235 | 9235 | 10235 | 11235 |
| 8236 | 9236 | 10236 | 11236 |
| 8237 | 9237 | 10237 | 11237 |
| 8238 | 9238 | 10238 | 11238 |
| 8239 | 9239 | 10239 | 11239 |
| 8240 | 9240 | 10240 | 11240 |
| 8241 | 9241 | 10241 | 11241 |
| 8242 | 9242 | 10242 | 11242 |
| 8243 | 9243 | 10243 | 11243 |
| 8244 | 9244 | 10244 | 11244 |
| 8245 | 9245 | 10245 | 11245 |
| 8246 | 9246 | 10246 | 11246 |
| 8247 | 9247 | 10247 | 11247 |
| 8248 | 9248 | 10248 | 11248 |
| 8249 | 9249 | 10249 | 11249 |
| 8250 | 9250 | 10250 | 11250 |
| 8251 | 9251 | 10251 | 11251 |
| 8252 | 9252 | 10252 | 11252 |
| 8253 | 9253 | 10253 | 11253 |
| 8254 | 9254 | 10254 | 11254 |
| 8255 | 9255 | 10255 | 11255 |
| 8256 | 9256 | 10256 | 11256 |
| 8257 | 9257 | 10257 | 11257 |
| 8258 | 9258 | 10258 | 11258 |
| 8259 | 9259 | 10259 | 11259 |
| 8260 | 9260 | 10260 | 11260 |
| 8261 | 9261 | 10261 | 11261 |
| 8262 | 9262 | 10262 | 11262 |
| 8263 | 9263 | 10263 | 11263 |
| 8264 | 9264 | 10264 | 11264 |
| 8265 | 9265 | 10265 | 11265 |
| 8266 | 9266 | 10266 | 11266 |
| 8267 | 9267 | 10267 | 11267 |
| 8268 | 9268 | 10268 | 11268 |
| 8269 | 9269 | 10269 | 11269 |
| 8270 | 9270 | 10270 | 11270 |
| 8271 | 9271 | 10271 | 11271 |
| 8272 | 9272 | 10272 | 11272 |
| 8273 | 9273 | 10273 | 11273 |
| 8274 | 9274 | 10274 | 11274 |
| 8275 | 9275 | 10275 | 11275 |
| 8276 | 9276 | 10276 | 11276 |
| 8277 | 9277 | 10277 | 11277 |
| 8278 | 9278 | 10278 | 11278 |
| 8279 | 9279 | 10279 | 11279 |
| 8280 | 9280 | 10280 | 11280 |
| 8281 | 9281 | 10281 | 11281 |
| 8282 | 9282 | 10282 | 11282 |
| 8283 | 9283 | 10283 | 11283 |
| 8284 | 9284 | 10284 | 11284 |
| 8285 | 9285 | 10285 | 11285 |
| 8286 | 9286 | 10286 | 11286 |
| 8287 | 9287 | 10287 | 11287 |
| 8288 | 9288 | 10288 | 11288 |
| 8289 | 9289 | 10289 | 11289 |
| 8290 | 9290 | 10290 | 11290 |
| 8291 | 9291 | 10291 | 11291 |
| 8292 | 9292 | 10292 | 11292 |
| 8293 | 9293 | 10293 | 11293 |
| 8294 | 9294 | 10294 | 11294 |
| 8295 | 9295 | 10295 | 11295 |
| 8296 | 9296 | 10296 | 11296 |
| 8297 | 9297 | 10297 | 11297 |
| 8298 | 9298 | 10298 | 11298 |
| 8299 | 9299 | 10299 | 11299 |
| 8300 | 9300 | 10300 | 11300 |
| 8301 | 9301 | 10301 | 11301 |
| 8302 | 9302 | 10302 | 11302 |
| 8303 | 9303 | 10303 | 11303 |
| 8304 | 9304 | 10304 | 11304 |
| 8305 | 9305 | 10305 | 11305 |
| 8306 | 9306 | 10306 | 11306 |
| 8307 | 9307 | 10307 | 11307 |
| 8308 | 9308 | 10308 | 11308 |
| 8309 | 9309 | 10309 | 11309 |
| 8310 | 9310 | 10310 | 11310 |
| 8311 | 9311 | 10311 | 11311 |
| 8312 | 9312 | 10312 | 11312 |
| 8313 | 9313 | 10313 | 11313 |
| 8314 | 9314 | 10314 | 11314 |
| 8315 | 9315 | 10315 | 11315 |
| 8316 | 9316 | 10316 | 11316 |
| 8317 | 9317 | 10317 | 11317 |
| 8318 | 9318 | 10318 | 11318 |
| 8319 | 9319 | 10319 | 11319 |
| 8320 | 9320 | 10320 | 11320 |
| 8321 | 9321 | 10321 | 11321 |
| 8322 | 9322 | 10322 | 11322 |
| 8323 | 9323 | 10323 | 11323 |
| 8324 | 9324 | 10324 | 11324 |
| 8325 | 9325 | 10325 | 11325 |
| 8326 | 9326 | 10326 | 11326 |
| 8327 | 9327 | 10327 | 11327 |
| 8328 | 9328 | 10328 | 11328 |
| 8329 | 9329 | 10329 | 11329 |
| 8330 | 9330 | 10330 | 11330 |
| 8331 | 9331 | 10331 | 11331 |
| 8332 | 9332 | 10332 | 11332 |
| 8333 | 9333 | 10333 | 11333 |
| 8334 | 9334 | 10334 | 11334 |
| 8335 | 9335 | 10335 | 11335 |
| 8336 | 9336 | 10336 | 11336 |
| 8337 | 9337 | 10337 | 11337 |
| 8338 | 9338 | 10338 | 11338 |
| 8339 | 9339 | 10339 | 11339 |
| 8340 | 9340 | 10340 | 11340 |
| 8341 | 9341 | 10341 | 11341 |
| 8342 | 9342 | 10342 | 11342 |
| 8343 | 9343 | 10343 | 11343 |
| 8344 | 9344 | 10344 | 11344 |
| 8345 | 9345 | 10345 | 11345 |
| 8346 | 9346 | 10346 | 11346 |
| 8347 | 9347 | 10347 | 11347 |
| 8348 | 9348 | 10348 | 11348 |
| 8349 | 9349 | 10349 | 11349 |
| 8350 | 9350 | 10350 | 11350 |
| 8351 | 9351 | 10351 | 11351 |
| 8352 | 9352 | 10352 | 11352 |
| 8353 | 9353 | 10353 | 11353 |
| 8354 | 9354 | 10354 | 11354 |
| 8355 | 9355 | 10355 | 11355 |
| 8356 | 9356 | 10356 | 11356 |
| 8357 | 9357 | 10357 | 11357 |
| 8358 | 9358 | 10358 | 11358 |
| 8359 | 9359 | 10359 | 11359 |
| 8360 | 9360 | 10360 | 11360 |
| 8361 | 9361 | 10361 | 11361 |
| 8362 | 9362 | 10362 | 11362 |
| 8363 | 9363 | 10363 | 11363 |
| 8364 | 9364 | 10364 | 11364 |
| 8365 | 9365 | 10365 | 11365 |
| 8366 | 9366 | 10366 | 11366 |
| 8367 | 9367 | 10367 | 11367 |
| 8368 | 9368 | 10368 | 11368 |
| 8369 | 9369 | 10369 | 11369 |
| 8370 | 9370 | 10370 | 11370 |
| 8371 | 9371 | 10371 | 11371 |
| 8372 | 9372 | 10372 | 11372 |
| 8373 | 9373 | 10373 | 11373 |
| 8374 | 9374 | 10374 | 11374 |
| 8375 | 9375 | 10375 | 11375 |
| 8376 | 9376 | 10376 | 11376 |
| 8377 | 9377 | 10377 | 11377 |
| 8378 | 9378 | 10378 | 11378 |
| 8379 | 9379 | 10379 | 11379 |
| 8380 | 9380 | 10380 | 11380 |
| 8381 | 9381 | 10381 | 11381 |
| 8382 | 9382 | 10382 | 11382 |
| 8383 | 9383 | 10383 | 11383 |
| 8384 | 9384 | 10384 | 11384 |
| 8385 | 9385 | 10385 | 11385 |
| 8386 | 9386 | 10386 | 11386 |
| 8387 | 9387 | 10387 | 11387 |
| 8388 | 9388 | 10388 | 11388 |
| 8389 | 9389 | 10389 | 11389 |
| 8390 | 9390 | 10390 | 11390 |
| 8391 | 9391 | 10391 | 11391 |
| 8392 | 9392 | 10392 | 11392 |
| 8393 | 9393 | 10393 | 11393 |
| 8394 | 9394 | 10394 | 11394 |
| 8395 | 9395 | 10395 | 11395 |
| 8396 | 9396 | 10396 | 11396 |
| 8397 | 9397 | 10397 | 11397 |
| 8398 | 9398 | 10398 | 11398 |
| 8399 | 9399 | 10399 | 11399 |
| 8400 | 9400 | 10400 | 11400 |
| 8401 | 9401 | 10401 | 11401 |
| 8402 | 9402 | 10402 | 11402 |
| 8403 | 9403 | 10403 | 11403 |
| 8404 | 9404 | 10404 | 11404 |
| 8405 | 9405 | 10405 | 11405 |
| 8406 | 9406 | 10406 | 11406 |
| 8407 | 9407 | 10407 | 11407 |
| 8408 | 9408 | 10408 | 11408 |
| 8409 | 9409 | 10409 | 11409 |
| 8410 | 9410 | 10410 | 11410 |
| 8411 | 9411 | 10411 | 11411 |
| 8412 | 9412 | 10412 | 11412 |
| 8413 | 9413 | 10413 | 11413 |
| 8414 | 9414 | 10414 | 11414 |
| 8415 | 9415 | 10415 | 11415 |
| 8416 | 9416 | 10416 | 11416 |
| 8417 | 9417 | 10417 | 11417 |
| 8418 | 9418 | 10418 | 11418 |
| 8419 | 9419 | 10419 | 11419 |
| 8420 | 9420 | 10420 | 11420 |
| 8421 | 9421 | 10421 | 11421 |
| 8422 | 9422 | 10422 | 11422 |
| 8423 | 9423 | 10423 | 11423 |
| 8424 | 9424 | 10424 | 11424 |
| 8425 | 9425 | 10425 | 11425 |
| 8426 | 9426 | 10426 | 11426 |
| 8427 | 9427 | 10427 | 11427 |
| 8428 | 9428 | 10428 | 11428 |
| 8429 | 9429 | 10429 | 11429 |
| 8430 | 9430 | 10430 | 11430 |
| 8431 | 9431 | 10431 | 11431 |
| 8432 | 9432 | 10432 | 11432 |
| 8433 | 9433 | 10433 | 11433 |
| 8434 | 9434 | 10434 | 11434 |
| 8435 | 9435 | 10435 | 11435 |
| 8436 | 9436 | 10436 | 11436 |
| 8437 | 9437 | 10437 | 11437 |
| 8438 | 9438 | 10438 | 11438 |
| 8439 | 9439 | 10439 | 11439 |
| 8440 | 9440 | 10440 | 11440 |
| 8441 | 9441 | 10441 | 11441 |
| 8442 | 9442 | 10442 | 11442 |
| 8443 | 9443 | 10443 | 11443 |
| 8444 | 9444 | 10444 | 11444 |
| 8445 | 9445 | 10445 | 11445 |
| 8446 | 9446 | 10446 | 11446 |
| 8447 | 9447 | 10447 | 11447 |
| 8448 | 9448 | 10448 | 11448 |
| 8449 | 9449 | 10449 | 11449 |
| 8450 | 9450 | 10450 | 11450 |
| 8451 | 9451 | 10451 | 11451 |
| 8452 | 9452 | 10452 | 11452 |
| 8453 | 9453 | 10453 | 11453 |
| 8454 | 9454 | 10454 | 11454 |
| 8455 | 9455 | 10455 | 11455 |
| 8456 | 9456 | 10456 | 11456 |
| 8457 | 9457 | 10457 | 11457 |
| 8458 | 9458 | 10458 | 11458 |
| 8459 | 9459 | 10459 | 11459 |
| 8460 | 9460 | 10460 | 11460 |
| 8461 | 9461 | 10461 | 11461 |
| 8462 | 9462 | 10462 | 11462 |
| 8463 | 9463 | 10463 | 11463 |
| 8464 | 9464 | 10464 | 11464 |
| 8465 | 9465 | 10465 | 11465 |
| 8466 | 9466 | 10466 | 11466 |
| 8467 | 9467 | 10467 | 11467 |
| 8468 | 9468 | 10468 | 11468 |
| 8469 | 9469 | 10469 | 11469 |
| 8470 | 9470 | 10470 | 11470 |
| 8471 | 9471 | 10471 | 11471 |
| 8472 | 9472 | 10472 | 11472 |
| 8473 | 9473 | 10473 | 11473 |
| 8474 | 9474 | 10474 | 11474 |
| 8475 | 9475 | 10475 | 11475 |
| 8476 | 9476 | 10476 | 11476 |
| 8477 | 9477 | 10477 | 11477 |
| 8478 | 9478 | 10478 | 11478 |
| 8479 | 9479 | 10479 | 11479 |
| 8480 | 9480 | 10480 | 11480 |
| 8481 | 9481 | 10481 | 11481 |
| 8482 | 9482 | 10482 | 11482 |
| 8483 | 9483 | 10483 | 11483 |
| 8484 | 9484 | 10484 | 11484 |
| 8485 | 9485 | 10485 | 11485 |
| 8486 | 9486 | 10486 | 11486 |
| 8487 | 9487 | 10487 | 11487 |
| 8488 | 9488 | 10488 | 11488 |
| 8489 | 9489 | 10489 | 11489 |
| 8490 | 9490 | 10490 | 11490 |
| 8491 | 9491 | 10491 | 11491 |
| 8492 | 9492 | 10492 | 11492 |
| 8493 | 9493 | 10493 | 11493 |
| 8494 | 9494 | 10494 | 11494 |
| 8495 | 9495 | 10495 | 11495 |
| 8496 | 9496 | 10496 | 11496 |
| 8497 | 9497 | 10497 | 11497 |
| 8498 | 9498 | 10498 | 11498 |
| 8499 | 9499 | 10499 | 11499 |
| 8500 | 9500 | 10500 | 11500 |
| 8501 | 9501 | 10501 | 11501 |
| 8502 | 9502 | 10502 | 11502 |
| 8503 | 9503 | 10503 | 11503 |
| 8504 | 9504 | 10504 | 11504 |
| 8505 | 9505 | 10505 | 11505 |
| 8506 | 9506 | 10506 | 11506 |
| 8507 | 9507 | 10507 | 11507 |
| 8508 | 9508 | 10508 | 11508 |
| 8509 | 9509 | 10509 | 11509 |
| 8510 | 9510 | 10510 | 11510 |
| 8511 | 9511 | 10511 | 11511 |
| 8512 | 9512 | 10512 | 11512 |
| 8513 | 9513 | 10513 | 11513 |
| 8514 | 9514 | 10514 | 11514 |
| 8515 | 9515 | 10515 | 11515 |
| 8516 | 9516 | 10516 | 11516 |
| 8517 | 9517 | 10517 | 11517 |
| 8518 | 9518 | 10518 | 11518 |
| 8519 | 9519 | 10519 | 11519 |
| 8520 | 9520 | 10520 | 11520 |
| 8521 | 9521 | 10521 | 11521 |
| 8522 | 9522 | 10522 | 11522 |
| 8523 | 9523 | 10523 | 11523 |
| 8524 | 9524 | 10524 | 11524 |
| 8525 | 9525 | 10525 | 11525 |
| 8526 | 9526 | 10526 | 11526 |
| 8527 | 9527 | 10527 | 11527 |
| 8528 | 9528 | 10528 | 11528 |
| 8529 | 9529 | 10529 | 11529 |
| 8530 | 9530 | 10530 | 11530 |
| 8531 | 9531 | 10531 | 11531 |
| 8532 | 9532 | 10532 | 11532 |
| 8533 | 9533 | 10533 | 11533 |
| 8534 | 9534 | 10534 | 11534 |
| 8535 | 9535 | 10535 | 11535 |
| 8536 | 9536 | 10536 | 11536 |
| 8537 | 9537 | 10537 | 11537 |
| 8538 | 9538 | 10538 | 11538 |
| 8539 | 9539 | 10539 | 11539 |
| 8540 | 9540 | 10540 | 11540 |
| 8541 | 9541 | 10541 | 11541 |
| 8542 | 9542 | 10542 | 11542 |
| 8543 | 9543 | 10543 | 11543 |
| 8544 | 9544 | 10544 | 11544 |
| 8545 | 9545 | 10545 | 11545 |
| 8546 | 9546 | 10546 | 11546 |
| 8547 | 9547 | 10547 | 11547 |
| 8548 | 9548 | 10548 | 11548 |
| 8549 | 9549 | 10549 | 11549 |
| 8550 | 9550 | 10550 | 11550 |
| 8551 | 9551 | 10551 | 11551 |
| 8552 | 9552 | 10552 | 11552 |
| 8553 | 9553 | 10553 | 11553 |
| 8554 | 9554 | 10554 | 11554 |
| 8555 | 9555 | 10555 | 11555 |
| 8556 | 9556 | 10556 | 11556 |
| 8557 | 9557 | 10557 | 11557 |
| 8558 | 9558 | 10558 | 11558 |
| 8559 | 9559 | 10559 | 11559 |
| 8560 | 9560 | 10560 | 11560 |
| 8561 | 9561 | 10561 | 11561 |
| 8562 | 9562 | 10562 | 11562 |
| 8563 | 9563 | 10563 | 11563 |
| 8564 | 9564 | 10564 | 11564 |
| 8565 | 9565 | 10565 | 11565 |
| 8566 | 9566 | 10566 | 11566 |
| 8567 | 9567 | 10567 | 11567 |
| 8568 | 9568 | 10568 | 11568 |
| 8569 | 9569 | 10569 | 11569 |
| 8570 | 9570 | 10570 | 11570 |
| 8571 | 9571 | 10571 | 11571 |
| 8572 | 9572 | 10572 | 11572 |
| 8573 | 9573 | 10573 | 11573 |
| 8574 | 9574 | 10574 | 11574 |
| 8575 | 9575 | 10575 | 11575 |
| 8576 | 9576 | 10576 | 11576 |
| 8577 | 9577 | 10577 | 11577 |
| 8578 | 9578 | 10578 | 11578 |
| 8579 | 9579 | 10579 | 11579 |
| 8580 | 9580 | 10580 | 11580 |
| 8581 | 9581 | 10581 | 11581 |
| 8582 | 9582 | 10582 | 11582 |
| 8583 | 9583 | 10583 | 11583 |
| 8584 | 9584 | 10584 | 11584 |
| 8585 | 9585 | 10585 | 11585 |
| 8586 | 9586 | 10586 | 11586 |
| 8587 | 9587 | 10587 | 11587 |
| 8588 | 9588 | 10588 | 11588 |
| 8589 | 9589 | 10589 | 11589 |
| 8590 | 9590 | 10590 | 11590 |
| 8591 | 9591 | 10591 | 11591 |
| 8592 | 9592 | 10592 | 11592 |
| 8593 | 9593 | 10593 | 11593 |
| 8594 | 9594 | 10594 | 11594 |
| 8595 | 9595 | 10595 | 11595 |
| 8596 | 9596 | 10596 | 11596 |
| 8597 | 9597 | 10597 | 11597 |
| 8598 | 9598 | 10598 | 11598 |
| 8599 | 9599 | 10599 | 11599 |
| 8600 | 9600 | 10600 | 11600 |
| 8601 | 9601 | 10601 | 11601 |
| 8602 | 9602 | 10602 | 11602 |
| 8603 | 9603 | 10603 | 11603 |
| 8604 | 9604 | 10604 | 11604 |
| 8605 | 9605 | 10605 | 11605 |
| 8606 | 9606 | 10606 | 11606 |
| 8607 | 9607 | 10607 | 11607 |
| 8608 | 9608 | 10608 | 11608 |
| 8609 | 9609 | 10609 | 11609 |
| 8610 | 9610 | 10610 | 11610 |
| 8611 | 9611 | 10611 | 11611 |
| 8612 | 9612 | 10612 | 11612 |
| 8613 | 9613 | 10613 | 11613 |
| 8614 | 9614 | 10614 | 11614 |
| 8615 | 9615 | 10615 | 11615 |
| 8616 | 9616 | 10616 | 11616 |
| 8617 | 9617 | 10617 | 11617 |
| 8618 | 9618 | 10618 | 11618 |
| 8619 | 9619 | 10619 | 11619 |
| 8620 | 9620 | 10620 | 11620 |
| 8621 | 9621 | 10621 | 11621 |
| 8622 | 9622 | 10622 | 11622 |
| 8623 | 9623 | 10623 | 11623 |
| 8624 | 9624 | 10624 | 11624 |
| 8625 | 9625 | 10625 | 11625 |
| 8626 | 9626 | 10626 | 11626 |
| 8627 | 9627 | 10627 | 11627 |
| 8628 | 9628 | 10628 | 11628 |
| 8629 | 9629 | 10629 | 11629 |
| 8630 | 9630 | 10630 | 11630 |
| 8631 | 9631 | 10631 | 11631 |
| 8632 | 9632 | 10632 | 11632 |
| 8633 | 9633 | 10633 | 11633 |
| 8634 | 9634 | 10634 | 11634 |
| 8635 | 9635 | 10635 | 11635 |
| 8636 | 9636 | 10636 | 11636 |
| 8637 | 9637 | 10637 | 11637 |
| 8638 | 9638 | 10638 | 11638 |
| 8639 | 9639 | 10639 | 11639 |
| 8640 | 9640 | 10640 | 11640 |
| 8641 | 9641 | 10641 | 11641 |
| 8642 | 9642 | 10642 | 11642 |
| 8643 | 9643 | 10643 | 11643 |
| 8644 | 9644 | 10644 | 11644 |
| 8645 | 9645 | 10645 | 11645 |
| 8646 | 9646 | 10646 | 11646 |
| 8647 | 9647 | 10647 | 11647 |
| 8648 | 9648 | 10648 | 11648 |
| 8649 | 9649 | 10649 | 11649 |
| 8650 | 9650 | 10650 | 11650 |
| 8651 | 9651 | 10651 | 11651 |
| 8652 | 9652 | 10652 | 11652 |
| 8653 | 9653 | 10653 | 11653 |
| 8654 | 9654 | 10654 | 11654 |
| 8655 | 9655 | 10655 | 11655 |
| 8656 | 9656 | 10656 | 11656 |
| 8657 | 9657 | 10657 | 11657 |
| 8658 | 9658 | 10658 | 11658 |
| 8659 | 9659 | 10659 | 11659 |
| 8660 | 9660 | 10660 | 11660 |
| 8661 | 9661 | 10661 | 11661 |
| 8662 | 9662 | 10662 | 11662 |
| 8663 | 9663 | 10663 | 11663 |
| 8664 | 9664 | 10664 | 11664 |
| 8665 | 9665 | 10665 | 11665 |
| 8666 | 9666 | 10666 | 11666 |
| 8667 | 9667 | 10667 | 11667 |
| 8668 | 9668 | 10668 | 11668 |
| 8669 | 9669 | 10669 | 11669 |
| 8670 | 9670 | 10670 | 11670 |
| 8671 | 9671 | 10671 | 11671 |
| 8672 | 9672 | 10672 | 11672 |
| 8673 | 9673 | 10673 | 11673 |
| 8674 | 9674 | 10674 | 11674 |
| 8675 | 9675 | 10675 | 11675 |
| 8676 | 9676 | 10676 | 11676 |
| 8677 | 9677 | 10677 | 11677 |
| 8678 | 9678 | 10678 | 11678 |
| 8679 | 9679 | 10679 | 11679 |
| 8680 | 9680 | 10680 | 11680 |
| 8681 | 9681 | 10681 | 11681 |
| 8682 | 9682 | 10682 | 11682 |
| 8683 | 9683 | 10683 | 11683 |
| 8684 | 9684 | 10684 | 11684 |
| 8685 | 9685 | 10685 | 11685 |
| 8686 | 9686 | 10686 | 11686 |
| 8687 | 9687 | 10687 | 11687 |
| 8688 | 9688 | 10688 | 11688 |
| 8689 | 9689 | 10689 | 11689 |
| 8690 | 9690 | 10690 | 11690 |
| 8691 | 9691 | 10691 | 11691 |
| 8692 | 9692 | 10692 | 11692 |
| 8693 | 9693 | 10693 | 11693 |
| 8694 | 9694 | 10694 | 11694 |
| 8695 | 9695 | 10695 | 11695 |
| 8696 | 9696 | 10696 | 11696 |
| 8697 | 9697 | 10697 | 11697 |
| 8698 | 9698 | 10698 | 11698 |
| 8699 | 9699 | 10699 | 11699 |
| 8700 | 9700 | 10700 | 11700 |
| 8701 | 9701 | 10701 | 11701 |
| 8702 | 9702 | 10702 | 11702 |
| 8703 | 9703 | 10703 | 11703 |
| 8704 | 9704 | 10704 | 11704 |
| 8705 | 9705 | 10705 | 11705 |
| 8706 | 9706 | 10706 | 11706 |
| 8707 | 9707 | 10707 | 11707 |
| 8708 | 9708 | 10708 | 11708 |
| 8709 | 9709 | 10709 | 11709 |
| 8710 | 9710 | 10710 | 11710 |
| 8711 | 9711 | 10711 | 11711 |
| 8712 | 9712 | 10712 | 11712 |
| 8713 | 9713 | 10713 | 11713 |
| 8714 | 9714 | 10714 | 11714 |
| 8715 | 9715 | 10715 | 11715 |
| 8716 | 9716 | 10716 | 11716 |
| 8717 | 9717 | 10717 | 11717 |
| 8718 | 9718 | 10718 | 11718 |
| 8719 | 9719 | 10719 | 11719 |
| 8720 | 9720 | 10720 | 11720 |
| 8721 | 9721 | 10721 | 11721 |
| 8722 | 9722 | 10722 | 11722 |
| 8723 | 9723 | 10723 | 11723 |
| 8724 | 9724 | 10724 | 11724 |
| 8725 | 9725 | 10725 | 11725 |
| 8726 | 9726 | 10726 | 11726 |
| 8727 | 9727 | 10727 | 11727 |
| 8728 | 9728 | 10728 | 11728 |
| 8729 | 9729 | 10729 | 11729 |
| 8730 | 9730 | 10730 | 11730 |
| 8731 | 9731 | 10731 | 11731 |
| 8732 | 9732 | 10732 | 11732 |
| 8733 | 9733 | 10733 | 11733 |
| 8734 | 9734 | 10734 | 11734 |
| 8735 | 9735 | 10735 | 11735 |
| 8736 | 9736 | 10736 | 11736 |
| 8737 | 9737 | 10737 | 11737 |
| 8738 | 9738 | 10738 | 11738 |
| 8739 | 9739 | 10739 | 11739 |
| 8740 | 9740 | 10740 | 11740 |
| 8741 | 9741 | 10741 | 11741 |
| 8742 | 9742 | 10742 | 11742 |
| 8743 | 9743 | 10743 | 11743 |
| 8744 | 9744 | 10744 | 11744 |
| 8745 | 9745 | 10745 | 11745 |
| 8746 | 9746 | 10746 | 11746 |
| 8747 | 9747 | 10747 | 11747 |
| 8748 | 9748 | 10748 | 11748 |
| 8749 | 9749 | 10749 | 11749 |
| 8750 | 9750 | 10750 | 11750 |
| 8751 | 9751 | 10751 | 11751 |
| 8752 | 9752 | 10752 | 11752 |
| 8753 | 9753 | 10753 | 11753 |
| 8754 | 9754 | 10754 | 11754 |
| 8755 | 9755 | 10755 | 11755 |
| 8756 | 9756 | 10756 | 11756 |
| 8757 | 9757 | 10757 | 11757 |
| 8758 | 9758 | 10758 | 11758 |
| 8759 | 9759 | 10759 | 11759 |
| 8760 | 9760 | 10760 | 11760 |
| 8761 | 9761 | 10761 | 11761 |
| 8762 | 9762 | 10762 | 11762 |
| 8763 | 9763 | 10763 | 11763 |
| 8764 | 9764 | 10764 | 11764 |
| 8765 | 9765 | 10765 | 11765 |
| 8766 | 9766 | 10766 | 11766 |
| 8767 | 9767 | 10767 | 11767 |
| 8768 | 9768 | 10768 | 11768 |
| 8769 | 9769 | 10769 | 11769 |
| 8770 | 9770 | 10770 | 11770 |
| 8771 | 9771 | 10771 | 11771 |
| 8772 | 9772 | 10772 | 11772 |
| 8773 | 9773 | 10773 | 11773 |
| 8774 | 9774 | 10774 | 11774 |
| 8775 | 9775 | 10775 | 11775 |
| 8776 | 9776 | 10776 | 11776 |
| 8777 | 9777 | 10777 | 11777 |
| 8778 | 9778 | 10778 | 11778 |
| 8779 | 9779 | 10779 | 11779 |
| 8780 | 9780 | 10780 | 11780 |
| 8781 | 9781 | 10781 | 11781 |
| 8782 | 9782 | 10782 | 11782 |
| 8783 | 9783 | 10783 | 11783 |
| 8784 | 9784 | 10784 | 11784 |
| 8785 | 9785 | 10785 | 11785 |
| 8786 | 9786 | 10786 | 11786 |
| 8787 | 9787 | 10787 | 11787 |
| 8788 | 9788 | 10788 | 11788 |
| 8789 | 9789 | 10789 | 11789 |
| 8790 | 9790 | 10790 | 11790 |
| 8791 | 9791 | 10791 | 11791 |
| 8792 | 9792 | 10792 | 11792 |
| 8793 | 9793 | 10793 | 11793 |
| 8794 | 9794 | 10794 | 11794 |
| 8795 | 9795 | 10795 | 11795 |
| 8796 | 9796 | 10796 | 11796 |
| 8797 | 9797 | 10797 | 11797 |
| 8798 | 9798 | 10798 | 11798 |
| 8799 | 9799 | 10799 | 11799 |
| 8800 | 9800 | 10800 | 11800 |
| 8801 | 9801 | 10801 | 11801 |
| 8802 | 9802 | 10802 | 11802 |
| 8803 | 9803 | 10803 | 11803 |
| 8804 | 9804 | 10804 | 11804 |
| 8805 | 9805 | 10805 | 11805 |
| 8806 | 9806 | 10806 | 11806 |
| 8807 | 9807 | 10807 | 11807 |
| 8808 | 9808 | 10808 | 11808 |
| 8809 | 9809 | 10809 | 11809 |
| 8810 | 9810 | 10810 | 11810 |
| 8811 | 9811 | 10811 | 11811 |
| 8812 | 9812 | 10812 | 11812 |
| 8813 | 9813 | 10813 | 11813 |
| 8814 | 9814 | 10814 | 11814 |
| 8815 | 9815 | 10815 | 11815 |
| 8816 | 9816 | 10816 | 11816 |
| 8817 | 9817 | 10817 | 11817 |
| 8818 | 9818 | 10818 | 11818 |
| 8819 | 9819 | 10819 | 11819 |
| 8820 | 9820 | 10820 | 11820 |
| 8821 | 9821 | 10821 | 11821 |
| 8822 | 9822 | 10822 | 11822 |
| 8823 | 9823 | 10823 | 11823 |
| 8824 | 9824 | 10824 | 11824 |
| 8825 | 9825 | 10825 | 11825 |
| 8826 | 9826 | 10826 | 11826 |
| 8827 | 9827 | 10827 | 11827 |
| 8828 | 9828 | 10828 | 11828 |
| 8829 | 9829 | 10829 | 11829 |
| 8830 | 9830 | 10830 | 11830 |
| 8831 | 9831 | 10831 | 11831 |
| 8832 | 9832 | 10832 | 11832 |
| 8833 | 9833 | 10833 | 11833 |
| 8834 | 9834 | 10834 | 11834 |
| 8835 | 9835 | 10835 | 11835 |
| 8836 | 9836 | 10836 | 11836 |
| 8837 | 9837 | 10837 | 11837 |
| 8838 | 9838 | 10838 | 11838 |
| 8839 | 9839 | 10839 | 11839 |
| 8840 | 9840 | 10840 | 11840 |
| 8841 | 9841 | 10841 | 11841 |
| 8842 | 9842 | 10842 | 11842 |
| 8843 | 9843 | 10843 | 11843 |
| 8844 | 9844 | 10844 | 11844 |
| 8845 | 9845 | 10845 | 11845 |
| 8846 | 9846 | 10846 | 11846 |
| 8847 | 9847 | 10847 | 11847 |
| 8848 | 9848 | 10848 | 11848 |
| 8849 | 9849 | 10849 | 11849 |
| 8850 | 9850 | 10850 | 11850 |
| 8851 | 9851 | 10851 | 11851 |
| 8852 | 9852 | 10852 | 11852 |
| 8853 | 9853 | 10853 | 11853 |
| 8854 | 9854 | 10854 | 11854 |
| 8855 | 9855 | 10855 | 11855 |
| 8856 | 9856 | 10856 | 11856 |
| 8857 | 9857 | 10857 | 11857 |
| 8858 | 9858 | 10858 | 11858 |
| 8859 | 9859 | 10859 | 11859 |
| 8860 | 9860 | 10860 | 11860 |
| 8861 | 9861 | 10861 | 11861 |
| 8862 | 9862 | 10862 | 11862 |
| 8863 | 9863 | 10863 | 11863 |
| 8864 | 9864 | 10864 | 11864 |
| 8865 | 9865 | 10865 | 11865 |
| 8866 | 9866 | 10866 | 11866 |
| 8867 | 9867 | 10867 | 11867 |
| 8868 | 9868 | 10868 | 11868 |
| 8869 | 9869 | 10869 | 11869 |
| 8870 | 9870 | 10870 | 11870 |
| 8871 | 9871 | 10871 | 11871 |
| 8872 | 9872 | 10872 | 11872 |
| 8873 | 9873 | 10873 | 11873 |
| 8874 | 9874 | 10874 | 11874 |
| 8875 | 9875 | 10875 | 11875 |
| 8876 | 9876 | 10876 | 11876 |
| 8877 | 9877 | 10877 | 11877 |
| 8878 | 9878 | 10878 | 11878 |
| 8879 | 9879 | 10879 | 11879 |
| 8880 | 9880 | 10880 | 11880 |
| 8881 | 9881 | 10881 | 11881 |
| 8882 | 9882 | 10882 | 11882 |
| 8883 | 9883 | 10883 | 11883 |
| 8884 | 9884 | 10884 | 11884 |
| 8885 | 9885 | 10885 | 11885 |
| 8886 | 9886 | 10886 | 11886 |
| 8887 | 9887 | 10887 | 11887 |
| 8888 | 9888 | 10888 | 11888 |
| 8889 | 9889 | 10889 | 11889 |
| 8890 | 9890 | 10890 | 11890 |
| 8891 | 9891 | 10891 | 11891 |
| 8892 | 9892 | 10892 | 11892 |
| 8893 | 9893 | 10893 | 11893 |
| 8894 | 9894 | 10894 | 11894 |
| 8895 | 9895 | 10895 | 11895 |
| 8896 | 9896 | 10896 | 11896 |
| 8897 | 9897 | 10897 | 11897 |
| 8898 | 9898 | 10898 | 11898 |
| 8899 | 9899 | 10899 | 11899 |
| 8900 | 9900 | 10900 | 11900 |
| 8901 | 9901 | 10901 | 11901 |
| 8902 | 9902 | 10902 | 11902 |
| 8903 | 9903 | 10903 | 11903 |
| 8904 | 9904 | 10904 | 11904 |
| 8905 | 9905 | 10905 | 11905 |
| 8906 | 9906 | 10906 | 11906 |
| 8907 | 9907 | 10907 | 11907 |
| 8908 | 9908 | 10908 | 11908 |
| 8909 | 9909 | 10909 | 11909 |
| 8910 | 9910 | 10910 | 11910 |
| 8911 | 9911 | 10911 | 11911 |
| 8912 | 9912 | 10912 | 11912 |
| 8913 | 9913 | 10913 | 11913 |
| 8914 | 9914 | 10914 | 11914 |
| 8915 | 9915 | 10915 | 11915 |
| 8916 | 9916 | 10916 | 11916 |
| 8917 | 9917 | 10917 | 11917 |
| 8918 | 9918 | 10918 | 11918 |
| 8919 | 9919 | 10919 | 11919 |
| 8920 | 9920 | 10920 | 11920 |
| 8921 | 9921 | 10921 | 11921 |
| 8922 | 9922 | 10922 | 11922 |
| 8923 | 9923 | 10923 | 11923 |
| 8924 | 9924 | 10924 | 11924 |
| 8925 | 9925 | 10925 | 11925 |
| 8926 | 9926 | 10926 | 11926 |
| 8927 | 9927 | 10927 | 11927 |
| 8928 | 9928 | 10928 | 11928 |
| 8929 | 9929 | 10929 | 11929 |
| 8930 | 9930 | 10930 | 11930 |
| 8931 | 9931 | 10931 | 11931 |
| 8932 | 9932 | 10932 | 11932 |
| 8933 | 9933 | 10933 | 11933 |
| 8934 | 9934 | 10934 | 11934 |
| 8935 | 9935 | 10935 | 11935 |
| 8936 | 9936 | 10936 | 11936 |
| 8937 | 9937 | 10937 | 11937 |
| 8938 | 9938 | 10938 | 11938 |
| 8939 | 9939 | 10939 | 11939 |
| 8940 | 9940 | 10940 | 11940 |
| 8941 | 9941 | 10941 | 11941 |
| 8942 | 9942 | 10942 | 11942 |
| 8943 | 9943 | 10943 | 11943 |
| 8944 | 9944 | 10944 | 11944 |
| 8945 | 9945 | 10945 | 11945 |
| 8946 | 9946 | 10946 | 11946 |
| 8947 | 9947 | 10947 | 11947 |
| 8948 | 9948 | 10948 | 11948 |
| 8949 | 9949 | 10949 | 11949 |
| 8950 | 9950 | 10950 | 11950 |
| 8951 | 9951 | 10951 | 11951 |
| 8952 | 9952 | 10952 | 11952 |
| 8953 | 9953 | 10953 | 11953 |
| 8954 | 9954 | 10954 | 11954 |
| 8955 | 9955 | 10955 | 11955 |
| 8956 | 9956 | 10956 | 11956 |
| 8957 | 9957 | 10957 | 11957 |
| 8958 | 9958 | 10958 | 11958 |
| 8959 | 9959 | 10959 | 11959 |
| 8960 | 9960 | 10960 | 11960 |
| 8961 | 9961 | 10961 | 11961 |
| 8962 | 9962 | 10962 | 11962 |
| 8963 | 9963 | 10963 | 11963 |
| 8964 | 9964 | 10964 | 11964 |
| 8965 | 9965 | 10965 | 11965 |
| 8966 | 9966 | 10966 | 11966 |
| 8967 | 9967 | 10967 | 11967 |
| 8968 | 9968 | 10968 | 11968 |
| 8969 | 9969 | 10969 | 11969 |
| 8970 | 9970 | 10970 | 11970 |
| 8971 | 9971 | 10971 | 11971 |
| 8972 | 9972 | 10972 | 11972 |
| 8973 | 9973 | 10973 | 11973 |
| 8974 | 9974 | 10974 | 11974 |
| 8975 | 9975 | 10975 | 11975 |
| 8976 | 9976 | 10976 | 11976 |
| 8977 | 9977 | 10977 | 11977 |
| 8978 | 9978 | 10978 | 11978 |
| 8979 | 9979 | 10979 | 11979 |
| 8980 | 9980 | 10980 | 11980 |
| 8981 | 9981 | 10981 | 11981 |
| 8982 | 9982 | 10982 | 11982 |
| 8983 | 9983 | 10983 | 11983 |
| 8984 | 9984 | 10984 | 11984 |
| 8985 | 9985 | 10985 | 11985 |
| 8986 | 9986 | 10986 | 11986 |
| 8987 | 9987 | 10987 | 11987 |
| 8988 | 9988 | 10988 | 11988 |
| 8989 | 9989 | 10989 | 11989 |
| 8990 | 9990 | 10990 | 11990 |
| 8991 | 9991 | 10991 | 11991 |
| 8992 | 9992 | 10992 | 11992 |
| 8993 | 9993 | 10993 | 11993 |
| 8994 | 9994 | 10994 | 11994 |
| 8995 | 9995 | 10995 | 11995 |
| 8996 | 9996 | 10996 | 11996 |
| 8997 | 9997 | 10997 | 11997 |
| 8998 | 9998 | 10998 | 11998 |
| 8999 | 9999 | 10999 | 11999 |
| 9000 | 10000 | 11000 | 12000 |
| 9001 | 10001 | 11001 | 12001 |
| 9002 | 10002 | 11002 | 12002 |

In some embodiments, the sequences that specifically bind to cashew comprise an inner sequence selected from the group consisting of the nucleic acid sequences of SEQ ID NOs. 12003 to 13002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to cashew may comprise a nucleic acid sequence selected from the group consisting of SEQ ID NOs. 13003 to 14002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to cashew may comprise a nucleic acid sequence selected from the group consisting of SEQ ID NOs. 14003 to 15002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to cashew may comprise a nucleic acid sequence selected from the group consisting of SEQ ID NOs. 15003 to 16002. In one embodiment, the aptamer of the present disclosure that binds to cashew may comprise a nucleic acid sequence selected from the group consisting of SEQ ID NOs. 12003 to 16002 listed in Table 5, or variant thereof.

TABLE 5
Aptamer sequences against cashew
Aptamer Sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 12003 | 13003 | 14003 | 15003 |
| 12004 | 13004 | 14004 | 15004 |
| 12005 | 13005 | 14005 | 15005 |
| 12006 | 13006 | 14006 | 15006 |
| 12007 | 13007 | 14007 | 15007 |
| 12008 | 13008 | 14008 | 15008 |
| 12009 | 13009 | 14009 | 15009 |
| 12010 | 13010 | 14010 | 15010 |
| 12011 | 13011 | 14011 | 15011 |
| 12012 | 13012 | 14012 | 15012 |
| 12013 | 13013 | 14013 | 15013 |
| 12014 | 13014 | 14014 | 15014 |
| 12015 | 13015 | 14015 | 15015 |
| 12016 | 13016 | 14016 | 15016 |
| 12017 | 13017 | 14017 | 15017 |
| 12018 | 13018 | 14018 | 15018 |
| 12019 | 13019 | 14019 | 15019 |
| 12020 | 13020 | 14020 | 15020 |
| 12021 | 13021 | 14021 | 15021 |
| 12022 | 13022 | 14022 | 15022 |
| 12023 | 13023 | 14023 | 15023 |
| 12024 | 13024 | 14024 | 15024 |
| 12025 | 13025 | 14025 | 15025 |
| 12026 | 13026 | 14026 | 15026 |
| 12027 | 13027 | 14027 | 15027 |
| 12028 | 13028 | 14028 | 15028 |
| 12029 | 13029 | 14029 | 15029 |
| 12030 | 13030 | 14030 | 15030 |
| 12031 | 13031 | 14031 | 15031 |
| 12032 | 13032 | 14032 | 15032 |
| 12033 | 13033 | 14033 | 15033 |
| 12034 | 13034 | 14034 | 15034 |
| 12035 | 13035 | 14035 | 15035 |
| 12036 | 13036 | 14036 | 15036 |
| 12037 | 13037 | 14037 | 15037 |
| 12038 | 13038 | 14038 | 15038 |
| 12039 | 13039 | 14039 | 15039 |
| 12040 | 13040 | 14040 | 15040 |
| 12041 | 13041 | 14041 | 15041 |
| 12042 | 13042 | 14042 | 15042 |
| 12043 | 13043 | 14043 | 15043 |
| 12044 | 13044 | 14044 | 15044 |
| 12045 | 13045 | 14045 | 15045 |
| 12046 | 13046 | 14046 | 15046 |
| 12047 | 13047 | 14047 | 15047 |
| 12048 | 13048 | 14048 | 15048 |
| 12049 | 13049 | 14049 | 15049 |
| 12050 | 13050 | 14050 | 15050 |
| 12051 | 13051 | 14051 | 15051 |
| 12052 | 13052 | 14052 | 15052 |
| 12053 | 13053 | 14053 | 15053 |
| 12054 | 13054 | 14054 | 15054 |
| 12055 | 13055 | 14055 | 15055 |
| 12056 | 13056 | 14056 | 15056 |
| 12057 | 13057 | 14057 | 15057 |
| 12058 | 13058 | 14058 | 15058 |
| 12059 | 13059 | 14059 | 15059 |
| 12060 | 13060 | 14060 | 15060 |
| 12061 | 13061 | 14061 | 15061 |
| 12062 | 13062 | 14062 | 15062 |
| 12063 | 13063 | 14063 | 15063 |
| 12064 | 13064 | 14064 | 15064 |
| 12065 | 13065 | 14065 | 15065 |
| 12066 | 13066 | 14066 | 15066 |
| 12067 | 13067 | 14067 | 15067 |
| 12068 | 13068 | 14068 | 15068 |
| 12069 | 13069 | 14069 | 15069 |
| 12070 | 13070 | 14070 | 15070 |
| 12071 | 13071 | 14071 | 15071 |
| 12072 | 13072 | 14072 | 15072 |
| 12073 | 13073 | 14073 | 15073 |
| 12074 | 13074 | 14074 | 15074 |
| 12075 | 13075 | 14075 | 15075 |
| 12076 | 13076 | 14076 | 15076 |
| 12077 | 13077 | 14077 | 15077 |
| 12078 | 13078 | 14078 | 15078 |
| 12079 | 13079 | 14079 | 15079 |
| 12080 | 13080 | 14080 | 15080 |
| 12081 | 13081 | 14081 | 15081 |
| 12082 | 13082 | 14082 | 15082 |
| 12083 | 13083 | 14083 | 15083 |
| 12084 | 13084 | 14084 | 15084 |
| 12085 | 13085 | 14085 | 15085 |
| 12086 | 13086 | 14086 | 15086 |
| 12087 | 13087 | 14087 | 15087 |
| 12088 | 13088 | 14088 | 15088 |
| 12089 | 13089 | 14089 | 15089 |
| 12090 | 13090 | 14090 | 15090 |
| 12091 | 13091 | 14091 | 15091 |
| 12092 | 13092 | 14092 | 15092 |
| 12093 | 13093 | 14093 | 15093 |
| 12094 | 13094 | 14094 | 15094 |
| 12095 | 13095 | 14095 | 15095 |
| 12096 | 13096 | 14096 | 15096 |
| 12097 | 13097 | 14097 | 15097 |
| 12098 | 13098 | 14098 | 15098 |
| 12099 | 13099 | 14099 | 15099 |
| 12100 | 13100 | 14100 | 15100 |
| 12101 | 13101 | 14101 | 15101 |
| 12102 | 13102 | 14102 | 15102 |
| 12103 | 13103 | 14103 | 15103 |
| 12104 | 13104 | 14104 | 15104 |
| 12105 | 13105 | 14105 | 15105 |
| 12106 | 13106 | 14106 | 15106 |
| 12107 | 13107 | 14107 | 15107 |
| 12108 | 13108 | 14108 | 15108 |
| 12109 | 13109 | 14109 | 15109 |
| 12110 | 13110 | 14110 | 15110 |
| 12111 | 13111 | 14111 | 15111 |
| 12112 | 13112 | 14112 | 15112 |
| 12113 | 13113 | 14113 | 15113 |
| 12114 | 13114 | 14114 | 15114 |
| 12115 | 13115 | 14115 | 15115 |
| 12116 | 13116 | 14116 | 15116 |
| 12117 | 13117 | 14117 | 15117 |
| 12118 | 13118 | 14118 | 15118 |
| 12119 | 13119 | 14119 | 15119 |
| 12120 | 13120 | 14120 | 15120 |
| 12121 | 13121 | 14121 | 15121 |
| 12122 | 13122 | 14122 | 15122 |
| 12123 | 13123 | 14123 | 15123 |
| 12124 | 13124 | 14124 | 15124 |
| 12125 | 13125 | 14125 | 15125 |
| 12126 | 13126 | 14126 | 15126 |
| 12127 | 13127 | 14127 | 15127 |
| 12128 | 13128 | 14128 | 15128 |
| 12129 | 13129 | 14129 | 15129 |
| 12130 | 13130 | 14130 | 15130 |
| 12131 | 13131 | 14131 | 15131 |
| 12132 | 13132 | 14132 | 15132 |
| 12133 | 13133 | 14133 | 15133 |
| 12134 | 13134 | 14134 | 15134 |
| 12135 | 13135 | 14135 | 15135 |
| 12136 | 13136 | 14136 | 15136 |
| 12137 | 13137 | 14137 | 15137 |
| 12138 | 13138 | 14138 | 15138 |
| 12139 | 13139 | 14139 | 15139 |
| 12140 | 13140 | 14140 | 15140 |
| 12141 | 13141 | 14141 | 15141 |
| 12142 | 13142 | 14142 | 15142 |
| 12143 | 13143 | 14143 | 15143 |
| 12144 | 13144 | 14144 | 15144 |
| 12145 | 13145 | 14145 | 15145 |
| 12146 | 13146 | 14146 | 15146 |
| 12147 | 13147 | 14147 | 15147 |
| 12148 | 13148 | 14148 | 15148 |
| 12149 | 13149 | 14149 | 15149 |
| 12150 | 13150 | 14150 | 15150 |
| 12151 | 13151 | 14151 | 15151 |
| 12152 | 13152 | 14152 | 15152 |
| 12153 | 13153 | 14153 | 15153 |
| 12154 | 13154 | 14154 | 15154 |
| 12155 | 13155 | 14155 | 15155 |
| 12156 | 13156 | 14156 | 15156 |
| 12157 | 13157 | 14157 | 15157 |
| 12158 | 13158 | 14158 | 15158 |
| 12159 | 13159 | 14159 | 15159 |
| 12160 | 13160 | 14160 | 15160 |
| 12161 | 13161 | 14161 | 15161 |
| 12162 | 13162 | 14162 | 15162 |
| 12163 | 13163 | 14163 | 15163 |
| 12164 | 13164 | 14164 | 15164 |
| 12165 | 13165 | 14165 | 15165 |
| 12166 | 13166 | 14166 | 15166 |
| 12167 | 13167 | 14167 | 15167 |
| 12168 | 13168 | 14168 | 15168 |
| 12169 | 13169 | 14169 | 15169 |
| 12170 | 13170 | 14170 | 15170 |
| 12171 | 13171 | 14171 | 15171 |
| 12172 | 13172 | 14172 | 15172 |
| 12173 | 13173 | 14173 | 15173 |
| 12174 | 13174 | 14174 | 15174 |
| 12175 | 13175 | 14175 | 15175 |
| 12176 | 13176 | 14176 | 15176 |
| 12177 | 13177 | 14177 | 15177 |
| 12178 | 13178 | 14178 | 15178 |
| 12179 | 13179 | 14179 | 15179 |
| 12180 | 13180 | 14180 | 15180 |
| 12181 | 13181 | 14181 | 15181 |
| 12182 | 13182 | 14182 | 15182 |
| 12183 | 13183 | 14183 | 15183 |
| 12184 | 13184 | 14184 | 15184 |
| 12185 | 13185 | 14185 | 15185 |
| 12186 | 13186 | 14186 | 15186 |
| 12187 | 13187 | 14187 | 15187 |
| 12188 | 13188 | 14188 | 15188 |
| 12189 | 13189 | 14189 | 15189 |
| 12190 | 13190 | 14190 | 15190 |
| 12191 | 13191 | 14191 | 15191 |
| 12192 | 13192 | 14192 | 15192 |
| 12193 | 13193 | 14193 | 15193 |
| 12194 | 13194 | 14194 | 15194 |
| 12195 | 13195 | 14195 | 15195 |
| 12196 | 13196 | 14196 | 15196 |
| 12197 | 13197 | 14197 | 15197 |
| 12198 | 13198 | 14198 | 15198 |
| 12199 | 13199 | 14199 | 15199 |
| 12200 | 13200 | 14200 | 15200 |
| 12201 | 13201 | 14201 | 15201 |
| 12202 | 13202 | 14202 | 15202 |
| 12203 | 13203 | 14203 | 15203 |
| 12204 | 13204 | 14204 | 15204 |
| 12205 | 13205 | 14205 | 15205 |
| 12206 | 13206 | 14206 | 15206 |
| 12207 | 13207 | 14207 | 15207 |
| 12208 | 13208 | 14208 | 15208 |
| 12209 | 13209 | 14209 | 15209 |
| 12210 | 13210 | 14210 | 15210 |
| 12211 | 13211 | 14211 | 15211 |
| 12212 | 13212 | 14212 | 15212 |
| 12213 | 13213 | 14213 | 15213 |
| 12214 | 13214 | 14214 | 15214 |
| 12215 | 13215 | 14215 | 15215 |
| 12216 | 13216 | 14216 | 15216 |
| 12217 | 13217 | 14217 | 15217 |
| 12218 | 13218 | 14218 | 15218 |
| 12219 | 13219 | 14219 | 15219 |
| 12220 | 13220 | 14220 | 15220 |
| 12221 | 13221 | 14221 | 15221 |
| 12222 | 13222 | 14222 | 15222 |
| 12223 | 13223 | 14223 | 15223 |
| 12224 | 13224 | 14224 | 15224 |
| 12225 | 13225 | 14225 | 15225 |
| 12226 | 13226 | 14226 | 15226 |
| 12227 | 13227 | 14227 | 15227 |
| 12228 | 13228 | 14228 | 15228 |
| 12229 | 13229 | 14229 | 15229 |
| 12230 | 13230 | 14230 | 15230 |
| 12231 | 13231 | 14231 | 15231 |
| 12232 | 13232 | 14232 | 15232 |
| 12233 | 13233 | 14233 | 15233 |
| 12234 | 13234 | 14234 | 15234 |
| 12235 | 13235 | 14235 | 15235 |
| 12236 | 13236 | 14236 | 15236 |
| 12237 | 13237 | 14237 | 15237 |
| 12238 | 13238 | 14238 | 15238 |
| 12239 | 13239 | 14239 | 15239 |
| 12240 | 13240 | 14240 | 15240 |
| 12241 | 13241 | 14241 | 15241 |
| 12242 | 13242 | 14242 | 15242 |
| 12243 | 13243 | 14243 | 15243 |
| 12244 | 13244 | 14244 | 15244 |
| 12245 | 13245 | 14245 | 15245 |
| 12246 | 13246 | 14246 | 15246 |
| 12247 | 13247 | 14247 | 15247 |
| 12248 | 13248 | 14248 | 15248 |
| 12249 | 13249 | 14249 | 15249 |
| 12250 | 13250 | 14250 | 15250 |
| 12251 | 13251 | 14251 | 15251 |
| 12252 | 13252 | 14252 | 15252 |
| 12253 | 13253 | 14253 | 15253 |
| 12254 | 13254 | 14254 | 15254 |
| 12255 | 13255 | 14255 | 15255 |
| 12256 | 13256 | 14256 | 15256 |
| 12257 | 13257 | 14257 | 15257 |
| 12258 | 13258 | 14258 | 15258 |
| 12259 | 13259 | 14259 | 15259 |
| 12260 | 13260 | 14260 | 15260 |
| 12261 | 13261 | 14261 | 15261 |
| 12262 | 13262 | 14262 | 15262 |
| 12263 | 13263 | 14263 | 15263 |
| 12264 | 13264 | 14264 | 15264 |
| 12265 | 13265 | 14265 | 15265 |
| 12266 | 13266 | 14266 | 15266 |
| 12267 | 13267 | 14267 | 15267 |
| 12268 | 13268 | 14268 | 15268 |
| 12269 | 13269 | 14269 | 15269 |
| 12270 | 13270 | 14270 | 15270 |
| 12271 | 13271 | 14271 | 15271 |
| 12272 | 13272 | 14272 | 15272 |
| 12273 | 13273 | 14273 | 15273 |
| 12274 | 13274 | 14274 | 15274 |
| 12275 | 13275 | 14275 | 15275 |
| 12276 | 13276 | 14276 | 15276 |
| 12277 | 13277 | 14277 | 15277 |
| 12278 | 13278 | 14278 | 15278 |
| 12279 | 13279 | 14279 | 15279 |
| 12280 | 13280 | 14280 | 15280 |
| 12281 | 13281 | 14281 | 15281 |
| 12282 | 13282 | 14282 | 15282 |
| 12283 | 13283 | 14283 | 15283 |
| 12284 | 13284 | 14284 | 15284 |
| 12285 | 13285 | 14285 | 15285 |
| 12286 | 13286 | 14286 | 15286 |
| 12287 | 13287 | 14287 | 15287 |
| 12288 | 13288 | 14288 | 15288 |
| 12289 | 13289 | 14289 | 15289 |
| 12290 | 13290 | 14290 | 15290 |
| 12291 | 13291 | 14291 | 15291 |
| 12292 | 13292 | 14292 | 15292 |
| 12293 | 13293 | 14293 | 15293 |
| 12294 | 13294 | 14294 | 15294 |
| 12295 | 13295 | 14295 | 15295 |
| 12296 | 13296 | 14296 | 15296 |
| 12297 | 13297 | 14297 | 15297 |
| 12298 | 13298 | 14298 | 15298 |
| 12299 | 13299 | 14299 | 15299 |
| 12300 | 13300 | 14300 | 15300 |
| 12301 | 13301 | 14301 | 15301 |
| 12302 | 13302 | 14302 | 15302 |
| 12303 | 13303 | 14303 | 15303 |
| 12304 | 13304 | 14304 | 15304 |
| 12305 | 13305 | 14305 | 15305 |
| 12306 | 13306 | 14306 | 15306 |
| 12307 | 13307 | 14307 | 15307 |
| 12308 | 13308 | 14308 | 15308 |
| 12309 | 13309 | 14309 | 15309 |
| 12310 | 13310 | 14310 | 15310 |
| 12311 | 13311 | 14311 | 15311 |
| 12312 | 13312 | 14312 | 15312 |
| 12313 | 13313 | 14313 | 15313 |
| 12314 | 13314 | 14314 | 15314 |
| 12315 | 13315 | 14315 | 15315 |
| 12316 | 13316 | 14316 | 15316 |
| 12317 | 13317 | 14317 | 15317 |
| 12318 | 13318 | 14318 | 15318 |
| 12319 | 13319 | 14319 | 15319 |
| 12320 | 13320 | 14320 | 15320 |
| 12321 | 13321 | 14321 | 15321 |
| 12322 | 13322 | 14322 | 15322 |
| 12323 | 13323 | 14323 | 15323 |
| 12324 | 13324 | 14324 | 15324 |
| 12325 | 13325 | 14325 | 15325 |
| 12326 | 13326 | 14326 | 15326 |
| 12327 | 13327 | 14327 | 15327 |
| 12328 | 13328 | 14328 | 15328 |
| 12329 | 13329 | 14329 | 15329 |
| 12330 | 13330 | 14330 | 15330 |
| 12331 | 13331 | 14331 | 15331 |
| 12332 | 13332 | 14332 | 15332 |
| 12333 | 13333 | 14333 | 15333 |
| 12334 | 13334 | 14334 | 15334 |
| 12335 | 13335 | 14335 | 15335 |
| 12336 | 13336 | 14336 | 15336 |
| 12337 | 13337 | 14337 | 15337 |
| 12338 | 13338 | 14338 | 15338 |
| 12339 | 13339 | 14339 | 15339 |
| 12340 | 13340 | 14340 | 15340 |
| 12341 | 13341 | 14341 | 15341 |
| 12342 | 13342 | 14342 | 15342 |
| 12343 | 13343 | 14343 | 15343 |
| 12344 | 13344 | 14344 | 15344 |
| 12345 | 13345 | 14345 | 15345 |
| 12346 | 13346 | 14346 | 15346 |
| 12347 | 13347 | 14347 | 15347 |
| 12348 | 13348 | 14348 | 15348 |
| 12349 | 13349 | 14349 | 15349 |
| 12350 | 13350 | 14350 | 15350 |
| 12351 | 13351 | 14351 | 15351 |
| 12352 | 13352 | 14352 | 15352 |
| 12353 | 13353 | 14353 | 15353 |
| 12354 | 13354 | 14354 | 15354 |
| 12355 | 13355 | 14355 | 15355 |
| 12356 | 13356 | 14356 | 15356 |
| 12357 | 13357 | 14357 | 15357 |
| 12358 | 13358 | 14358 | 15358 |
| 12359 | 13359 | 14359 | 15359 |
| 12360 | 13360 | 14360 | 15360 |
| 12361 | 13361 | 14361 | 15361 |
| 12362 | 13362 | 14362 | 15362 |
| 12363 | 13363 | 14363 | 15363 |
| 12364 | 13364 | 14364 | 15364 |
| 12365 | 13365 | 14365 | 15365 |
| 12366 | 13366 | 14366 | 15366 |
| 12367 | 13367 | 14367 | 15367 |
| 12368 | 13368 | 14368 | 15368 |
| 12369 | 13369 | 14369 | 15369 |
| 12370 | 13370 | 14370 | 15370 |
| 12371 | 13371 | 14371 | 15371 |
| 12372 | 13372 | 14372 | 15372 |
| 12373 | 13373 | 14373 | 15373 |
| 12374 | 13374 | 14374 | 15374 |
| 12375 | 13375 | 14375 | 15375 |
| 12376 | 13376 | 14376 | 15376 |
| 12377 | 13377 | 14377 | 15377 |
| 12378 | 13378 | 14378 | 15378 |
| 12379 | 13379 | 14379 | 15379 |
| 12380 | 13380 | 14380 | 15380 |
| 12381 | 13381 | 14381 | 15381 |
| 12382 | 13382 | 14382 | 15382 |
| 12383 | 13383 | 14383 | 15383 |
| 12384 | 13384 | 14384 | 15384 |
| 12385 | 13385 | 14385 | 15385 |
| 12386 | 13386 | 14386 | 15386 |
| 12387 | 13387 | 14387 | 15387 |
| 12388 | 13388 | 14388 | 15388 |
| 12389 | 13389 | 14389 | 15389 |
| 12390 | 13390 | 14390 | 15390 |
| 12391 | 13391 | 14391 | 15391 |
| 12392 | 13392 | 14392 | 15392 |
| 12393 | 13393 | 14393 | 15393 |
| 12394 | 13394 | 14394 | 15394 |
| 12395 | 13395 | 14395 | 15395 |
| 12396 | 13396 | 14396 | 15396 |
| 12397 | 13397 | 14397 | 15397 |
| 12398 | 13398 | 14398 | 15398 |
| 12399 | 13399 | 14399 | 15399 |
| 12400 | 13400 | 14400 | 15400 |
| 12401 | 13401 | 14401 | 15401 |
| 12402 | 13402 | 14402 | 15402 |
| 12403 | 13403 | 14403 | 15403 |
| 12404 | 13404 | 14404 | 15404 |
| 12405 | 13405 | 14405 | 15405 |
| 12406 | 13406 | 14406 | 15406 |
| 12407 | 13407 | 14407 | 15407 |
| 12408 | 13408 | 14408 | 15408 |
| 12409 | 13409 | 14409 | 15409 |
| 12410 | 13410 | 14410 | 15410 |
| 12411 | 13411 | 14411 | 15411 |
| 12412 | 13412 | 14412 | 15412 |
| 12413 | 13413 | 14413 | 15413 |
| 12414 | 13414 | 14414 | 15414 |
| 12415 | 13415 | 14415 | 15415 |
| 12416 | 13416 | 14416 | 15416 |
| 12417 | 13417 | 14417 | 15417 |
| 12418 | 13418 | 14418 | 15418 |
| 12419 | 13419 | 14419 | 15419 |
| 12420 | 13420 | 14420 | 15420 |
| 12421 | 13421 | 14421 | 15421 |
| 12422 | 13422 | 14422 | 15422 |
| 12423 | 13423 | 14423 | 15423 |
| 12424 | 13424 | 14424 | 15424 |
| 12425 | 13425 | 14425 | 15425 |
| 12426 | 13426 | 14426 | 15426 |
| 12427 | 13427 | 14427 | 15427 |
| 12428 | 13428 | 14428 | 15428 |
| 12429 | 13429 | 14429 | 15429 |
| 12430 | 13430 | 14430 | 15430 |
| 12431 | 13431 | 14431 | 15431 |
| 12432 | 13432 | 14432 | 15432 |
| 12433 | 13433 | 14433 | 15433 |
| 12434 | 13434 | 14434 | 15434 |
| 12435 | 13435 | 14435 | 15435 |
| 12436 | 13436 | 14436 | 15436 |
| 12437 | 13437 | 14437 | 15437 |
| 12438 | 13438 | 14438 | 15438 |
| 12439 | 13439 | 14439 | 15439 |
| 12440 | 13440 | 14440 | 15440 |
| 12441 | 13441 | 14441 | 15441 |
| 12442 | 13442 | 14442 | 15442 |
| 12443 | 13443 | 14443 | 15443 |
| 12444 | 13444 | 14444 | 15444 |
| 12445 | 13445 | 14445 | 15445 |
| 12446 | 13446 | 14446 | 15446 |
| 12447 | 13447 | 14447 | 15447 |
| 12448 | 13448 | 14448 | 15448 |
| 12449 | 13449 | 14449 | 15449 |
| 12450 | 13450 | 14450 | 15450 |
| 12451 | 13451 | 14451 | 15451 |
| 12452 | 13452 | 14452 | 15452 |
| 12453 | 13453 | 14453 | 15453 |
| 12454 | 13454 | 14454 | 15454 |
| 12455 | 13455 | 14455 | 15455 |
| 12456 | 13456 | 14456 | 15456 |
| 12457 | 13457 | 14457 | 15457 |
| 12458 | 13458 | 14458 | 15458 |
| 12459 | 13459 | 14459 | 15459 |
| 12460 | 13460 | 14460 | 15460 |
| 12461 | 13461 | 14461 | 15461 |
| 12462 | 13462 | 14462 | 15462 |
| 12463 | 13463 | 14463 | 15463 |
| 12464 | 13464 | 14464 | 15464 |
| 12465 | 13465 | 14465 | 15465 |
| 12466 | 13466 | 14466 | 15466 |
| 12467 | 13467 | 14467 | 15467 |
| 12468 | 13468 | 14468 | 15468 |
| 12469 | 13469 | 14469 | 15469 |
| 12470 | 13470 | 14470 | 15470 |
| 12471 | 13471 | 14471 | 15471 |
| 12472 | 13472 | 14472 | 15472 |
| 12473 | 13473 | 14473 | 15473 |
| 12474 | 13474 | 14474 | 15474 |
| 12475 | 13475 | 14475 | 15475 |
| 12476 | 13476 | 14476 | 15476 |
| 12477 | 13477 | 14477 | 15477 |
| 12478 | 13478 | 14478 | 15478 |
| 12479 | 13479 | 14479 | 15479 |
| 12480 | 13480 | 14480 | 15480 |
| 12481 | 13481 | 14481 | 15481 |
| 12482 | 13482 | 14482 | 15482 |
| 12483 | 13483 | 14483 | 15483 |
| 12484 | 13484 | 14484 | 15484 |
| 12485 | 13485 | 14485 | 15485 |
| 12486 | 13486 | 14486 | 15486 |
| 12487 | 13487 | 14487 | 15487 |
| 12488 | 13488 | 14488 | 15488 |
| 12489 | 13489 | 14489 | 15489 |
| 12490 | 13490 | 14490 | 15490 |
| 12491 | 13491 | 14491 | 15491 |
| 12492 | 13492 | 14492 | 15492 |
| 12493 | 13493 | 14493 | 15493 |
| 12494 | 13494 | 14494 | 15494 |
| 12495 | 13495 | 14495 | 15495 |
| 12496 | 13496 | 14496 | 15496 |
| 12497 | 13497 | 14497 | 15497 |
| 12498 | 13498 | 14498 | 15498 |
| 12499 | 13499 | 14499 | 15499 |
| 12500 | 13500 | 14500 | 15500 |
| 12501 | 13501 | 14501 | 15501 |
| 12502 | 13502 | 14502 | 15502 |
| 12503 | 13503 | 14503 | 15503 |
| 12504 | 13504 | 14504 | 15504 |
| 12505 | 13505 | 14505 | 15505 |
| 12506 | 13506 | 14506 | 15506 |
| 12507 | 13507 | 14507 | 15507 |
| 12508 | 13508 | 14508 | 15508 |
| 12509 | 13509 | 14509 | 15509 |
| 12510 | 13510 | 14510 | 15510 |
| 12511 | 13511 | 14511 | 15511 |
| 12512 | 13512 | 14512 | 15512 |
| 12513 | 13513 | 14513 | 15513 |
| 12514 | 13514 | 14514 | 15514 |
| 12515 | 13515 | 14515 | 15515 |
| 12516 | 13516 | 14516 | 15516 |
| 12517 | 13517 | 14517 | 15517 |
| 12518 | 13518 | 14518 | 15518 |
| 12519 | 13519 | 14519 | 15519 |
| 12520 | 13520 | 14520 | 15520 |
| 12521 | 13521 | 14521 | 15521 |
| 12522 | 13522 | 14522 | 15522 |
| 12523 | 13523 | 14523 | 15523 |
| 12524 | 13524 | 14524 | 15524 |
| 12525 | 13525 | 14525 | 15525 |
| 12526 | 13526 | 14526 | 15526 |
| 12527 | 13527 | 14527 | 15527 |
| 12528 | 13528 | 14528 | 15528 |
| 12529 | 13529 | 14529 | 15529 |
| 12530 | 13530 | 14530 | 15530 |
| 12531 | 13531 | 14531 | 15531 |
| 12532 | 13532 | 14532 | 15532 |
| 12533 | 13533 | 14533 | 15533 |
| 12534 | 13534 | 14534 | 15534 |
| 12535 | 13535 | 14535 | 15535 |
| 12536 | 13536 | 14536 | 15536 |
| 12537 | 13537 | 14537 | 15537 |
| 12538 | 13538 | 14538 | 15538 |
| 12539 | 13539 | 14539 | 15539 |
| 12540 | 13540 | 14540 | 15540 |
| 12541 | 13541 | 14541 | 15541 |
| 12542 | 13542 | 14542 | 15542 |
| 12543 | 13543 | 14543 | 15543 |
| 12544 | 13544 | 14544 | 15544 |
| 12545 | 13545 | 14545 | 15545 |
| 12546 | 13546 | 14546 | 15546 |
| 12547 | 13547 | 14547 | 15547 |
| 12548 | 13548 | 14548 | 15548 |
| 12549 | 13549 | 14549 | 15549 |
| 12550 | 13550 | 14550 | 15550 |
| 12551 | 13551 | 14551 | 15551 |
| 12552 | 13552 | 14552 | 15552 |
| 12553 | 13553 | 14553 | 15553 |
| 12554 | 13554 | 14554 | 15554 |
| 12555 | 13555 | 14555 | 15555 |
| 12556 | 13556 | 14556 | 15556 |
| 12557 | 13557 | 14557 | 15557 |
| 12558 | 13558 | 14558 | 15558 |
| 12559 | 13559 | 14559 | 15559 |
| 12560 | 13560 | 14560 | 15560 |
| 12561 | 13561 | 14561 | 15561 |
| 12562 | 13562 | 14562 | 15562 |
| 12563 | 13563 | 14563 | 15563 |
| 12564 | 13564 | 14564 | 15564 |
| 12565 | 13565 | 14565 | 15565 |
| 12566 | 13566 | 14566 | 15566 |
| 12567 | 13567 | 14567 | 15567 |
| 12568 | 13568 | 14568 | 15568 |
| 12569 | 13569 | 14569 | 15569 |
| 12570 | 13570 | 14570 | 15570 |
| 12571 | 13571 | 14571 | 15571 |
| 12572 | 13572 | 14572 | 15572 |
| 12573 | 13573 | 14573 | 15573 |
| 12574 | 13574 | 14574 | 15574 |
| 12575 | 13575 | 14575 | 15575 |
| 12576 | 13576 | 14576 | 15576 |
| 12577 | 13577 | 14577 | 15577 |
| 12578 | 13578 | 14578 | 15578 |
| 12579 | 13579 | 14579 | 15579 |
| 12580 | 13580 | 14580 | 15580 |
| 12581 | 13581 | 14581 | 15581 |
| 12582 | 13582 | 14582 | 15582 |
| 12583 | 13583 | 14583 | 15583 |
| 12584 | 13584 | 14584 | 15584 |
| 12585 | 13585 | 14585 | 15585 |
| 12586 | 13586 | 14586 | 15586 |
| 12587 | 13587 | 14587 | 15587 |
| 12588 | 13588 | 14588 | 15588 |
| 12589 | 13589 | 14589 | 15589 |
| 12590 | 13590 | 14590 | 15590 |
| 12591 | 13591 | 14591 | 15591 |
| 12592 | 13592 | 14592 | 15592 |
| 12593 | 13593 | 14593 | 15593 |
| 12594 | 13594 | 14594 | 15594 |
| 12595 | 13595 | 14595 | 15595 |
| 12596 | 13596 | 14596 | 15596 |
| 12597 | 13597 | 14597 | 15597 |
| 12598 | 13598 | 14598 | 15598 |
| 12599 | 13599 | 14599 | 15599 |
| 12600 | 13600 | 14600 | 15600 |
| 12601 | 13601 | 14601 | 15601 |
| 12602 | 13602 | 14602 | 15602 |
| 12603 | 13603 | 14603 | 15603 |
| 12604 | 13604 | 14604 | 15604 |
| 12605 | 13605 | 14605 | 15605 |
| 12606 | 13606 | 14606 | 15606 |
| 12607 | 13607 | 14607 | 15607 |
| 12608 | 13608 | 14608 | 15608 |
| 12609 | 13609 | 14609 | 15609 |
| 12610 | 13610 | 14610 | 15610 |
| 12611 | 13611 | 14611 | 15611 |
| 12612 | 13612 | 14612 | 15612 |
| 12613 | 13613 | 14613 | 15613 |
| 12614 | 13614 | 14614 | 15614 |
| 12615 | 13615 | 14615 | 15615 |
| 12616 | 13616 | 14616 | 15616 |
| 12617 | 13617 | 14617 | 15617 |
| 12618 | 13618 | 14618 | 15618 |
| 12619 | 13619 | 14619 | 15619 |
| 12620 | 13620 | 14620 | 15620 |
| 12621 | 13621 | 14621 | 15621 |
| 12622 | 13622 | 14622 | 15622 |
| 12623 | 13623 | 14623 | 15623 |
| 12624 | 13624 | 14624 | 15624 |
| 12625 | 13625 | 14625 | 15625 |
| 12626 | 13626 | 14626 | 15626 |
| 12627 | 13627 | 14627 | 15627 |
| 12628 | 13628 | 14628 | 15628 |
| 12629 | 13629 | 14629 | 15629 |
| 12630 | 13630 | 14630 | 15630 |
| 12631 | 13631 | 14631 | 15631 |
| 12632 | 13632 | 14632 | 15632 |
| 12633 | 13633 | 14633 | 15633 |
| 12634 | 13634 | 14634 | 15634 |
| 12635 | 13635 | 14635 | 15635 |
| 12636 | 13636 | 14636 | 15636 |
| 12637 | 13637 | 14637 | 15637 |
| 12638 | 13638 | 14638 | 15638 |
| 12639 | 13639 | 14639 | 15639 |
| 12640 | 13640 | 14640 | 15640 |
| 12641 | 13641 | 14641 | 15641 |
| 12642 | 13642 | 14642 | 15642 |
| 12643 | 13643 | 14643 | 15643 |
| 12644 | 13644 | 14644 | 15644 |
| 12645 | 13645 | 14645 | 15645 |
| 12646 | 13646 | 14646 | 15646 |
| 12647 | 13647 | 14647 | 15647 |
| 12648 | 13648 | 14648 | 15648 |
| 12649 | 13649 | 14649 | 15649 |
| 12650 | 13650 | 14650 | 15650 |
| 12651 | 13651 | 14651 | 15651 |
| 12652 | 13652 | 14652 | 15652 |
| 12653 | 13653 | 14653 | 15653 |
| 12654 | 13654 | 14654 | 15654 |
| 12655 | 13655 | 14655 | 15655 |
| 12656 | 13656 | 14656 | 15656 |
| 12657 | 13657 | 14657 | 15657 |
| 12658 | 13658 | 14658 | 15658 |
| 12659 | 13659 | 14659 | 15659 |
| 12660 | 13660 | 14660 | 15660 |
| 12661 | 13661 | 14661 | 15661 |
| 12662 | 13662 | 14662 | 15662 |
| 12663 | 13663 | 14663 | 15663 |
| 12664 | 13664 | 14664 | 15664 |
| 12665 | 13665 | 14665 | 15665 |
| 12666 | 13666 | 14666 | 15666 |
| 12667 | 13667 | 14667 | 15667 |
| 12668 | 13668 | 14668 | 15668 |
| 12669 | 13669 | 14669 | 15669 |
| 12670 | 13670 | 14670 | 15670 |
| 12671 | 13671 | 14671 | 15671 |
| 12672 | 13672 | 14672 | 15672 |
| 12673 | 13673 | 14673 | 15673 |
| 12674 | 13674 | 14674 | 15674 |
| 12675 | 13675 | 14675 | 15675 |
| 12676 | 13676 | 14676 | 15676 |
| 12677 | 13677 | 14677 | 15677 |
| 12678 | 13678 | 14678 | 15678 |
| 12679 | 13679 | 14679 | 15679 |
| 12680 | 13680 | 14680 | 15680 |
| 12681 | 13681 | 14681 | 15681 |
| 12682 | 13682 | 14682 | 15682 |
| 12683 | 13683 | 14683 | 15683 |
| 12684 | 13684 | 14684 | 15684 |
| 12685 | 13685 | 14685 | 15685 |
| 12686 | 13686 | 14686 | 15686 |
| 12687 | 13687 | 14687 | 15687 |
| 12688 | 13688 | 14688 | 15688 |
| 12689 | 13689 | 14689 | 15689 |
| 12690 | 13690 | 14690 | 15690 |
| 12691 | 13691 | 14691 | 15691 |
| 12692 | 13692 | 14692 | 15692 |
| 12693 | 13693 | 14693 | 15693 |
| 12694 | 13694 | 14694 | 15694 |
| 12695 | 13695 | 14695 | 15695 |
| 12696 | 13696 | 14696 | 15696 |
| 12697 | 13697 | 14697 | 15697 |
| 12698 | 13698 | 14698 | 15698 |
| 12699 | 13699 | 14699 | 15699 |
| 12700 | 13700 | 14700 | 15700 |
| 12701 | 13701 | 14701 | 15701 |
| 12702 | 13702 | 14702 | 15702 |
| 12703 | 13703 | 14703 | 15703 |
| 12704 | 13704 | 14704 | 15704 |
| 12705 | 13705 | 14705 | 15705 |
| 12706 | 13706 | 14706 | 15706 |
| 12707 | 13707 | 14707 | 15707 |
| 12708 | 13708 | 14708 | 15708 |
| 12709 | 13709 | 14709 | 15709 |
| 12710 | 13710 | 14710 | 15710 |
| 12711 | 13711 | 14711 | 15711 |
| 12712 | 13712 | 14712 | 15712 |
| 12713 | 13713 | 14713 | 15713 |
| 12714 | 13714 | 14714 | 15714 |
| 12715 | 13715 | 14715 | 15715 |
| 12716 | 13716 | 14716 | 15716 |
| 12717 | 13717 | 14717 | 15717 |
| 12718 | 13718 | 14718 | 15718 |
| 12719 | 13719 | 14719 | 15719 |
| 12720 | 13720 | 14720 | 15720 |
| 12721 | 13721 | 14721 | 15721 |
| 12722 | 13722 | 14722 | 15722 |
| 12723 | 13723 | 14723 | 15723 |
| 12724 | 13724 | 14724 | 15724 |
| 12725 | 13725 | 14725 | 15725 |
| 12726 | 13726 | 14726 | 15726 |
| 12727 | 13727 | 14727 | 15727 |
| 12728 | 13728 | 14728 | 15728 |
| 12729 | 13729 | 14729 | 15729 |
| 12730 | 13730 | 14730 | 15730 |
| 12731 | 13731 | 14731 | 15731 |
| 12732 | 13732 | 14732 | 15732 |
| 12733 | 13733 | 14733 | 15733 |
| 12734 | 13734 | 14734 | 15734 |
| 12735 | 13735 | 14735 | 15735 |
| 12736 | 13736 | 14736 | 15736 |
| 12737 | 13737 | 14737 | 15737 |
| 12738 | 13738 | 14738 | 15738 |
| 12739 | 13739 | 14739 | 15739 |
| 12740 | 13740 | 14740 | 15740 |
| 12741 | 13741 | 14741 | 15741 |
| 12742 | 13742 | 14742 | 15742 |
| 12743 | 13743 | 14743 | 15743 |
| 12744 | 13744 | 14744 | 15744 |
| 12745 | 13745 | 14745 | 15745 |
| 12746 | 13746 | 14746 | 15746 |
| 12747 | 13747 | 14747 | 15747 |
| 12748 | 13748 | 14748 | 15748 |
| 12749 | 13749 | 14749 | 15749 |
| 12750 | 13750 | 14750 | 15750 |
| 12751 | 13751 | 14751 | 15751 |
| 12752 | 13752 | 14752 | 15752 |
| 12753 | 13753 | 14753 | 15753 |
| 12754 | 13754 | 14754 | 15754 |
| 12755 | 13755 | 14755 | 15755 |
| 12756 | 13756 | 14756 | 15756 |
| 12757 | 13757 | 14757 | 15757 |
| 12758 | 13758 | 14758 | 15758 |
| 12759 | 13759 | 14759 | 15759 |
| 12760 | 13760 | 14760 | 15760 |
| 12761 | 13761 | 14761 | 15761 |
| 12762 | 13762 | 14762 | 15762 |
| 12763 | 13763 | 14763 | 15763 |
| 12764 | 13764 | 14764 | 15764 |
| 12765 | 13765 | 14765 | 15765 |
| 12766 | 13766 | 14766 | 15766 |
| 12767 | 13767 | 14767 | 15767 |
| 12768 | 13768 | 14768 | 15768 |
| 12769 | 13769 | 14769 | 15769 |
| 12770 | 13770 | 14770 | 15770 |
| 12771 | 13771 | 14771 | 15771 |
| 12772 | 13772 | 14772 | 15772 |
| 12773 | 13773 | 14773 | 15773 |
| 12774 | 13774 | 14774 | 15774 |
| 12775 | 13775 | 14775 | 15775 |
| 12776 | 13776 | 14776 | 15776 |
| 12777 | 13777 | 14777 | 15777 |
| 12778 | 13778 | 14778 | 15778 |
| 12779 | 13779 | 14779 | 15779 |
| 12780 | 13780 | 14780 | 15780 |
| 12781 | 13781 | 14781 | 15781 |
| 12782 | 13782 | 14782 | 15782 |
| 12783 | 13783 | 14783 | 15783 |
| 12784 | 13784 | 14784 | 15784 |
| 12785 | 13785 | 14785 | 15785 |
| 12786 | 13786 | 14786 | 15786 |
| 12787 | 13787 | 14787 | 15787 |
| 12788 | 13788 | 14788 | 15788 |
| 12789 | 13789 | 14789 | 15789 |
| 12790 | 13790 | 14790 | 15790 |
| 12791 | 13791 | 14791 | 15791 |
| 12792 | 13792 | 14792 | 15792 |
| 12793 | 13793 | 14793 | 15793 |
| 12794 | 13794 | 14794 | 15794 |
| 12795 | 13795 | 14795 | 15795 |
| 12796 | 13796 | 14796 | 15796 |
| 12797 | 13797 | 14797 | 15797 |
| 12798 | 13798 | 14798 | 15798 |
| 12799 | 13799 | 14799 | 15799 |
| 12800 | 13800 | 14800 | 15800 |
| 12801 | 13801 | 14801 | 15801 |
| 12802 | 13802 | 14802 | 15802 |
| 12803 | 13803 | 14803 | 15803 |
| 12804 | 13804 | 14804 | 15804 |
| 12805 | 13805 | 14805 | 15805 |
| 12806 | 13806 | 14806 | 15806 |
| 12807 | 13807 | 14807 | 15807 |
| 12808 | 13808 | 14808 | 15808 |
| 12809 | 13809 | 14809 | 15809 |
| 12810 | 13810 | 14810 | 15810 |
| 12811 | 13811 | 14811 | 15811 |
| 12812 | 13812 | 14812 | 15812 |
| 12813 | 13813 | 14813 | 15813 |
| 12814 | 13814 | 14814 | 15814 |
| 12815 | 13815 | 14815 | 15815 |
| 12816 | 13816 | 14816 | 15816 |
| 12817 | 13817 | 14817 | 15817 |
| 12818 | 13818 | 14818 | 15818 |
| 12819 | 13819 | 14819 | 15819 |
| 12820 | 13820 | 14820 | 15820 |
| 12821 | 13821 | 14821 | 15821 |
| 12822 | 13822 | 14822 | 15822 |
| 12823 | 13823 | 14823 | 15823 |
| 12824 | 13824 | 14824 | 15824 |
| 12825 | 13825 | 14825 | 15825 |
| 12826 | 13826 | 14826 | 15826 |
| 12827 | 13827 | 14827 | 15827 |
| 12828 | 13828 | 14828 | 15828 |
| 12829 | 13829 | 14829 | 15829 |
| 12830 | 13830 | 14830 | 15830 |
| 12831 | 13831 | 14831 | 15831 |
| 12832 | 13832 | 14832 | 15832 |
| 12833 | 13833 | 14833 | 15833 |
| 12834 | 13834 | 14834 | 15834 |
| 12835 | 13835 | 14835 | 15835 |
| 12836 | 13836 | 14836 | 15836 |
| 12837 | 13837 | 14837 | 15837 |
| 12838 | 13838 | 14838 | 15838 |
| 12839 | 13839 | 14839 | 15839 |
| 12840 | 13840 | 14840 | 15840 |
| 12841 | 13841 | 14841 | 15841 |
| 12842 | 13842 | 14842 | 15842 |
| 12843 | 13843 | 14843 | 15843 |
| 12844 | 13844 | 14844 | 15844 |
| 12845 | 13845 | 14845 | 15845 |
| 12846 | 13846 | 14846 | 15846 |
| 12847 | 13847 | 14847 | 15847 |
| 12848 | 13848 | 14848 | 15848 |
| 12849 | 13849 | 14849 | 15849 |
| 12850 | 13850 | 14850 | 15850 |
| 12851 | 13851 | 14851 | 15851 |
| 12852 | 13852 | 14852 | 15852 |
| 12853 | 13853 | 14853 | 15853 |
| 12854 | 13854 | 14854 | 15854 |
| 12855 | 13855 | 14855 | 15855 |
| 12856 | 13856 | 14856 | 15856 |
| 12857 | 13857 | 14857 | 15857 |
| 12858 | 13858 | 14858 | 15858 |
| 12859 | 13859 | 14859 | 15859 |
| 12860 | 13860 | 14860 | 15860 |
| 12861 | 13861 | 14861 | 15861 |
| 12862 | 13862 | 14862 | 15862 |
| 12863 | 13863 | 14863 | 15863 |
| 12864 | 13864 | 14864 | 15864 |
| 12865 | 13865 | 14865 | 15865 |
| 12866 | 13866 | 14866 | 15866 |
| 12867 | 13867 | 14867 | 15867 |
| 12868 | 13868 | 14868 | 15868 |
| 12869 | 13869 | 14869 | 15869 |
| 12870 | 13870 | 14870 | 15870 |
| 12871 | 13871 | 14871 | 15871 |
| 12872 | 13872 | 14872 | 15872 |
| 12873 | 13873 | 14873 | 15873 |
| 12874 | 13874 | 14874 | 15874 |
| 12875 | 13875 | 14875 | 15875 |
| 12876 | 13876 | 14876 | 15876 |
| 12877 | 13877 | 14877 | 15877 |
| 12878 | 13878 | 14878 | 15878 |
| 12879 | 13879 | 14879 | 15879 |
| 12880 | 13880 | 14880 | 15880 |
| 12881 | 13881 | 14881 | 15881 |
| 12882 | 13882 | 14882 | 15882 |
| 12883 | 13883 | 14883 | 15883 |
| 12884 | 13884 | 14884 | 15884 |
| 12885 | 13885 | 14885 | 15885 |
| 12886 | 13886 | 14886 | 15886 |
| 12887 | 13887 | 14887 | 15887 |
| 12888 | 13888 | 14888 | 15888 |
| 12889 | 13889 | 14889 | 15889 |
| 12890 | 13890 | 14890 | 15890 |
| 12891 | 13891 | 14891 | 15891 |
| 12892 | 13892 | 14892 | 15892 |
| 12893 | 13893 | 14893 | 15893 |
| 12894 | 13894 | 14894 | 15894 |
| 12895 | 13895 | 14895 | 15895 |
| 12896 | 13896 | 14896 | 15896 |
| 12897 | 13897 | 14897 | 15897 |
| 12898 | 13898 | 14898 | 15898 |
| 12899 | 13899 | 14899 | 15899 |
| 12900 | 13900 | 14900 | 15900 |
| 12901 | 13901 | 14901 | 15901 |
| 12902 | 13902 | 14902 | 15902 |
| 12903 | 13903 | 14903 | 15903 |
| 12904 | 13904 | 14904 | 15904 |
| 12905 | 13905 | 14905 | 15905 |
| 12906 | 13906 | 14906 | 15906 |
| 12907 | 13907 | 14907 | 15907 |
| 12908 | 13908 | 14908 | 15908 |
| 12909 | 13909 | 14909 | 15909 |
| 12910 | 13910 | 14910 | 15910 |
| 12911 | 13911 | 14911 | 15911 |
| 12912 | 13912 | 14912 | 15912 |
| 12913 | 13913 | 14913 | 15913 |
| 12914 | 13914 | 14914 | 15914 |
| 12915 | 13915 | 14915 | 15915 |
| 12916 | 13916 | 14916 | 15916 |
| 12917 | 13917 | 14917 | 15917 |
| 12918 | 13918 | 14918 | 15918 |
| 12919 | 13919 | 14919 | 15919 |
| 12920 | 13920 | 14920 | 15920 |
| 12921 | 13921 | 14921 | 15921 |
| 12922 | 13922 | 14922 | 15922 |
| 12923 | 13923 | 14923 | 15923 |
| 12924 | 13924 | 14924 | 15924 |
| 12925 | 13925 | 14925 | 15925 |
| 12926 | 13926 | 14926 | 15926 |
| 12927 | 13927 | 14927 | 15927 |
| 12928 | 13928 | 14928 | 15928 |
| 12929 | 13929 | 14929 | 15929 |
| 12930 | 13930 | 14930 | 15930 |
| 12931 | 13931 | 14931 | 15931 |
| 12932 | 13932 | 14932 | 15932 |
| 12933 | 13933 | 14933 | 15933 |
| 12934 | 13934 | 14934 | 15934 |
| 12935 | 13935 | 14935 | 15935 |
| 12936 | 13936 | 14936 | 15936 |
| 12937 | 13937 | 14937 | 15937 |
| 12938 | 13938 | 14938 | 15938 |
| 12939 | 13939 | 14939 | 15939 |
| 12940 | 13940 | 14940 | 15940 |
| 12941 | 13941 | 14941 | 15941 |
| 12942 | 13942 | 14942 | 15942 |
| 12943 | 13943 | 14943 | 15943 |
| 12944 | 13944 | 14944 | 15944 |
| 12945 | 13945 | 14945 | 15945 |
| 12946 | 13946 | 14946 | 15946 |
| 12947 | 13947 | 14947 | 15947 |
| 12948 | 13948 | 14948 | 15948 |
| 12949 | 13949 | 14949 | 15949 |
| 12950 | 13950 | 14950 | 15950 |
| 12951 | 13951 | 14951 | 15951 |
| 12952 | 13952 | 14952 | 15952 |
| 12953 | 13953 | 14953 | 15953 |
| 12954 | 13954 | 14954 | 15954 |
| 12955 | 13955 | 14955 | 15955 |
| 12956 | 13956 | 14956 | 15956 |
| 12957 | 13957 | 14957 | 15957 |
| 12958 | 13958 | 14958 | 15958 |
| 12959 | 13959 | 14959 | 15959 |
| 12960 | 13960 | 14960 | 15960 |
| 12961 | 13961 | 14961 | 15961 |
| 12962 | 13962 | 14962 | 15962 |
| 12963 | 13963 | 14963 | 15963 |
| 12964 | 13964 | 14964 | 15964 |
| 12965 | 13965 | 14965 | 15965 |
| 12966 | 13966 | 14966 | 15966 |
| 12967 | 13967 | 14967 | 15967 |
| 12968 | 13968 | 14968 | 15968 |
| 12969 | 13969 | 14969 | 15969 |
| 12970 | 13970 | 14970 | 15970 |
| 12971 | 13971 | 14971 | 15971 |
| 12972 | 13972 | 14972 | 15972 |
| 12973 | 13973 | 14973 | 15973 |
| 12974 | 13974 | 14974 | 15974 |
| 12975 | 13975 | 14975 | 15975 |
| 12976 | 13976 | 14976 | 15976 |
| 12977 | 13977 | 14977 | 15977 |
| 12978 | 13978 | 14978 | 15978 |
| 12979 | 13979 | 14979 | 15979 |
| 12980 | 13980 | 14980 | 15980 |
| 12981 | 13981 | 14981 | 15981 |
| 12982 | 13982 | 14982 | 15982 |
| 12983 | 13983 | 14983 | 15983 |
| 12984 | 13984 | 14984 | 15984 |
| 12985 | 13985 | 14985 | 15985 |
| 12986 | 13986 | 14986 | 15986 |
| 12987 | 13987 | 14987 | 15987 |
| 12988 | 13988 | 14988 | 15988 |
| 12989 | 13989 | 14989 | 15989 |
| 12990 | 13990 | 14990 | 15990 |
| 12991 | 13991 | 14991 | 15991 |
| 12992 | 13992 | 14992 | 15992 |
| 12993 | 13993 | 14993 | 15993 |
| 12994 | 13994 | 14994 | 15994 |
| 12995 | 13995 | 14995 | 15995 |
| 12996 | 13996 | 14996 | 15996 |
| 12997 | 13997 | 14997 | 15997 |
| 12998 | 13998 | 14998 | 15998 |
| 12999 | 13999 | 14999 | 15999 |
| 13000 | 14000 | 15000 | 16000 |
| 13001 | 14001 | 15001 | 16001 |
| 13002 | 14002 | 15002 | 16002 |

In some embodiments, the sequences that specifically bind to hazelnut comprise an inner sequence selected from the group consisting of the nucleic acid sequences of SEQ ID NOs. 16003 to 17002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to hazel nut may comprise a nucleic acid sequence selected from the group consisting of SEQ ID NOs. 17003 to 18002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to hazel nut may comprise a nucleic acid sequence selected from the group consisting of SEQ ID NOs. 18003 to 19002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to hazel nut may comprise a nucleic acid sequence selected from the group consisting of SEQ ID NOs. 19003 to 20002. In one embodiment, the aptamer of the present disclosure that specifically binds to hazelnut may comprise a nucleic acid sequence selected from the group consisting of SEQ ID NOs. 16003 to 20002 listed in Table 6, or variant thereof.

TABLE 6
Aptamer sequences against hazel nut
Aptamer sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 16003 | 17003 | 18003 | 19003 |
| 16004 | 17004 | 18004 | 19004 |
| 16005 | 17005 | 18005 | 19005 |
| 16006 | 17006 | 18006 | 19006 |
| 16007 | 17007 | 18007 | 19007 |
| 16008 | 17008 | 18008 | 19008 |
| 16009 | 17009 | 18009 | 19009 |
| 16010 | 17010 | 18010 | 19010 |
| 16011 | 17011 | 18011 | 19011 |
| 16012 | 17012 | 18012 | 19012 |
| 16013 | 17013 | 18013 | 19013 |
| 16014 | 17014 | 18014 | 19014 |
| 16015 | 17015 | 18015 | 19015 |
| 16016 | 17016 | 18016 | 19016 |
| 16017 | 17017 | 18017 | 19017 |
| 16018 | 17018 | 18018 | 19018 |
| 16019 | 17019 | 18019 | 19019 |
| 16020 | 17020 | 18020 | 19020 |
| 16021 | 17021 | 18021 | 19021 |
| 16022 | 17022 | 18022 | 19022 |
| 16023 | 17023 | 18023 | 19023 |
| 16024 | 17024 | 18024 | 19024 |
| 16025 | 17025 | 18025 | 19025 |
| 16026 | 17026 | 18026 | 19026 |
| 16027 | 17027 | 18027 | 19027 |
| 16028 | 17028 | 18028 | 19028 |
| 16029 | 17029 | 18029 | 19029 |
| 16030 | 17030 | 18030 | 19030 |
| 16031 | 17031 | 18031 | 19031 |
| 16032 | 17032 | 18032 | 19032 |
| 16033 | 17033 | 18033 | 19033 |
| 16034 | 17034 | 18034 | 19034 |
| 16035 | 17035 | 18035 | 19035 |
| 16036 | 17036 | 18036 | 19036 |
| 16037 | 17037 | 18037 | 19037 |
| 16038 | 17038 | 18038 | 19038 |
| 16039 | 17039 | 18039 | 19039 |
| 16040 | 17040 | 18040 | 19040 |
| 16041 | 17041 | 18041 | 19041 |
| 16042 | 17042 | 18042 | 19042 |
| 16043 | 17043 | 18043 | 19043 |
| 16044 | 17044 | 18044 | 19044 |
| 16045 | 17045 | 18045 | 19045 |
| 16046 | 17046 | 18046 | 19046 |
| 16047 | 17047 | 18047 | 19047 |
| 16048 | 17048 | 18048 | 19048 |
| 16049 | 17049 | 18049 | 19049 |
| 16050 | 17050 | 18050 | 19050 |
| 16051 | 17051 | 18051 | 19051 |
| 16052 | 17052 | 18052 | 19052 |
| 16053 | 17053 | 18053 | 19053 |
| 16054 | 17054 | 18054 | 19054 |
| 16055 | 17055 | 18055 | 19055 |
| 16056 | 17056 | 18056 | 19056 |
| 16057 | 17057 | 18057 | 19057 |
| 16058 | 17058 | 18058 | 19058 |
| 16059 | 17059 | 18059 | 19059 |
| 16060 | 17060 | 18060 | 19060 |
| 16061 | 17061 | 18061 | 19061 |
| 16062 | 17062 | 18062 | 19062 |
| 16063 | 17063 | 18063 | 19063 |
| 16064 | 17064 | 18064 | 19064 |
| 16065 | 17065 | 18065 | 19065 |
| 16066 | 17066 | 18066 | 19066 |
| 16067 | 17067 | 18067 | 19067 |
| 16068 | 17068 | 18068 | 19068 |
| 16069 | 17069 | 18069 | 19069 |
| 16070 | 17070 | 18070 | 19070 |
| 16071 | 17071 | 18071 | 19071 |
| 16072 | 17072 | 18072 | 19072 |
| 16073 | 17073 | 18073 | 19073 |
| 16074 | 17074 | 18074 | 19074 |
| 16075 | 17075 | 18075 | 19075 |
| 16076 | 17076 | 18076 | 19076 |
| 16077 | 17077 | 18077 | 19077 |
| 16078 | 17078 | 18078 | 19078 |
| 16079 | 17079 | 18079 | 19079 |
| 16080 | 17080 | 18080 | 19080 |
| 16081 | 17081 | 18081 | 19081 |
| 16082 | 17082 | 18082 | 19082 |
| 16083 | 17083 | 18083 | 19083 |
| 16084 | 17084 | 18084 | 19084 |
| 16085 | 17085 | 18085 | 19085 |
| 16086 | 17086 | 18086 | 19086 |
| 16087 | 17087 | 18087 | 19087 |
| 16088 | 17088 | 18088 | 19088 |
| 16089 | 17089 | 18089 | 19089 |
| 16090 | 17090 | 18090 | 19090 |
| 16091 | 17091 | 18091 | 19091 |
| 16092 | 17092 | 18092 | 19092 |
| 16093 | 17093 | 18093 | 19093 |
| 16094 | 17094 | 18094 | 19094 |
| 16095 | 17095 | 18095 | 19095 |
| 16096 | 17096 | 18096 | 19096 |
| 16097 | 17097 | 18097 | 19097 |
| 16098 | 17098 | 18098 | 19098 |
| 16099 | 17099 | 18099 | 19099 |
| 16100 | 17100 | 18100 | 19100 |
| 16101 | 17101 | 18101 | 19101 |
| 16102 | 17102 | 18102 | 19102 |
| 16103 | 17103 | 18103 | 19103 |
| 16104 | 17104 | 18104 | 19104 |
| 16105 | 17105 | 18105 | 19105 |
| 16106 | 17106 | 18106 | 19106 |
| 16107 | 17107 | 18107 | 19107 |
| 16108 | 17108 | 18108 | 19108 |
| 16109 | 17109 | 18109 | 19109 |
| 16110 | 17110 | 18110 | 19110 |
| 16111 | 17111 | 18111 | 19111 |
| 16112 | 17112 | 18112 | 19112 |
| 16113 | 17113 | 18113 | 19113 |
| 16114 | 17114 | 18114 | 19114 |
| 16115 | 17115 | 18115 | 19115 |
| 16116 | 17116 | 18116 | 19116 |
| 16117 | 17117 | 18117 | 19117 |
| 16118 | 17118 | 18118 | 19118 |
| 16119 | 17119 | 18119 | 19119 |
| 16120 | 17120 | 18120 | 19120 |
| 16121 | 17121 | 18121 | 19121 |
| 16122 | 17122 | 18122 | 19122 |
| 16123 | 17123 | 18123 | 19123 |
| 16124 | 17124 | 18124 | 19124 |
| 16125 | 17125 | 18125 | 19125 |
| 16126 | 17126 | 18126 | 19126 |
| 16127 | 17127 | 18127 | 19127 |
| 16128 | 17128 | 18128 | 19128 |
| 16129 | 17129 | 18129 | 19129 |
| 16130 | 17130 | 18130 | 19130 |
| 16131 | 17131 | 18131 | 19131 |
| 16132 | 17132 | 18132 | 19132 |
| 16133 | 17133 | 18133 | 19133 |
| 16134 | 17134 | 18134 | 19134 |
| 16135 | 17135 | 18135 | 19135 |
| 16136 | 17136 | 18136 | 19136 |
| 16137 | 17137 | 18137 | 19137 |
| 16138 | 17138 | 18138 | 19138 |
| 16139 | 17139 | 18139 | 19139 |
| 16140 | 17140 | 18140 | 19140 |
| 16141 | 17141 | 18141 | 19141 |
| 16142 | 17142 | 18142 | 19142 |
| 16143 | 17143 | 18143 | 19143 |
| 16144 | 17144 | 18144 | 19144 |
| 16145 | 17145 | 18145 | 19145 |
| 16146 | 17146 | 18146 | 19146 |
| 16147 | 17147 | 18147 | 19147 |
| 16148 | 17148 | 18148 | 19148 |
| 16149 | 17149 | 18149 | 19149 |
| 16150 | 17150 | 18150 | 19150 |
| 16151 | 17151 | 18151 | 19151 |
| 16152 | 17152 | 18152 | 19152 |
| 16153 | 17153 | 18153 | 19153 |
| 16154 | 17154 | 18154 | 19154 |
| 16155 | 17155 | 18155 | 19155 |
| 16156 | 17156 | 18156 | 19156 |
| 16157 | 17157 | 18157 | 19157 |
| 16158 | 17158 | 18158 | 19158 |
| 16159 | 17159 | 18159 | 19159 |
| 16160 | 17160 | 18160 | 19160 |
| 16161 | 17161 | 18161 | 19161 |
| 16162 | 17162 | 18162 | 19162 |
| 16163 | 17163 | 18163 | 19163 |
| 16164 | 17164 | 18164 | 19164 |
| 16165 | 17165 | 18165 | 19165 |
| 16166 | 17166 | 18166 | 19166 |
| 16167 | 17167 | 18167 | 19167 |
| 16168 | 17168 | 18168 | 19168 |
| 16169 | 17169 | 18169 | 19169 |
| 16170 | 17170 | 18170 | 19170 |
| 16171 | 17171 | 18171 | 19171 |
| 16172 | 17172 | 18172 | 19172 |
| 16173 | 17173 | 18173 | 19173 |
| 16174 | 17174 | 18174 | 19174 |
| 16175 | 17175 | 18175 | 19175 |
| 16176 | 17176 | 18176 | 19176 |
| 16177 | 17177 | 18177 | 19177 |
| 16178 | 17178 | 18178 | 19178 |
| 16179 | 17179 | 18179 | 19179 |
| 16180 | 17180 | 18180 | 19180 |
| 16181 | 17181 | 18181 | 19181 |
| 16182 | 17182 | 18182 | 19182 |
| 16183 | 17183 | 18183 | 19183 |
| 16184 | 17184 | 18184 | 19184 |
| 16185 | 17185 | 18185 | 19185 |
| 16186 | 17186 | 18186 | 19186 |
| 16187 | 17187 | 18187 | 19187 |
| 16188 | 17188 | 18188 | 19188 |
| 16189 | 17189 | 18189 | 19189 |
| 16190 | 17190 | 18190 | 19190 |
| 16191 | 17191 | 18191 | 19191 |
| 16192 | 17192 | 18192 | 19192 |
| 16193 | 17193 | 18193 | 19193 |
| 16194 | 17194 | 18194 | 19194 |
| 16195 | 17195 | 18195 | 19195 |
| 16196 | 17196 | 18196 | 19196 |
| 16197 | 17197 | 18197 | 19197 |
| 16198 | 17198 | 18198 | 19198 |
| 16199 | 17199 | 18199 | 19199 |
| 16200 | 17200 | 18200 | 19200 |
| 16201 | 17201 | 18201 | 19201 |
| 16202 | 17202 | 18202 | 19202 |
| 16203 | 17203 | 18203 | 19203 |
| 16204 | 17204 | 18204 | 19204 |
| 16205 | 17205 | 18205 | 19205 |
| 16206 | 17206 | 18206 | 19206 |
| 16207 | 17207 | 18207 | 19207 |
| 16208 | 17208 | 18208 | 19208 |
| 16209 | 17209 | 18209 | 19209 |
| 16210 | 17210 | 18210 | 19210 |
| 16211 | 17211 | 18211 | 19211 |
| 16212 | 17212 | 18212 | 19212 |
| 16213 | 17213 | 18213 | 19213 |
| 16214 | 17214 | 18214 | 19214 |
| 16215 | 17215 | 18215 | 19215 |
| 16216 | 17216 | 18216 | 19216 |
| 16217 | 17217 | 18217 | 19217 |
| 16218 | 17218 | 18218 | 19218 |
| 16219 | 17219 | 18219 | 19219 |
| 16220 | 17220 | 18220 | 19220 |
| 16221 | 17221 | 18221 | 19221 |
| 16222 | 17222 | 18222 | 19222 |
| 16223 | 17223 | 18223 | 19223 |
| 16224 | 17224 | 18224 | 19224 |
| 16225 | 17225 | 18225 | 19225 |
| 16226 | 17226 | 18226 | 19226 |
| 16227 | 17227 | 18227 | 19227 |
| 16228 | 17228 | 18228 | 19228 |
| 16229 | 17229 | 18229 | 19229 |
| 16230 | 17230 | 18230 | 19230 |
| 16231 | 17231 | 18231 | 19231 |
| 16232 | 17232 | 18232 | 19232 |
| 16233 | 17233 | 18233 | 19233 |
| 16234 | 17234 | 18234 | 19234 |
| 16235 | 17235 | 18235 | 19235 |
| 16236 | 17236 | 18236 | 19236 |
| 16237 | 17237 | 18237 | 19237 |
| 16238 | 17238 | 18238 | 19238 |
| 16239 | 17239 | 18239 | 19239 |
| 16240 | 17240 | 18240 | 19240 |
| 16241 | 17241 | 18241 | 19241 |
| 16242 | 17242 | 18242 | 19242 |
| 16243 | 17243 | 18243 | 19243 |
| 16244 | 17244 | 18244 | 19244 |
| 16245 | 17245 | 18245 | 19245 |
| 16246 | 17246 | 18246 | 19246 |
| 16247 | 17247 | 18247 | 19247 |
| 16248 | 17248 | 18248 | 19248 |
| 16249 | 17249 | 18249 | 19249 |
| 16250 | 17250 | 18250 | 19250 |
| 16251 | 17251 | 18251 | 19251 |
| 16252 | 17252 | 18252 | 19252 |
| 16253 | 17253 | 18253 | 19253 |
| 16254 | 17254 | 18254 | 19254 |
| 16255 | 17255 | 18255 | 19255 |
| 16256 | 17256 | 18256 | 19256 |
| 16257 | 17257 | 18257 | 19257 |
| 16258 | 17258 | 18258 | 19258 |
| 16259 | 17259 | 18259 | 19259 |
| 16260 | 17260 | 18260 | 19260 |
| 16261 | 17261 | 18261 | 19261 |
| 16262 | 17262 | 18262 | 19262 |
| 16263 | 17263 | 18263 | 19263 |
| 16264 | 17264 | 18264 | 19264 |
| 16265 | 17265 | 18265 | 19265 |
| 16266 | 17266 | 18266 | 19266 |
| 16267 | 17267 | 18267 | 19267 |
| 16268 | 17268 | 18268 | 19268 |
| 16269 | 17269 | 18269 | 19269 |
| 16270 | 17270 | 18270 | 19270 |
| 16271 | 17271 | 18271 | 19271 |
| 16272 | 17272 | 18272 | 19272 |
| 16273 | 17273 | 18273 | 19273 |
| 16274 | 17274 | 18274 | 19274 |
| 16275 | 17275 | 18275 | 19275 |
| 16276 | 17276 | 18276 | 19276 |
| 16277 | 17277 | 18277 | 19277 |
| 16278 | 17278 | 18278 | 19278 |
| 16279 | 17279 | 18279 | 19279 |
| 16280 | 17280 | 18280 | 19280 |
| 16281 | 17281 | 18281 | 19281 |
| 16282 | 17282 | 18282 | 19282 |
| 16283 | 17283 | 18283 | 19283 |
| 16284 | 17284 | 18284 | 19284 |
| 16285 | 17285 | 18285 | 19285 |
| 16286 | 17286 | 18286 | 19286 |
| 16287 | 17287 | 18287 | 19287 |
| 16288 | 17288 | 18288 | 19288 |
| 16289 | 17289 | 18289 | 19289 |
| 16290 | 17290 | 18290 | 19290 |
| 16291 | 17291 | 18291 | 19291 |
| 16292 | 17292 | 18292 | 19292 |
| 16293 | 17293 | 18293 | 19293 |
| 16294 | 17294 | 18294 | 19294 |
| 16295 | 17295 | 18295 | 19295 |
| 16296 | 17296 | 18296 | 19296 |
| 16297 | 17297 | 18297 | 19297 |
| 16298 | 17298 | 18298 | 19298 |
| 16299 | 17299 | 18299 | 19299 |
| 16300 | 17300 | 18300 | 19300 |
| 16301 | 17301 | 18301 | 19301 |
| 16302 | 17302 | 18302 | 19302 |
| 16303 | 17303 | 18303 | 19303 |
| 16304 | 17304 | 18304 | 19304 |
| 16305 | 17305 | 18305 | 19305 |
| 16306 | 17306 | 18306 | 19306 |
| 16307 | 17307 | 18307 | 19307 |
| 16308 | 17308 | 18308 | 19308 |
| 16309 | 17309 | 18309 | 19309 |
| 16310 | 17310 | 18310 | 19310 |
| 16311 | 17311 | 18311 | 19311 |
| 16312 | 17312 | 18312 | 19312 |
| 16313 | 17313 | 18313 | 19313 |
| 16314 | 17314 | 18314 | 19314 |
| 16315 | 17315 | 18315 | 19315 |
| 16316 | 17316 | 18316 | 19316 |
| 16317 | 17317 | 18317 | 19317 |
| 16318 | 17318 | 18318 | 19318 |
| 16319 | 17319 | 18319 | 19319 |
| 16320 | 17320 | 18320 | 19320 |
| 16321 | 17321 | 18321 | 19321 |
| 16322 | 17322 | 18322 | 19322 |
| 16323 | 17323 | 18323 | 19323 |
| 16324 | 17324 | 18324 | 19324 |
| 16325 | 17325 | 18325 | 19325 |
| 16326 | 17326 | 18326 | 19326 |
| 16327 | 17327 | 18327 | 19327 |
| 16328 | 17328 | 18328 | 19328 |
| 16329 | 17329 | 18329 | 19329 |
| 16330 | 17330 | 18330 | 19330 |
| 16331 | 17331 | 18331 | 19331 |
| 16332 | 17332 | 18332 | 19332 |
| 16333 | 17333 | 18333 | 19333 |
| 16334 | 17334 | 18334 | 19334 |
| 16335 | 17335 | 18335 | 19335 |
| 16336 | 17336 | 18336 | 19336 |
| 16337 | 17337 | 18337 | 19337 |
| 16338 | 17338 | 18338 | 19338 |
| 16339 | 17339 | 18339 | 19339 |
| 16340 | 17340 | 18340 | 19340 |
| 16341 | 17341 | 18341 | 19341 |
| 16342 | 17342 | 18342 | 19342 |
| 16343 | 17343 | 18343 | 19343 |
| 16344 | 17344 | 18344 | 19344 |
| 16345 | 17345 | 18345 | 19345 |
| 16346 | 17346 | 18346 | 19346 |
| 16347 | 17347 | 18347 | 19347 |
| 16348 | 17348 | 18348 | 19348 |
| 16349 | 17349 | 18349 | 19349 |
| 16350 | 17350 | 18350 | 19350 |
| 16351 | 17351 | 18351 | 19351 |
| 16352 | 17352 | 18352 | 19352 |
| 16353 | 17353 | 18353 | 19353 |
| 16354 | 17354 | 18354 | 19354 |
| 16355 | 17355 | 18355 | 19355 |
| 16356 | 17356 | 18356 | 19356 |
| 16357 | 17357 | 18357 | 19357 |
| 16358 | 17358 | 18358 | 19358 |
| 16359 | 17359 | 18359 | 19359 |
| 16360 | 17360 | 18360 | 19360 |
| 16361 | 17361 | 18361 | 19361 |
| 16362 | 17362 | 18362 | 19362 |
| 16363 | 17363 | 18363 | 19363 |
| 16364 | 17364 | 18364 | 19364 |
| 16365 | 17365 | 18365 | 19365 |
| 16366 | 17366 | 18366 | 19366 |
| 16367 | 17367 | 18367 | 19367 |
| 16368 | 17368 | 18368 | 19368 |
| 16369 | 17369 | 18369 | 19369 |
| 16370 | 17370 | 18370 | 19370 |
| 16371 | 17371 | 18371 | 19371 |
| 16372 | 17372 | 18372 | 19372 |
| 16373 | 17373 | 18373 | 19373 |
| 16374 | 17374 | 18374 | 19374 |
| 16375 | 17375 | 18375 | 19375 |
| 16376 | 17376 | 18376 | 19376 |
| 16377 | 17377 | 18377 | 19377 |
| 16378 | 17378 | 18378 | 19378 |
| 16379 | 17379 | 18379 | 19379 |
| 16380 | 17380 | 18380 | 19380 |
| 16381 | 17381 | 18381 | 19381 |
| 16382 | 17382 | 18382 | 19382 |
| 16383 | 17383 | 18383 | 19383 |
| 16384 | 17384 | 18384 | 19384 |
| 16385 | 17385 | 18385 | 19385 |
| 16386 | 17386 | 18386 | 19386 |
| 16387 | 17387 | 18387 | 19387 |
| 16388 | 17388 | 18388 | 19388 |
| 16389 | 17389 | 18389 | 19389 |
| 16390 | 17390 | 18390 | 19390 |
| 16391 | 17391 | 18391 | 19391 |
| 16392 | 17392 | 18392 | 19392 |
| 16393 | 17393 | 18393 | 19393 |
| 16394 | 17394 | 18394 | 19394 |
| 16395 | 17395 | 18395 | 19395 |
| 16396 | 17396 | 18396 | 19396 |
| 16397 | 17397 | 18397 | 19397 |
| 16398 | 17398 | 18398 | 19398 |
| 16399 | 17399 | 18399 | 19399 |
| 16400 | 17400 | 18400 | 19400 |
| 16401 | 17401 | 18401 | 19401 |
| 16402 | 17402 | 18402 | 19402 |
| 16403 | 17403 | 18403 | 19403 |
| 16404 | 17404 | 18404 | 19404 |
| 16405 | 17405 | 18405 | 19405 |
| 16406 | 17406 | 18406 | 19406 |
| 16407 | 17407 | 18407 | 19407 |
| 16408 | 17408 | 18408 | 19408 |
| 16409 | 17409 | 18409 | 19409 |
| 16410 | 17410 | 18410 | 19410 |
| 16411 | 17411 | 18411 | 19411 |
| 16412 | 17412 | 18412 | 19412 |
| 16413 | 17413 | 18413 | 19413 |
| 16414 | 17414 | 18414 | 19414 |
| 16415 | 17415 | 18415 | 19415 |
| 16416 | 17416 | 18416 | 19416 |
| 16417 | 17417 | 18417 | 19417 |
| 16418 | 17418 | 18418 | 19418 |
| 16419 | 17419 | 18419 | 19419 |
| 16420 | 17420 | 18420 | 19420 |
| 16421 | 17421 | 18421 | 19421 |
| 16422 | 17422 | 18422 | 19422 |
| 16423 | 17423 | 18423 | 19423 |
| 16424 | 17424 | 18424 | 19424 |
| 16425 | 17425 | 18425 | 19425 |
| 16426 | 17426 | 18426 | 19426 |
| 16427 | 17427 | 18427 | 19427 |
| 16428 | 17428 | 18428 | 19428 |
| 16429 | 17429 | 18429 | 19429 |
| 16430 | 17430 | 18430 | 19430 |
| 16431 | 17431 | 18431 | 19431 |
| 16432 | 17432 | 18432 | 19432 |
| 16433 | 17433 | 18433 | 19433 |
| 16434 | 17434 | 18434 | 19434 |
| 16435 | 17435 | 18435 | 19435 |
| 16436 | 17436 | 18436 | 19436 |
| 16437 | 17437 | 18437 | 19437 |
| 16438 | 17438 | 18438 | 19438 |
| 16439 | 17439 | 18439 | 19439 |
| 16440 | 17440 | 18440 | 19440 |
| 16441 | 17441 | 18441 | 19441 |
| 16442 | 17442 | 18442 | 19442 |
| 16443 | 17443 | 18443 | 19443 |
| 16444 | 17444 | 18444 | 19444 |
| 16445 | 17445 | 18445 | 19445 |
| 16446 | 17446 | 18446 | 19446 |
| 16447 | 17447 | 18447 | 19447 |
| 16448 | 17448 | 18448 | 19448 |
| 16449 | 17449 | 18449 | 19449 |
| 16450 | 17450 | 18450 | 19450 |
| 16451 | 17451 | 18451 | 19451 |
| 16452 | 17452 | 18452 | 19452 |
| 16453 | 17453 | 18453 | 19453 |
| 16454 | 17454 | 18454 | 19454 |
| 16455 | 17455 | 18455 | 19455 |
| 16456 | 17456 | 18456 | 19456 |
| 16457 | 17457 | 18457 | 19457 |
| 16458 | 17458 | 18458 | 19458 |
| 16459 | 17459 | 18459 | 19459 |
| 16460 | 17460 | 18460 | 19460 |
| 16461 | 17461 | 18461 | 19461 |
| 16462 | 17462 | 18462 | 19462 |
| 16463 | 17463 | 18463 | 19463 |
| 16464 | 17464 | 18464 | 19464 |
| 16465 | 17465 | 18465 | 19465 |
| 16466 | 17466 | 18466 | 19466 |
| 16467 | 17467 | 18467 | 19467 |
| 16468 | 17468 | 18468 | 19468 |
| 16469 | 17469 | 18469 | 19469 |
| 16470 | 17470 | 18470 | 19470 |
| 16471 | 17471 | 18471 | 19471 |
| 16472 | 17472 | 18472 | 19472 |
| 16473 | 17473 | 18473 | 19473 |
| 16474 | 17474 | 18474 | 19474 |
| 16475 | 17475 | 18475 | 19475 |
| 16476 | 17476 | 18476 | 19476 |
| 16477 | 17477 | 18477 | 19477 |
| 16478 | 17478 | 18478 | 19478 |
| 16479 | 17479 | 18479 | 19479 |
| 16480 | 17480 | 18480 | 19480 |
| 16481 | 17481 | 18481 | 19481 |
| 16482 | 17482 | 18482 | 19482 |
| 16483 | 17483 | 18483 | 19483 |
| 16484 | 17484 | 18484 | 19484 |
| 16485 | 17485 | 18485 | 19485 |
| 16486 | 17486 | 18486 | 19486 |
| 16487 | 17487 | 18487 | 19487 |
| 16488 | 17488 | 18488 | 19488 |
| 16489 | 17489 | 18489 | 19489 |
| 16490 | 17490 | 18490 | 19490 |
| 16491 | 17491 | 18491 | 19491 |
| 16492 | 17492 | 18492 | 19492 |
| 16493 | 17493 | 18493 | 19493 |
| 16494 | 17494 | 18494 | 19494 |
| 16495 | 17495 | 18495 | 19495 |
| 16496 | 17496 | 18496 | 19496 |
| 16497 | 17497 | 18497 | 19497 |
| 16498 | 17498 | 18498 | 19498 |
| 16499 | 17499 | 18499 | 19499 |
| 16500 | 17500 | 18500 | 19500 |
| 16501 | 17501 | 18501 | 19501 |
| 16502 | 17502 | 18502 | 19502 |
| 16503 | 17503 | 18503 | 19503 |
| 16504 | 17504 | 18504 | 19504 |
| 16505 | 17505 | 18505 | 19505 |
| 16506 | 17506 | 18506 | 19506 |
| 16507 | 17507 | 18507 | 19507 |
| 16508 | 17508 | 18508 | 19508 |
| 16509 | 17509 | 18509 | 19509 |
| 16510 | 17510 | 18510 | 19510 |
| 16511 | 17511 | 18511 | 19511 |
| 16512 | 17512 | 18512 | 19512 |
| 16513 | 17513 | 18513 | 19513 |
| 16514 | 17514 | 18514 | 19514 |
| 16515 | 17515 | 18515 | 19515 |
| 16516 | 17516 | 18516 | 19516 |
| 16517 | 17517 | 18517 | 19517 |
| 16518 | 17518 | 18518 | 19518 |
| 16519 | 17519 | 18519 | 19519 |
| 16520 | 17520 | 18520 | 19520 |
| 16521 | 17521 | 18521 | 19521 |
| 16522 | 17522 | 18522 | 19522 |
| 16523 | 17523 | 18523 | 19523 |
| 16524 | 17524 | 18524 | 19524 |
| 16525 | 17525 | 18525 | 19525 |
| 16526 | 17526 | 18526 | 19526 |
| 16527 | 17527 | 18527 | 19527 |
| 16528 | 17528 | 18528 | 19528 |
| 16529 | 17529 | 18529 | 19529 |
| 16530 | 17530 | 18530 | 19530 |
| 16531 | 17531 | 18531 | 19531 |
| 16532 | 17532 | 18532 | 19532 |
| 16533 | 17533 | 18533 | 19533 |
| 16534 | 17534 | 18534 | 19534 |
| 16535 | 17535 | 18535 | 19535 |
| 16536 | 17536 | 18536 | 19536 |
| 16537 | 17537 | 18537 | 19537 |
| 16538 | 17538 | 18538 | 19538 |
| 16539 | 17539 | 18539 | 19539 |
| 16540 | 17540 | 18540 | 19540 |
| 16541 | 17541 | 18541 | 19541 |
| 16542 | 17542 | 18542 | 19542 |
| 16543 | 17543 | 18543 | 19543 |
| 16544 | 17544 | 18544 | 19544 |
| 16545 | 17545 | 18545 | 19545 |
| 16546 | 17546 | 18546 | 19546 |
| 16547 | 17547 | 18547 | 19547 |
| 16548 | 17548 | 18548 | 19548 |
| 16549 | 17549 | 18549 | 19549 |
| 16550 | 17550 | 18550 | 19550 |
| 16551 | 17551 | 18551 | 19551 |
| 16552 | 17552 | 18552 | 19552 |
| 16553 | 17553 | 18553 | 19553 |
| 16554 | 17554 | 18554 | 19554 |
| 16555 | 17555 | 18555 | 19555 |
| 16556 | 17556 | 18556 | 19556 |
| 16557 | 17557 | 18557 | 19557 |
| 16558 | 17558 | 18558 | 19558 |
| 16559 | 17559 | 18559 | 19559 |
| 16560 | 17560 | 18560 | 19560 |
| 16561 | 17561 | 18561 | 19561 |
| 16562 | 17562 | 18562 | 19562 |
| 16563 | 17563 | 18563 | 19563 |
| 16564 | 17564 | 18564 | 19564 |
| 16565 | 17565 | 18565 | 19565 |
| 16566 | 17566 | 18566 | 19566 |
| 16567 | 17567 | 18567 | 19567 |
| 16568 | 17568 | 18568 | 19568 |
| 16569 | 17569 | 18569 | 19569 |
| 16570 | 17570 | 18570 | 19570 |
| 16571 | 17571 | 18571 | 19571 |
| 16572 | 17572 | 18572 | 19572 |
| 16573 | 17573 | 18573 | 19573 |
| 16574 | 17574 | 18574 | 19574 |
| 16575 | 17575 | 18575 | 19575 |
| 16576 | 17576 | 18576 | 19576 |
| 16577 | 17577 | 18577 | 19577 |
| 16578 | 17578 | 18578 | 19578 |
| 16579 | 17579 | 18579 | 19579 |
| 16580 | 17580 | 18580 | 19580 |
| 16581 | 17581 | 18581 | 19581 |
| 16582 | 17582 | 18582 | 19582 |
| 16583 | 17583 | 18583 | 19583 |
| 16584 | 17584 | 18584 | 19584 |
| 16585 | 17585 | 18585 | 19585 |
| 16586 | 17586 | 18586 | 19586 |
| 16587 | 17587 | 18587 | 19587 |
| 16588 | 17588 | 18588 | 19588 |
| 16589 | 17589 | 18589 | 19589 |
| 16590 | 17590 | 18590 | 19590 |
| 16591 | 17591 | 18591 | 19591 |
| 16592 | 17592 | 18592 | 19592 |
| 16593 | 17593 | 18593 | 19593 |
| 16594 | 17594 | 18594 | 19594 |
| 16595 | 17595 | 18595 | 19595 |
| 16596 | 17596 | 18596 | 19596 |
| 16597 | 17597 | 18597 | 19597 |
| 16598 | 17598 | 18598 | 19598 |
| 16599 | 17599 | 18599 | 19599 |
| 16600 | 17600 | 18600 | 19600 |
| 16601 | 17601 | 18601 | 19601 |
| 16602 | 17602 | 18602 | 19602 |
| 16603 | 17603 | 18603 | 19603 |
| 16604 | 17604 | 18604 | 19604 |
| 16605 | 17605 | 18605 | 19605 |
| 16606 | 17606 | 18606 | 19606 |
| 16607 | 17607 | 18607 | 19607 |
| 16608 | 17608 | 18608 | 19608 |
| 16609 | 17609 | 18609 | 19609 |
| 16610 | 17610 | 18610 | 19610 |
| 16611 | 17611 | 18611 | 19611 |
| 16612 | 17612 | 18612 | 19612 |
| 16613 | 17613 | 18613 | 19613 |
| 16614 | 17614 | 18614 | 19614 |
| 16615 | 17615 | 18615 | 19615 |
| 16616 | 17616 | 18616 | 19616 |
| 16617 | 17617 | 18617 | 19617 |
| 16618 | 17618 | 18618 | 19618 |
| 16619 | 17619 | 18619 | 19619 |
| 16620 | 17620 | 18620 | 19620 |
| 16621 | 17621 | 18621 | 19621 |
| 16622 | 17622 | 18622 | 19622 |
| 16623 | 17623 | 18623 | 19623 |
| 16624 | 17624 | 18624 | 19624 |
| 16625 | 17625 | 18625 | 19625 |
| 16626 | 17626 | 18626 | 19626 |
| 16627 | 17627 | 18627 | 19627 |
| 16628 | 17628 | 18628 | 19628 |
| 16629 | 17629 | 18629 | 19629 |
| 16630 | 17630 | 18630 | 19630 |
| 16631 | 17631 | 18631 | 19631 |
| 16632 | 17632 | 18632 | 19632 |
| 16633 | 17633 | 18633 | 19633 |
| 16634 | 17634 | 18634 | 19634 |
| 16635 | 17635 | 18635 | 19635 |
| 16636 | 17636 | 18636 | 19636 |
| 16637 | 17637 | 18637 | 19637 |
| 16638 | 17638 | 18638 | 19638 |
| 16639 | 17639 | 18639 | 19639 |
| 16640 | 17640 | 18640 | 19640 |
| 16641 | 17641 | 18641 | 19641 |
| 16642 | 17642 | 18642 | 19642 |
| 16643 | 17643 | 18643 | 19643 |
| 16644 | 17644 | 18644 | 19644 |
| 16645 | 17645 | 18645 | 19645 |
| 16646 | 17646 | 18646 | 19646 |
| 16647 | 17647 | 18647 | 19647 |
| 16648 | 17648 | 18648 | 19648 |
| 16649 | 17649 | 18649 | 19649 |
| 16650 | 17650 | 18650 | 19650 |
| 16651 | 17651 | 18651 | 19651 |
| 16652 | 17652 | 18652 | 19652 |
| 16653 | 17653 | 18653 | 19653 |
| 16654 | 17654 | 18654 | 19654 |
| 16655 | 17655 | 18655 | 19655 |
| 16656 | 17656 | 18656 | 19656 |
| 16657 | 17657 | 18657 | 19657 |
| 16658 | 17658 | 18658 | 19658 |
| 16659 | 17659 | 18659 | 19659 |
| 16660 | 17660 | 18660 | 19660 |
| 16661 | 17661 | 18661 | 19661 |
| 16662 | 17662 | 18662 | 19662 |
| 16663 | 17663 | 18663 | 19663 |
| 16664 | 17664 | 18664 | 19664 |
| 16665 | 17665 | 18665 | 19665 |
| 16666 | 17666 | 18666 | 19666 |
| 16667 | 17667 | 18667 | 19667 |
| 16668 | 17668 | 18668 | 19668 |
| 16669 | 17669 | 18669 | 19669 |
| 16670 | 17670 | 18670 | 19670 |
| 16671 | 17671 | 18671 | 19671 |
| 16672 | 17672 | 18672 | 19672 |
| 16673 | 17673 | 18673 | 19673 |
| 16674 | 17674 | 18674 | 19674 |
| 16675 | 17675 | 18675 | 19675 |
| 16676 | 17676 | 18676 | 19676 |
| 16677 | 17677 | 18677 | 19677 |
| 16678 | 17678 | 18678 | 19678 |
| 16679 | 17679 | 18679 | 19679 |
| 16680 | 17680 | 18680 | 19680 |
| 16681 | 17681 | 18681 | 19681 |
| 16682 | 17682 | 18682 | 19682 |
| 16683 | 17683 | 18683 | 19683 |
| 16684 | 17684 | 18684 | 19684 |
| 16685 | 17685 | 18685 | 19685 |
| 16686 | 17686 | 18686 | 19686 |
| 16687 | 17687 | 18687 | 19687 |
| 16688 | 17688 | 18688 | 19688 |
| 16689 | 17689 | 18689 | 19689 |
| 16690 | 17690 | 18690 | 19690 |
| 16691 | 17691 | 18691 | 19691 |
| 16692 | 17692 | 18692 | 19692 |
| 16693 | 17693 | 18693 | 19693 |
| 16694 | 17694 | 18694 | 19694 |
| 16695 | 17695 | 18695 | 19695 |
| 16696 | 17696 | 18696 | 19696 |
| 16697 | 17697 | 18697 | 19697 |
| 16698 | 17698 | 18698 | 19698 |
| 16699 | 17699 | 18699 | 19699 |
| 16700 | 17700 | 18700 | 19700 |
| 16701 | 17701 | 18701 | 19701 |
| 16702 | 17702 | 18702 | 19702 |
| 16703 | 17703 | 18703 | 19703 |
| 16704 | 17704 | 18704 | 19704 |
| 16705 | 17705 | 18705 | 19705 |
| 16706 | 17706 | 18706 | 19706 |
| 16707 | 17707 | 18707 | 19707 |
| 16708 | 17708 | 18708 | 19708 |
| 16709 | 17709 | 18709 | 19709 |
| 16710 | 17710 | 18710 | 19710 |
| 16711 | 17711 | 18711 | 19711 |
| 16712 | 17712 | 18712 | 19712 |
| 16713 | 17713 | 18713 | 19713 |
| 16714 | 17714 | 18714 | 19714 |
| 16715 | 17715 | 18715 | 19715 |
| 16716 | 17716 | 18716 | 19716 |
| 16717 | 17717 | 18717 | 19717 |
| 16718 | 17718 | 18718 | 19718 |
| 16719 | 17719 | 18719 | 19719 |
| 16720 | 17720 | 18720 | 19720 |
| 16721 | 17721 | 18721 | 19721 |
| 16722 | 17722 | 18722 | 19722 |
| 16723 | 17723 | 18723 | 19723 |
| 16724 | 17724 | 18724 | 19724 |
| 16725 | 17725 | 18725 | 19725 |
| 16726 | 17726 | 18726 | 19726 |
| 16727 | 17727 | 18727 | 19727 |
| 16728 | 17728 | 18728 | 19728 |
| 16729 | 17729 | 18729 | 19729 |
| 16730 | 17730 | 18730 | 19730 |
| 16731 | 17731 | 18731 | 19731 |
| 16732 | 17732 | 18732 | 19732 |
| 16733 | 17733 | 18733 | 19733 |
| 16734 | 17734 | 18734 | 19734 |
| 16735 | 17735 | 18735 | 19735 |
| 16736 | 17736 | 18736 | 19736 |
| 16737 | 17737 | 18737 | 19737 |
| 16738 | 17738 | 18738 | 19738 |
| 16739 | 17739 | 18739 | 19739 |
| 16740 | 17740 | 18740 | 19740 |
| 16741 | 17741 | 18741 | 19741 |
| 16742 | 17742 | 18742 | 19742 |
| 16743 | 17743 | 18743 | 19743 |
| 16744 | 17744 | 18744 | 19744 |
| 16745 | 17745 | 18745 | 19745 |
| 16746 | 17746 | 18746 | 19746 |
| 16747 | 17747 | 18747 | 19747 |
| 16748 | 17748 | 18748 | 19748 |
| 16749 | 17749 | 18749 | 19749 |
| 16750 | 17750 | 18750 | 19750 |
| 16751 | 17751 | 18751 | 19751 |
| 16752 | 17752 | 18752 | 19752 |
| 16753 | 17753 | 18753 | 19753 |
| 16754 | 17754 | 18754 | 19754 |
| 16755 | 17755 | 18755 | 19755 |
| 16756 | 17756 | 18756 | 19756 |
| 16757 | 17757 | 18757 | 19757 |
| 16758 | 17758 | 18758 | 19758 |
| 16759 | 17759 | 18759 | 19759 |
| 16760 | 17760 | 18760 | 19760 |
| 16761 | 17761 | 18761 | 19761 |
| 16762 | 17762 | 18762 | 19762 |
| 16763 | 17763 | 18763 | 19763 |
| 16764 | 17764 | 18764 | 19764 |
| 16765 | 17765 | 18765 | 19765 |
| 16766 | 17766 | 18766 | 19766 |
| 16767 | 17767 | 18767 | 19767 |
| 16768 | 17768 | 18768 | 19768 |
| 16769 | 17769 | 18769 | 19769 |
| 16770 | 17770 | 18770 | 19770 |
| 16771 | 17771 | 18771 | 19771 |
| 16772 | 17772 | 18772 | 19772 |
| 16773 | 17773 | 18773 | 19773 |
| 16774 | 17774 | 18774 | 19774 |
| 16775 | 17775 | 18775 | 19775 |
| 16776 | 17776 | 18776 | 19776 |
| 16777 | 17777 | 18777 | 19777 |
| 16778 | 17778 | 18778 | 19778 |
| 16779 | 17779 | 18779 | 19779 |
| 16780 | 17780 | 18780 | 19780 |
| 16781 | 17781 | 18781 | 19781 |
| 16782 | 17782 | 18782 | 19782 |
| 16783 | 17783 | 18783 | 19783 |
| 16784 | 17784 | 18784 | 19784 |
| 16785 | 17785 | 18785 | 19785 |
| 16786 | 17786 | 18786 | 19786 |
| 16787 | 17787 | 18787 | 19787 |
| 16788 | 17788 | 18788 | 19788 |
| 16789 | 17789 | 18789 | 19789 |
| 16790 | 17790 | 18790 | 19790 |
| 16791 | 17791 | 18791 | 19791 |
| 16792 | 17792 | 18792 | 19792 |
| 16793 | 17793 | 18793 | 19793 |
| 16794 | 17794 | 18794 | 19794 |
| 16795 | 17795 | 18795 | 19795 |
| 16796 | 17796 | 18796 | 19796 |
| 16797 | 17797 | 18797 | 19797 |
| 16798 | 17798 | 18798 | 19798 |
| 16799 | 17799 | 18799 | 19799 |
| 16800 | 17800 | 18800 | 19800 |
| 16801 | 17801 | 18801 | 19801 |
| 16802 | 17802 | 18802 | 19802 |
| 16803 | 17803 | 18803 | 19803 |
| 16804 | 17804 | 18804 | 19804 |
| 16805 | 17805 | 18805 | 19805 |
| 16806 | 17806 | 18806 | 19806 |
| 16807 | 17807 | 18807 | 19807 |
| 16808 | 17808 | 18808 | 19808 |
| 16809 | 17809 | 18809 | 19809 |
| 16810 | 17810 | 18810 | 19810 |
| 16811 | 17811 | 18811 | 19811 |
| 16812 | 17812 | 18812 | 19812 |
| 16813 | 17813 | 18813 | 19813 |
| 16814 | 17814 | 18814 | 19814 |
| 16815 | 17815 | 18815 | 19815 |
| 16816 | 17816 | 18816 | 19816 |
| 16817 | 17817 | 18817 | 19817 |
| 16818 | 17818 | 18818 | 19818 |
| 16819 | 17819 | 18819 | 19819 |
| 16820 | 17820 | 18820 | 19820 |
| 16821 | 17821 | 18821 | 19821 |
| 16822 | 17822 | 18822 | 19822 |
| 16823 | 17823 | 18823 | 19823 |
| 16824 | 17824 | 18824 | 19824 |
| 16825 | 17825 | 18825 | 19825 |
| 16826 | 17826 | 18826 | 19826 |
| 16827 | 17827 | 18827 | 19827 |
| 16828 | 17828 | 18828 | 19828 |
| 16829 | 17829 | 18829 | 19829 |
| 16830 | 17830 | 18830 | 19830 |
| 16831 | 17831 | 18831 | 19831 |
| 16832 | 17832 | 18832 | 19832 |
| 16833 | 17833 | 18833 | 19833 |
| 16834 | 17834 | 18834 | 19834 |
| 16835 | 17835 | 18835 | 19835 |
| 16836 | 17836 | 18836 | 19836 |
| 16837 | 17837 | 18837 | 19837 |
| 16838 | 17838 | 18838 | 19838 |
| 16839 | 17839 | 18839 | 19839 |
| 16840 | 17840 | 18840 | 19840 |
| 16841 | 17841 | 18841 | 19841 |
| 16842 | 17842 | 18842 | 19842 |
| 16843 | 17843 | 18843 | 19843 |
| 16844 | 17844 | 18844 | 19844 |
| 16845 | 17845 | 18845 | 19845 |
| 16846 | 17846 | 18846 | 19846 |
| 16847 | 17847 | 18847 | 19847 |
| 16848 | 17848 | 18848 | 19848 |
| 16849 | 17849 | 18849 | 19849 |
| 16850 | 17850 | 18850 | 19850 |
| 16851 | 17851 | 18851 | 19851 |
| 16852 | 17852 | 18852 | 19852 |
| 16853 | 17853 | 18853 | 19853 |
| 16854 | 17854 | 18854 | 19854 |
| 16855 | 17855 | 18855 | 19855 |
| 16856 | 17856 | 18856 | 19856 |
| 16857 | 17857 | 18857 | 19857 |
| 16858 | 17858 | 18858 | 19858 |
| 16859 | 17859 | 18859 | 19859 |
| 16860 | 17860 | 18860 | 19860 |
| 16861 | 17861 | 18861 | 19861 |
| 16862 | 17862 | 18862 | 19862 |
| 16863 | 17863 | 18863 | 19863 |
| 16864 | 17864 | 18864 | 19864 |
| 16865 | 17865 | 18865 | 19865 |
| 16866 | 17866 | 18866 | 19866 |
| 16867 | 17867 | 18867 | 19867 |
| 16868 | 17868 | 18868 | 19868 |
| 16869 | 17869 | 18869 | 19869 |
| 16870 | 17870 | 18870 | 19870 |
| 16871 | 17871 | 18871 | 19871 |
| 16872 | 17872 | 18872 | 19872 |
| 16873 | 17873 | 18873 | 19873 |
| 16874 | 17874 | 18874 | 19874 |
| 16875 | 17875 | 18875 | 19875 |
| 16876 | 17876 | 18876 | 19876 |
| 16877 | 17877 | 18877 | 19877 |
| 16878 | 17878 | 18878 | 19878 |
| 16879 | 17879 | 18879 | 19879 |
| 16880 | 17880 | 18880 | 19880 |
| 16881 | 17881 | 18881 | 19881 |
| 16882 | 17882 | 18882 | 19882 |
| 16883 | 17883 | 18883 | 19883 |
| 16884 | 17884 | 18884 | 19884 |
| 16885 | 17885 | 18885 | 19885 |
| 16886 | 17886 | 18886 | 19886 |
| 16887 | 17887 | 18887 | 19887 |
| 16888 | 17888 | 18888 | 19888 |
| 16889 | 17889 | 18889 | 19889 |
| 16890 | 17890 | 18890 | 19890 |
| 16891 | 17891 | 18891 | 19891 |
| 16892 | 17892 | 18892 | 19892 |
| 16893 | 17893 | 18893 | 19893 |
| 16894 | 17894 | 18894 | 19894 |
| 16895 | 17895 | 18895 | 19895 |
| 16896 | 17896 | 18896 | 19896 |
| 16897 | 17897 | 18897 | 19897 |
| 16898 | 17898 | 18898 | 19898 |
| 16899 | 17899 | 18899 | 19899 |
| 16900 | 17900 | 18900 | 19900 |
| 16901 | 17901 | 18901 | 19901 |
| 16902 | 17902 | 18902 | 19902 |
| 16903 | 17903 | 18903 | 19903 |
| 16904 | 17904 | 18904 | 19904 |
| 16905 | 17905 | 18905 | 19905 |
| 16906 | 17906 | 18906 | 19906 |
| 16907 | 17907 | 18907 | 19907 |
| 16908 | 17908 | 18908 | 19908 |
| 16909 | 17909 | 18909 | 19909 |
| 16910 | 17910 | 18910 | 19910 |
| 16911 | 17911 | 18911 | 19911 |
| 16912 | 17912 | 18912 | 19912 |
| 16913 | 17913 | 18913 | 19913 |
| 16914 | 17914 | 18914 | 19914 |
| 16915 | 17915 | 18915 | 19915 |
| 16916 | 17916 | 18916 | 19916 |
| 16917 | 17917 | 18917 | 19917 |
| 16918 | 17918 | 18918 | 19918 |
| 16919 | 17919 | 18919 | 19919 |
| 16920 | 17920 | 18920 | 19920 |
| 16921 | 17921 | 18921 | 19921 |
| 16922 | 17922 | 18922 | 19922 |
| 16923 | 17923 | 18923 | 19923 |
| 16924 | 17924 | 18924 | 19924 |
| 16925 | 17925 | 18925 | 19925 |
| 16926 | 17926 | 18926 | 19926 |
| 16927 | 17927 | 18927 | 19927 |
| 16928 | 17928 | 18928 | 19928 |
| 16929 | 17929 | 18929 | 19929 |
| 16930 | 17930 | 18930 | 19930 |
| 16931 | 17931 | 18931 | 19931 |
| 16932 | 17932 | 18932 | 19932 |
| 16933 | 17933 | 18933 | 19933 |
| 16934 | 17934 | 18934 | 19934 |
| 16935 | 17935 | 18935 | 19935 |
| 16936 | 17936 | 18936 | 19936 |
| 16937 | 17937 | 18937 | 19937 |
| 16938 | 17938 | 18938 | 19938 |
| 16939 | 17939 | 18939 | 19939 |
| 16940 | 17940 | 18940 | 19940 |
| 16941 | 17941 | 18941 | 19941 |
| 16942 | 17942 | 18942 | 19942 |
| 16943 | 17943 | 18943 | 19943 |
| 16944 | 17944 | 18944 | 19944 |
| 16945 | 17945 | 18945 | 19945 |
| 16946 | 17946 | 18946 | 19946 |
| 16947 | 17947 | 18947 | 19947 |
| 16948 | 17948 | 18948 | 19948 |
| 16949 | 17949 | 18949 | 19949 |
| 16950 | 17950 | 18950 | 19950 |
| 16951 | 17951 | 18951 | 19951 |
| 16952 | 17952 | 18952 | 19952 |
| 16953 | 17953 | 18953 | 19953 |
| 16954 | 17954 | 18954 | 19954 |
| 16955 | 17955 | 18955 | 19955 |
| 16956 | 17956 | 18956 | 19956 |
| 16957 | 17957 | 18957 | 19957 |
| 16958 | 17958 | 18958 | 19958 |
| 16959 | 17959 | 18959 | 19959 |
| 16960 | 17960 | 18960 | 19960 |
| 16961 | 17961 | 18961 | 19961 |
| 16962 | 17962 | 18962 | 19962 |
| 16963 | 17963 | 18963 | 19963 |
| 16964 | 17964 | 18964 | 19964 |
| 16965 | 17965 | 18965 | 19965 |
| 16966 | 17966 | 18966 | 19966 |
| 16967 | 17967 | 18967 | 19967 |
| 16968 | 17968 | 18968 | 19968 |
| 16969 | 17969 | 18969 | 19969 |
| 16970 | 17970 | 18970 | 19970 |
| 16971 | 17971 | 18971 | 19971 |
| 16972 | 17972 | 18972 | 19972 |
| 16973 | 17973 | 18973 | 19973 |
| 16974 | 17974 | 18974 | 19974 |
| 16975 | 17975 | 18975 | 19975 |
| 16976 | 17976 | 18976 | 19976 |
| 16977 | 17977 | 18977 | 19977 |
| 16978 | 17978 | 18978 | 19978 |
| 16979 | 17979 | 18979 | 19979 |
| 16980 | 17980 | 18980 | 19980 |
| 16981 | 17981 | 18981 | 19981 |
| 16982 | 17982 | 18982 | 19982 |
| 16983 | 17983 | 18983 | 19983 |
| 16984 | 17984 | 18984 | 19984 |
| 16985 | 17985 | 18985 | 19985 |
| 16986 | 17986 | 18986 | 19986 |
| 16987 | 17987 | 18987 | 19987 |
| 16988 | 17988 | 18988 | 19988 |
| 16989 | 17989 | 18989 | 19989 |
| 16990 | 17990 | 18990 | 19990 |
| 16991 | 17991 | 18991 | 19991 |
| 16992 | 17992 | 18992 | 19992 |
| 16993 | 17993 | 18993 | 19993 |
| 16994 | 17994 | 18994 | 19994 |
| 16995 | 17995 | 18995 | 19995 |
| 16996 | 17996 | 18996 | 19996 |
| 16997 | 17997 | 18997 | 19997 |
| 16998 | 17998 | 18998 | 19998 |
| 16999 | 17999 | 18999 | 19999 |
| 17000 | 18000 | 19000 | 20000 |
| 17001 | 18001 | 19001 | 20001 |
| 17002 | 18002 | 19002 | 20002 |

In some embodiments, the sequences that specifically bind to pecan comprise an inner sequence selected from the group consisting of the nucleic acid sequences of SEQ ID NOs. 20003 to 21002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to pecan may comprise a nucleic acid sequence selected from the group consisting of SEQ ID NOs. 21003 to 22002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to pecan may comprise a nucleic acid sequence selected from the group consisting of SEQ ID NOs. 22003 to 23002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to pecan may comprise a nucleic acid sequence selected from the group consisting of SEQ ID NOs. 23003 to 24002. In one embodiment, the aptamer of the present disclosure that specifically binds to pecan may comprise a nucleic acid sequence selected from the group consisting of SEQ ID NOs. 20003 to 24002 listed in Table 7, or variant thereof.

TABLE 7
Aptamer sequences against pecan
Aptamer sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 20003 | 21003 | 22003 | 23003 |
| 20004 | 21004 | 22004 | 23004 |
| 20005 | 21005 | 22005 | 23005 |
| 20006 | 21006 | 22006 | 23006 |
| 20007 | 21007 | 22007 | 23007 |
| 20008 | 21008 | 22008 | 23008 |
| 20009 | 21009 | 22009 | 23009 |
| 20010 | 21010 | 22010 | 23010 |
| 20011 | 21011 | 22011 | 23011 |
| 20012 | 21012 | 22012 | 23012 |
| 20013 | 21013 | 22013 | 23013 |
| 20014 | 21014 | 22014 | 23014 |
| 20015 | 21015 | 22015 | 23015 |
| 20016 | 21016 | 22016 | 23016 |
| 20017 | 21017 | 22017 | 23017 |
| 20018 | 21018 | 22018 | 23018 |
| 20019 | 21019 | 22019 | 23019 |
| 20020 | 21020 | 22020 | 23020 |
| 20021 | 21021 | 22021 | 23021 |
| 20022 | 21022 | 22022 | 23022 |
| 20023 | 21023 | 22023 | 23023 |
| 20024 | 21024 | 22024 | 23024 |
| 20025 | 21025 | 22025 | 23025 |
| 20026 | 21026 | 22026 | 23026 |
| 20027 | 21027 | 22027 | 23027 |
| 20028 | 21028 | 22028 | 23028 |
| 20029 | 21029 | 22029 | 23029 |
| 20030 | 21030 | 22030 | 23030 |
| 20031 | 21031 | 22031 | 23031 |
| 20032 | 21032 | 22032 | 23032 |
| 20033 | 21033 | 22033 | 23033 |
| 20034 | 21034 | 22034 | 23034 |
| 20035 | 21035 | 22035 | 23035 |
| 20036 | 21036 | 22036 | 23036 |
| 20037 | 21037 | 22037 | 23037 |
| 20038 | 21038 | 22038 | 23038 |
| 20039 | 21039 | 22039 | 23039 |
| 20040 | 21040 | 22040 | 23040 |
| 20041 | 21041 | 22041 | 23041 |
| 20042 | 21042 | 22042 | 23042 |
| 20043 | 21043 | 22043 | 23043 |
| 20044 | 21044 | 22044 | 23044 |
| 20045 | 21045 | 22045 | 23045 |
| 20046 | 21046 | 22046 | 23046 |
| 20047 | 21047 | 22047 | 23047 |
| 20048 | 21048 | 22048 | 23048 |
| 20049 | 21049 | 22049 | 23049 |
| 20050 | 21050 | 22050 | 23050 |
| 20051 | 21051 | 22051 | 23051 |
| 20052 | 21052 | 22052 | 23052 |
| 20053 | 21053 | 22053 | 23053 |
| 20054 | 21054 | 22054 | 23054 |
| 20055 | 21055 | 22055 | 23055 |
| 20056 | 21056 | 22056 | 23056 |
| 20057 | 21057 | 22057 | 23057 |
| 20058 | 21058 | 22058 | 23058 |
| 20059 | 21059 | 22059 | 23059 |
| 20060 | 21060 | 22060 | 23060 |
| 20061 | 21061 | 22061 | 23061 |
| 20062 | 21062 | 22062 | 23062 |
| 20063 | 21063 | 22063 | 23063 |
| 20064 | 21064 | 22064 | 23064 |
| 20065 | 21065 | 22065 | 23065 |
| 20066 | 21066 | 22066 | 23066 |
| 20067 | 21067 | 22067 | 23067 |
| 20068 | 21068 | 22068 | 23068 |
| 20069 | 21069 | 22069 | 23069 |
| 20070 | 21070 | 22070 | 23070 |
| 20071 | 21071 | 22071 | 23071 |
| 20072 | 21072 | 22072 | 23072 |
| 20073 | 21073 | 22073 | 23073 |
| 20074 | 21074 | 22074 | 23074 |
| 20075 | 21075 | 22075 | 23075 |
| 20076 | 21076 | 22076 | 23076 |
| 20077 | 21077 | 22077 | 23077 |
| 20078 | 21078 | 22078 | 23078 |
| 20079 | 21079 | 22079 | 23079 |
| 20080 | 21080 | 22080 | 23080 |
| 20081 | 21081 | 22081 | 23081 |
| 20082 | 21082 | 22082 | 23082 |
| 20083 | 21083 | 22083 | 23083 |
| 20084 | 21084 | 22084 | 23084 |
| 20085 | 21085 | 22085 | 23085 |
| 20086 | 21086 | 22086 | 23086 |
| 20087 | 21087 | 22087 | 23087 |
| 20088 | 21088 | 22088 | 23088 |
| 20089 | 21089 | 22089 | 23089 |
| 20090 | 21090 | 22090 | 23090 |
| 20091 | 21091 | 22091 | 23091 |
| 20092 | 21092 | 22092 | 23092 |
| 20093 | 21093 | 22093 | 23093 |
| 20094 | 21094 | 22094 | 23094 |
| 20095 | 21095 | 22095 | 23095 |
| 20096 | 21096 | 22096 | 23096 |
| 20097 | 21097 | 22097 | 23097 |
| 20098 | 21098 | 22098 | 23098 |
| 20099 | 21099 | 22099 | 23099 |
| 20100 | 21100 | 22100 | 23100 |
| 20101 | 21101 | 22101 | 23101 |
| 20102 | 21102 | 22102 | 23102 |
| 20103 | 21103 | 22103 | 23103 |
| 20104 | 21104 | 22104 | 23104 |
| 20105 | 21105 | 22105 | 23105 |
| 20106 | 21106 | 22106 | 23106 |
| 20107 | 21107 | 22107 | 23107 |
| 20108 | 21108 | 22108 | 23108 |
| 20109 | 21109 | 22109 | 23109 |
| 20110 | 21110 | 22110 | 23110 |
| 20111 | 21111 | 22111 | 23111 |
| 20112 | 21112 | 22112 | 23112 |
| 20113 | 21113 | 22113 | 23113 |
| 20114 | 21114 | 22114 | 23114 |
| 20115 | 21115 | 22115 | 23115 |
| 20116 | 21116 | 22116 | 23116 |
| 20117 | 21117 | 22117 | 23117 |
| 20118 | 21118 | 22118 | 23118 |
| 20119 | 21119 | 22119 | 23119 |
| 20120 | 21120 | 22120 | 23120 |
| 20121 | 21121 | 22121 | 23121 |
| 20122 | 21122 | 22122 | 23122 |
| 20123 | 21123 | 22123 | 23123 |
| 20124 | 21124 | 22124 | 23124 |
| 20125 | 21125 | 22125 | 23125 |
| 20126 | 21126 | 22126 | 23126 |
| 20127 | 21127 | 22127 | 23127 |
| 20128 | 21128 | 22128 | 23128 |
| 20129 | 21129 | 22129 | 23129 |
| 20130 | 21130 | 22130 | 23130 |
| 20131 | 21131 | 22131 | 23131 |
| 20132 | 21132 | 22132 | 23132 |
| 20133 | 21133 | 22133 | 23133 |
| 20134 | 21134 | 22134 | 23134 |
| 20135 | 21135 | 22135 | 23135 |
| 20136 | 21136 | 22136 | 23136 |
| 20137 | 21137 | 22137 | 23137 |
| 20138 | 21138 | 22138 | 23138 |
| 20139 | 21139 | 22139 | 23139 |
| 20140 | 21140 | 22140 | 23140 |
| 20141 | 21141 | 22141 | 23141 |
| 20142 | 21142 | 22142 | 23142 |
| 20143 | 21143 | 22143 | 23143 |
| 20144 | 21144 | 22144 | 23144 |
| 20145 | 21145 | 22145 | 23145 |
| 20146 | 21146 | 22146 | 23146 |
| 20147 | 21147 | 22147 | 23147 |
| 20148 | 21148 | 22148 | 23148 |
| 20149 | 21149 | 22149 | 23149 |
| 20150 | 21150 | 22150 | 23150 |
| 20151 | 21151 | 22151 | 23151 |
| 20152 | 21152 | 22152 | 23152 |
| 20153 | 21153 | 22153 | 23153 |
| 20154 | 21154 | 22154 | 23154 |
| 20155 | 21155 | 22155 | 23155 |
| 20156 | 21156 | 22156 | 23156 |
| 20157 | 21157 | 22157 | 23157 |
| 20158 | 21158 | 22158 | 23158 |
| 20159 | 21159 | 22159 | 23159 |
| 20160 | 21160 | 22160 | 23160 |
| 20161 | 21161 | 22161 | 23161 |
| 20162 | 21162 | 22162 | 23162 |
| 20163 | 21163 | 22163 | 23163 |
| 20164 | 21164 | 22164 | 23164 |
| 20165 | 21165 | 22165 | 23165 |
| 20166 | 21166 | 22166 | 23166 |
| 20167 | 21167 | 22167 | 23167 |
| 20168 | 21168 | 22168 | 23168 |
| 20169 | 21169 | 22169 | 23169 |
| 20170 | 21170 | 22170 | 23170 |
| 20171 | 21171 | 22171 | 23171 |
| 20172 | 21172 | 22172 | 23172 |
| 20173 | 21173 | 22173 | 23173 |
| 20174 | 21174 | 22174 | 23174 |
| 20175 | 21175 | 22175 | 23175 |
| 20176 | 21176 | 22176 | 23176 |
| 20177 | 21177 | 22177 | 23177 |
| 20178 | 21178 | 22178 | 23178 |
| 20179 | 21179 | 22179 | 23179 |
| 20180 | 21180 | 22180 | 23180 |
| 20181 | 21181 | 22181 | 23181 |
| 20182 | 21182 | 22182 | 23182 |
| 20183 | 21183 | 22183 | 23183 |
| 20184 | 21184 | 22184 | 23184 |
| 20185 | 21185 | 22185 | 23185 |
| 20186 | 21186 | 22186 | 23186 |
| 20187 | 21187 | 22187 | 23187 |
| 20188 | 21188 | 22188 | 23188 |
| 20189 | 21189 | 22189 | 23189 |
| 20190 | 21190 | 22190 | 23190 |
| 20191 | 21191 | 22191 | 23191 |
| 20192 | 21192 | 22192 | 23192 |
| 20193 | 21193 | 22193 | 23193 |
| 20194 | 21194 | 22194 | 23194 |
| 20195 | 21195 | 22195 | 23195 |
| 20196 | 21196 | 22196 | 23196 |
| 20197 | 21197 | 22197 | 23197 |
| 20198 | 21198 | 22198 | 23198 |
| 20199 | 21199 | 22199 | 23199 |
| 20200 | 21200 | 22200 | 23200 |
| 20201 | 21201 | 22201 | 23201 |
| 20202 | 21202 | 22202 | 23202 |
| 20203 | 21203 | 22203 | 23203 |
| 20204 | 21204 | 22204 | 23204 |
| 20205 | 21205 | 22205 | 23205 |
| 20206 | 21206 | 22206 | 23206 |
| 20207 | 21207 | 22207 | 23207 |
| 20208 | 21208 | 22208 | 23208 |
| 20209 | 21209 | 22209 | 23209 |
| 20210 | 21210 | 22210 | 23210 |
| 20211 | 21211 | 22211 | 23211 |
| 20212 | 21212 | 22212 | 23212 |
| 20213 | 21213 | 22213 | 23213 |
| 20214 | 21214 | 22214 | 23214 |
| 20215 | 21215 | 22215 | 23215 |
| 20216 | 21216 | 22216 | 23216 |
| 20217 | 21217 | 22217 | 23217 |
| 20218 | 21218 | 22218 | 23218 |
| 20219 | 21219 | 22219 | 23219 |
| 20220 | 21220 | 22220 | 23220 |
| 20221 | 21221 | 22221 | 23221 |
| 20222 | 21222 | 22222 | 23222 |
| 20223 | 21223 | 22223 | 23223 |
| 20224 | 21224 | 22224 | 23224 |
| 20225 | 21225 | 22225 | 23225 |
| 20226 | 21226 | 22226 | 23226 |
| 20227 | 21227 | 22227 | 23227 |
| 20228 | 21228 | 22228 | 23228 |
| 20229 | 21229 | 22229 | 23229 |
| 20230 | 21230 | 22230 | 23230 |
| 20231 | 21231 | 22231 | 23231 |
| 20232 | 21232 | 22232 | 23232 |
| 20233 | 21233 | 22233 | 23233 |
| 20234 | 21234 | 22234 | 23234 |
| 20235 | 21235 | 22235 | 23235 |
| 20236 | 21236 | 22236 | 23236 |
| 20237 | 21237 | 22237 | 23237 |
| 20238 | 21238 | 22238 | 23238 |
| 20239 | 21239 | 22239 | 23239 |
| 20240 | 21240 | 22240 | 23240 |
| 20241 | 21241 | 22241 | 23241 |
| 20242 | 21242 | 22242 | 23242 |
| 20243 | 21243 | 22243 | 23243 |
| 20244 | 21244 | 22244 | 23244 |
| 20245 | 21245 | 22245 | 23245 |
| 20246 | 21246 | 22246 | 23246 |
| 20247 | 21247 | 22247 | 23247 |
| 20248 | 21248 | 22248 | 23248 |
| 20249 | 21249 | 22249 | 23249 |
| 20250 | 21250 | 22250 | 23250 |
| 20251 | 21251 | 22251 | 23251 |
| 20252 | 21252 | 22252 | 23252 |
| 20253 | 21253 | 22253 | 23253 |
| 20254 | 21254 | 22254 | 23254 |
| 20255 | 21255 | 22255 | 23255 |
| 20256 | 21256 | 22256 | 23256 |
| 20257 | 21257 | 22257 | 23257 |
| 20258 | 21258 | 22258 | 23258 |
| 20259 | 21259 | 22259 | 23259 |
| 20260 | 21260 | 22260 | 23260 |
| 20261 | 21261 | 22261 | 23261 |
| 20262 | 21262 | 22262 | 23262 |
| 20263 | 21263 | 22263 | 23263 |
| 20264 | 21264 | 22264 | 23264 |
| 20265 | 21265 | 22265 | 23265 |
| 20266 | 21266 | 22266 | 23266 |
| 20267 | 21267 | 22267 | 23267 |
| 20268 | 21268 | 22268 | 23268 |
| 20269 | 21269 | 22269 | 23269 |
| 20270 | 21270 | 22270 | 23270 |
| 20271 | 21271 | 22271 | 23271 |
| 20272 | 21272 | 22272 | 23272 |
| 20273 | 21273 | 22273 | 23273 |
| 20274 | 21274 | 22274 | 23274 |
| 20275 | 21275 | 22275 | 23275 |
| 20276 | 21276 | 22276 | 23276 |
| 20277 | 21277 | 22277 | 23277 |
| 20278 | 21278 | 22278 | 23278 |
| 20279 | 21279 | 22279 | 23279 |
| 20280 | 21280 | 22280 | 23280 |
| 20281 | 21281 | 22281 | 23281 |
| 20282 | 21282 | 22282 | 23282 |
| 20283 | 21283 | 22283 | 23283 |
| 20284 | 21284 | 22284 | 23284 |
| 20285 | 21285 | 22285 | 23285 |
| 20286 | 21286 | 22286 | 23286 |
| 20287 | 21287 | 22287 | 23287 |
| 20288 | 21288 | 22288 | 23288 |
| 20289 | 21289 | 22289 | 23289 |
| 20290 | 21290 | 22290 | 23290 |
| 20291 | 21291 | 22291 | 23291 |
| 20292 | 21292 | 22292 | 23292 |
| 20293 | 21293 | 22293 | 23293 |
| 20294 | 21294 | 22294 | 23294 |
| 20295 | 21295 | 22295 | 23295 |
| 20296 | 21296 | 22296 | 23296 |
| 20297 | 21297 | 22297 | 23297 |
| 20298 | 21298 | 22298 | 23298 |
| 20299 | 21299 | 22299 | 23299 |
| 20300 | 21300 | 22300 | 23300 |
| 20301 | 21301 | 22301 | 23301 |
| 20302 | 21302 | 22302 | 23302 |
| 20303 | 21303 | 22303 | 23303 |
| 20304 | 21304 | 22304 | 23304 |
| 20305 | 21305 | 22305 | 23305 |
| 20306 | 21306 | 22306 | 23306 |
| 20307 | 21307 | 22307 | 23307 |
| 20308 | 21308 | 22308 | 23308 |
| 20309 | 21309 | 22309 | 23309 |
| 20310 | 21310 | 22310 | 23310 |
| 20311 | 21311 | 22311 | 23311 |
| 20312 | 21312 | 22312 | 23312 |
| 20313 | 21313 | 22313 | 23313 |
| 20314 | 21314 | 22314 | 23314 |
| 20315 | 21315 | 22315 | 23315 |
| 20316 | 21316 | 22316 | 23316 |
| 20317 | 21317 | 22317 | 23317 |
| 20318 | 21318 | 22318 | 23318 |
| 20319 | 21319 | 22319 | 23319 |
| 20320 | 21320 | 22320 | 23320 |
| 20321 | 21321 | 22321 | 23321 |
| 20322 | 21322 | 22322 | 23322 |
| 20323 | 21323 | 22323 | 23323 |
| 20324 | 21324 | 22324 | 23324 |
| 20325 | 21325 | 22325 | 23325 |
| 20326 | 21326 | 22326 | 23326 |
| 20327 | 21327 | 22327 | 23327 |
| 20328 | 21328 | 22328 | 23328 |
| 20329 | 21329 | 22329 | 23329 |
| 20330 | 21330 | 22330 | 23330 |
| 20331 | 21331 | 22331 | 23331 |
| 20332 | 21332 | 22332 | 23332 |
| 20333 | 21333 | 22333 | 23333 |
| 20334 | 21334 | 22334 | 23334 |
| 20335 | 21335 | 22335 | 23335 |
| 20336 | 21336 | 22336 | 23336 |
| 20337 | 21337 | 22337 | 23337 |
| 20338 | 21338 | 22338 | 23338 |
| 20339 | 21339 | 22339 | 23339 |
| 20340 | 21340 | 22340 | 23340 |
| 20341 | 21341 | 22341 | 23341 |
| 20342 | 21342 | 22342 | 23342 |
| 20343 | 21343 | 22343 | 23343 |
| 20344 | 21344 | 22344 | 23344 |
| 20345 | 21345 | 22345 | 23345 |
| 20346 | 21346 | 22346 | 23346 |
| 20347 | 21347 | 22347 | 23347 |
| 20348 | 21348 | 22348 | 23348 |
| 20349 | 21349 | 22349 | 23349 |
| 20350 | 21350 | 22350 | 23350 |
| 20351 | 21351 | 22351 | 23351 |
| 20352 | 21352 | 22352 | 23352 |
| 20353 | 21353 | 22353 | 23353 |
| 20354 | 21354 | 22354 | 23354 |
| 20355 | 21355 | 22355 | 23355 |
| 20356 | 21356 | 22356 | 23356 |
| 20357 | 21357 | 22357 | 23357 |
| 20358 | 21358 | 22358 | 23358 |
| 20359 | 21359 | 22359 | 23359 |
| 20360 | 21360 | 22360 | 23360 |
| 20361 | 21361 | 22361 | 23361 |
| 20362 | 21362 | 22362 | 23362 |
| 20363 | 21363 | 22363 | 23363 |
| 20364 | 21364 | 22364 | 23364 |
| 20365 | 21365 | 22365 | 23365 |
| 20366 | 21366 | 22366 | 23366 |
| 20367 | 21367 | 22367 | 23367 |
| 20368 | 21368 | 22368 | 23368 |
| 20369 | 21369 | 22369 | 23369 |
| 20370 | 21370 | 22370 | 23370 |
| 20371 | 21371 | 22371 | 23371 |
| 20372 | 21372 | 22372 | 23372 |
| 20373 | 21373 | 22373 | 23373 |
| 20374 | 21374 | 22374 | 23374 |
| 20375 | 21375 | 22375 | 23375 |
| 20376 | 21376 | 22376 | 23376 |
| 20377 | 21377 | 22377 | 23377 |
| 20378 | 21378 | 22378 | 23378 |
| 20379 | 21379 | 22379 | 23379 |
| 20380 | 21380 | 22380 | 23380 |
| 20381 | 21381 | 22381 | 23381 |
| 20382 | 21382 | 22382 | 23382 |
| 20383 | 21383 | 22383 | 23383 |
| 20384 | 21384 | 22384 | 23384 |
| 20385 | 21385 | 22385 | 23385 |
| 20386 | 21386 | 22386 | 23386 |
| 20387 | 21387 | 22387 | 23387 |
| 20388 | 21388 | 22388 | 23388 |
| 20389 | 21389 | 22389 | 23389 |
| 20390 | 21390 | 22390 | 23390 |
| 20391 | 21391 | 22391 | 23391 |
| 20392 | 21392 | 22392 | 23392 |
| 20393 | 21393 | 22393 | 23393 |
| 20394 | 21394 | 22394 | 23394 |
| 20395 | 21395 | 22395 | 23395 |
| 20396 | 21396 | 22396 | 23396 |
| 20397 | 21397 | 22397 | 23397 |
| 20398 | 21398 | 22398 | 23398 |
| 20399 | 21399 | 22399 | 23399 |
| 20400 | 21400 | 22400 | 23400 |
| 20401 | 21401 | 22401 | 23401 |
| 20402 | 21402 | 22402 | 23402 |
| 20403 | 21403 | 22403 | 23403 |
| 20404 | 21404 | 22404 | 23404 |
| 20405 | 21405 | 22405 | 23405 |
| 20406 | 21406 | 22406 | 23406 |
| 20407 | 21407 | 22407 | 23407 |
| 20408 | 21408 | 22408 | 23408 |
| 20409 | 21409 | 22409 | 23409 |
| 20410 | 21410 | 22410 | 23410 |
| 20411 | 21411 | 22411 | 23411 |
| 20412 | 21412 | 22412 | 23412 |
| 20413 | 21413 | 22413 | 23413 |
| 20414 | 21414 | 22414 | 23414 |
| 20415 | 21415 | 22415 | 23415 |
| 20416 | 21416 | 22416 | 23416 |
| 20417 | 21417 | 22417 | 23417 |
| 20418 | 21418 | 22418 | 23418 |
| 20419 | 21419 | 22419 | 23419 |
| 20420 | 21420 | 22420 | 23420 |
| 20421 | 21421 | 22421 | 23421 |
| 20422 | 21422 | 22422 | 23422 |
| 20423 | 21423 | 22423 | 23423 |
| 20424 | 21424 | 22424 | 23424 |
| 20425 | 21425 | 22425 | 23425 |
| 20426 | 21426 | 22426 | 23426 |
| 20427 | 21427 | 22427 | 23427 |
| 20428 | 21428 | 22428 | 23428 |
| 20429 | 21429 | 22429 | 23429 |
| 20430 | 21430 | 22430 | 23430 |
| 20431 | 21431 | 22431 | 23431 |
| 20432 | 21432 | 22432 | 23432 |
| 20433 | 21433 | 22433 | 23433 |
| 20434 | 21434 | 22434 | 23434 |
| 20435 | 21435 | 22435 | 23435 |
| 20436 | 21436 | 22436 | 23436 |
| 20437 | 21437 | 22437 | 23437 |
| 20438 | 21438 | 22438 | 23438 |
| 20439 | 21439 | 22439 | 23439 |
| 20440 | 21440 | 22440 | 23440 |
| 20441 | 21441 | 22441 | 23441 |
| 20442 | 21442 | 22442 | 23442 |
| 20443 | 21443 | 22443 | 23443 |
| 20444 | 21444 | 22444 | 23444 |
| 20445 | 21445 | 22445 | 23445 |
| 20446 | 21446 | 22446 | 23446 |
| 20447 | 21447 | 22447 | 23447 |
| 20448 | 21448 | 22448 | 23448 |
| 20449 | 21449 | 22449 | 23449 |
| 20450 | 21450 | 22450 | 23450 |
| 20451 | 21451 | 22451 | 23451 |
| 20452 | 21452 | 22452 | 23452 |
| 20453 | 21453 | 22453 | 23453 |
| 20454 | 21454 | 22454 | 23454 |
| 20455 | 21455 | 22455 | 23455 |
| 20456 | 21456 | 22456 | 23456 |
| 20457 | 21457 | 22457 | 23457 |
| 20458 | 21458 | 22458 | 23458 |
| 20459 | 21459 | 22459 | 23459 |
| 20460 | 21460 | 22460 | 23460 |
| 20461 | 21461 | 22461 | 23461 |
| 20462 | 21462 | 22462 | 23462 |
| 20463 | 21463 | 22463 | 23463 |
| 20464 | 21464 | 22464 | 23464 |
| 20465 | 21465 | 22465 | 23465 |
| 20466 | 21466 | 22466 | 23466 |
| 20467 | 21467 | 22467 | 23467 |
| 20468 | 21468 | 22468 | 23468 |
| 20469 | 21469 | 22469 | 23469 |
| 20470 | 21470 | 22470 | 23470 |
| 20471 | 21471 | 22471 | 23471 |
| 20472 | 21472 | 22472 | 23472 |
| 20473 | 21473 | 22473 | 23473 |
| 20474 | 21474 | 22474 | 23474 |
| 20475 | 21475 | 22475 | 23475 |
| 20476 | 21476 | 22476 | 23476 |
| 20477 | 21477 | 22477 | 23477 |
| 20478 | 21478 | 22478 | 23478 |
| 20479 | 21479 | 22479 | 23479 |
| 20480 | 21480 | 22480 | 23480 |
| 20481 | 21481 | 22481 | 23481 |
| 20482 | 21482 | 22482 | 23482 |
| 20483 | 21483 | 22483 | 23483 |
| 20484 | 21484 | 22484 | 23484 |
| 20485 | 21485 | 22485 | 23485 |
| 20486 | 21486 | 22486 | 23486 |
| 20487 | 21487 | 22487 | 23487 |
| 20488 | 21488 | 22488 | 23488 |
| 20489 | 21489 | 22489 | 23489 |
| 20490 | 21490 | 22490 | 23490 |
| 20491 | 21491 | 22491 | 23491 |
| 20492 | 21492 | 22492 | 23492 |
| 20493 | 21493 | 22493 | 23493 |
| 20494 | 21494 | 22494 | 23494 |
| 20495 | 21495 | 22495 | 23495 |
| 20496 | 21496 | 22496 | 23496 |
| 20497 | 21497 | 22497 | 23497 |
| 20498 | 21498 | 22498 | 23498 |
| 20499 | 21499 | 22499 | 23499 |
| 20500 | 21500 | 22500 | 23500 |
| 20501 | 21501 | 22501 | 23501 |
| 20502 | 21502 | 22502 | 23502 |
| 20503 | 21503 | 22503 | 23503 |
| 20504 | 21504 | 22504 | 23504 |
| 20505 | 21505 | 22505 | 23505 |
| 20506 | 21506 | 22506 | 23506 |
| 20507 | 21507 | 22507 | 23507 |
| 20508 | 21508 | 22508 | 23508 |
| 20509 | 21509 | 22509 | 23509 |
| 20510 | 21510 | 22510 | 23510 |
| 20511 | 21511 | 22511 | 23511 |
| 20512 | 21512 | 22512 | 23512 |
| 20513 | 21513 | 22513 | 23513 |
| 20514 | 21514 | 22514 | 23514 |
| 20515 | 21515 | 22515 | 23515 |
| 20516 | 21516 | 22516 | 23516 |
| 20517 | 21517 | 22517 | 23517 |
| 20518 | 21518 | 22518 | 23518 |
| 20519 | 21519 | 22519 | 23519 |
| 20520 | 21520 | 22520 | 23520 |
| 20521 | 21521 | 22521 | 23521 |
| 20522 | 21522 | 22522 | 23522 |
| 20523 | 21523 | 22523 | 23523 |
| 20524 | 21524 | 22524 | 23524 |
| 20525 | 21525 | 22525 | 23525 |
| 20526 | 21526 | 22526 | 23526 |
| 20527 | 21527 | 22527 | 23527 |
| 20528 | 21528 | 22528 | 23528 |
| 20529 | 21529 | 22529 | 23529 |
| 20530 | 21530 | 22530 | 23530 |
| 20531 | 21531 | 22531 | 23531 |
| 20532 | 21532 | 22532 | 23532 |
| 20533 | 21533 | 22533 | 23533 |
| 20534 | 21534 | 22534 | 23534 |
| 20535 | 21535 | 22535 | 23535 |
| 20536 | 21536 | 22536 | 23536 |
| 20537 | 21537 | 22537 | 23537 |
| 20538 | 21538 | 22538 | 23538 |
| 20539 | 21539 | 22539 | 23539 |
| 20540 | 21540 | 22540 | 23540 |
| 20541 | 21541 | 22541 | 23541 |
| 20542 | 21542 | 22542 | 23542 |
| 20543 | 21543 | 22543 | 23543 |
| 20544 | 21544 | 22544 | 23544 |
| 20545 | 21545 | 22545 | 23545 |
| 20546 | 21546 | 22546 | 23546 |
| 20547 | 21547 | 22547 | 23547 |
| 20548 | 21548 | 22548 | 23548 |
| 20549 | 21549 | 22549 | 23549 |
| 20550 | 21550 | 22550 | 23550 |
| 20551 | 21551 | 22551 | 23551 |
| 20552 | 21552 | 22552 | 23552 |
| 20553 | 21553 | 22553 | 23553 |
| 20554 | 21554 | 22554 | 23554 |
| 20555 | 21555 | 22555 | 23555 |
| 20556 | 21556 | 22556 | 23556 |
| 20557 | 21557 | 22557 | 23557 |
| 20558 | 21558 | 22558 | 23558 |
| 20559 | 21559 | 22559 | 23559 |
| 20560 | 21560 | 22560 | 23560 |
| 20561 | 21561 | 22561 | 23561 |
| 20562 | 21562 | 22562 | 23562 |
| 20563 | 21563 | 22563 | 23563 |
| 20564 | 21564 | 22564 | 23564 |
| 20565 | 21565 | 22565 | 23565 |
| 20566 | 21566 | 22566 | 23566 |
| 20567 | 21567 | 22567 | 23567 |
| 20568 | 21568 | 22568 | 23568 |
| 20569 | 21569 | 22569 | 23569 |
| 20570 | 21570 | 22570 | 23570 |
| 20571 | 21571 | 22571 | 23571 |
| 20572 | 21572 | 22572 | 23572 |
| 20573 | 21573 | 22573 | 23573 |
| 20574 | 21574 | 22574 | 23574 |
| 20575 | 21575 | 22575 | 23575 |
| 20576 | 21576 | 22576 | 23576 |
| 20577 | 21577 | 22577 | 23577 |
| 20578 | 21578 | 22578 | 23578 |
| 20579 | 21579 | 22579 | 23579 |
| 20580 | 21580 | 22580 | 23580 |
| 20581 | 21581 | 22581 | 23581 |
| 20582 | 21582 | 22582 | 23582 |
| 20583 | 21583 | 22583 | 23583 |
| 20584 | 21584 | 22584 | 23584 |
| 20585 | 21585 | 22585 | 23585 |
| 20586 | 21586 | 22586 | 23586 |
| 20587 | 21587 | 22587 | 23587 |
| 20588 | 21588 | 22588 | 23588 |
| 20589 | 21589 | 22589 | 23589 |
| 20590 | 21590 | 22590 | 23590 |
| 20591 | 21591 | 22591 | 23591 |
| 20592 | 21592 | 22592 | 23592 |
| 20593 | 21593 | 22593 | 23593 |
| 20594 | 21594 | 22594 | 23594 |
| 20595 | 21595 | 22595 | 23595 |
| 20596 | 21596 | 22596 | 23596 |
| 20597 | 21597 | 22597 | 23597 |
| 20598 | 21598 | 22598 | 23598 |
| 20599 | 21599 | 22599 | 23599 |
| 20600 | 21600 | 22600 | 23600 |
| 20601 | 21601 | 22601 | 23601 |
| 20602 | 21602 | 22602 | 23602 |
| 20603 | 21603 | 22603 | 23603 |
| 20604 | 21604 | 22604 | 23604 |
| 20605 | 21605 | 22605 | 23605 |
| 20606 | 21606 | 22606 | 23606 |
| 20607 | 21607 | 22607 | 23607 |
| 20608 | 21608 | 22608 | 23608 |
| 20609 | 21609 | 22609 | 23609 |
| 20610 | 21610 | 22610 | 23610 |
| 20611 | 21611 | 22611 | 23611 |
| 20612 | 21612 | 22612 | 23612 |
| 20613 | 21613 | 22613 | 23613 |
| 20614 | 21614 | 22614 | 23614 |
| 20615 | 21615 | 22615 | 23615 |
| 20616 | 21616 | 22616 | 23616 |
| 20617 | 21617 | 22617 | 23617 |
| 20618 | 21618 | 22618 | 23618 |
| 20619 | 21619 | 22619 | 23619 |
| 20620 | 21620 | 22620 | 23620 |
| 20621 | 21621 | 22621 | 23621 |
| 20622 | 21622 | 22622 | 23622 |
| 20623 | 21623 | 22623 | 23623 |
| 20624 | 21624 | 22624 | 23624 |
| 20625 | 21625 | 22625 | 23625 |
| 20626 | 21626 | 22626 | 23626 |
| 20627 | 21627 | 22627 | 23627 |
| 20628 | 21628 | 22628 | 23628 |
| 20629 | 21629 | 22629 | 23629 |
| 20630 | 21630 | 22630 | 23630 |
| 20631 | 21631 | 22631 | 23631 |
| 20632 | 21632 | 22632 | 23632 |
| 20633 | 21633 | 22633 | 23633 |
| 20634 | 21634 | 22634 | 23634 |
| 20635 | 21635 | 22635 | 23635 |
| 20636 | 21636 | 22636 | 23636 |
| 20637 | 21637 | 22637 | 23637 |
| 20638 | 21638 | 22638 | 23638 |
| 20639 | 21639 | 22639 | 23639 |
| 20640 | 21640 | 22640 | 23640 |
| 20641 | 21641 | 22641 | 23641 |
| 20642 | 21642 | 22642 | 23642 |
| 20643 | 21643 | 22643 | 23643 |
| 20644 | 21644 | 22644 | 23644 |
| 20645 | 21645 | 22645 | 23645 |
| 20646 | 21646 | 22646 | 23646 |
| 20647 | 21647 | 22647 | 23647 |
| 20648 | 21648 | 22648 | 23648 |
| 20649 | 21649 | 22649 | 23649 |
| 20650 | 21650 | 22650 | 23650 |
| 20651 | 21651 | 22651 | 23651 |
| 20652 | 21652 | 22652 | 23652 |
| 20653 | 21653 | 22653 | 23653 |
| 20654 | 21654 | 22654 | 23654 |
| 20655 | 21655 | 22655 | 23655 |
| 20656 | 21656 | 22656 | 23656 |
| 20657 | 21657 | 22657 | 23657 |
| 20658 | 21658 | 22658 | 23658 |
| 20659 | 21659 | 22659 | 23659 |
| 20660 | 21660 | 22660 | 23660 |
| 20661 | 21661 | 22661 | 23661 |
| 20662 | 21662 | 22662 | 23662 |
| 20663 | 21663 | 22663 | 23663 |
| 20664 | 21664 | 22664 | 23664 |
| 20665 | 21665 | 22665 | 23665 |
| 20666 | 21666 | 22666 | 23666 |
| 20667 | 21667 | 22667 | 23667 |
| 20668 | 21668 | 22668 | 23668 |
| 20669 | 21669 | 22669 | 23669 |
| 20670 | 21670 | 22670 | 23670 |
| 20671 | 21671 | 22671 | 23671 |
| 20672 | 21672 | 22672 | 23672 |
| 20673 | 21673 | 22673 | 23673 |
| 20674 | 21674 | 22674 | 23674 |
| 20675 | 21675 | 22675 | 23675 |
| 20676 | 21676 | 22676 | 23676 |
| 20677 | 21677 | 22677 | 23677 |
| 20678 | 21678 | 22678 | 23678 |
| 20679 | 21679 | 22679 | 23679 |
| 20680 | 21680 | 22680 | 23680 |
| 20681 | 21681 | 22681 | 23681 |
| 20682 | 21682 | 22682 | 23682 |
| 20683 | 21683 | 22683 | 23683 |
| 20684 | 21684 | 22684 | 23684 |
| 20685 | 21685 | 22685 | 23685 |
| 20686 | 21686 | 22686 | 23686 |
| 20687 | 21687 | 22687 | 23687 |
| 20688 | 21688 | 22688 | 23688 |
| 20689 | 21689 | 22689 | 23689 |
| 20690 | 21690 | 22690 | 23690 |
| 20691 | 21691 | 22691 | 23691 |
| 20692 | 21692 | 22692 | 23692 |
| 20693 | 21693 | 22693 | 23693 |
| 20694 | 21694 | 22694 | 23694 |
| 20695 | 21695 | 22695 | 23695 |
| 20696 | 21696 | 22696 | 23696 |
| 20697 | 21697 | 22697 | 23697 |
| 20698 | 21698 | 22698 | 23698 |
| 20699 | 21699 | 22699 | 23699 |
| 20700 | 21700 | 22700 | 23700 |
| 20701 | 21701 | 22701 | 23701 |
| 20702 | 21702 | 22702 | 23702 |
| 20703 | 21703 | 22703 | 23703 |
| 20704 | 21704 | 22704 | 23704 |
| 20705 | 21705 | 22705 | 23705 |
| 20706 | 21706 | 22706 | 23706 |
| 20707 | 21707 | 22707 | 23707 |
| 20708 | 21708 | 22708 | 23708 |
| 20709 | 21709 | 22709 | 23709 |
| 20710 | 21710 | 22710 | 23710 |
| 20711 | 21711 | 22711 | 23711 |
| 20712 | 21712 | 22712 | 23712 |
| 20713 | 21713 | 22713 | 23713 |
| 20714 | 21714 | 22714 | 23714 |
| 20715 | 21715 | 22715 | 23715 |
| 20716 | 21716 | 22716 | 23716 |
| 20717 | 21717 | 22717 | 23717 |
| 20718 | 21718 | 22718 | 23718 |
| 20719 | 21719 | 22719 | 23719 |
| 20720 | 21720 | 22720 | 23720 |
| 20721 | 21721 | 22721 | 23721 |
| 20722 | 21722 | 22722 | 23722 |
| 20723 | 21723 | 22723 | 23723 |
| 20724 | 21724 | 22724 | 23724 |
| 20725 | 21725 | 22725 | 23725 |
| 20726 | 21726 | 22726 | 23726 |
| 20727 | 21727 | 22727 | 23727 |
| 20728 | 21728 | 22728 | 23728 |
| 20729 | 21729 | 22729 | 23729 |
| 20730 | 21730 | 22730 | 23730 |
| 20731 | 21731 | 22731 | 23731 |
| 20732 | 21732 | 22732 | 23732 |
| 20733 | 21733 | 22733 | 23733 |
| 20734 | 21734 | 22734 | 23734 |
| 20735 | 21735 | 22735 | 23735 |
| 20736 | 21736 | 22736 | 23736 |
| 20737 | 21737 | 22737 | 23737 |
| 20738 | 21738 | 22738 | 23738 |
| 20739 | 21739 | 22739 | 23739 |
| 20740 | 21740 | 22740 | 23740 |
| 20741 | 21741 | 22741 | 23741 |
| 20742 | 21742 | 22742 | 23742 |
| 20743 | 21743 | 22743 | 23743 |
| 20744 | 21744 | 22744 | 23744 |
| 20745 | 21745 | 22745 | 23745 |
| 20746 | 21746 | 22746 | 23746 |
| 20747 | 21747 | 22747 | 23747 |
| 20748 | 21748 | 22748 | 23748 |
| 20749 | 21749 | 22749 | 23749 |
| 20750 | 21750 | 22750 | 23750 |
| 20751 | 21751 | 22751 | 23751 |
| 20752 | 21752 | 22752 | 23752 |
| 20753 | 21753 | 22753 | 23753 |
| 20754 | 21754 | 22754 | 23754 |
| 20755 | 21755 | 22755 | 23755 |
| 20756 | 21756 | 22756 | 23756 |
| 20757 | 21757 | 22757 | 23757 |
| 20758 | 21758 | 22758 | 23758 |
| 20759 | 21759 | 22759 | 23759 |
| 20760 | 21760 | 22760 | 23760 |
| 20761 | 21761 | 22761 | 23761 |
| 20762 | 21762 | 22762 | 23762 |
| 20763 | 21763 | 22763 | 23763 |
| 20764 | 21764 | 22764 | 23764 |
| 20765 | 21765 | 22765 | 23765 |
| 20766 | 21766 | 22766 | 23766 |
| 20767 | 21767 | 22767 | 23767 |
| 20768 | 21768 | 22768 | 23768 |
| 20769 | 21769 | 22769 | 23769 |
| 20770 | 21770 | 22770 | 23770 |
| 20771 | 21771 | 22771 | 23771 |
| 20772 | 21772 | 22772 | 23772 |
| 20773 | 21773 | 22773 | 23773 |
| 20774 | 21774 | 22774 | 23774 |
| 20775 | 21775 | 22775 | 23775 |
| 20776 | 21776 | 22776 | 23776 |
| 20777 | 21777 | 22777 | 23777 |
| 20778 | 21778 | 22778 | 23778 |
| 20779 | 21779 | 22779 | 23779 |
| 20780 | 21780 | 22780 | 23780 |
| 20781 | 21781 | 22781 | 23781 |
| 20782 | 21782 | 22782 | 23782 |
| 20783 | 21783 | 22783 | 23783 |
| 20784 | 21784 | 22784 | 23784 |
| 20785 | 21785 | 22785 | 23785 |
| 20786 | 21786 | 22786 | 23786 |
| 20787 | 21787 | 22787 | 23787 |
| 20788 | 21788 | 22788 | 23788 |
| 20789 | 21789 | 22789 | 23789 |
| 20790 | 21790 | 22790 | 23790 |
| 20791 | 21791 | 22791 | 23791 |
| 20792 | 21792 | 22792 | 23792 |
| 20793 | 21793 | 22793 | 23793 |
| 20794 | 21794 | 22794 | 23794 |
| 20795 | 21795 | 22795 | 23795 |
| 20796 | 21796 | 22796 | 23796 |
| 20797 | 21797 | 22797 | 23797 |
| 20798 | 21798 | 22798 | 23798 |
| 20799 | 21799 | 22799 | 23799 |
| 20800 | 21800 | 22800 | 23800 |
| 20801 | 21801 | 22801 | 23801 |
| 20802 | 21802 | 22802 | 23802 |
| 20803 | 21803 | 22803 | 23803 |
| 20804 | 21804 | 22804 | 23804 |
| 20805 | 21805 | 22805 | 23805 |
| 20806 | 21806 | 22806 | 23806 |
| 20807 | 21807 | 22807 | 23807 |
| 20808 | 21808 | 22808 | 23808 |
| 20809 | 21809 | 22809 | 23809 |
| 20810 | 21810 | 22810 | 23810 |
| 20811 | 21811 | 22811 | 23811 |
| 20812 | 21812 | 22812 | 23812 |
| 20813 | 21813 | 22813 | 23813 |
| 20814 | 21814 | 22814 | 23814 |
| 20815 | 21815 | 22815 | 23815 |
| 20816 | 21816 | 22816 | 23816 |
| 20817 | 21817 | 22817 | 23817 |
| 20818 | 21818 | 22818 | 23818 |
| 20819 | 21819 | 22819 | 23819 |
| 20820 | 21820 | 22820 | 23820 |
| 20821 | 21821 | 22821 | 23821 |
| 20822 | 21822 | 22822 | 23822 |
| 20823 | 21823 | 22823 | 23823 |
| 20824 | 21824 | 22824 | 23824 |
| 20825 | 21825 | 22825 | 23825 |
| 20826 | 21826 | 22826 | 23826 |
| 20827 | 21827 | 22827 | 23827 |
| 20828 | 21828 | 22828 | 23828 |
| 20829 | 21829 | 22829 | 23829 |
| 20830 | 21830 | 22830 | 23830 |
| 20831 | 21831 | 22831 | 23831 |
| 20832 | 21832 | 22832 | 23832 |
| 20833 | 21833 | 22833 | 23833 |
| 20834 | 21834 | 22834 | 23834 |
| 20835 | 21835 | 22835 | 23835 |
| 20836 | 21836 | 22836 | 23836 |
| 20837 | 21837 | 22837 | 23837 |
| 20838 | 21838 | 22838 | 23838 |
| 20839 | 21839 | 22839 | 23839 |
| 20840 | 21840 | 22840 | 23840 |
| 20841 | 21841 | 22841 | 23841 |
| 20842 | 21842 | 22842 | 23842 |
| 20843 | 21843 | 22843 | 23843 |
| 20844 | 21844 | 22844 | 23844 |
| 20845 | 21845 | 22845 | 23845 |
| 20846 | 21846 | 22846 | 23846 |
| 20847 | 21847 | 22847 | 23847 |
| 20848 | 21848 | 22848 | 23848 |
| 20849 | 21849 | 22849 | 23849 |
| 20850 | 21850 | 22850 | 23850 |
| 20851 | 21851 | 22851 | 23851 |
| 20852 | 21852 | 22852 | 23852 |
| 20853 | 21853 | 22853 | 23853 |
| 20854 | 21854 | 22854 | 23854 |
| 20855 | 21855 | 22855 | 23855 |
| 20856 | 21856 | 22856 | 23856 |
| 20857 | 21857 | 22857 | 23857 |
| 20858 | 21858 | 22858 | 23858 |
| 20859 | 21859 | 22859 | 23859 |
| 20860 | 21860 | 22860 | 23860 |
| 20861 | 21861 | 22861 | 23861 |
| 20862 | 21862 | 22862 | 23862 |
| 20863 | 21863 | 22863 | 23863 |
| 20864 | 21864 | 22864 | 23864 |
| 20865 | 21865 | 22865 | 23865 |
| 20866 | 21866 | 22866 | 23866 |
| 20867 | 21867 | 22867 | 23867 |
| 20868 | 21868 | 22868 | 23868 |
| 20869 | 21869 | 22869 | 23869 |
| 20870 | 21870 | 22870 | 23870 |
| 20871 | 21871 | 22871 | 23871 |
| 20872 | 21872 | 22872 | 23872 |
| 20873 | 21873 | 22873 | 23873 |
| 20874 | 21874 | 22874 | 23874 |
| 20875 | 21875 | 22875 | 23875 |
| 20876 | 21876 | 22876 | 23876 |
| 20877 | 21877 | 22877 | 23877 |
| 20878 | 21878 | 22878 | 23878 |
| 20879 | 21879 | 22879 | 23879 |
| 20880 | 21880 | 22880 | 23880 |
| 20881 | 21881 | 22881 | 23881 |
| 20882 | 21882 | 22882 | 23882 |
| 20883 | 21883 | 22883 | 23883 |
| 20884 | 21884 | 22884 | 23884 |
| 20885 | 21885 | 22885 | 23885 |
| 20886 | 21886 | 22886 | 23886 |
| 20887 | 21887 | 22887 | 23887 |
| 20888 | 21888 | 22888 | 23888 |
| 20889 | 21889 | 22889 | 23889 |
| 20890 | 21890 | 22890 | 23890 |
| 20891 | 21891 | 22891 | 23891 |
| 20892 | 21892 | 22892 | 23892 |
| 20893 | 21893 | 22893 | 23893 |
| 20894 | 21894 | 22894 | 23894 |
| 20895 | 21895 | 22895 | 23895 |
| 20896 | 21896 | 22896 | 23896 |
| 20897 | 21897 | 22897 | 23897 |
| 20898 | 21898 | 22898 | 23898 |
| 20899 | 21899 | 22899 | 23899 |
| 20900 | 21900 | 22900 | 23900 |
| 20901 | 21901 | 22901 | 23901 |
| 20902 | 21902 | 22902 | 23902 |
| 20903 | 21903 | 22903 | 23903 |
| 20904 | 21904 | 22904 | 23904 |
| 20905 | 21905 | 22905 | 23905 |
| 20906 | 21906 | 22906 | 23906 |
| 20907 | 21907 | 22907 | 23907 |
| 20908 | 21908 | 22908 | 23908 |
| 20909 | 21909 | 22909 | 23909 |
| 20910 | 21910 | 22910 | 23910 |
| 20911 | 21911 | 22911 | 23911 |
| 20912 | 21912 | 22912 | 23912 |
| 20913 | 21913 | 22913 | 23913 |
| 20914 | 21914 | 22914 | 23914 |
| 20915 | 21915 | 22915 | 23915 |
| 20916 | 21916 | 22916 | 23916 |
| 20917 | 21917 | 22917 | 23917 |
| 20918 | 21918 | 22918 | 23918 |
| 20919 | 21919 | 22919 | 23919 |
| 20920 | 21920 | 22920 | 23920 |
| 20921 | 21921 | 22921 | 23921 |
| 20922 | 21922 | 22922 | 23922 |
| 20923 | 21923 | 22923 | 23923 |
| 20924 | 21924 | 22924 | 23924 |
| 20925 | 21925 | 22925 | 23925 |
| 20926 | 21926 | 22926 | 23926 |
| 20927 | 21927 | 22927 | 23927 |
| 20928 | 21928 | 22928 | 23928 |
| 20929 | 21929 | 22929 | 23929 |
| 20930 | 21930 | 22930 | 23930 |
| 20931 | 21931 | 22931 | 23931 |
| 20932 | 21932 | 22932 | 23932 |
| 20933 | 21933 | 22933 | 23933 |
| 20934 | 21934 | 22934 | 23934 |
| 20935 | 21935 | 22935 | 23935 |
| 20936 | 21936 | 22936 | 23936 |
| 20937 | 21937 | 22937 | 23937 |
| 20938 | 21938 | 22938 | 23938 |
| 20939 | 21939 | 22939 | 23939 |
| 20940 | 21940 | 22940 | 23940 |
| 20941 | 21941 | 22941 | 23941 |
| 20942 | 21942 | 22942 | 23942 |
| 20943 | 21943 | 22943 | 23943 |
| 20944 | 21944 | 22944 | 23944 |
| 20945 | 21945 | 22945 | 23945 |
| 20946 | 21946 | 22946 | 23946 |
| 20947 | 21947 | 22947 | 23947 |
| 20948 | 21948 | 22948 | 23948 |
| 20949 | 21949 | 22949 | 23949 |
| 20950 | 21950 | 22950 | 23950 |
| 20951 | 21951 | 22951 | 23951 |
| 20952 | 21952 | 22952 | 23952 |
| 20953 | 21953 | 22953 | 23953 |
| 20954 | 21954 | 22954 | 23954 |
| 20955 | 21955 | 22955 | 23955 |
| 20956 | 21956 | 22956 | 23956 |
| 20957 | 21957 | 22957 | 23957 |
| 20958 | 21958 | 22958 | 23958 |
| 20959 | 21959 | 22959 | 23959 |
| 20960 | 21960 | 22960 | 23960 |
| 20961 | 21961 | 22961 | 23961 |
| 20962 | 21962 | 22962 | 23962 |
| 20963 | 21963 | 22963 | 23963 |
| 20964 | 21964 | 22964 | 23964 |
| 20965 | 21965 | 22965 | 23965 |
| 20966 | 21966 | 22966 | 23966 |
| 20967 | 21967 | 22967 | 23967 |
| 20968 | 21968 | 22968 | 23968 |
| 20969 | 21969 | 22969 | 23969 |
| 20970 | 21970 | 22970 | 23970 |
| 20971 | 21971 | 22971 | 23971 |
| 20972 | 21972 | 22972 | 23972 |
| 20973 | 21973 | 22973 | 23973 |
| 20974 | 21974 | 22974 | 23974 |
| 20975 | 21975 | 22975 | 23975 |
| 20976 | 21976 | 22976 | 23976 |
| 20977 | 21977 | 22977 | 23977 |
| 20978 | 21978 | 22978 | 23978 |
| 20979 | 21979 | 22979 | 23979 |
| 20980 | 21980 | 22980 | 23980 |
| 20981 | 21981 | 22981 | 23981 |
| 20982 | 21982 | 22982 | 23982 |
| 20983 | 21983 | 22983 | 23983 |
| 20984 | 21984 | 22984 | 23984 |
| 20985 | 21985 | 22985 | 23985 |
| 20986 | 21986 | 22986 | 23986 |
| 20987 | 21987 | 22987 | 23987 |
| 20988 | 21988 | 22988 | 23988 |
| 20989 | 21989 | 22989 | 23989 |
| 20990 | 21990 | 22990 | 23990 |
| 20991 | 21991 | 22991 | 23991 |
| 20992 | 21992 | 22992 | 23992 |
| 20993 | 21993 | 22993 | 23993 |
| 20994 | 21994 | 22994 | 23994 |
| 20995 | 21995 | 22995 | 23995 |
| 20996 | 21996 | 22996 | 23996 |
| 20997 | 21997 | 22997 | 23997 |
| 20998 | 21998 | 22998 | 23998 |
| 20999 | 21999 | 22999 | 23999 |
| 21000 | 22000 | 23000 | 24000 |
| 21001 | 22001 | 23001 | 24001 |
| 21002 | 22002 | 23002 | 24002 |

In some embodiments, the sequences that specifically bind to pistachio comprise an inner sequence selected from the group consisting of the nucleic acid sequences of SEQ ID NOs. 24003 to 25002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to pistachio may comprise a nucleic acid sequence selected from the group consisting of SEQ ID NOs. 25003 to 26002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to pistachio may comprise a nucleic acid sequence selected from the group consisting of SEQ ID NOs. 26003 to 27002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to pistachio may comprise a nucleic acid sequence selected from the group consisting of SEQ ID NOs. 27003 to 28002. In one embodiment, the aptamer of the present disclosure that specifically binds to pistachio may comprise a nucleic acid sequence selected from the group consisting of SEQ ID NOs. 24003 to 28002 listed in Table 8, or variant thereof.

TABLE 8
Aptamer sequences against pistachio
Aptamer sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 24003 | 25003 | 26003 | 27003 |
| 24004 | 25004 | 26004 | 27004 |
| 24005 | 25005 | 26005 | 27005 |
| 24006 | 25006 | 26006 | 27006 |
| 24007 | 25007 | 26007 | 27007 |
| 24008 | 25008 | 26008 | 27008 |
| 24009 | 25009 | 26009 | 27009 |
| 24010 | 25010 | 26010 | 27010 |
| 24011 | 25011 | 26011 | 27011 |
| 24012 | 25012 | 26012 | 27012 |
| 24013 | 25013 | 26013 | 27013 |
| 24014 | 25014 | 26014 | 27014 |
| 24015 | 25015 | 26015 | 27015 |
| 24016 | 25016 | 26016 | 27016 |
| 24017 | 25017 | 26017 | 27017 |
| 24018 | 25018 | 26018 | 27018 |
| 24019 | 25019 | 26019 | 27019 |
| 24020 | 25020 | 26020 | 27020 |
| 24021 | 25021 | 26021 | 27021 |
| 24022 | 25022 | 26022 | 27022 |
| 24023 | 25023 | 26023 | 27023 |
| 24024 | 25024 | 26024 | 27024 |
| 24025 | 25025 | 26025 | 27025 |
| 24026 | 25026 | 26026 | 27026 |
| 24027 | 25027 | 26027 | 27027 |
| 24028 | 25028 | 26028 | 27028 |
| 24029 | 25029 | 26029 | 27029 |
| 24030 | 25030 | 26030 | 27030 |
| 24031 | 25031 | 26031 | 27031 |
| 24032 | 25032 | 26032 | 27032 |
| 24033 | 25033 | 26033 | 27033 |
| 24034 | 25034 | 26034 | 27034 |
| 24035 | 25035 | 26035 | 27035 |
| 24036 | 25036 | 26036 | 27036 |
| 24037 | 25037 | 26037 | 27037 |
| 24038 | 25038 | 26038 | 27038 |
| 24039 | 25039 | 26039 | 27039 |
| 24040 | 25040 | 26040 | 27040 |
| 24041 | 25041 | 26041 | 27041 |
| 24042 | 25042 | 26042 | 27042 |
| 24043 | 25043 | 26043 | 27043 |
| 24044 | 25044 | 26044 | 27044 |
| 24045 | 25045 | 26045 | 27045 |
| 24046 | 25046 | 26046 | 27046 |
| 24047 | 25047 | 26047 | 27047 |
| 24048 | 25048 | 26048 | 27048 |
| 24049 | 25049 | 26049 | 27049 |
| 24050 | 25050 | 26050 | 27050 |
| 24051 | 25051 | 26051 | 27051 |
| 24052 | 25052 | 26052 | 27052 |
| 24053 | 25053 | 26053 | 27053 |
| 24054 | 25054 | 26054 | 27054 |
| 24055 | 25055 | 26055 | 27055 |
| 24056 | 25056 | 26056 | 27056 |
| 24057 | 25057 | 26057 | 27057 |
| 24058 | 25058 | 26058 | 27058 |
| 24059 | 25059 | 26059 | 27059 |
| 24060 | 25060 | 26060 | 27060 |
| 24061 | 25061 | 26061 | 27061 |
| 24062 | 25062 | 26062 | 27062 |
| 24063 | 25063 | 26063 | 27063 |
| 24064 | 25064 | 26064 | 27064 |
| 24065 | 25065 | 26065 | 27065 |
| 24066 | 25066 | 26066 | 27066 |
| 24067 | 25067 | 26067 | 27067 |
| 24068 | 25068 | 26068 | 27068 |
| 24069 | 25069 | 26069 | 27069 |
| 24070 | 25070 | 26070 | 27070 |
| 24071 | 25071 | 26071 | 27071 |
| 24072 | 25072 | 26072 | 27072 |
| 24073 | 25073 | 26073 | 27073 |
| 24074 | 25074 | 26074 | 27074 |
| 24075 | 25075 | 26075 | 27075 |
| 24076 | 25076 | 26076 | 27076 |
| 24077 | 25077 | 26077 | 27077 |
| 24078 | 25078 | 26078 | 27078 |
| 24079 | 25079 | 26079 | 27079 |
| 24080 | 25080 | 26080 | 27080 |
| 24081 | 25081 | 26081 | 27081 |
| 24082 | 25082 | 26082 | 27082 |
| 24083 | 25083 | 26083 | 27083 |
| 24084 | 25084 | 26084 | 27084 |
| 24085 | 25085 | 26085 | 27085 |
| 24086 | 25086 | 26086 | 27086 |
| 24087 | 25087 | 26087 | 27087 |
| 24088 | 25088 | 26088 | 27088 |
| 24089 | 25089 | 26089 | 27089 |
| 24090 | 25090 | 26090 | 27090 |
| 24091 | 25091 | 26091 | 27091 |
| 24092 | 25092 | 26092 | 27092 |
| 24093 | 25093 | 26093 | 27093 |
| 24094 | 25094 | 26094 | 27094 |
| 24095 | 25095 | 26095 | 27095 |
| 24096 | 25096 | 26096 | 27096 |
| 24097 | 25097 | 26097 | 27097 |
| 24098 | 25098 | 26098 | 27098 |
| 24099 | 25099 | 26099 | 27099 |
| 24100 | 25100 | 26100 | 27100 |
| 24101 | 25101 | 26101 | 27101 |
| 24102 | 25102 | 26102 | 27102 |
| 24103 | 25103 | 26103 | 27103 |
| 24104 | 25104 | 26104 | 27104 |
| 24105 | 25105 | 26105 | 27105 |
| 24106 | 25106 | 26106 | 27106 |
| 24107 | 25107 | 26107 | 27107 |
| 24108 | 25108 | 26108 | 27108 |
| 24109 | 25109 | 26109 | 27109 |
| 24110 | 25110 | 26110 | 27110 |
| 24111 | 25111 | 26111 | 27111 |
| 24112 | 25112 | 26112 | 27112 |
| 24113 | 25113 | 26113 | 27113 |
| 24114 | 25114 | 26114 | 27114 |
| 24115 | 25115 | 26115 | 27115 |
| 24116 | 25116 | 26116 | 27116 |
| 24117 | 25117 | 26117 | 27117 |
| 24118 | 25118 | 26118 | 27118 |
| 24119 | 25119 | 26119 | 27119 |
| 24120 | 25120 | 26120 | 27120 |
| 24121 | 25121 | 26121 | 27121 |
| 24122 | 25122 | 26122 | 27122 |
| 24123 | 25123 | 26123 | 27123 |
| 24124 | 25124 | 26124 | 27124 |
| 24125 | 25125 | 26125 | 27125 |
| 24126 | 25126 | 26126 | 27126 |
| 24127 | 25127 | 26127 | 27127 |
| 24128 | 25128 | 26128 | 27128 |
| 24129 | 25129 | 26129 | 27129 |
| 24130 | 25130 | 26130 | 27130 |
| 24131 | 25131 | 26131 | 27131 |
| 24132 | 25132 | 26132 | 27132 |
| 24133 | 25133 | 26133 | 27133 |
| 24134 | 25134 | 26134 | 27134 |
| 24135 | 25135 | 26135 | 27135 |
| 24136 | 25136 | 26136 | 27136 |
| 24137 | 25137 | 26137 | 27137 |
| 24138 | 25138 | 26138 | 27138 |
| 24139 | 25139 | 26139 | 27139 |
| 24140 | 25140 | 26140 | 27140 |
| 24141 | 25141 | 26141 | 27141 |
| 24142 | 25142 | 26142 | 27142 |
| 24143 | 25143 | 26143 | 27143 |
| 24144 | 25144 | 26144 | 27144 |
| 24145 | 25145 | 26145 | 27145 |
| 24146 | 25146 | 26146 | 27146 |
| 24147 | 25147 | 26147 | 27147 |
| 24148 | 25148 | 26148 | 27148 |
| 24149 | 25149 | 26149 | 27149 |
| 24150 | 25150 | 26150 | 27150 |
| 24151 | 25151 | 26151 | 27151 |
| 24152 | 25152 | 26152 | 27152 |
| 24153 | 25153 | 26153 | 27153 |
| 24154 | 25154 | 26154 | 27154 |
| 24155 | 25155 | 26155 | 27155 |
| 24156 | 25156 | 26156 | 27156 |
| 24157 | 25157 | 26157 | 27157 |
| 24158 | 25158 | 26158 | 27158 |
| 24159 | 25159 | 26159 | 27159 |
| 24160 | 25160 | 26160 | 27160 |
| 24161 | 25161 | 26161 | 27161 |
| 24162 | 25162 | 26162 | 27162 |
| 24163 | 25163 | 26163 | 27163 |
| 24164 | 25164 | 26164 | 27164 |
| 24165 | 25165 | 26165 | 27165 |
| 24166 | 25166 | 26166 | 27166 |
| 24167 | 25167 | 26167 | 27167 |
| 24168 | 25168 | 26168 | 27168 |
| 24169 | 25169 | 26169 | 27169 |
| 24170 | 25170 | 26170 | 27170 |
| 24171 | 25171 | 26171 | 27171 |
| 24172 | 25172 | 26172 | 27172 |
| 24173 | 25173 | 26173 | 27173 |
| 24174 | 25174 | 26174 | 27174 |
| 24175 | 25175 | 26175 | 27175 |
| 24176 | 25176 | 26176 | 27176 |
| 24177 | 25177 | 26177 | 27177 |
| 24178 | 25178 | 26178 | 27178 |
| 24179 | 25179 | 26179 | 27179 |
| 24180 | 25180 | 26180 | 27180 |
| 24181 | 25181 | 26181 | 27181 |
| 24182 | 25182 | 26182 | 27182 |
| 24183 | 25183 | 26183 | 27183 |
| 24184 | 25184 | 26184 | 27184 |
| 24185 | 25185 | 26185 | 27185 |
| 24186 | 25186 | 26186 | 27186 |
| 24187 | 25187 | 26187 | 27187 |
| 24188 | 25188 | 26188 | 27188 |
| 24189 | 25189 | 26189 | 27189 |
| 24190 | 25190 | 26190 | 27190 |
| 24191 | 25191 | 26191 | 27191 |
| 24192 | 25192 | 26192 | 27192 |
| 24193 | 25193 | 26193 | 27193 |
| 24194 | 25194 | 26194 | 27194 |
| 24195 | 25195 | 26195 | 27195 |
| 24196 | 25196 | 26196 | 27196 |
| 24197 | 25197 | 26197 | 27197 |
| 24198 | 25198 | 26198 | 27198 |
| 24199 | 25199 | 26199 | 27199 |
| 24200 | 25200 | 26200 | 27200 |
| 24201 | 25201 | 26201 | 27201 |
| 24202 | 25202 | 26202 | 27202 |
| 24203 | 25203 | 26203 | 27203 |
| 24204 | 25204 | 26204 | 27204 |
| 24205 | 25205 | 26205 | 27205 |
| 24206 | 25206 | 26206 | 27206 |
| 24207 | 25207 | 26207 | 27207 |
| 24208 | 25208 | 26208 | 27208 |
| 24209 | 25209 | 26209 | 27209 |
| 24210 | 25210 | 26210 | 27210 |
| 24211 | 25211 | 26211 | 27211 |
| 24212 | 25212 | 26212 | 27212 |
| 24213 | 25213 | 26213 | 27213 |
| 24214 | 25214 | 26214 | 27214 |
| 24215 | 25215 | 26215 | 27215 |
| 24216 | 25216 | 26216 | 27216 |
| 24217 | 25217 | 26217 | 27217 |
| 24218 | 25218 | 26218 | 27218 |
| 24219 | 25219 | 26219 | 27219 |
| 24220 | 25220 | 26220 | 27220 |
| 24221 | 25221 | 26221 | 27221 |
| 24222 | 25222 | 26222 | 27222 |
| 24223 | 25223 | 26223 | 27223 |
| 24224 | 25224 | 26224 | 27224 |
| 24225 | 25225 | 26225 | 27225 |
| 24226 | 25226 | 26226 | 27226 |
| 24227 | 25227 | 26227 | 27227 |
| 24228 | 25228 | 26228 | 27228 |
| 24229 | 25229 | 26229 | 27229 |
| 24230 | 25230 | 26230 | 27230 |
| 24231 | 25231 | 26231 | 27231 |
| 24232 | 25232 | 26232 | 27232 |
| 24233 | 25233 | 26233 | 27233 |
| 24234 | 25234 | 26234 | 27234 |
| 24235 | 25235 | 26235 | 27235 |
| 24236 | 25236 | 26236 | 27236 |
| 24237 | 25237 | 26237 | 27237 |
| 24238 | 25238 | 26238 | 27238 |
| 24239 | 25239 | 26239 | 27239 |
| 24240 | 25240 | 26240 | 27240 |
| 24241 | 25241 | 26241 | 27241 |
| 24242 | 25242 | 26242 | 27242 |
| 24243 | 25243 | 26243 | 27243 |
| 24244 | 25244 | 26244 | 27244 |
| 24245 | 25245 | 26245 | 27245 |
| 24246 | 25246 | 26246 | 27246 |
| 24247 | 25247 | 26247 | 27247 |
| 24248 | 25248 | 26248 | 27248 |
| 24249 | 25249 | 26249 | 27249 |
| 24250 | 25250 | 26250 | 27250 |
| 24251 | 25251 | 26251 | 27251 |
| 24252 | 25252 | 26252 | 27252 |
| 24253 | 25253 | 26253 | 27253 |
| 24254 | 25254 | 26254 | 27254 |
| 24255 | 25255 | 26255 | 27255 |
| 24256 | 25256 | 26256 | 27256 |
| 24257 | 25257 | 26257 | 27257 |
| 24258 | 25258 | 26258 | 27258 |
| 24259 | 25259 | 26259 | 27259 |
| 24260 | 25260 | 26260 | 27260 |
| 24261 | 25261 | 26261 | 27261 |
| 24262 | 25262 | 26262 | 27262 |
| 24263 | 25263 | 26263 | 27263 |
| 24264 | 25264 | 26264 | 27264 |
| 24265 | 25265 | 26265 | 27265 |
| 24266 | 25266 | 26266 | 27266 |
| 24267 | 25267 | 26267 | 27267 |
| 24268 | 25268 | 26268 | 27268 |
| 24269 | 25269 | 26269 | 27269 |
| 24270 | 25270 | 26270 | 27270 |
| 24271 | 25271 | 26271 | 27271 |
| 24272 | 25272 | 26272 | 27272 |
| 24273 | 25273 | 26273 | 27273 |
| 24274 | 25274 | 26274 | 27274 |
| 24275 | 25275 | 26275 | 27275 |
| 24276 | 25276 | 26276 | 27276 |
| 24277 | 25277 | 26277 | 27277 |
| 24278 | 25278 | 26278 | 27278 |
| 24279 | 25279 | 26279 | 27279 |
| 24280 | 25280 | 26280 | 27280 |
| 24281 | 25281 | 26281 | 27281 |
| 24282 | 25282 | 26282 | 27282 |
| 24283 | 25283 | 26283 | 27283 |
| 24284 | 25284 | 26284 | 27284 |
| 24285 | 25285 | 26285 | 27285 |
| 24286 | 25286 | 26286 | 27286 |
| 24287 | 25287 | 26287 | 27287 |
| 24288 | 25288 | 26288 | 27288 |
| 24289 | 25289 | 26289 | 27289 |
| 24290 | 25290 | 26290 | 27290 |
| 24291 | 25291 | 26291 | 27291 |
| 24292 | 25292 | 26292 | 27292 |
| 24293 | 25293 | 26293 | 27293 |
| 24294 | 25294 | 26294 | 27294 |
| 24295 | 25295 | 26295 | 27295 |
| 24296 | 25296 | 26296 | 27296 |
| 24297 | 25297 | 26297 | 27297 |
| 24298 | 25298 | 26298 | 27298 |
| 24299 | 25299 | 26299 | 27299 |
| 24300 | 25300 | 26300 | 27300 |
| 24301 | 25301 | 26301 | 27301 |
| 24302 | 25302 | 26302 | 27302 |
| 24303 | 25303 | 26303 | 27303 |
| 24304 | 25304 | 26304 | 27304 |
| 24305 | 25305 | 26305 | 27305 |
| 24306 | 25306 | 26306 | 27306 |
| 24307 | 25307 | 26307 | 27307 |
| 24308 | 25308 | 26308 | 27308 |
| 24309 | 25309 | 26309 | 27309 |
| 24310 | 25310 | 26310 | 27310 |
| 24311 | 25311 | 26311 | 27311 |
| 24312 | 25312 | 26312 | 27312 |
| 24313 | 25313 | 26313 | 27313 |
| 24314 | 25314 | 26314 | 27314 |
| 24315 | 25315 | 26315 | 27315 |
| 24316 | 25316 | 26316 | 27316 |
| 24317 | 25317 | 26317 | 27317 |
| 24318 | 25318 | 26318 | 27318 |
| 24319 | 25319 | 26319 | 27319 |
| 24320 | 25320 | 26320 | 27320 |
| 24321 | 25321 | 26321 | 27321 |
| 24322 | 25322 | 26322 | 27322 |
| 24323 | 25323 | 26323 | 27323 |
| 24324 | 25324 | 26324 | 27324 |
| 24325 | 25325 | 26325 | 27325 |
| 24326 | 25326 | 26326 | 27326 |
| 24327 | 25327 | 26327 | 27327 |
| 24328 | 25328 | 26328 | 27328 |
| 24329 | 25329 | 26329 | 27329 |
| 24330 | 25330 | 26330 | 27330 |
| 24331 | 25331 | 26331 | 27331 |
| 24332 | 25332 | 26332 | 27332 |
| 24333 | 25333 | 26333 | 27333 |
| 24334 | 25334 | 26334 | 27334 |
| 24335 | 25335 | 26335 | 27335 |
| 24336 | 25336 | 26336 | 27336 |
| 24337 | 25337 | 26337 | 27337 |
| 24338 | 25338 | 26338 | 27338 |
| 24339 | 25339 | 26339 | 27339 |
| 24340 | 25340 | 26340 | 27340 |
| 24341 | 25341 | 26341 | 27341 |
| 24342 | 25342 | 26342 | 27342 |
| 24343 | 25343 | 26343 | 27343 |
| 24344 | 25344 | 26344 | 27344 |
| 24345 | 25345 | 26345 | 27345 |
| 24346 | 25346 | 26346 | 27346 |
| 24347 | 25347 | 26347 | 27347 |
| 24348 | 25348 | 26348 | 27348 |
| 24349 | 25349 | 26349 | 27349 |
| 24350 | 25350 | 26350 | 27350 |
| 24351 | 25351 | 26351 | 27351 |
| 24352 | 25352 | 26352 | 27352 |
| 24353 | 25353 | 26353 | 27353 |
| 24354 | 25354 | 26354 | 27354 |
| 24355 | 25355 | 26355 | 27355 |
| 24356 | 25356 | 26356 | 27356 |
| 24357 | 25357 | 26357 | 27357 |
| 24358 | 25358 | 26358 | 27358 |
| 24359 | 25359 | 26359 | 27359 |
| 24360 | 25360 | 26360 | 27360 |
| 24361 | 25361 | 26361 | 27361 |
| 24362 | 25362 | 26362 | 27362 |
| 24363 | 25363 | 26363 | 27363 |
| 24364 | 25364 | 26364 | 27364 |
| 24365 | 25365 | 26365 | 27365 |
| 24366 | 25366 | 26366 | 27366 |
| 24367 | 25367 | 26367 | 27367 |
| 24368 | 25368 | 26368 | 27368 |
| 24369 | 25369 | 26369 | 27369 |
| 24370 | 25370 | 26370 | 27370 |
| 24371 | 25371 | 26371 | 27371 |
| 24372 | 25372 | 26372 | 27372 |
| 24373 | 25373 | 26373 | 27373 |
| 24374 | 25374 | 26374 | 27374 |
| 24375 | 25375 | 26375 | 27375 |
| 24376 | 25376 | 26376 | 27376 |
| 24377 | 25377 | 26377 | 27377 |
| 24378 | 25378 | 26378 | 27378 |
| 24379 | 25379 | 26379 | 27379 |
| 24380 | 25380 | 26380 | 27380 |
| 24381 | 25381 | 26381 | 27381 |
| 24382 | 25382 | 26382 | 27382 |
| 24383 | 25383 | 26383 | 27383 |
| 24384 | 25384 | 26384 | 27384 |
| 24385 | 25385 | 26385 | 27385 |
| 24386 | 25386 | 26386 | 27386 |
| 24387 | 25387 | 26387 | 27387 |
| 24388 | 25388 | 26388 | 27388 |
| 24389 | 25389 | 26389 | 27389 |
| 24390 | 25390 | 26390 | 27390 |
| 24391 | 25391 | 26391 | 27391 |
| 24392 | 25392 | 26392 | 27392 |
| 24393 | 25393 | 26393 | 27393 |
| 24394 | 25394 | 26394 | 27394 |
| 24395 | 25395 | 26395 | 27395 |
| 24396 | 25396 | 26396 | 27396 |
| 24397 | 25397 | 26397 | 27397 |
| 24398 | 25398 | 26398 | 27398 |
| 24399 | 25399 | 26399 | 27399 |
| 24400 | 25400 | 26400 | 27400 |
| 24401 | 25401 | 26401 | 27401 |
| 24402 | 25402 | 26402 | 27402 |
| 24403 | 25403 | 26403 | 27403 |
| 24404 | 25404 | 26404 | 27404 |
| 24405 | 25405 | 26405 | 27405 |
| 24406 | 25406 | 26406 | 27406 |
| 24407 | 25407 | 26407 | 27407 |
| 24408 | 25408 | 26408 | 27408 |
| 24409 | 25409 | 26409 | 27409 |
| 24410 | 25410 | 26410 | 27410 |
| 24411 | 25411 | 26411 | 27411 |
| 24412 | 25412 | 26412 | 27412 |
| 24413 | 25413 | 26413 | 27413 |
| 24414 | 25414 | 26414 | 27414 |
| 24415 | 25415 | 26415 | 27415 |
| 24416 | 25416 | 26416 | 27416 |
| 24417 | 25417 | 26417 | 27417 |
| 24418 | 25418 | 26418 | 27418 |
| 24419 | 25419 | 26419 | 27419 |
| 24420 | 25420 | 26420 | 27420 |
| 24421 | 25421 | 26421 | 27421 |
| 24422 | 25422 | 26422 | 27422 |
| 24423 | 25423 | 26423 | 27423 |
| 24424 | 25424 | 26424 | 27424 |
| 24425 | 25425 | 26425 | 27425 |
| 24426 | 25426 | 26426 | 27426 |
| 24427 | 25427 | 26427 | 27427 |
| 24428 | 25428 | 26428 | 27428 |
| 24429 | 25429 | 26429 | 27429 |
| 24430 | 25430 | 26430 | 27430 |
| 24431 | 25431 | 26431 | 27431 |
| 24432 | 25432 | 26432 | 27432 |
| 24433 | 25433 | 26433 | 27433 |
| 24434 | 25434 | 26434 | 27434 |
| 24435 | 25435 | 26435 | 27435 |
| 24436 | 25436 | 26436 | 27436 |
| 24437 | 25437 | 26437 | 27437 |
| 24438 | 25438 | 26438 | 27438 |
| 24439 | 25439 | 26439 | 27439 |
| 24440 | 25440 | 26440 | 27440 |
| 24441 | 25441 | 26441 | 27441 |
| 24442 | 25442 | 26442 | 27442 |
| 24443 | 25443 | 26443 | 27443 |
| 24444 | 25444 | 26444 | 27444 |
| 24445 | 25445 | 26445 | 27445 |
| 24446 | 25446 | 26446 | 27446 |
| 24447 | 25447 | 26447 | 27447 |
| 24448 | 25448 | 26448 | 27448 |
| 24449 | 25449 | 26449 | 27449 |
| 24450 | 25450 | 26450 | 27450 |
| 24451 | 25451 | 26451 | 27451 |
| 24452 | 25452 | 26452 | 27452 |
| 24453 | 25453 | 26453 | 27453 |
| 24454 | 25454 | 26454 | 27454 |
| 24455 | 25455 | 26455 | 27455 |
| 24456 | 25456 | 26456 | 27456 |
| 24457 | 25457 | 26457 | 27457 |
| 24458 | 25458 | 26458 | 27458 |
| 24459 | 25459 | 26459 | 27459 |
| 24460 | 25460 | 26460 | 27460 |
| 24461 | 25461 | 26461 | 27461 |
| 24462 | 25462 | 26462 | 27462 |
| 24463 | 25463 | 26463 | 27463 |
| 24464 | 25464 | 26464 | 27464 |
| 24465 | 25465 | 26465 | 27465 |
| 24466 | 25466 | 26466 | 27466 |
| 24467 | 25467 | 26467 | 27467 |
| 24468 | 25468 | 26468 | 27468 |
| 24469 | 25469 | 26469 | 27469 |
| 24470 | 25470 | 26470 | 27470 |
| 24471 | 25471 | 26471 | 27471 |
| 24472 | 25472 | 26472 | 27472 |
| 24473 | 25473 | 26473 | 27473 |
| 24474 | 25474 | 26474 | 27474 |
| 24475 | 25475 | 26475 | 27475 |
| 24476 | 25476 | 26476 | 27476 |
| 24477 | 25477 | 26477 | 27477 |
| 24478 | 25478 | 26478 | 27478 |
| 24479 | 25479 | 26479 | 27479 |
| 24480 | 25480 | 26480 | 27480 |
| 24481 | 25481 | 26481 | 27481 |
| 24482 | 25482 | 26482 | 27482 |
| 24483 | 25483 | 26483 | 27483 |
| 24484 | 25484 | 26484 | 27484 |
| 24485 | 25485 | 26485 | 27485 |
| 24486 | 25486 | 26486 | 27486 |
| 24487 | 25487 | 26487 | 27487 |
| 24488 | 25488 | 26488 | 27488 |
| 24489 | 25489 | 26489 | 27489 |
| 24490 | 25490 | 26490 | 27490 |
| 24491 | 25491 | 26491 | 27491 |
| 24492 | 25492 | 26492 | 27492 |
| 24493 | 25493 | 26493 | 27493 |
| 24494 | 25494 | 26494 | 27494 |
| 24495 | 25495 | 26495 | 27495 |
| 24496 | 25496 | 26496 | 27496 |
| 24497 | 25497 | 26497 | 27497 |
| 24498 | 25498 | 26498 | 27498 |
| 24499 | 25499 | 26499 | 27499 |
| 24500 | 25500 | 26500 | 27500 |
| 24501 | 25501 | 26501 | 27501 |
| 24502 | 25502 | 26502 | 27502 |
| 24503 | 25503 | 26503 | 27503 |
| 24504 | 25504 | 26504 | 27504 |
| 24505 | 25505 | 26505 | 27505 |
| 24506 | 25506 | 26506 | 27506 |
| 24507 | 25507 | 26507 | 27507 |
| 24508 | 25508 | 26508 | 27508 |
| 24509 | 25509 | 26509 | 27509 |
| 24510 | 25510 | 26510 | 27510 |
| 24511 | 25511 | 26511 | 27511 |
| 24512 | 25512 | 26512 | 27512 |
| 24513 | 25513 | 26513 | 27513 |
| 24514 | 25514 | 26514 | 27514 |
| 24515 | 25515 | 26515 | 27515 |
| 24516 | 25516 | 26516 | 27516 |
| 24517 | 25517 | 26517 | 27517 |
| 24518 | 25518 | 26518 | 27518 |
| 24519 | 25519 | 26519 | 27519 |
| 24520 | 25520 | 26520 | 27520 |
| 24521 | 25521 | 26521 | 27521 |
| 24522 | 25522 | 26522 | 27522 |
| 24523 | 25523 | 26523 | 27523 |
| 24524 | 25524 | 26524 | 27524 |
| 24525 | 25525 | 26525 | 27525 |
| 24526 | 25526 | 26526 | 27526 |
| 24527 | 25527 | 26527 | 27527 |
| 24528 | 25528 | 26528 | 27528 |
| 24529 | 25529 | 26529 | 27529 |
| 24530 | 25530 | 26530 | 27530 |
| 24531 | 25531 | 26531 | 27531 |
| 24532 | 25532 | 26532 | 27532 |
| 24533 | 25533 | 26533 | 27533 |
| 24534 | 25534 | 26534 | 27534 |
| 24535 | 25535 | 26535 | 27535 |
| 24536 | 25536 | 26536 | 27536 |
| 24537 | 25537 | 26537 | 27537 |
| 24538 | 25538 | 26538 | 27538 |
| 24539 | 25539 | 26539 | 27539 |
| 24540 | 25540 | 26540 | 27540 |
| 24541 | 25541 | 26541 | 27541 |
| 24542 | 25542 | 26542 | 27542 |
| 24543 | 25543 | 26543 | 27543 |
| 24544 | 25544 | 26544 | 27544 |
| 24545 | 25545 | 26545 | 27545 |
| 24546 | 25546 | 26546 | 27546 |
| 24547 | 25547 | 26547 | 27547 |
| 24548 | 25548 | 26548 | 27548 |
| 24549 | 25549 | 26549 | 27549 |
| 24550 | 25550 | 26550 | 27550 |
| 24551 | 25551 | 26551 | 27551 |
| 24552 | 25552 | 26552 | 27552 |
| 24553 | 25553 | 26553 | 27553 |
| 24554 | 25554 | 26554 | 27554 |
| 24555 | 25555 | 26555 | 27555 |
| 24556 | 25556 | 26556 | 27556 |
| 24557 | 25557 | 26557 | 27557 |
| 24558 | 25558 | 26558 | 27558 |
| 24559 | 25559 | 26559 | 27559 |
| 24560 | 25560 | 26560 | 27560 |
| 24561 | 25561 | 26561 | 27561 |
| 24562 | 25562 | 26562 | 27562 |
| 24563 | 25563 | 26563 | 27563 |
| 24564 | 25564 | 26564 | 27564 |
| 24565 | 25565 | 26565 | 27565 |
| 24566 | 25566 | 26566 | 27566 |
| 24567 | 25567 | 26567 | 27567 |
| 24568 | 25568 | 26568 | 27568 |
| 24569 | 25569 | 26569 | 27569 |
| 24570 | 25570 | 26570 | 27570 |
| 24571 | 25571 | 26571 | 27571 |
| 24572 | 25572 | 26572 | 27572 |
| 24573 | 25573 | 26573 | 27573 |
| 24574 | 25574 | 26574 | 27574 |
| 24575 | 25575 | 26575 | 27575 |
| 24576 | 25576 | 26576 | 27576 |
| 24577 | 25577 | 26577 | 27577 |
| 24578 | 25578 | 26578 | 27578 |
| 24579 | 25579 | 26579 | 27579 |
| 24580 | 25580 | 26580 | 27580 |
| 24581 | 25581 | 26581 | 27581 |
| 24582 | 25582 | 26582 | 27582 |
| 24583 | 25583 | 26583 | 27583 |
| 24584 | 25584 | 26584 | 27584 |
| 24585 | 25585 | 26585 | 27585 |
| 24586 | 25586 | 26586 | 27586 |
| 24587 | 25587 | 26587 | 27587 |
| 24588 | 25588 | 26588 | 27588 |
| 24589 | 25589 | 26589 | 27589 |
| 24590 | 25590 | 26590 | 27590 |
| 24591 | 25591 | 26591 | 27591 |
| 24592 | 25592 | 26592 | 27592 |
| 24593 | 25593 | 26593 | 27593 |
| 24594 | 25594 | 26594 | 27594 |
| 24595 | 25595 | 26595 | 27595 |
| 24596 | 25596 | 26596 | 27596 |
| 24597 | 25597 | 26597 | 27597 |
| 24598 | 25598 | 26598 | 27598 |
| 24599 | 25599 | 26599 | 27599 |
| 24600 | 25600 | 26600 | 27600 |
| 24601 | 25601 | 26601 | 27601 |
| 24602 | 25602 | 26602 | 27602 |
| 24603 | 25603 | 26603 | 27603 |
| 24604 | 25604 | 26604 | 27604 |
| 24605 | 25605 | 26605 | 27605 |
| 24606 | 25606 | 26606 | 27606 |
| 24607 | 25607 | 26607 | 27607 |
| 24608 | 25608 | 26608 | 27608 |
| 24609 | 25609 | 26609 | 27609 |
| 24610 | 25610 | 26610 | 27610 |
| 24611 | 25611 | 26611 | 27611 |
| 24612 | 25612 | 26612 | 27612 |
| 24613 | 25613 | 26613 | 27613 |
| 24614 | 25614 | 26614 | 27614 |
| 24615 | 25615 | 26615 | 27615 |
| 24616 | 25616 | 26616 | 27616 |
| 24617 | 25617 | 26617 | 27617 |
| 24618 | 25618 | 26618 | 27618 |
| 24619 | 25619 | 26619 | 27619 |
| 24620 | 25620 | 26620 | 27620 |
| 24621 | 25621 | 26621 | 27621 |
| 24622 | 25622 | 26622 | 27622 |
| 24623 | 25623 | 26623 | 27623 |
| 24624 | 25624 | 26624 | 27624 |
| 24625 | 25625 | 26625 | 27625 |
| 24626 | 25626 | 26626 | 27626 |
| 24627 | 25627 | 26627 | 27627 |
| 24628 | 25628 | 26628 | 27628 |
| 24629 | 25629 | 26629 | 27629 |
| 24630 | 25630 | 26630 | 27630 |
| 24631 | 25631 | 26631 | 27631 |
| 24632 | 25632 | 26632 | 27632 |
| 24633 | 25633 | 26633 | 27633 |
| 24634 | 25634 | 26634 | 27634 |
| 24635 | 25635 | 26635 | 27635 |
| 24636 | 25636 | 26636 | 27636 |
| 24637 | 25637 | 26637 | 27637 |
| 24638 | 25638 | 26638 | 27638 |
| 24639 | 25639 | 26639 | 27639 |
| 24640 | 25640 | 26640 | 27640 |
| 24641 | 25641 | 26641 | 27641 |
| 24642 | 25642 | 26642 | 27642 |
| 24643 | 25643 | 26643 | 27643 |
| 24644 | 25644 | 26644 | 27644 |
| 24645 | 25645 | 26645 | 27645 |
| 24646 | 25646 | 26646 | 27646 |
| 24647 | 25647 | 26647 | 27647 |
| 24648 | 25648 | 26648 | 27648 |
| 24649 | 25649 | 26649 | 27649 |
| 24650 | 25650 | 26650 | 27650 |
| 24651 | 25651 | 26651 | 27651 |
| 24652 | 25652 | 26652 | 27652 |
| 24653 | 25653 | 26653 | 27653 |
| 24654 | 25654 | 26654 | 27654 |
| 24655 | 25655 | 26655 | 27655 |
| 24656 | 25656 | 26656 | 27656 |
| 24657 | 25657 | 26657 | 27657 |
| 24658 | 25658 | 26658 | 27658 |
| 24659 | 25659 | 26659 | 27659 |
| 24660 | 25660 | 26660 | 27660 |
| 24661 | 25661 | 26661 | 27661 |
| 24662 | 25662 | 26662 | 27662 |
| 24663 | 25663 | 26663 | 27663 |
| 24664 | 25664 | 26664 | 27664 |
| 24665 | 25665 | 26665 | 27665 |
| 24666 | 25666 | 26666 | 27666 |
| 24667 | 25667 | 26667 | 27667 |
| 24668 | 25668 | 26668 | 27668 |
| 24669 | 25669 | 26669 | 27669 |
| 24670 | 25670 | 26670 | 27670 |
| 24671 | 25671 | 26671 | 27671 |
| 24672 | 25672 | 26672 | 27672 |
| 24673 | 25673 | 26673 | 27673 |
| 24674 | 25674 | 26674 | 27674 |
| 24675 | 25675 | 26675 | 27675 |
| 24676 | 25676 | 26676 | 27676 |
| 24677 | 25677 | 26677 | 27677 |
| 24678 | 25678 | 26678 | 27678 |
| 24679 | 25679 | 26679 | 27679 |
| 24680 | 25680 | 26680 | 27680 |
| 24681 | 25681 | 26681 | 27681 |
| 24682 | 25682 | 26682 | 27682 |
| 24683 | 25683 | 26683 | 27683 |
| 24684 | 25684 | 26684 | 27684 |
| 24685 | 25685 | 26685 | 27685 |
| 24686 | 25686 | 26686 | 27686 |
| 24687 | 25687 | 26687 | 27687 |
| 24688 | 25688 | 26688 | 27688 |
| 24689 | 25689 | 26689 | 27689 |
| 24690 | 25690 | 26690 | 27690 |
| 24691 | 25691 | 26691 | 27691 |
| 24692 | 25692 | 26692 | 27692 |
| 24693 | 25693 | 26693 | 27693 |
| 24694 | 25694 | 26694 | 27694 |
| 24695 | 25695 | 26695 | 27695 |
| 24696 | 25696 | 26696 | 27696 |
| 24697 | 25697 | 26697 | 27697 |
| 24698 | 25698 | 26698 | 27698 |
| 24699 | 25699 | 26699 | 27699 |
| 24700 | 25700 | 26700 | 27700 |
| 24701 | 25701 | 26701 | 27701 |
| 24702 | 25702 | 26702 | 27702 |
| 24703 | 25703 | 26703 | 27703 |
| 24704 | 25704 | 26704 | 27704 |
| 24705 | 25705 | 26705 | 27705 |
| 24706 | 25706 | 26706 | 27706 |
| 24707 | 25707 | 26707 | 27707 |
| 24708 | 25708 | 26708 | 27708 |
| 24709 | 25709 | 26709 | 27709 |
| 24710 | 25710 | 26710 | 27710 |
| 24711 | 25711 | 26711 | 27711 |
| 24712 | 25712 | 26712 | 27712 |
| 24713 | 25713 | 26713 | 27713 |
| 24714 | 25714 | 26714 | 27714 |
| 24715 | 25715 | 26715 | 27715 |
| 24716 | 25716 | 26716 | 27716 |
| 24717 | 25717 | 26717 | 27717 |
| 24718 | 25718 | 26718 | 27718 |
| 24719 | 25719 | 26719 | 27719 |
| 24720 | 25720 | 26720 | 27720 |
| 24721 | 25721 | 26721 | 27721 |
| 24722 | 25722 | 26722 | 27722 |
| 24723 | 25723 | 26723 | 27723 |
| 24724 | 25724 | 26724 | 27724 |
| 24725 | 25725 | 26725 | 27725 |
| 24726 | 25726 | 26726 | 27726 |
| 24727 | 25727 | 26727 | 27727 |
| 24728 | 25728 | 26728 | 27728 |
| 24729 | 25729 | 26729 | 27729 |
| 24730 | 25730 | 26730 | 27730 |
| 24731 | 25731 | 26731 | 27731 |
| 24732 | 25732 | 26732 | 27732 |
| 24733 | 25733 | 26733 | 27733 |
| 24734 | 25734 | 26734 | 27734 |
| 24735 | 25735 | 26735 | 27735 |
| 24736 | 25736 | 26736 | 27736 |
| 24737 | 25737 | 26737 | 27737 |
| 24738 | 25738 | 26738 | 27738 |
| 24739 | 25739 | 26739 | 27739 |
| 24740 | 25740 | 26740 | 27740 |
| 24741 | 25741 | 26741 | 27741 |
| 24742 | 25742 | 26742 | 27742 |
| 24743 | 25743 | 26743 | 27743 |
| 24744 | 25744 | 26744 | 27744 |
| 24745 | 25745 | 26745 | 27745 |
| 24746 | 25746 | 26746 | 27746 |
| 24747 | 25747 | 26747 | 27747 |
| 24748 | 25748 | 26748 | 27748 |
| 24749 | 25749 | 26749 | 27749 |
| 24750 | 25750 | 26750 | 27750 |
| 24751 | 25751 | 26751 | 27751 |
| 24752 | 25752 | 26752 | 27752 |
| 24753 | 25753 | 26753 | 27753 |
| 24754 | 25754 | 26754 | 27754 |
| 24755 | 25755 | 26755 | 27755 |
| 24756 | 25756 | 26756 | 27756 |
| 24757 | 25757 | 26757 | 27757 |
| 24758 | 25758 | 26758 | 27758 |
| 24759 | 25759 | 26759 | 27759 |
| 24760 | 25760 | 26760 | 27760 |
| 24761 | 25761 | 26761 | 27761 |
| 24762 | 25762 | 26762 | 27762 |
| 24763 | 25763 | 26763 | 27763 |
| 24764 | 25764 | 26764 | 27764 |
| 24765 | 25765 | 26765 | 27765 |
| 24766 | 25766 | 26766 | 27766 |
| 24767 | 25767 | 26767 | 27767 |
| 24768 | 25768 | 26768 | 27768 |
| 24769 | 25769 | 26769 | 27769 |
| 24770 | 25770 | 26770 | 27770 |
| 24771 | 25771 | 26771 | 27771 |
| 24772 | 25772 | 26772 | 27772 |
| 24773 | 25773 | 26773 | 27773 |
| 24774 | 25774 | 26774 | 27774 |
| 24775 | 25775 | 26775 | 27775 |
| 24776 | 25776 | 26776 | 27776 |
| 24777 | 25777 | 26777 | 27777 |
| 24778 | 25778 | 26778 | 27778 |
| 24779 | 25779 | 26779 | 27779 |
| 24780 | 25780 | 26780 | 27780 |
| 24781 | 25781 | 26781 | 27781 |
| 24782 | 25782 | 26782 | 27782 |
| 24783 | 25783 | 26783 | 27783 |
| 24784 | 25784 | 26784 | 27784 |
| 24785 | 25785 | 26785 | 27785 |
| 24786 | 25786 | 26786 | 27786 |
| 24787 | 25787 | 26787 | 27787 |
| 24788 | 25788 | 26788 | 27788 |
| 24789 | 25789 | 26789 | 27789 |
| 24790 | 25790 | 26790 | 27790 |
| 24791 | 25791 | 26791 | 27791 |
| 24792 | 25792 | 26792 | 27792 |
| 24793 | 25793 | 26793 | 27793 |
| 24794 | 25794 | 26794 | 27794 |
| 24795 | 25795 | 26795 | 27795 |
| 24796 | 25796 | 26796 | 27796 |
| 24797 | 25797 | 26797 | 27797 |
| 24798 | 25798 | 26798 | 27798 |
| 24799 | 25799 | 26799 | 27799 |
| 24800 | 25800 | 26800 | 27800 |
| 24801 | 25801 | 26801 | 27801 |
| 24802 | 25802 | 26802 | 27802 |
| 24803 | 25803 | 26803 | 27803 |
| 24804 | 25804 | 26804 | 27804 |
| 24805 | 25805 | 26805 | 27805 |
| 24806 | 25806 | 26806 | 27806 |
| 24807 | 25807 | 26807 | 27807 |
| 24808 | 25808 | 26808 | 27808 |
| 24809 | 25809 | 26809 | 27809 |
| 24810 | 25810 | 26810 | 27810 |
| 24811 | 25811 | 26811 | 27811 |
| 24812 | 25812 | 26812 | 27812 |
| 24813 | 25813 | 26813 | 27813 |
| 24814 | 25814 | 26814 | 27814 |
| 24815 | 25815 | 26815 | 27815 |
| 24816 | 25816 | 26816 | 27816 |
| 24817 | 25817 | 26817 | 27817 |
| 24818 | 25818 | 26818 | 27818 |
| 24819 | 25819 | 26819 | 27819 |
| 24820 | 25820 | 26820 | 27820 |
| 24821 | 25821 | 26821 | 27821 |
| 24822 | 25822 | 26822 | 27822 |
| 24823 | 25823 | 26823 | 27823 |
| 24824 | 25824 | 26824 | 27824 |
| 24825 | 25825 | 26825 | 27825 |
| 24826 | 25826 | 26826 | 27826 |
| 24827 | 25827 | 26827 | 27827 |
| 24828 | 25828 | 26828 | 27828 |
| 24829 | 25829 | 26829 | 27829 |
| 24830 | 25830 | 26830 | 27830 |
| 24831 | 25831 | 26831 | 27831 |
| 24832 | 25832 | 26832 | 27832 |
| 24833 | 25833 | 26833 | 27833 |
| 24834 | 25834 | 26834 | 27834 |
| 24835 | 25835 | 26835 | 27835 |
| 24836 | 25836 | 26836 | 27836 |
| 24837 | 25837 | 26837 | 27837 |
| 24838 | 25838 | 26838 | 27838 |
| 24839 | 25839 | 26839 | 27839 |
| 24840 | 25840 | 26840 | 27840 |
| 24841 | 25841 | 26841 | 27841 |
| 24842 | 25842 | 26842 | 27842 |
| 24843 | 25843 | 26843 | 27843 |
| 24844 | 25844 | 26844 | 27844 |
| 24845 | 25845 | 26845 | 27845 |
| 24846 | 25846 | 26846 | 27846 |
| 24847 | 25847 | 26847 | 27847 |
| 24848 | 25848 | 26848 | 27848 |
| 24849 | 25849 | 26849 | 27849 |
| 24850 | 25850 | 26850 | 27850 |
| 24851 | 25851 | 26851 | 27851 |
| 24852 | 25852 | 26852 | 27852 |
| 24853 | 25853 | 26853 | 27853 |
| 24854 | 25854 | 26854 | 27854 |
| 24855 | 25855 | 26855 | 27855 |
| 24856 | 25856 | 26856 | 27856 |
| 24857 | 25857 | 26857 | 27857 |
| 24858 | 25858 | 26858 | 27858 |
| 24859 | 25859 | 26859 | 27859 |
| 24860 | 25860 | 26860 | 27860 |
| 24861 | 25861 | 26861 | 27861 |
| 24862 | 25862 | 26862 | 27862 |
| 24863 | 25863 | 26863 | 27863 |
| 24864 | 25864 | 26864 | 27864 |
| 24865 | 25865 | 26865 | 27865 |
| 24866 | 25866 | 26866 | 27866 |
| 24867 | 25867 | 26867 | 27867 |
| 24868 | 25868 | 26868 | 27868 |
| 24869 | 25869 | 26869 | 27869 |
| 24870 | 25870 | 26870 | 27870 |
| 24871 | 25871 | 26871 | 27871 |
| 24872 | 25872 | 26872 | 27872 |
| 24873 | 25873 | 26873 | 27873 |
| 24874 | 25874 | 26874 | 27874 |
| 24875 | 25875 | 26875 | 27875 |
| 24876 | 25876 | 26876 | 27876 |
| 24877 | 25877 | 26877 | 27877 |
| 24878 | 25878 | 26878 | 27878 |
| 24879 | 25879 | 26879 | 27879 |
| 24880 | 25880 | 26880 | 27880 |
| 24881 | 25881 | 26881 | 27881 |
| 24882 | 25882 | 26882 | 27882 |
| 24883 | 25883 | 26883 | 27883 |
| 24884 | 25884 | 26884 | 27884 |
| 24885 | 25885 | 26885 | 27885 |
| 24886 | 25886 | 26886 | 27886 |
| 24887 | 25887 | 26887 | 27887 |
| 24888 | 25888 | 26888 | 27888 |
| 24889 | 25889 | 26889 | 27889 |
| 24890 | 25890 | 26890 | 27890 |
| 24891 | 25891 | 26891 | 27891 |
| 24892 | 25892 | 26892 | 27892 |
| 24893 | 25893 | 26893 | 27893 |
| 24894 | 25894 | 26894 | 27894 |
| 24895 | 25895 | 26895 | 27895 |
| 24896 | 25896 | 26896 | 27896 |
| 24897 | 25897 | 26897 | 27897 |
| 24898 | 25898 | 26898 | 27898 |
| 24899 | 25899 | 26899 | 27899 |
| 24900 | 25900 | 26900 | 27900 |
| 24901 | 25901 | 26901 | 27901 |
| 24902 | 25902 | 26902 | 27902 |
| 24903 | 25903 | 26903 | 27903 |
| 24904 | 25904 | 26904 | 27904 |
| 24905 | 25905 | 26905 | 27905 |
| 24906 | 25906 | 26906 | 27906 |
| 24907 | 25907 | 26907 | 27907 |
| 24908 | 25908 | 26908 | 27908 |
| 24909 | 25909 | 26909 | 27909 |
| 24910 | 25910 | 26910 | 27910 |
| 24911 | 25911 | 26911 | 27911 |
| 24912 | 25912 | 26912 | 27912 |
| 24913 | 25913 | 26913 | 27913 |
| 24914 | 25914 | 26914 | 27914 |
| 24915 | 25915 | 26915 | 27915 |
| 24916 | 25916 | 26916 | 27916 |
| 24917 | 25917 | 26917 | 27917 |
| 24918 | 25918 | 26918 | 27918 |
| 24919 | 25919 | 26919 | 27919 |
| 24920 | 25920 | 26920 | 27920 |
| 24921 | 25921 | 26921 | 27921 |
| 24922 | 25922 | 26922 | 27922 |
| 24923 | 25923 | 26923 | 27923 |
| 24924 | 25924 | 26924 | 27924 |
| 24925 | 25925 | 26925 | 27925 |
| 24926 | 25926 | 26926 | 27926 |
| 24927 | 25927 | 26927 | 27927 |
| 24928 | 25928 | 26928 | 27928 |
| 24929 | 25929 | 26929 | 27929 |
| 24930 | 25930 | 26930 | 27930 |
| 24931 | 25931 | 26931 | 27931 |
| 24932 | 25932 | 26932 | 27932 |
| 24933 | 25933 | 26933 | 27933 |
| 24934 | 25934 | 26934 | 27934 |
| 24935 | 25935 | 26935 | 27935 |
| 24936 | 25936 | 26936 | 27936 |
| 24937 | 25937 | 26937 | 27937 |
| 24938 | 25938 | 26938 | 27938 |
| 24939 | 25939 | 26939 | 27939 |
| 24940 | 25940 | 26940 | 27940 |
| 24941 | 25941 | 26941 | 27941 |
| 24942 | 25942 | 26942 | 27942 |
| 24943 | 25943 | 26943 | 27943 |
| 24944 | 25944 | 26944 | 27944 |
| 24945 | 25945 | 26945 | 27945 |
| 24946 | 25946 | 26946 | 27946 |
| 24947 | 25947 | 26947 | 27947 |
| 24948 | 25948 | 26948 | 27948 |
| 24949 | 25949 | 26949 | 27949 |
| 24950 | 25950 | 26950 | 27950 |
| 24951 | 25951 | 26951 | 27951 |
| 24952 | 25952 | 26952 | 27952 |
| 24953 | 25953 | 26953 | 27953 |
| 24954 | 25954 | 26954 | 27954 |
| 24955 | 25955 | 26955 | 27955 |
| 24956 | 25956 | 26956 | 27956 |
| 24957 | 25957 | 26957 | 27957 |
| 24958 | 25958 | 26958 | 27958 |
| 24959 | 25959 | 26959 | 27959 |
| 24960 | 25960 | 26960 | 27960 |
| 24961 | 25961 | 26961 | 27961 |
| 24962 | 25962 | 26962 | 27962 |
| 24963 | 25963 | 26963 | 27963 |
| 24964 | 25964 | 26964 | 27964 |
| 24965 | 25965 | 26965 | 27965 |
| 24966 | 25966 | 26966 | 27966 |
| 24967 | 25967 | 26967 | 27967 |
| 24968 | 25968 | 26968 | 27968 |
| 24969 | 25969 | 26969 | 27969 |
| 24970 | 25970 | 26970 | 27970 |
| 24971 | 25971 | 26971 | 27971 |
| 24972 | 25972 | 26972 | 27972 |
| 24973 | 25973 | 26973 | 27973 |
| 24974 | 25974 | 26974 | 27974 |
| 24975 | 25975 | 26975 | 27975 |
| 24976 | 25976 | 26976 | 27976 |
| 24977 | 25977 | 26977 | 27977 |
| 24978 | 25978 | 26978 | 27978 |
| 24979 | 25979 | 26979 | 27979 |
| 24980 | 25980 | 26980 | 27980 |
| 24981 | 25981 | 26981 | 27981 |
| 24982 | 25982 | 26982 | 27982 |
| 24983 | 25983 | 26983 | 27983 |
| 24984 | 25984 | 26984 | 27984 |
| 24985 | 25985 | 26985 | 27985 |
| 24986 | 25986 | 26986 | 27986 |
| 24987 | 25987 | 26987 | 27987 |
| 24988 | 25988 | 26988 | 27988 |
| 24989 | 25989 | 26989 | 27989 |
| 24990 | 25990 | 26990 | 27990 |
| 24991 | 25991 | 26991 | 27991 |
| 24992 | 25992 | 26992 | 27992 |
| 24993 | 25993 | 26993 | 27993 |
| 24994 | 25994 | 26994 | 27994 |
| 24995 | 25995 | 26995 | 27995 |
| 24996 | 25996 | 26996 | 27996 |
| 24997 | 25997 | 26997 | 27997 |
| 24998 | 25998 | 26998 | 27998 |
| 24999 | 25999 | 26999 | 27999 |
| 25000 | 26000 | 27000 | 28000 |
| 25001 | 26001 | 27001 | 28001 |
| 25002 | 26002 | 27002 | 28002 |

In some embodiments, the sequences that specifically bind to walnut comprise an inner sequence selected from the group consisting of the nucleic acid sequences of SEQ ID NOs. 28003 to 29002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to walnut may comprise a nucleic acid sequence selected from the group consisting of SEQ ID NOs. 29003 to 30002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to walnut may comprise a nucleic acid sequence selected from the group consisting of SEQ ID NOs. 30003 to 31002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to walnut may comprise a nucleic acid sequence selected from the group consisting of SEQ ID NOs. 31003 to 32002. In one embodiment, the aptamer of the present disclosure that specifically binds to walnut may comprise a nucleic acid sequence selected from the group consisting of SEQ ID NOs. 28003 to 32002 listed in Table 9, or variant thereof.

TABLE 9
Aptamer sequences against walnut
Aptamer sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 28003 | 29003 | 30003 | 31003 |
| 28004 | 29004 | 30004 | 31004 |
| 28005 | 29005 | 30005 | 31005 |
| 28006 | 29006 | 30006 | 31006 |
| 28007 | 29007 | 30007 | 31007 |
| 28008 | 29008 | 30008 | 31008 |
| 28009 | 29009 | 30009 | 31009 |
| 28010 | 29010 | 30010 | 31010 |
| 28011 | 29011 | 30011 | 31011 |
| 28012 | 29012 | 30012 | 31012 |
| 28013 | 29013 | 30013 | 31013 |
| 28014 | 29014 | 30014 | 31014 |
| 28015 | 29015 | 30015 | 31015 |
| 28016 | 29016 | 30016 | 31016 |
| 28017 | 29017 | 30017 | 31017 |
| 28018 | 29018 | 30018 | 31018 |
| 28019 | 29019 | 30019 | 31019 |
| 28020 | 29020 | 30020 | 31020 |
| 28021 | 29021 | 30021 | 31021 |
| 28022 | 29022 | 30022 | 31022 |
| 28023 | 29023 | 30023 | 31023 |
| 28024 | 29024 | 30024 | 31024 |
| 28025 | 29025 | 30025 | 31025 |
| 28026 | 29026 | 30026 | 31026 |
| 28027 | 29027 | 30027 | 31027 |
| 28028 | 29028 | 30028 | 31028 |
| 28029 | 29029 | 30029 | 31029 |
| 28030 | 29030 | 30030 | 31030 |
| 28031 | 29031 | 30031 | 31031 |
| 28032 | 29032 | 30032 | 31032 |
| 28033 | 29033 | 30033 | 31033 |
| 28034 | 29034 | 30034 | 31034 |
| 28035 | 29035 | 30035 | 31035 |
| 28036 | 29036 | 30036 | 31036 |
| 28037 | 29037 | 30037 | 31037 |
| 28038 | 29038 | 30038 | 31038 |
| 28039 | 29039 | 30039 | 31039 |
| 28040 | 29040 | 30040 | 31040 |
| 28041 | 29041 | 30041 | 31041 |
| 28042 | 29042 | 30042 | 31042 |
| 28043 | 29043 | 30043 | 31043 |
| 28044 | 29044 | 30044 | 31044 |
| 28045 | 29045 | 30045 | 31045 |
| 28046 | 29046 | 30046 | 31046 |
| 28047 | 29047 | 30047 | 31047 |
| 28048 | 29048 | 30048 | 31048 |
| 28049 | 29049 | 30049 | 31049 |
| 28050 | 29050 | 30050 | 31050 |
| 28051 | 29051 | 30051 | 31051 |
| 28052 | 29052 | 30052 | 31052 |
| 28053 | 29053 | 30053 | 31053 |
| 28054 | 29054 | 30054 | 31054 |
| 28055 | 29055 | 30055 | 31055 |
| 28056 | 29056 | 30056 | 31056 |
| 28057 | 29057 | 30057 | 31057 |
| 28058 | 29058 | 30058 | 31058 |
| 28059 | 29059 | 30059 | 31059 |
| 28060 | 29060 | 30060 | 31060 |
| 28061 | 29061 | 30061 | 31061 |
| 28062 | 29062 | 30062 | 31062 |
| 28063 | 29063 | 30063 | 31063 |
| 28064 | 29064 | 30064 | 31064 |
| 28065 | 29065 | 30065 | 31065 |
| 28066 | 29066 | 30066 | 31066 |
| 28067 | 29067 | 30067 | 31067 |
| 28068 | 29068 | 30068 | 31068 |
| 28069 | 29069 | 30069 | 31069 |
| 28070 | 29070 | 30070 | 31070 |
| 28071 | 29071 | 30071 | 31071 |
| 28072 | 29072 | 30072 | 31072 |
| 28073 | 29073 | 30073 | 31073 |
| 28074 | 29074 | 30074 | 31074 |
| 28075 | 29075 | 30075 | 31075 |
| 28076 | 29076 | 30076 | 31076 |
| 28077 | 29077 | 30077 | 31077 |
| 28078 | 29078 | 30078 | 31078 |
| 28079 | 29079 | 30079 | 31079 |
| 28080 | 29080 | 30080 | 31080 |
| 28081 | 29081 | 30081 | 31081 |
| 28082 | 29082 | 30082 | 31082 |
| 28083 | 29083 | 30083 | 31083 |
| 28084 | 29084 | 30084 | 31084 |
| 28085 | 29085 | 30085 | 31085 |
| 28086 | 29086 | 30086 | 31086 |
| 28087 | 29087 | 30087 | 31087 |
| 28088 | 29088 | 30088 | 31088 |
| 28089 | 29089 | 30089 | 31089 |
| 28090 | 29090 | 30090 | 31090 |
| 28091 | 29091 | 30091 | 31091 |
| 28092 | 29092 | 30092 | 31092 |
| 28093 | 29093 | 30093 | 31093 |
| 28094 | 29094 | 30094 | 31094 |
| 28095 | 29095 | 30095 | 31095 |
| 28096 | 29096 | 30096 | 31096 |
| 28097 | 29097 | 30097 | 31097 |
| 28098 | 29098 | 30098 | 31098 |
| 28099 | 29099 | 30099 | 31099 |
| 28100 | 29100 | 30100 | 31100 |
| 28101 | 29101 | 30101 | 31101 |
| 28102 | 29102 | 30102 | 31102 |
| 28103 | 29103 | 30103 | 31103 |
| 28104 | 29104 | 30104 | 31104 |
| 28105 | 29105 | 30105 | 31105 |
| 28106 | 29106 | 30106 | 31106 |
| 28107 | 29107 | 30107 | 31107 |
| 28108 | 29108 | 30108 | 31108 |
| 28109 | 29109 | 30109 | 31109 |
| 28110 | 29110 | 30110 | 31110 |
| 28111 | 29111 | 30111 | 31111 |
| 28112 | 29112 | 30112 | 31112 |
| 28113 | 29113 | 30113 | 31113 |
| 28114 | 29114 | 30114 | 31114 |
| 28115 | 29115 | 30115 | 31115 |
| 28116 | 29116 | 30116 | 31116 |
| 28117 | 29117 | 30117 | 31117 |
| 28118 | 29118 | 30118 | 31118 |
| 28119 | 29119 | 30119 | 31119 |
| 28120 | 29120 | 30120 | 31120 |
| 28121 | 29121 | 30121 | 31121 |
| 28122 | 29122 | 30122 | 31122 |
| 28123 | 29123 | 30123 | 31123 |
| 28124 | 29124 | 30124 | 31124 |
| 28125 | 29125 | 30125 | 31125 |
| 28126 | 29126 | 30126 | 31126 |
| 28127 | 29127 | 30127 | 31127 |
| 28128 | 29128 | 30128 | 31128 |
| 28129 | 29129 | 30129 | 31129 |
| 28130 | 29130 | 30130 | 31130 |
| 28131 | 29131 | 30131 | 31131 |
| 28132 | 29132 | 30132 | 31132 |
| 28133 | 29133 | 30133 | 31133 |
| 28134 | 29134 | 30134 | 31134 |
| 28135 | 29135 | 30135 | 31135 |
| 28136 | 29136 | 30136 | 31136 |
| 28137 | 29137 | 30137 | 31137 |
| 28138 | 29138 | 30138 | 31138 |
| 28139 | 29139 | 30139 | 31139 |
| 28140 | 29140 | 30140 | 31140 |
| 28141 | 29141 | 30141 | 31141 |
| 28142 | 29142 | 30142 | 31142 |
| 28143 | 29143 | 30143 | 31143 |
| 28144 | 29144 | 30144 | 31144 |
| 28145 | 29145 | 30145 | 31145 |
| 28146 | 29146 | 30146 | 31146 |
| 28147 | 29147 | 30147 | 31147 |
| 28148 | 29148 | 30148 | 31148 |
| 28149 | 29149 | 30149 | 31149 |
| 28150 | 29150 | 30150 | 31150 |
| 28151 | 29151 | 30151 | 31151 |
| 28152 | 29152 | 30152 | 31152 |
| 28153 | 29153 | 30153 | 31153 |
| 28154 | 29154 | 30154 | 31154 |
| 28155 | 29155 | 30155 | 31155 |
| 28156 | 29156 | 30156 | 31156 |
| 28157 | 29157 | 30157 | 31157 |
| 28158 | 29158 | 30158 | 31158 |
| 28159 | 29159 | 30159 | 31159 |
| 28160 | 29160 | 30160 | 31160 |
| 28161 | 29161 | 30161 | 31161 |
| 28162 | 29162 | 30162 | 31162 |
| 28163 | 29163 | 30163 | 31163 |
| 28164 | 29164 | 30164 | 31164 |
| 28165 | 29165 | 30165 | 31165 |
| 28166 | 29166 | 30166 | 31166 |
| 28167 | 29167 | 30167 | 31167 |
| 28168 | 29168 | 30168 | 31168 |
| 28169 | 29169 | 30169 | 31169 |
| 28170 | 29170 | 30170 | 31170 |
| 28171 | 29171 | 30171 | 31171 |
| 28172 | 29172 | 30172 | 31172 |
| 28173 | 29173 | 30173 | 31173 |
| 28174 | 29174 | 30174 | 31174 |
| 28175 | 29175 | 30175 | 31175 |
| 28176 | 29176 | 30176 | 31176 |
| 28177 | 29177 | 30177 | 31177 |
| 28178 | 29178 | 30178 | 31178 |
| 28179 | 29179 | 30179 | 31179 |
| 28180 | 29180 | 30180 | 31180 |
| 28181 | 29181 | 30181 | 31181 |
| 28182 | 29182 | 30182 | 31182 |
| 28183 | 29183 | 30183 | 31183 |
| 28184 | 29184 | 30184 | 31184 |
| 28185 | 29185 | 30185 | 31185 |
| 28186 | 29186 | 30186 | 31186 |
| 28187 | 29187 | 30187 | 31187 |
| 28188 | 29188 | 30188 | 31188 |
| 28189 | 29189 | 30189 | 31189 |
| 28190 | 29190 | 30190 | 31190 |
| 28191 | 29191 | 30191 | 31191 |
| 28192 | 29192 | 30192 | 31192 |
| 28193 | 29193 | 30193 | 31193 |
| 28194 | 29194 | 30194 | 31194 |
| 28195 | 29195 | 30195 | 31195 |
| 28196 | 29196 | 30196 | 31196 |
| 28197 | 29197 | 30197 | 31197 |
| 28198 | 29198 | 30198 | 31198 |
| 28199 | 29199 | 30199 | 31199 |
| 28200 | 29200 | 30200 | 31200 |
| 28201 | 29201 | 30201 | 31201 |
| 28202 | 29202 | 30202 | 31202 |
| 28203 | 29203 | 30203 | 31203 |
| 28204 | 29204 | 30204 | 31204 |
| 28205 | 29205 | 30205 | 31205 |
| 28206 | 29206 | 30206 | 31206 |
| 28207 | 29207 | 30207 | 31207 |
| 28208 | 29208 | 30208 | 31208 |
| 28209 | 29209 | 30209 | 31209 |
| 28210 | 29210 | 30210 | 31210 |
| 28211 | 29211 | 30211 | 31211 |
| 28212 | 29212 | 30212 | 31212 |
| 28213 | 29213 | 30213 | 31213 |
| 28214 | 29214 | 30214 | 31214 |
| 28215 | 29215 | 30215 | 31215 |
| 28216 | 29216 | 30216 | 31216 |
| 28217 | 29217 | 30217 | 31217 |
| 28218 | 29218 | 30218 | 31218 |
| 28219 | 29219 | 30219 | 31219 |
| 28220 | 29220 | 30220 | 31220 |
| 28221 | 29221 | 30221 | 31221 |
| 28222 | 29222 | 30222 | 31222 |
| 28223 | 29223 | 30223 | 31223 |
| 28224 | 29224 | 30224 | 31224 |
| 28225 | 29225 | 30225 | 31225 |
| 28226 | 29226 | 30226 | 31226 |
| 28227 | 29227 | 30227 | 31227 |
| 28228 | 29228 | 30228 | 31228 |
| 28229 | 29229 | 30229 | 31229 |
| 28230 | 29230 | 30230 | 31230 |
| 28231 | 29231 | 30231 | 31231 |
| 28232 | 29232 | 30232 | 31232 |
| 28233 | 29233 | 30233 | 31233 |
| 28234 | 29234 | 30234 | 31234 |
| 28235 | 29235 | 30235 | 31235 |
| 28236 | 29236 | 30236 | 31236 |
| 28237 | 29237 | 30237 | 31237 |
| 28238 | 29238 | 30238 | 31238 |
| 28239 | 29239 | 30239 | 31239 |
| 28240 | 29240 | 30240 | 31240 |
| 28241 | 29241 | 30241 | 31241 |
| 28242 | 29242 | 30242 | 31242 |
| 28243 | 29243 | 30243 | 31243 |
| 28244 | 29244 | 30244 | 31244 |
| 28245 | 29245 | 30245 | 31245 |
| 28246 | 29246 | 30246 | 31246 |
| 28247 | 29247 | 30247 | 31247 |
| 28248 | 29248 | 30248 | 31248 |
| 28249 | 29249 | 30249 | 31249 |
| 28250 | 29250 | 30250 | 31250 |
| 28251 | 29251 | 30251 | 31251 |
| 28252 | 29252 | 30252 | 31252 |
| 28253 | 29253 | 30253 | 31253 |
| 28254 | 29254 | 30254 | 31254 |
| 28255 | 29255 | 30255 | 31255 |
| 28256 | 29256 | 30256 | 31256 |
| 28257 | 29257 | 30257 | 31257 |
| 28258 | 29258 | 30258 | 31258 |
| 28259 | 29259 | 30259 | 31259 |
| 28260 | 29260 | 30260 | 31260 |
| 28261 | 29261 | 30261 | 31261 |
| 28262 | 29262 | 30262 | 31262 |
| 28263 | 29263 | 30263 | 31263 |
| 28264 | 29264 | 30264 | 31264 |
| 28265 | 29265 | 30265 | 31265 |
| 28266 | 29266 | 30266 | 31266 |
| 28267 | 29267 | 30267 | 31267 |
| 28268 | 29268 | 30268 | 31268 |
| 28269 | 29269 | 30269 | 31269 |
| 28270 | 29270 | 30270 | 31270 |
| 28271 | 29271 | 30271 | 31271 |
| 28272 | 29272 | 30272 | 31272 |
| 28273 | 29273 | 30273 | 31273 |
| 28274 | 29274 | 30274 | 31274 |
| 28275 | 29275 | 30275 | 31275 |
| 28276 | 29276 | 30276 | 31276 |
| 28277 | 29277 | 30277 | 31277 |
| 28278 | 29278 | 30278 | 31278 |
| 28279 | 29279 | 30279 | 31279 |
| 28280 | 29280 | 30280 | 31280 |
| 28281 | 29281 | 30281 | 31281 |
| 28282 | 29282 | 30282 | 31282 |
| 28283 | 29283 | 30283 | 31283 |
| 28284 | 29284 | 30284 | 31284 |
| 28285 | 29285 | 30285 | 31285 |
| 28286 | 29286 | 30286 | 31286 |
| 28287 | 29287 | 30287 | 31287 |
| 28288 | 29288 | 30288 | 31288 |
| 28289 | 29289 | 30289 | 31289 |
| 28290 | 29290 | 30290 | 31290 |
| 28291 | 29291 | 30291 | 31291 |
| 28292 | 29292 | 30292 | 31292 |
| 28293 | 29293 | 30293 | 31293 |
| 28294 | 29294 | 30294 | 31294 |
| 28295 | 29295 | 30295 | 31295 |
| 28296 | 29296 | 30296 | 31296 |
| 28297 | 29297 | 30297 | 31297 |
| 28298 | 29298 | 30298 | 31298 |
| 28299 | 29299 | 30299 | 31299 |
| 28300 | 29300 | 30300 | 31300 |
| 28301 | 29301 | 30301 | 31301 |
| 28302 | 29302 | 30302 | 31302 |
| 28303 | 29303 | 30303 | 31303 |
| 28304 | 29304 | 30304 | 31304 |
| 28305 | 29305 | 30305 | 31305 |
| 28306 | 29306 | 30306 | 31306 |
| 28307 | 29307 | 30307 | 31307 |
| 28308 | 29308 | 30308 | 31308 |
| 28309 | 29309 | 30309 | 31309 |
| 28310 | 29310 | 30310 | 31310 |
| 28311 | 29311 | 30311 | 31311 |
| 28312 | 29312 | 30312 | 31312 |
| 28313 | 29313 | 30313 | 31313 |
| 28314 | 29314 | 30314 | 31314 |
| 28315 | 29315 | 30315 | 31315 |
| 28316 | 29316 | 30316 | 31316 |
| 28317 | 29317 | 30317 | 31317 |
| 28318 | 29318 | 30318 | 31318 |
| 28319 | 29319 | 30319 | 31319 |
| 28320 | 29320 | 30320 | 31320 |
| 28321 | 29321 | 30321 | 31321 |
| 28322 | 29322 | 30322 | 31322 |
| 28323 | 29323 | 30323 | 31323 |
| 28324 | 29324 | 30324 | 31324 |
| 28325 | 29325 | 30325 | 31325 |
| 28326 | 29326 | 30326 | 31326 |
| 28327 | 29327 | 30327 | 31327 |
| 28328 | 29328 | 30328 | 31328 |
| 28329 | 29329 | 30329 | 31329 |
| 28330 | 29330 | 30330 | 31330 |
| 28331 | 29331 | 30331 | 31331 |
| 28332 | 29332 | 30332 | 31332 |
| 28333 | 29333 | 30333 | 31333 |
| 28334 | 29334 | 30334 | 31334 |
| 28335 | 29335 | 30335 | 31335 |
| 28336 | 29336 | 30336 | 31336 |
| 28337 | 29337 | 30337 | 31337 |
| 28338 | 29338 | 30338 | 31338 |
| 28339 | 29339 | 30339 | 31339 |
| 28340 | 29340 | 30340 | 31340 |
| 28341 | 29341 | 30341 | 31341 |
| 28342 | 29342 | 30342 | 31342 |
| 28343 | 29343 | 30343 | 31343 |
| 28344 | 29344 | 30344 | 31344 |
| 28345 | 29345 | 30345 | 31345 |
| 28346 | 29346 | 30346 | 31346 |
| 28347 | 29347 | 30347 | 31347 |
| 28348 | 29348 | 30348 | 31348 |
| 28349 | 29349 | 30349 | 31349 |
| 28350 | 29350 | 30350 | 31350 |
| 28351 | 29351 | 30351 | 31351 |
| 28352 | 29352 | 30352 | 31352 |
| 28353 | 29353 | 30353 | 31353 |
| 28354 | 29354 | 30354 | 31354 |
| 28355 | 29355 | 30355 | 31355 |
| 28356 | 29356 | 30356 | 31356 |
| 28357 | 29357 | 30357 | 31357 |
| 28358 | 29358 | 30358 | 31358 |
| 28359 | 29359 | 30359 | 31359 |
| 28360 | 29360 | 30360 | 31360 |
| 28361 | 29361 | 30361 | 31361 |
| 28362 | 29362 | 30362 | 31362 |
| 28363 | 29363 | 30363 | 31363 |
| 28364 | 29364 | 30364 | 31364 |
| 28365 | 29365 | 30365 | 31365 |
| 28366 | 29366 | 30366 | 31366 |
| 28367 | 29367 | 30367 | 31367 |
| 28368 | 29368 | 30368 | 31368 |
| 28369 | 29369 | 30369 | 31369 |
| 28370 | 29370 | 30370 | 31370 |
| 28371 | 29371 | 30371 | 31371 |
| 28372 | 29372 | 30372 | 31372 |
| 28373 | 29373 | 30373 | 31373 |
| 28374 | 29374 | 30374 | 31374 |
| 28375 | 29375 | 30375 | 31375 |
| 28376 | 29376 | 30376 | 31376 |
| 28377 | 29377 | 30377 | 31377 |
| 28378 | 29378 | 30378 | 31378 |
| 28379 | 29379 | 30379 | 31379 |
| 28380 | 29380 | 30380 | 31380 |
| 28381 | 29381 | 30381 | 31381 |
| 28382 | 29382 | 30382 | 31382 |
| 28383 | 29383 | 30383 | 31383 |
| 28384 | 29384 | 30384 | 31384 |
| 28385 | 29385 | 30385 | 31385 |
| 28386 | 29386 | 30386 | 31386 |
| 28387 | 29387 | 30387 | 31387 |
| 28388 | 29388 | 30388 | 31388 |
| 28389 | 29389 | 30389 | 31389 |
| 28390 | 29390 | 30390 | 31390 |
| 28391 | 29391 | 30391 | 31391 |
| 28392 | 29392 | 30392 | 31392 |
| 28393 | 29393 | 30393 | 31393 |
| 28394 | 29394 | 30394 | 31394 |
| 28395 | 29395 | 30395 | 31395 |
| 28396 | 29396 | 30396 | 31396 |
| 28397 | 29397 | 30397 | 31397 |
| 28398 | 29398 | 30398 | 31398 |
| 28399 | 29399 | 30399 | 31399 |
| 28400 | 29400 | 30400 | 31400 |
| 28401 | 29401 | 30401 | 31401 |
| 28402 | 29402 | 30402 | 31402 |
| 28403 | 29403 | 30403 | 31403 |
| 28404 | 29404 | 30404 | 31404 |
| 28405 | 29405 | 30405 | 31405 |
| 28406 | 29406 | 30406 | 31406 |
| 28407 | 29407 | 30407 | 31407 |
| 28408 | 29408 | 30408 | 31408 |
| 28409 | 29409 | 30409 | 31409 |
| 28410 | 29410 | 30410 | 31410 |
| 28411 | 29411 | 30411 | 31411 |
| 28412 | 29412 | 30412 | 31412 |
| 28413 | 29413 | 30413 | 31413 |
| 28414 | 29414 | 30414 | 31414 |
| 28415 | 29415 | 30415 | 31415 |
| 28416 | 29416 | 30416 | 31416 |
| 28417 | 29417 | 30417 | 31417 |
| 28418 | 29418 | 30418 | 31418 |
| 28419 | 29419 | 30419 | 31419 |
| 28420 | 29420 | 30420 | 31420 |
| 28421 | 29421 | 30421 | 31421 |
| 28422 | 29422 | 30422 | 31422 |
| 28423 | 29423 | 30423 | 31423 |
| 28424 | 29424 | 30424 | 31424 |
| 28425 | 29425 | 30425 | 31425 |
| 28426 | 29426 | 30426 | 31426 |
| 28427 | 29427 | 30427 | 31427 |
| 28428 | 29428 | 30428 | 31428 |
| 28429 | 29429 | 30429 | 31429 |
| 28430 | 29430 | 30430 | 31430 |
| 28431 | 29431 | 30431 | 31431 |
| 28432 | 29432 | 30432 | 31432 |
| 28433 | 29433 | 30433 | 31433 |
| 28434 | 29434 | 30434 | 31434 |
| 28435 | 29435 | 30435 | 31435 |
| 28436 | 29436 | 30436 | 31436 |
| 28437 | 29437 | 30437 | 31437 |
| 28438 | 29438 | 30438 | 31438 |
| 28439 | 29439 | 30439 | 31439 |
| 28440 | 29440 | 30440 | 31440 |
| 28441 | 29441 | 30441 | 31441 |
| 28442 | 29442 | 30442 | 31442 |
| 28443 | 29443 | 30443 | 31443 |
| 28444 | 29444 | 30444 | 31444 |
| 28445 | 29445 | 30445 | 31445 |
| 28446 | 29446 | 30446 | 31446 |
| 28447 | 29447 | 30447 | 31447 |
| 28448 | 29448 | 30448 | 31448 |
| 28449 | 29449 | 30449 | 31449 |
| 28450 | 29450 | 30450 | 31450 |
| 28451 | 29451 | 30451 | 31451 |
| 28452 | 29452 | 30452 | 31452 |
| 28453 | 29453 | 30453 | 31453 |
| 28454 | 29454 | 30454 | 31454 |
| 28455 | 29455 | 30455 | 31455 |
| 28456 | 29456 | 30456 | 31456 |
| 28457 | 29457 | 30457 | 31457 |
| 28458 | 29458 | 30458 | 31458 |
| 28459 | 29459 | 30459 | 31459 |
| 28460 | 29460 | 30460 | 31460 |
| 28461 | 29461 | 30461 | 31461 |
| 28462 | 29462 | 30462 | 31462 |
| 28463 | 29463 | 30463 | 31463 |
| 28464 | 29464 | 30464 | 31464 |
| 28465 | 29465 | 30465 | 31465 |
| 28466 | 29466 | 30466 | 31466 |
| 28467 | 29467 | 30467 | 31467 |
| 28468 | 29468 | 30468 | 31468 |
| 28469 | 29469 | 30469 | 31469 |
| 28470 | 29470 | 30470 | 31470 |
| 28471 | 29471 | 30471 | 31471 |
| 28472 | 29472 | 30472 | 31472 |
| 28473 | 29473 | 30473 | 31473 |
| 28474 | 29474 | 30474 | 31474 |
| 28475 | 29475 | 30475 | 31475 |
| 28476 | 29476 | 30476 | 31476 |
| 28477 | 29477 | 30477 | 31477 |
| 28478 | 29478 | 30478 | 31478 |
| 28479 | 29479 | 30479 | 31479 |
| 28480 | 29480 | 30480 | 31480 |
| 28481 | 29481 | 30481 | 31481 |
| 28482 | 29482 | 30482 | 31482 |
| 28483 | 29483 | 30483 | 31483 |
| 28484 | 29484 | 30484 | 31484 |
| 28485 | 29485 | 30485 | 31485 |
| 28486 | 29486 | 30486 | 31486 |
| 28487 | 29487 | 30487 | 31487 |
| 28488 | 29488 | 30488 | 31488 |
| 28489 | 29489 | 30489 | 31489 |
| 28490 | 29490 | 30490 | 31490 |
| 28491 | 29491 | 30491 | 31491 |
| 28492 | 29492 | 30492 | 31492 |
| 28493 | 29493 | 30493 | 31493 |
| 28494 | 29494 | 30494 | 31494 |
| 28495 | 29495 | 30495 | 31495 |
| 28496 | 29496 | 30496 | 31496 |
| 28497 | 29497 | 30497 | 31497 |
| 28498 | 29498 | 30498 | 31498 |
| 28499 | 29499 | 30499 | 31499 |
| 28500 | 29500 | 30500 | 31500 |
| 28501 | 29501 | 30501 | 31501 |
| 28502 | 29502 | 30502 | 31502 |
| 28503 | 29503 | 30503 | 31503 |
| 28504 | 29504 | 30504 | 31504 |
| 28505 | 29505 | 30505 | 31505 |
| 28506 | 29506 | 30506 | 31506 |
| 28507 | 29507 | 30507 | 31507 |
| 28508 | 29508 | 30508 | 31508 |
| 28509 | 29509 | 30509 | 31509 |
| 28510 | 29510 | 30510 | 31510 |
| 28511 | 29511 | 30511 | 31511 |
| 28512 | 29512 | 30512 | 31512 |
| 28513 | 29513 | 30513 | 31513 |
| 28514 | 29514 | 30514 | 31514 |
| 28515 | 29515 | 30515 | 31515 |
| 28516 | 29516 | 30516 | 31516 |
| 28517 | 29517 | 30517 | 31517 |
| 28518 | 29518 | 30518 | 31518 |
| 28519 | 29519 | 30519 | 31519 |
| 28520 | 29520 | 30520 | 31520 |
| 28521 | 29521 | 30521 | 31521 |
| 28522 | 29522 | 30522 | 31522 |
| 28523 | 29523 | 30523 | 31523 |
| 28524 | 29524 | 30524 | 31524 |
| 28525 | 29525 | 30525 | 31525 |
| 28526 | 29526 | 30526 | 31526 |
| 28527 | 29527 | 30527 | 31527 |
| 28528 | 29528 | 30528 | 31528 |
| 28529 | 29529 | 30529 | 31529 |
| 28530 | 29530 | 30530 | 31530 |
| 28531 | 29531 | 30531 | 31531 |
| 28532 | 29532 | 30532 | 31532 |
| 28533 | 29533 | 30533 | 31533 |
| 28534 | 29534 | 30534 | 31534 |
| 28535 | 29535 | 30535 | 31535 |
| 28536 | 29536 | 30536 | 31536 |
| 28537 | 29537 | 30537 | 31537 |
| 28538 | 29538 | 30538 | 31538 |
| 28539 | 29539 | 30539 | 31539 |
| 28540 | 29540 | 30540 | 31540 |
| 28541 | 29541 | 30541 | 31541 |
| 28542 | 29542 | 30542 | 31542 |
| 28543 | 29543 | 30543 | 31543 |
| 28544 | 29544 | 30544 | 31544 |
| 28545 | 29545 | 30545 | 31545 |
| 28546 | 29546 | 30546 | 31546 |
| 28547 | 29547 | 30547 | 31547 |
| 28548 | 29548 | 30548 | 31548 |
| 28549 | 29549 | 30549 | 31549 |
| 28550 | 29550 | 30550 | 31550 |
| 28551 | 29551 | 30551 | 31551 |
| 28552 | 29552 | 30552 | 31552 |
| 28553 | 29553 | 30553 | 31553 |
| 28554 | 29554 | 30554 | 31554 |
| 28555 | 29555 | 30555 | 31555 |
| 28556 | 29556 | 30556 | 31556 |
| 28557 | 29557 | 30557 | 31557 |
| 28558 | 29558 | 30558 | 31558 |
| 28559 | 29559 | 30559 | 31559 |
| 28560 | 29560 | 30560 | 31560 |
| 28561 | 29561 | 30561 | 31561 |
| 28562 | 29562 | 30562 | 31562 |
| 28563 | 29563 | 30563 | 31563 |
| 28564 | 29564 | 30564 | 31564 |
| 28565 | 29565 | 30565 | 31565 |
| 28566 | 29566 | 30566 | 31566 |
| 28567 | 29567 | 30567 | 31567 |
| 28568 | 29568 | 30568 | 31568 |
| 28569 | 29569 | 30569 | 31569 |
| 28570 | 29570 | 30570 | 31570 |
| 28571 | 29571 | 30571 | 31571 |
| 28572 | 29572 | 30572 | 31572 |
| 28573 | 29573 | 30573 | 31573 |
| 28574 | 29574 | 30574 | 31574 |
| 28575 | 29575 | 30575 | 31575 |
| 28576 | 29576 | 30576 | 31576 |
| 28577 | 29577 | 30577 | 31577 |
| 28578 | 29578 | 30578 | 31578 |
| 28579 | 29579 | 30579 | 31579 |
| 28580 | 29580 | 30580 | 31580 |
| 28581 | 29581 | 30581 | 31581 |
| 28582 | 29582 | 30582 | 31582 |
| 28583 | 29583 | 30583 | 31583 |
| 28584 | 29584 | 30584 | 31584 |
| 28585 | 29585 | 30585 | 31585 |
| 28586 | 29586 | 30586 | 31586 |
| 28587 | 29587 | 30587 | 31587 |
| 28588 | 29588 | 30588 | 31588 |
| 28589 | 29589 | 30589 | 31589 |
| 28590 | 29590 | 30590 | 31590 |
| 28591 | 29591 | 30591 | 31591 |
| 28592 | 29592 | 30592 | 31592 |
| 28593 | 29593 | 30593 | 31593 |
| 28594 | 29594 | 30594 | 31594 |
| 28595 | 29595 | 30595 | 31595 |
| 28596 | 29596 | 30596 | 31596 |
| 28597 | 29597 | 30597 | 31597 |
| 28598 | 29598 | 30598 | 31598 |
| 28599 | 29599 | 30599 | 31599 |
| 28600 | 29600 | 30600 | 31600 |
| 28601 | 29601 | 30601 | 31601 |
| 28602 | 29602 | 30602 | 31602 |
| 28603 | 29603 | 30603 | 31603 |
| 28604 | 29604 | 30604 | 31604 |
| 28605 | 29605 | 30605 | 31605 |
| 28606 | 29606 | 30606 | 31606 |
| 28607 | 29607 | 30607 | 31607 |
| 28608 | 29608 | 30608 | 31608 |
| 28609 | 29609 | 30609 | 31609 |
| 28610 | 29610 | 30610 | 31610 |
| 28611 | 29611 | 30611 | 31611 |
| 28612 | 29612 | 30612 | 31612 |
| 28613 | 29613 | 30613 | 31613 |
| 28614 | 29614 | 30614 | 31614 |
| 28615 | 29615 | 30615 | 31615 |
| 28616 | 29616 | 30616 | 31616 |
| 28617 | 29617 | 30617 | 31617 |
| 28618 | 29618 | 30618 | 31618 |
| 28619 | 29619 | 30619 | 31619 |
| 28620 | 29620 | 30620 | 31620 |
| 28621 | 29621 | 30621 | 31621 |
| 28622 | 29622 | 30622 | 31622 |
| 28623 | 29623 | 30623 | 31623 |
| 28624 | 29624 | 30624 | 31624 |
| 28625 | 29625 | 30625 | 31625 |
| 28626 | 29626 | 30626 | 31626 |
| 28627 | 29627 | 30627 | 31627 |
| 28628 | 29628 | 30628 | 31628 |
| 28629 | 29629 | 30629 | 31629 |
| 28630 | 29630 | 30630 | 31630 |
| 28631 | 29631 | 30631 | 31631 |
| 28632 | 29632 | 30632 | 31632 |
| 28633 | 29633 | 30633 | 31633 |
| 28634 | 29634 | 30634 | 31634 |
| 28635 | 29635 | 30635 | 31635 |
| 28636 | 29636 | 30636 | 31636 |
| 28637 | 29637 | 30637 | 31637 |
| 28638 | 29638 | 30638 | 31638 |
| 28639 | 29639 | 30639 | 31639 |
| 28640 | 29640 | 30640 | 31640 |
| 28641 | 29641 | 30641 | 31641 |
| 28642 | 29642 | 30642 | 31642 |
| 28643 | 29643 | 30643 | 31643 |
| 28644 | 29644 | 30644 | 31644 |
| 28645 | 29645 | 30645 | 31645 |
| 28646 | 29646 | 30646 | 31646 |
| 28647 | 29647 | 30647 | 31647 |
| 28648 | 29648 | 30648 | 31648 |
| 28649 | 29649 | 30649 | 31649 |
| 28650 | 29650 | 30650 | 31650 |
| 28651 | 29651 | 30651 | 31651 |
| 28652 | 29652 | 30652 | 31652 |
| 28653 | 29653 | 30653 | 31653 |
| 28654 | 29654 | 30654 | 31654 |
| 28655 | 29655 | 30655 | 31655 |
| 28656 | 29656 | 30656 | 31656 |
| 28657 | 29657 | 30657 | 31657 |
| 28658 | 29658 | 30658 | 31658 |
| 28659 | 29659 | 30659 | 31659 |
| 28660 | 29660 | 30660 | 31660 |
| 28661 | 29661 | 30661 | 31661 |
| 28662 | 29662 | 30662 | 31662 |
| 28663 | 29663 | 30663 | 31663 |
| 28664 | 29664 | 30664 | 31664 |
| 28665 | 29665 | 30665 | 31665 |
| 28666 | 29666 | 30666 | 31666 |
| 28667 | 29667 | 30667 | 31667 |
| 28668 | 29668 | 30668 | 31668 |
| 28669 | 29669 | 30669 | 31669 |
| 28670 | 29670 | 30670 | 31670 |
| 28671 | 29671 | 30671 | 31671 |
| 28672 | 29672 | 30672 | 31672 |
| 28673 | 29673 | 30673 | 31673 |
| 28674 | 29674 | 30674 | 31674 |
| 28675 | 29675 | 30675 | 31675 |
| 28676 | 29676 | 30676 | 31676 |
| 28677 | 29677 | 30677 | 31677 |
| 28678 | 29678 | 30678 | 31678 |
| 28679 | 29679 | 30679 | 31679 |
| 28680 | 29680 | 30680 | 31680 |
| 28681 | 29681 | 30681 | 31681 |
| 28682 | 29682 | 30682 | 31682 |
| 28683 | 29683 | 30683 | 31683 |
| 28684 | 29684 | 30684 | 31684 |
| 28685 | 29685 | 30685 | 31685 |
| 28686 | 29686 | 30686 | 31686 |
| 28687 | 29687 | 30687 | 31687 |
| 28688 | 29688 | 30688 | 31688 |
| 28689 | 29689 | 30689 | 31689 |
| 28690 | 29690 | 30690 | 31690 |
| 28691 | 29691 | 30691 | 31691 |
| 28692 | 29692 | 30692 | 31692 |
| 28693 | 29693 | 30693 | 31693 |
| 28694 | 29694 | 30694 | 31694 |
| 28695 | 29695 | 30695 | 31695 |
| 28696 | 29696 | 30696 | 31696 |
| 28697 | 29697 | 30697 | 31697 |
| 28698 | 29698 | 30698 | 31698 |
| 28699 | 29699 | 30699 | 31699 |
| 28700 | 29700 | 30700 | 31700 |
| 28701 | 29701 | 30701 | 31701 |
| 28702 | 29702 | 30702 | 31702 |
| 28703 | 29703 | 30703 | 31703 |
| 28704 | 29704 | 30704 | 31704 |
| 28705 | 29705 | 30705 | 31705 |
| 28706 | 29706 | 30706 | 31706 |
| 28707 | 29707 | 30707 | 31707 |
| 28708 | 29708 | 30708 | 31708 |
| 28709 | 29709 | 30709 | 31709 |
| 28710 | 29710 | 30710 | 31710 |
| 28711 | 29711 | 30711 | 31711 |
| 28712 | 29712 | 30712 | 31712 |
| 28713 | 29713 | 30713 | 31713 |
| 28714 | 29714 | 30714 | 31714 |
| 28715 | 29715 | 30715 | 31715 |
| 28716 | 29716 | 30716 | 31716 |
| 28717 | 29717 | 30717 | 31717 |
| 28718 | 29718 | 30718 | 31718 |
| 28719 | 29719 | 30719 | 31719 |
| 28720 | 29720 | 30720 | 31720 |
| 28721 | 29721 | 30721 | 31721 |
| 28722 | 29722 | 30722 | 31722 |
| 28723 | 29723 | 30723 | 31723 |
| 28724 | 29724 | 30724 | 31724 |
| 28725 | 29725 | 30725 | 31725 |
| 28726 | 29726 | 30726 | 31726 |
| 28727 | 29727 | 30727 | 31727 |
| 28728 | 29728 | 30728 | 31728 |
| 28729 | 29729 | 30729 | 31729 |
| 28730 | 29730 | 30730 | 31730 |
| 28731 | 29731 | 30731 | 31731 |
| 28732 | 29732 | 30732 | 31732 |
| 28733 | 29733 | 30733 | 31733 |
| 28734 | 29734 | 30734 | 31734 |
| 28735 | 29735 | 30735 | 31735 |
| 28736 | 29736 | 30736 | 31736 |
| 28737 | 29737 | 30737 | 31737 |
| 28738 | 29738 | 30738 | 31738 |
| 28739 | 29739 | 30739 | 31739 |
| 28740 | 29740 | 30740 | 31740 |
| 28741 | 29741 | 30741 | 31741 |
| 28742 | 29742 | 30742 | 31742 |
| 28743 | 29743 | 30743 | 31743 |
| 28744 | 29744 | 30744 | 31744 |
| 28745 | 29745 | 30745 | 31745 |
| 28746 | 29746 | 30746 | 31746 |
| 28747 | 29747 | 30747 | 31747 |
| 28748 | 29748 | 30748 | 31748 |
| 28749 | 29749 | 30749 | 31749 |
| 28750 | 29750 | 30750 | 31750 |
| 28751 | 29751 | 30751 | 31751 |
| 28752 | 29752 | 30752 | 31752 |
| 28753 | 29753 | 30753 | 31753 |
| 28754 | 29754 | 30754 | 31754 |
| 28755 | 29755 | 30755 | 31755 |
| 28756 | 29756 | 30756 | 31756 |
| 28757 | 29757 | 30757 | 31757 |
| 28758 | 29758 | 30758 | 31758 |
| 28759 | 29759 | 30759 | 31759 |
| 28760 | 29760 | 30760 | 31760 |
| 28761 | 29761 | 30761 | 31761 |
| 28762 | 29762 | 30762 | 31762 |
| 28763 | 29763 | 30763 | 31763 |
| 28764 | 29764 | 30764 | 31764 |
| 28765 | 29765 | 30765 | 31765 |
| 28766 | 29766 | 30766 | 31766 |
| 28767 | 29767 | 30767 | 31767 |
| 28768 | 29768 | 30768 | 31768 |
| 28769 | 29769 | 30769 | 31769 |
| 28770 | 29770 | 30770 | 31770 |
| 28771 | 29771 | 30771 | 31771 |
| 28772 | 29772 | 30772 | 31772 |
| 28773 | 29773 | 30773 | 31773 |
| 28774 | 29774 | 30774 | 31774 |
| 28775 | 29775 | 30775 | 31775 |
| 28776 | 29776 | 30776 | 31776 |
| 28777 | 29777 | 30777 | 31777 |
| 28778 | 29778 | 30778 | 31778 |
| 28779 | 29779 | 30779 | 31779 |
| 28780 | 29780 | 30780 | 31780 |
| 28781 | 29781 | 30781 | 31781 |
| 28782 | 29782 | 30782 | 31782 |
| 28783 | 29783 | 30783 | 31783 |
| 28784 | 29784 | 30784 | 31784 |
| 28785 | 29785 | 30785 | 31785 |
| 28786 | 29786 | 30786 | 31786 |
| 28787 | 29787 | 30787 | 31787 |
| 28788 | 29788 | 30788 | 31788 |
| 28789 | 29789 | 30789 | 31789 |
| 28790 | 29790 | 30790 | 31790 |
| 28791 | 29791 | 30791 | 31791 |
| 28792 | 29792 | 30792 | 31792 |
| 28793 | 29793 | 30793 | 31793 |
| 28794 | 29794 | 30794 | 31794 |
| 28795 | 29795 | 30795 | 31795 |
| 28796 | 29796 | 30796 | 31796 |
| 28797 | 29797 | 30797 | 31797 |
| 28798 | 29798 | 30798 | 31798 |
| 28799 | 29799 | 30799 | 31799 |
| 28800 | 29800 | 30800 | 31800 |
| 28801 | 29801 | 30801 | 31801 |
| 28802 | 29802 | 30802 | 31802 |
| 28803 | 29803 | 30803 | 31803 |
| 28804 | 29804 | 30804 | 31804 |
| 28805 | 29805 | 30805 | 31805 |
| 28806 | 29806 | 30806 | 31806 |
| 28807 | 29807 | 30807 | 31807 |
| 28808 | 29808 | 30808 | 31808 |
| 28809 | 29809 | 30809 | 31809 |
| 28810 | 29810 | 30810 | 31810 |
| 28811 | 29811 | 30811 | 31811 |
| 28812 | 29812 | 30812 | 31812 |
| 28813 | 29813 | 30813 | 31813 |
| 28814 | 29814 | 30814 | 31814 |
| 28815 | 29815 | 30815 | 31815 |
| 28816 | 29816 | 30816 | 31816 |
| 28817 | 29817 | 30817 | 31817 |
| 28818 | 29818 | 30818 | 31818 |
| 28819 | 29819 | 30819 | 31819 |
| 28820 | 29820 | 30820 | 31820 |
| 28821 | 29821 | 30821 | 31821 |
| 28822 | 29822 | 30822 | 31822 |
| 28823 | 29823 | 30823 | 31823 |
| 28824 | 29824 | 30824 | 31824 |
| 28825 | 29825 | 30825 | 31825 |
| 28826 | 29826 | 30826 | 31826 |
| 28827 | 29827 | 30827 | 31827 |
| 28828 | 29828 | 30828 | 31828 |
| 28829 | 29829 | 30829 | 31829 |
| 28830 | 29830 | 30830 | 31830 |
| 28831 | 29831 | 30831 | 31831 |
| 28832 | 29832 | 30832 | 31832 |
| 28833 | 29833 | 30833 | 31833 |
| 28834 | 29834 | 30834 | 31834 |
| 28835 | 29835 | 30835 | 31835 |
| 28836 | 29836 | 30836 | 31836 |
| 28837 | 29837 | 30837 | 31837 |
| 28838 | 29838 | 30838 | 31838 |
| 28839 | 29839 | 30839 | 31839 |
| 28840 | 29840 | 30840 | 31840 |
| 28841 | 29841 | 30841 | 31841 |
| 28842 | 29842 | 30842 | 31842 |
| 28843 | 29843 | 30843 | 31843 |
| 28844 | 29844 | 30844 | 31844 |
| 28845 | 29845 | 30845 | 31845 |
| 28846 | 29846 | 30846 | 31846 |
| 28847 | 29847 | 30847 | 31847 |
| 28848 | 29848 | 30848 | 31848 |
| 28849 | 29849 | 30849 | 31849 |
| 28850 | 29850 | 30850 | 31850 |
| 28851 | 29851 | 30851 | 31851 |
| 28852 | 29852 | 30852 | 31852 |
| 28853 | 29853 | 30853 | 31853 |
| 28854 | 29854 | 30854 | 31854 |
| 28855 | 29855 | 30855 | 31855 |
| 28856 | 29856 | 30856 | 31856 |
| 28857 | 29857 | 30857 | 31857 |
| 28858 | 29858 | 30858 | 31858 |
| 28859 | 29859 | 30859 | 31859 |
| 28860 | 29860 | 30860 | 31860 |
| 28861 | 29861 | 30861 | 31861 |
| 28862 | 29862 | 30862 | 31862 |
| 28863 | 29863 | 30863 | 31863 |
| 28864 | 29864 | 30864 | 31864 |
| 28865 | 29865 | 30865 | 31865 |
| 28866 | 29866 | 30866 | 31866 |
| 28867 | 29867 | 30867 | 31867 |
| 28868 | 29868 | 30868 | 31868 |
| 28869 | 29869 | 30869 | 31869 |
| 28870 | 29870 | 30870 | 31870 |
| 28871 | 29871 | 30871 | 31871 |
| 28872 | 29872 | 30872 | 31872 |
| 28873 | 29873 | 30873 | 31873 |
| 28874 | 29874 | 30874 | 31874 |
| 28875 | 29875 | 30875 | 31875 |
| 28876 | 29876 | 30876 | 31876 |
| 28877 | 29877 | 30877 | 31877 |
| 28878 | 29878 | 30878 | 31878 |
| 28879 | 29879 | 30879 | 31879 |
| 28880 | 29880 | 30880 | 31880 |
| 28881 | 29881 | 30881 | 31881 |
| 28882 | 29882 | 30882 | 31882 |
| 28883 | 29883 | 30883 | 31883 |
| 28884 | 29884 | 30884 | 31884 |
| 28885 | 29885 | 30885 | 31885 |
| 28886 | 29886 | 30886 | 31886 |
| 28887 | 29887 | 30887 | 31887 |
| 28888 | 29888 | 30888 | 31888 |
| 28889 | 29889 | 30889 | 31889 |
| 28890 | 29890 | 30890 | 31890 |
| 28891 | 29891 | 30891 | 31891 |
| 28892 | 29892 | 30892 | 31892 |
| 28893 | 29893 | 30893 | 31893 |
| 28894 | 29894 | 30894 | 31894 |
| 28895 | 29895 | 30895 | 31895 |
| 28896 | 29896 | 30896 | 31896 |
| 28897 | 29897 | 30897 | 31897 |
| 28898 | 29898 | 30898 | 31898 |
| 28899 | 29899 | 30899 | 31899 |
| 28900 | 29900 | 30900 | 31900 |
| 28901 | 29901 | 30901 | 31901 |
| 28902 | 29902 | 30902 | 31902 |
| 28903 | 29903 | 30903 | 31903 |
| 28904 | 29904 | 30904 | 31904 |
| 28905 | 29905 | 30905 | 31905 |
| 28906 | 29906 | 30906 | 31906 |
| 28907 | 29907 | 30907 | 31907 |
| 28908 | 29908 | 30908 | 31908 |
| 28909 | 29909 | 30909 | 31909 |
| 28910 | 29910 | 30910 | 31910 |
| 28911 | 29911 | 30911 | 31911 |
| 28912 | 29912 | 30912 | 31912 |
| 28913 | 29913 | 30913 | 31913 |
| 28914 | 29914 | 30914 | 31914 |
| 28915 | 29915 | 30915 | 31915 |
| 28916 | 29916 | 30916 | 31916 |
| 28917 | 29917 | 30917 | 31917 |
| 28918 | 29918 | 30918 | 31918 |
| 28919 | 29919 | 30919 | 31919 |
| 28920 | 29920 | 30920 | 31920 |
| 28921 | 29921 | 30921 | 31921 |
| 28922 | 29922 | 30922 | 31922 |
| 28923 | 29923 | 30923 | 31923 |
| 28924 | 29924 | 30924 | 31924 |
| 28925 | 29925 | 30925 | 31925 |
| 28926 | 29926 | 30926 | 31926 |
| 28927 | 29927 | 30927 | 31927 |
| 28928 | 29928 | 30928 | 31928 |
| 28929 | 29929 | 30929 | 31929 |
| 28930 | 29930 | 30930 | 31930 |
| 28931 | 29931 | 30931 | 31931 |
| 28932 | 29932 | 30932 | 31932 |
| 28933 | 29933 | 30933 | 31933 |
| 28934 | 29934 | 30934 | 31934 |
| 28935 | 29935 | 30935 | 31935 |
| 28936 | 29936 | 30936 | 31936 |
| 28937 | 29937 | 30937 | 31937 |
| 28938 | 29938 | 30938 | 31938 |
| 28939 | 29939 | 30939 | 31939 |
| 28940 | 29940 | 30940 | 31940 |
| 28941 | 29941 | 30941 | 31941 |
| 28942 | 29942 | 30942 | 31942 |
| 28943 | 29943 | 30943 | 31943 |
| 28944 | 29944 | 30944 | 31944 |
| 28945 | 29945 | 30945 | 31945 |
| 28946 | 29946 | 30946 | 31946 |
| 28947 | 29947 | 30947 | 31947 |
| 28948 | 29948 | 30948 | 31948 |
| 28949 | 29949 | 30949 | 31949 |
| 28950 | 29950 | 30950 | 31950 |
| 28951 | 29951 | 30951 | 31951 |
| 28952 | 29952 | 30952 | 31952 |
| 28953 | 29953 | 30953 | 31953 |
| 28954 | 29954 | 30954 | 31954 |
| 28955 | 29955 | 30955 | 31955 |
| 28956 | 29956 | 30956 | 31956 |
| 28957 | 29957 | 30957 | 31957 |
| 28958 | 29958 | 30958 | 31958 |
| 28959 | 29959 | 30959 | 31959 |
| 28960 | 29960 | 30960 | 31960 |
| 28961 | 29961 | 30961 | 31961 |
| 28962 | 29962 | 30962 | 31962 |
| 28963 | 29963 | 30963 | 31963 |
| 28964 | 29964 | 30964 | 31964 |
| 28965 | 29965 | 30965 | 31965 |
| 28966 | 29966 | 30966 | 31966 |
| 28967 | 29967 | 30967 | 31967 |
| 28968 | 29968 | 30968 | 31968 |
| 28969 | 29969 | 30969 | 31969 |
| 28970 | 29970 | 30970 | 31970 |
| 28971 | 29971 | 30971 | 31971 |
| 28972 | 29972 | 30972 | 31972 |
| 28973 | 29973 | 30973 | 31973 |
| 28974 | 29974 | 30974 | 31974 |
| 28975 | 29975 | 30975 | 31975 |
| 28976 | 29976 | 30976 | 31976 |
| 28977 | 29977 | 30977 | 31977 |
| 28978 | 29978 | 30978 | 31978 |
| 28979 | 29979 | 30979 | 31979 |
| 28980 | 29980 | 30980 | 31980 |
| 28981 | 29981 | 30981 | 31981 |
| 28982 | 29982 | 30982 | 31982 |
| 28983 | 29983 | 30983 | 31983 |
| 28984 | 29984 | 30984 | 31984 |
| 28985 | 29985 | 30985 | 31985 |
| 28986 | 29986 | 30986 | 31986 |
| 28987 | 29987 | 30987 | 31987 |
| 28988 | 29988 | 30988 | 31988 |
| 28989 | 29989 | 30989 | 31989 |
| 28990 | 29990 | 30990 | 31990 |
| 28991 | 29991 | 30991 | 31991 |
| 28992 | 29992 | 30992 | 31992 |
| 28993 | 29993 | 30993 | 31993 |
| 28994 | 29994 | 30994 | 31994 |
| 28995 | 29995 | 30995 | 31995 |
| 28996 | 29996 | 30996 | 31996 |
| 28997 | 29997 | 30997 | 31997 |
| 28998 | 29998 | 30998 | 31998 |
| 28999 | 29999 | 30999 | 31999 |
| 29000 | 30000 | 31000 | 32000 |
| 29001 | 30001 | 31001 | 32001 |
| 29002 | 30002 | 31002 | 32002 |

In some embodiments, the sequences that specifically bind to all nuts comprise an inner sequence selected from the group consisting of the nucleic acid sequences of SEQ ID NOs. 32003 to 33002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to all nuts may comprise a nucleic acid sequence selected from the group consisting of SEQ ID NOs. 33003 to 34002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to all nuts may comprise a nucleic acid sequence selected from the group consisting of SEQ ID NOs. 34003 to 35002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to all nuts may comprise a nucleic acid sequence selected from the group consisting of SEQ ID NOs. 35003 to 36002. In one embodiment, the aptamer of the present disclosure that can bind to all nuts may comprise a nucleic acid sequence selected from the group consisting of SEQ ID NOs. 32003 to 36002 listed in Table 10, or variant thereof.

TABLE 10
Aptamer sequences against all nuts
Aptamer sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 32003 | 33003 | 34003 | 35003 |
| 32004 | 33004 | 34004 | 35004 |
| 32005 | 33005 | 34005 | 35005 |
| 32006 | 33006 | 34006 | 35006 |
| 32007 | 33007 | 34007 | 35007 |
| 32008 | 33008 | 34008 | 35008 |
| 32009 | 33009 | 34009 | 35009 |
| 32010 | 33010 | 34010 | 35010 |
| 32011 | 33011 | 34011 | 35011 |
| 32012 | 33012 | 34012 | 35012 |
| 32013 | 33013 | 34013 | 35013 |
| 32014 | 33014 | 34014 | 35014 |
| 32015 | 33015 | 34015 | 35015 |
| 32016 | 33016 | 34016 | 35016 |
| 32017 | 33017 | 34017 | 35017 |
| 32018 | 33018 | 34018 | 35018 |
| 32019 | 33019 | 34019 | 35019 |
| 32020 | 33020 | 34020 | 35020 |
| 32021 | 33021 | 34021 | 35021 |
| 32022 | 33022 | 34022 | 35022 |
| 32023 | 33023 | 34023 | 35023 |
| 32024 | 33024 | 34024 | 35024 |
| 32025 | 33025 | 34025 | 35025 |
| 32026 | 33026 | 34026 | 35026 |
| 32027 | 33027 | 34027 | 35027 |
| 32028 | 33028 | 34028 | 35028 |
| 32029 | 33029 | 34029 | 35029 |
| 32030 | 33030 | 34030 | 35030 |
| 32031 | 33031 | 34031 | 35031 |
| 32032 | 33032 | 34032 | 35032 |
| 32033 | 33033 | 34033 | 35033 |
| 32034 | 33034 | 34034 | 35034 |
| 32035 | 33035 | 34035 | 35035 |
| 32036 | 33036 | 34036 | 35036 |
| 32037 | 33037 | 34037 | 35037 |
| 32038 | 33038 | 34038 | 35038 |
| 32039 | 33039 | 34039 | 35039 |
| 32040 | 33040 | 34040 | 35040 |
| 32041 | 33041 | 34041 | 35041 |
| 32042 | 33042 | 34042 | 35042 |
| 32043 | 33043 | 34043 | 35043 |
| 32044 | 33044 | 34044 | 35044 |
| 32045 | 33045 | 34045 | 35045 |
| 32046 | 33046 | 34046 | 35046 |
| 32047 | 33047 | 34047 | 35047 |
| 32048 | 33048 | 34048 | 35048 |
| 32049 | 33049 | 34049 | 35049 |
| 32050 | 33050 | 34050 | 35050 |
| 32051 | 33051 | 34051 | 35051 |
| 32052 | 33052 | 34052 | 35052 |
| 32053 | 33053 | 34053 | 35053 |
| 32054 | 33054 | 34054 | 35054 |
| 32055 | 33055 | 34055 | 35055 |
| 32056 | 33056 | 34056 | 35056 |
| 32057 | 33057 | 34057 | 35057 |
| 32058 | 33058 | 34058 | 35058 |
| 32059 | 33059 | 34059 | 35059 |
| 32060 | 33060 | 34060 | 35060 |
| 32061 | 33061 | 34061 | 35061 |
| 32062 | 33062 | 34062 | 35062 |
| 32063 | 33063 | 34063 | 35063 |
| 32064 | 33064 | 34064 | 35064 |
| 32065 | 33065 | 34065 | 35065 |
| 32066 | 33066 | 34066 | 35066 |
| 32067 | 33067 | 34067 | 35067 |
| 32068 | 33068 | 34068 | 35068 |
| 32069 | 33069 | 34069 | 35069 |
| 32070 | 33070 | 34070 | 35070 |
| 32071 | 33071 | 34071 | 35071 |
| 32072 | 33072 | 34072 | 35072 |
| 32073 | 33073 | 34073 | 35073 |
| 32074 | 33074 | 34074 | 35074 |
| 32075 | 33075 | 34075 | 35075 |
| 32076 | 33076 | 34076 | 35076 |
| 32077 | 33077 | 34077 | 35077 |
| 32078 | 33078 | 34078 | 35078 |
| 32079 | 33079 | 34079 | 35079 |
| 32080 | 33080 | 34080 | 35080 |
| 32081 | 33081 | 34081 | 35081 |
| 32082 | 33082 | 34082 | 35082 |
| 32083 | 33083 | 34083 | 35083 |
| 32084 | 33084 | 34084 | 35084 |
| 32085 | 33085 | 34085 | 35085 |
| 32086 | 33086 | 34086 | 35086 |
| 32087 | 33087 | 34087 | 35087 |
| 32088 | 33088 | 34088 | 35088 |
| 32089 | 33089 | 34089 | 35089 |
| 32090 | 33090 | 34090 | 35090 |
| 32091 | 33091 | 34091 | 35091 |
| 32092 | 33092 | 34092 | 35092 |
| 32093 | 33093 | 34093 | 35093 |
| 32094 | 33094 | 34094 | 35094 |
| 32095 | 33095 | 34095 | 35095 |
| 32096 | 33096 | 34096 | 35096 |
| 32097 | 33097 | 34097 | 35097 |
| 32098 | 33098 | 34098 | 35098 |
| 32099 | 33099 | 34099 | 35099 |
| 32100 | 33100 | 34100 | 35100 |
| 32101 | 33101 | 34101 | 35101 |
| 32102 | 33102 | 34102 | 35102 |
| 32103 | 33103 | 34103 | 35103 |
| 32104 | 33104 | 34104 | 35104 |
| 32105 | 33105 | 34105 | 35105 |
| 32106 | 33106 | 34106 | 35106 |
| 32107 | 33107 | 34107 | 35107 |
| 32108 | 33108 | 34108 | 35108 |
| 32109 | 33109 | 34109 | 35109 |
| 32110 | 33110 | 34110 | 35110 |
| 32111 | 33111 | 34111 | 35111 |
| 32112 | 33112 | 34112 | 35112 |
| 32113 | 33113 | 34113 | 35113 |
| 32114 | 33114 | 34114 | 35114 |
| 32115 | 33115 | 34115 | 35115 |
| 32116 | 33116 | 34116 | 35116 |
| 32117 | 33117 | 34117 | 35117 |
| 32118 | 33118 | 34118 | 35118 |
| 32119 | 33119 | 34119 | 35119 |
| 32120 | 33120 | 34120 | 35120 |
| 32121 | 33121 | 34121 | 35121 |
| 32122 | 33122 | 34122 | 35122 |
| 32123 | 33123 | 34123 | 35123 |
| 32124 | 33124 | 34124 | 35124 |
| 32125 | 33125 | 34125 | 35125 |
| 32126 | 33126 | 34126 | 35126 |
| 32127 | 33127 | 34127 | 35127 |
| 32128 | 33128 | 34128 | 35128 |
| 32129 | 33129 | 34129 | 35129 |
| 32130 | 33130 | 34130 | 35130 |
| 32131 | 33131 | 34131 | 35131 |
| 32132 | 33132 | 34132 | 35132 |
| 32133 | 33133 | 34133 | 35133 |
| 32134 | 33134 | 34134 | 35134 |
| 32135 | 33135 | 34135 | 35135 |
| 32136 | 33136 | 34136 | 35136 |
| 32137 | 33137 | 34137 | 35137 |
| 32138 | 33138 | 34138 | 35138 |
| 32139 | 33139 | 34139 | 35139 |
| 32140 | 33140 | 34140 | 35140 |
| 32141 | 33141 | 34141 | 35141 |
| 32142 | 33142 | 34142 | 35142 |
| 32143 | 33143 | 34143 | 35143 |
| 32144 | 33144 | 34144 | 35144 |
| 32145 | 33145 | 34145 | 35145 |
| 32146 | 33146 | 34146 | 35146 |
| 32147 | 33147 | 34147 | 35147 |
| 32148 | 33148 | 34148 | 35148 |
| 32149 | 33149 | 34149 | 35149 |
| 32150 | 33150 | 34150 | 35150 |
| 32151 | 33151 | 34151 | 35151 |
| 32152 | 33152 | 34152 | 35152 |
| 32153 | 33153 | 34153 | 35153 |
| 32154 | 33154 | 34154 | 35154 |
| 32155 | 33155 | 34155 | 35155 |
| 32156 | 33156 | 34156 | 35156 |
| 32157 | 33157 | 34157 | 35157 |
| 32158 | 33158 | 34158 | 35158 |
| 32159 | 33159 | 34159 | 35159 |
| 32160 | 33160 | 34160 | 35160 |
| 32161 | 33161 | 34161 | 35161 |
| 32162 | 33162 | 34162 | 35162 |
| 32163 | 33163 | 34163 | 35163 |
| 32164 | 33164 | 34164 | 35164 |
| 32165 | 33165 | 34165 | 35165 |
| 32166 | 33166 | 34166 | 35166 |
| 32167 | 33167 | 34167 | 35167 |
| 32168 | 33168 | 34168 | 35168 |
| 32169 | 33169 | 34169 | 35169 |
| 32170 | 33170 | 34170 | 35170 |
| 32171 | 33171 | 34171 | 35171 |
| 32172 | 33172 | 34172 | 35172 |
| 32173 | 33173 | 34173 | 35173 |
| 32174 | 33174 | 34174 | 35174 |
| 32175 | 33175 | 34175 | 35175 |
| 32176 | 33176 | 34176 | 35176 |
| 32177 | 33177 | 34177 | 35177 |
| 32178 | 33178 | 34178 | 35178 |
| 32179 | 33179 | 34179 | 35179 |
| 32180 | 33180 | 34180 | 35180 |
| 32181 | 33181 | 34181 | 35181 |
| 32182 | 33182 | 34182 | 35182 |
| 32183 | 33183 | 34183 | 35183 |
| 32184 | 33184 | 34184 | 35184 |
| 32185 | 33185 | 34185 | 35185 |
| 32186 | 33186 | 34186 | 35186 |
| 32187 | 33187 | 34187 | 35187 |
| 32188 | 33188 | 34188 | 35188 |
| 32189 | 33189 | 34189 | 35189 |
| 32190 | 33190 | 34190 | 35190 |
| 32191 | 33191 | 34191 | 35191 |
| 32192 | 33192 | 34192 | 35192 |
| 32193 | 33193 | 34193 | 35193 |
| 32194 | 33194 | 34194 | 35194 |
| 32195 | 33195 | 34195 | 35195 |
| 32196 | 33196 | 34196 | 35196 |
| 32197 | 33197 | 34197 | 35197 |
| 32198 | 33198 | 34198 | 35198 |
| 32199 | 33199 | 34199 | 35199 |
| 32200 | 33200 | 34200 | 35200 |
| 32201 | 33201 | 34201 | 35201 |
| 32202 | 33202 | 34202 | 35202 |
| 32203 | 33203 | 34203 | 35203 |
| 32204 | 33204 | 34204 | 35204 |
| 32205 | 33205 | 34205 | 35205 |
| 32206 | 33206 | 34206 | 35206 |
| 32207 | 33207 | 34207 | 35207 |
| 32208 | 33208 | 34208 | 35208 |
| 32209 | 33209 | 34209 | 35209 |
| 32210 | 33210 | 34210 | 35210 |
| 32211 | 33211 | 34211 | 35211 |
| 32212 | 33212 | 34212 | 35212 |
| 32213 | 33213 | 34213 | 35213 |
| 32214 | 33214 | 34214 | 35214 |
| 32215 | 33215 | 34215 | 35215 |
| 32216 | 33216 | 34216 | 35216 |
| 32217 | 33217 | 34217 | 35217 |
| 32218 | 33218 | 34218 | 35218 |
| 32219 | 33219 | 34219 | 35219 |
| 32220 | 33220 | 34220 | 35220 |
| 32221 | 33221 | 34221 | 35221 |
| 32222 | 33222 | 34222 | 35222 |
| 32223 | 33223 | 34223 | 35223 |
| 32224 | 33224 | 34224 | 35224 |
| 32225 | 33225 | 34225 | 35225 |
| 32226 | 33226 | 34226 | 35226 |
| 32227 | 33227 | 34227 | 35227 |
| 32228 | 33228 | 34228 | 35228 |
| 32229 | 33229 | 34229 | 35229 |
| 32230 | 33230 | 34230 | 35230 |
| 32231 | 33231 | 34231 | 35231 |
| 32232 | 33232 | 34232 | 35232 |
| 32233 | 33233 | 34233 | 35233 |
| 32234 | 33234 | 34234 | 35234 |
| 32235 | 33235 | 34235 | 35235 |
| 32236 | 33236 | 34236 | 35236 |
| 32237 | 33237 | 34237 | 35237 |
| 32238 | 33238 | 34238 | 35238 |
| 32239 | 33239 | 34239 | 35239 |
| 32240 | 33240 | 34240 | 35240 |
| 32241 | 33241 | 34241 | 35241 |
| 32242 | 33242 | 34242 | 35242 |
| 32243 | 33243 | 34243 | 35243 |
| 32244 | 33244 | 34244 | 35244 |
| 32245 | 33245 | 34245 | 35245 |
| 32246 | 33246 | 34246 | 35246 |
| 32247 | 33247 | 34247 | 35247 |
| 32248 | 33248 | 34248 | 35248 |
| 32249 | 33249 | 34249 | 35249 |
| 32250 | 33250 | 34250 | 35250 |
| 32251 | 33251 | 34251 | 35251 |
| 32252 | 33252 | 34252 | 35252 |
| 32253 | 33253 | 34253 | 35253 |
| 32254 | 33254 | 34254 | 35254 |
| 32255 | 33255 | 34255 | 35255 |
| 32256 | 33256 | 34256 | 35256 |
| 32257 | 33257 | 34257 | 35257 |
| 32258 | 33258 | 34258 | 35258 |
| 32259 | 33259 | 34259 | 35259 |
| 32260 | 33260 | 34260 | 35260 |
| 32261 | 33261 | 34261 | 35261 |
| 32262 | 33262 | 34262 | 35262 |
| 32263 | 33263 | 34263 | 35263 |
| 32264 | 33264 | 34264 | 35264 |
| 32265 | 33265 | 34265 | 35265 |
| 32266 | 33266 | 34266 | 35266 |
| 32267 | 33267 | 34267 | 35267 |
| 32268 | 33268 | 34268 | 35268 |
| 32269 | 33269 | 34269 | 35269 |
| 32270 | 33270 | 34270 | 35270 |
| 32271 | 33271 | 34271 | 35271 |
| 32272 | 33272 | 34272 | 35272 |
| 32273 | 33273 | 34273 | 35273 |
| 32274 | 33274 | 34274 | 35274 |
| 32275 | 33275 | 34275 | 35275 |
| 32276 | 33276 | 34276 | 35276 |
| 32277 | 33277 | 34277 | 35277 |
| 32278 | 33278 | 34278 | 35278 |
| 32279 | 33279 | 34279 | 35279 |
| 32280 | 33280 | 34280 | 35280 |
| 32281 | 33281 | 34281 | 35281 |
| 32282 | 33282 | 34282 | 35282 |
| 32283 | 33283 | 34283 | 35283 |
| 32284 | 33284 | 34284 | 35284 |
| 32285 | 33285 | 34285 | 35285 |
| 32286 | 33286 | 34286 | 35286 |
| 32287 | 33287 | 34287 | 35287 |
| 32288 | 33288 | 34288 | 35288 |
| 32289 | 33289 | 34289 | 35289 |
| 32290 | 33290 | 34290 | 35290 |
| 32291 | 33291 | 34291 | 35291 |
| 32292 | 33292 | 34292 | 35292 |
| 32293 | 33293 | 34293 | 35293 |
| 32294 | 33294 | 34294 | 35294 |
| 32295 | 33295 | 34295 | 35295 |
| 32296 | 33296 | 34296 | 35296 |
| 32297 | 33297 | 34297 | 35297 |
| 32298 | 33298 | 34298 | 35298 |
| 32299 | 33299 | 34299 | 35299 |
| 32300 | 33300 | 34300 | 35300 |
| 32301 | 33301 | 34301 | 35301 |
| 32302 | 33302 | 34302 | 35302 |
| 32303 | 33303 | 34303 | 35303 |
| 32304 | 33304 | 34304 | 35304 |
| 32305 | 33305 | 34305 | 35305 |
| 32306 | 33306 | 34306 | 35306 |
| 32307 | 33307 | 34307 | 35307 |
| 32308 | 33308 | 34308 | 35308 |
| 32309 | 33309 | 34309 | 35309 |
| 32310 | 33310 | 34310 | 35310 |
| 32311 | 33311 | 34311 | 35311 |
| 32312 | 33312 | 34312 | 35312 |
| 32313 | 33313 | 34313 | 35313 |
| 32314 | 33314 | 34314 | 35314 |
| 32315 | 33315 | 34315 | 35315 |
| 32316 | 33316 | 34316 | 35316 |
| 32317 | 33317 | 34317 | 35317 |
| 32318 | 33318 | 34318 | 35318 |
| 32319 | 33319 | 34319 | 35319 |
| 32320 | 33320 | 34320 | 35320 |
| 32321 | 33321 | 34321 | 35321 |
| 32322 | 33322 | 34322 | 35322 |
| 32323 | 33323 | 34323 | 35323 |
| 32324 | 33324 | 34324 | 35324 |
| 32325 | 33325 | 34325 | 35325 |
| 32326 | 33326 | 34326 | 35326 |
| 32327 | 33327 | 34327 | 35327 |
| 32328 | 33328 | 34328 | 35328 |
| 32329 | 33329 | 34329 | 35329 |
| 32330 | 33330 | 34330 | 35330 |
| 32331 | 33331 | 34331 | 35331 |
| 32332 | 33332 | 34332 | 35332 |
| 32333 | 33333 | 34333 | 35333 |
| 32334 | 33334 | 34334 | 35334 |
| 32335 | 33335 | 34335 | 35335 |
| 32336 | 33336 | 34336 | 35336 |
| 32337 | 33337 | 34337 | 35337 |
| 32338 | 33338 | 34338 | 35338 |
| 32339 | 33339 | 34339 | 35339 |
| 32340 | 33340 | 34340 | 35340 |
| 32341 | 33341 | 34341 | 35341 |
| 32342 | 33342 | 34342 | 35342 |
| 32343 | 33343 | 34343 | 35343 |
| 32344 | 33344 | 34344 | 35344 |
| 32345 | 33345 | 34345 | 35345 |
| 32346 | 33346 | 34346 | 35346 |
| 32347 | 33347 | 34347 | 35347 |
| 32348 | 33348 | 34348 | 35348 |
| 32349 | 33349 | 34349 | 35349 |
| 32350 | 33350 | 34350 | 35350 |
| 32351 | 33351 | 34351 | 35351 |
| 32352 | 33352 | 34352 | 35352 |
| 32353 | 33353 | 34353 | 35353 |
| 32354 | 33354 | 34354 | 35354 |
| 32355 | 33355 | 34355 | 35355 |
| 32356 | 33356 | 34356 | 35356 |
| 32357 | 33357 | 34357 | 35357 |
| 32358 | 33358 | 34358 | 35358 |
| 32359 | 33359 | 34359 | 35359 |
| 32360 | 33360 | 34360 | 35360 |
| 32361 | 33361 | 34361 | 35361 |
| 32362 | 33362 | 34362 | 35362 |
| 32363 | 33363 | 34363 | 35363 |
| 32364 | 33364 | 34364 | 35364 |
| 32365 | 33365 | 34365 | 35365 |
| 32366 | 33366 | 34366 | 35366 |
| 32367 | 33367 | 34367 | 35367 |
| 32368 | 33368 | 34368 | 35368 |
| 32369 | 33369 | 34369 | 35369 |
| 32370 | 33370 | 34370 | 35370 |
| 32371 | 33371 | 34371 | 35371 |
| 32372 | 33372 | 34372 | 35372 |
| 32373 | 33373 | 34373 | 35373 |
| 32374 | 33374 | 34374 | 35374 |
| 32375 | 33375 | 34375 | 35375 |
| 32376 | 33376 | 34376 | 35376 |
| 32377 | 33377 | 34377 | 35377 |
| 32378 | 33378 | 34378 | 35378 |
| 32379 | 33379 | 34379 | 35379 |
| 32380 | 33380 | 34380 | 35380 |
| 32381 | 33381 | 34381 | 35381 |
| 32382 | 33382 | 34382 | 35382 |
| 32383 | 33383 | 34383 | 35383 |
| 32384 | 33384 | 34384 | 35384 |
| 32385 | 33385 | 34385 | 35385 |
| 32386 | 33386 | 34386 | 35386 |
| 32387 | 33387 | 34387 | 35387 |
| 32388 | 33388 | 34388 | 35388 |
| 32389 | 33389 | 34389 | 35389 |
| 32390 | 33390 | 34390 | 35390 |
| 32391 | 33391 | 34391 | 35391 |
| 32392 | 33392 | 34392 | 35392 |
| 32393 | 33393 | 34393 | 35393 |
| 32394 | 33394 | 34394 | 35394 |
| 32395 | 33395 | 34395 | 35395 |
| 32396 | 33396 | 34396 | 35396 |
| 32397 | 33397 | 34397 | 35397 |
| 32398 | 33398 | 34398 | 35398 |
| 32399 | 33399 | 34399 | 35399 |
| 32400 | 33400 | 34400 | 35400 |
| 32401 | 33401 | 34401 | 35401 |
| 32402 | 33402 | 34402 | 35402 |
| 32403 | 33403 | 34403 | 35403 |
| 32404 | 33404 | 34404 | 35404 |
| 32405 | 33405 | 34405 | 35405 |
| 32406 | 33406 | 34406 | 35406 |
| 32407 | 33407 | 34407 | 35407 |
| 32408 | 33408 | 34408 | 35408 |
| 32409 | 33409 | 34409 | 35409 |
| 32410 | 33410 | 34410 | 35410 |
| 32411 | 33411 | 34411 | 35411 |
| 32412 | 33412 | 34412 | 35412 |
| 32413 | 33413 | 34413 | 35413 |
| 32414 | 33414 | 34414 | 35414 |
| 32415 | 33415 | 34415 | 35415 |
| 32416 | 33416 | 34416 | 35416 |
| 32417 | 33417 | 34417 | 35417 |
| 32418 | 33418 | 34418 | 35418 |
| 32419 | 33419 | 34419 | 35419 |
| 32420 | 33420 | 34420 | 35420 |
| 32421 | 33421 | 34421 | 35421 |
| 32422 | 33422 | 34422 | 35422 |
| 32423 | 33423 | 34423 | 35423 |
| 32424 | 33424 | 34424 | 35424 |
| 32425 | 33425 | 34425 | 35425 |
| 32426 | 33426 | 34426 | 35426 |
| 32427 | 33427 | 34427 | 35427 |
| 32428 | 33428 | 34428 | 35428 |
| 32429 | 33429 | 34429 | 35429 |
| 32430 | 33430 | 34430 | 35430 |
| 32431 | 33431 | 34431 | 35431 |
| 32432 | 33432 | 34432 | 35432 |
| 32433 | 33433 | 34433 | 35433 |
| 32434 | 33434 | 34434 | 35434 |
| 32435 | 33435 | 34435 | 35435 |
| 32436 | 33436 | 34436 | 35436 |
| 32437 | 33437 | 34437 | 35437 |
| 32438 | 33438 | 34438 | 35438 |
| 32439 | 33439 | 34439 | 35439 |
| 32440 | 33440 | 34440 | 35440 |
| 32441 | 33441 | 34441 | 35441 |
| 32442 | 33442 | 34442 | 35442 |
| 32443 | 33443 | 34443 | 35443 |
| 32444 | 33444 | 34444 | 35444 |
| 32445 | 33445 | 34445 | 35445 |
| 32446 | 33446 | 34446 | 35446 |
| 32447 | 33447 | 34447 | 35447 |
| 32448 | 33448 | 34448 | 35448 |
| 32449 | 33449 | 34449 | 35449 |
| 32450 | 33450 | 34450 | 35450 |
| 32451 | 33451 | 34451 | 35451 |
| 32452 | 33452 | 34452 | 35452 |
| 32453 | 33453 | 34453 | 35453 |
| 32454 | 33454 | 34454 | 35454 |
| 32455 | 33455 | 34455 | 35455 |
| 32456 | 33456 | 34456 | 35456 |
| 32457 | 33457 | 34457 | 35457 |
| 32458 | 33458 | 34458 | 35458 |
| 32459 | 33459 | 34459 | 35459 |
| 32460 | 33460 | 34460 | 35460 |
| 32461 | 33461 | 34461 | 35461 |
| 32462 | 33462 | 34462 | 35462 |
| 32463 | 33463 | 34463 | 35463 |
| 32464 | 33464 | 34464 | 35464 |
| 32465 | 33465 | 34465 | 35465 |
| 32466 | 33466 | 34466 | 35466 |
| 32467 | 33467 | 34467 | 35467 |
| 32468 | 33468 | 34468 | 35468 |
| 32469 | 33469 | 34469 | 35469 |
| 32470 | 33470 | 34470 | 35470 |
| 32471 | 33471 | 34471 | 35471 |
| 32472 | 33472 | 34472 | 35472 |
| 32473 | 33473 | 34473 | 35473 |
| 32474 | 33474 | 34474 | 35474 |
| 32475 | 33475 | 34475 | 35475 |
| 32476 | 33476 | 34476 | 35476 |
| 32477 | 33477 | 34477 | 35477 |
| 32478 | 33478 | 34478 | 35478 |
| 32479 | 33479 | 34479 | 35479 |
| 32480 | 33480 | 34480 | 35480 |
| 32481 | 33481 | 34481 | 35481 |
| 32482 | 33482 | 34482 | 35482 |
| 32483 | 33483 | 34483 | 35483 |
| 32484 | 33484 | 34484 | 35484 |
| 32485 | 33485 | 34485 | 35485 |
| 32486 | 33486 | 34486 | 35486 |
| 32487 | 33487 | 34487 | 35487 |
| 32488 | 33488 | 34488 | 35488 |
| 32489 | 33489 | 34489 | 35489 |
| 32490 | 33490 | 34490 | 35490 |
| 32491 | 33491 | 34491 | 35491 |
| 32492 | 33492 | 34492 | 35492 |
| 32493 | 33493 | 34493 | 35493 |
| 32494 | 33494 | 34494 | 35494 |
| 32495 | 33495 | 34495 | 35495 |
| 32496 | 33496 | 34496 | 35496 |
| 32497 | 33497 | 34497 | 35497 |
| 32498 | 33498 | 34498 | 35498 |
| 32499 | 33499 | 34499 | 35499 |
| 32500 | 33500 | 34500 | 35500 |
| 32501 | 33501 | 34501 | 35501 |
| 32502 | 33502 | 34502 | 35502 |
| 32503 | 33503 | 34503 | 35503 |
| 32504 | 33504 | 34504 | 35504 |
| 32505 | 33505 | 34505 | 35505 |
| 32506 | 33506 | 34506 | 35506 |
| 32507 | 33507 | 34507 | 35507 |
| 32508 | 33508 | 34508 | 35508 |
| 32509 | 33509 | 34509 | 35509 |
| 32510 | 33510 | 34510 | 35510 |
| 32511 | 33511 | 34511 | 35511 |
| 32512 | 33512 | 34512 | 35512 |
| 32513 | 33513 | 34513 | 35513 |
| 32514 | 33514 | 34514 | 35514 |
| 32515 | 33515 | 34515 | 35515 |
| 32516 | 33516 | 34516 | 35516 |
| 32517 | 33517 | 34517 | 35517 |
| 32518 | 33518 | 34518 | 35518 |
| 32519 | 33519 | 34519 | 35519 |
| 32520 | 33520 | 34520 | 35520 |
| 32521 | 33521 | 34521 | 35521 |
| 32522 | 33522 | 34522 | 35522 |
| 32523 | 33523 | 34523 | 35523 |
| 32524 | 33524 | 34524 | 35524 |
| 32525 | 33525 | 34525 | 35525 |
| 32526 | 33526 | 34526 | 35526 |
| 32527 | 33527 | 34527 | 35527 |
| 32528 | 33528 | 34528 | 35528 |
| 32529 | 33529 | 34529 | 35529 |
| 32530 | 33530 | 34530 | 35530 |
| 32531 | 33531 | 34531 | 35531 |
| 32532 | 33532 | 34532 | 35532 |
| 32533 | 33533 | 34533 | 35533 |
| 32534 | 33534 | 34534 | 35534 |
| 32535 | 33535 | 34535 | 35535 |
| 32536 | 33536 | 34536 | 35536 |
| 32537 | 33537 | 34537 | 35537 |
| 32538 | 33538 | 34538 | 35538 |
| 32539 | 33539 | 34539 | 35539 |
| 32540 | 33540 | 34540 | 35540 |
| 32541 | 33541 | 34541 | 35541 |
| 32542 | 33542 | 34542 | 35542 |
| 32543 | 33543 | 34543 | 35543 |
| 32544 | 33544 | 34544 | 35544 |
| 32545 | 33545 | 34545 | 35545 |
| 32546 | 33546 | 34546 | 35546 |
| 32547 | 33547 | 34547 | 35547 |
| 32548 | 33548 | 34548 | 35548 |
| 32549 | 33549 | 34549 | 35549 |
| 32550 | 33550 | 34550 | 35550 |
| 32551 | 33551 | 34551 | 35551 |
| 32552 | 33552 | 34552 | 35552 |
| 32553 | 33553 | 34553 | 35553 |
| 32554 | 33554 | 34554 | 35554 |
| 32555 | 33555 | 34555 | 35555 |
| 32556 | 33556 | 34556 | 35556 |
| 32557 | 33557 | 34557 | 35557 |
| 32558 | 33558 | 34558 | 35558 |
| 32559 | 33559 | 34559 | 35559 |
| 32560 | 33560 | 34560 | 35560 |
| 32561 | 33561 | 34561 | 35561 |
| 32562 | 33562 | 34562 | 35562 |
| 32563 | 33563 | 34563 | 35563 |
| 32564 | 33564 | 34564 | 35564 |
| 32565 | 33565 | 34565 | 35565 |
| 32566 | 33566 | 34566 | 35566 |
| 32567 | 33567 | 34567 | 35567 |
| 32568 | 33568 | 34568 | 35568 |
| 32569 | 33569 | 34569 | 35569 |
| 32570 | 33570 | 34570 | 35570 |
| 32571 | 33571 | 34571 | 35571 |
| 32572 | 33572 | 34572 | 35572 |
| 32573 | 33573 | 34573 | 35573 |
| 32574 | 33574 | 34574 | 35574 |
| 32575 | 33575 | 34575 | 35575 |
| 32576 | 33576 | 34576 | 35576 |
| 32577 | 33577 | 34577 | 35577 |
| 32578 | 33578 | 34578 | 35578 |
| 32579 | 33579 | 34579 | 35579 |
| 32580 | 33580 | 34580 | 35580 |
| 32581 | 33581 | 34581 | 35581 |
| 32582 | 33582 | 34582 | 35582 |
| 32583 | 33583 | 34583 | 35583 |
| 32584 | 33584 | 34584 | 35584 |
| 32585 | 33585 | 34585 | 35585 |
| 32586 | 33586 | 34586 | 35586 |
| 32587 | 33587 | 34587 | 35587 |
| 32588 | 33588 | 34588 | 35588 |
| 32589 | 33589 | 34589 | 35589 |
| 32590 | 33590 | 34590 | 35590 |
| 32591 | 33591 | 34591 | 35591 |
| 32592 | 33592 | 34592 | 35592 |
| 32593 | 33593 | 34593 | 35593 |
| 32594 | 33594 | 34594 | 35594 |
| 32595 | 33595 | 34595 | 35595 |
| 32596 | 33596 | 34596 | 35596 |
| 32597 | 33597 | 34597 | 35597 |
| 32598 | 33598 | 34598 | 35598 |
| 32599 | 33599 | 34599 | 35599 |
| 32600 | 33600 | 34600 | 35600 |
| 32601 | 33601 | 34601 | 35601 |
| 32602 | 33602 | 34602 | 35602 |
| 32603 | 33603 | 34603 | 35603 |
| 32604 | 33604 | 34604 | 35604 |
| 32605 | 33605 | 34605 | 35605 |
| 32606 | 33606 | 34606 | 35606 |
| 32607 | 33607 | 34607 | 35607 |
| 32608 | 33608 | 34608 | 35608 |
| 32609 | 33609 | 34609 | 35609 |
| 32610 | 33610 | 34610 | 35610 |
| 32611 | 33611 | 34611 | 35611 |
| 32612 | 33612 | 34612 | 35612 |
| 32613 | 33613 | 34613 | 35613 |
| 32614 | 33614 | 34614 | 35614 |
| 32615 | 33615 | 34615 | 35615 |
| 32616 | 33616 | 34616 | 35616 |
| 32617 | 33617 | 34617 | 35617 |
| 32618 | 33618 | 34618 | 35618 |
| 32619 | 33619 | 34619 | 35619 |
| 32620 | 33620 | 34620 | 35620 |
| 32621 | 33621 | 34621 | 35621 |
| 32622 | 33622 | 34622 | 35622 |
| 32623 | 33623 | 34623 | 35623 |
| 32624 | 33624 | 34624 | 35624 |
| 32625 | 33625 | 34625 | 35625 |
| 32626 | 33626 | 34626 | 35626 |
| 32627 | 33627 | 34627 | 35627 |
| 32628 | 33628 | 34628 | 35628 |
| 32629 | 33629 | 34629 | 35629 |
| 32630 | 33630 | 34630 | 35630 |
| 32631 | 33631 | 34631 | 35631 |
| 32632 | 33632 | 34632 | 35632 |
| 32633 | 33633 | 34633 | 35633 |
| 32634 | 33634 | 34634 | 35634 |
| 32635 | 33635 | 34635 | 35635 |
| 32636 | 33636 | 34636 | 35636 |
| 32637 | 33637 | 34637 | 35637 |
| 32638 | 33638 | 34638 | 35638 |
| 32639 | 33639 | 34639 | 35639 |
| 32640 | 33640 | 34640 | 35640 |
| 32641 | 33641 | 34641 | 35641 |
| 32642 | 33642 | 34642 | 35642 |
| 32643 | 33643 | 34643 | 35643 |
| 32644 | 33644 | 34644 | 35644 |
| 32645 | 33645 | 34645 | 35645 |
| 32646 | 33646 | 34646 | 35646 |
| 32647 | 33647 | 34647 | 35647 |
| 32648 | 33648 | 34648 | 35648 |
| 32649 | 33649 | 34649 | 35649 |
| 32650 | 33650 | 34650 | 35650 |
| 32651 | 33651 | 34651 | 35651 |
| 32652 | 33652 | 34652 | 35652 |
| 32653 | 33653 | 34653 | 35653 |
| 32654 | 33654 | 34654 | 35654 |
| 32655 | 33655 | 34655 | 35655 |
| 32656 | 33656 | 34656 | 35656 |
| 32657 | 33657 | 34657 | 35657 |
| 32658 | 33658 | 34658 | 35658 |
| 32659 | 33659 | 34659 | 35659 |
| 32660 | 33660 | 34660 | 35660 |
| 32661 | 33661 | 34661 | 35661 |
| 32662 | 33662 | 34662 | 35662 |
| 32663 | 33663 | 34663 | 35663 |
| 32664 | 33664 | 34664 | 35664 |
| 32665 | 33665 | 34665 | 35665 |
| 32666 | 33666 | 34666 | 35666 |
| 32667 | 33667 | 34667 | 35667 |
| 32668 | 33668 | 34668 | 35668 |
| 32669 | 33669 | 34669 | 35669 |
| 32670 | 33670 | 34670 | 35670 |
| 32671 | 33671 | 34671 | 35671 |
| 32672 | 33672 | 34672 | 35672 |
| 32673 | 33673 | 34673 | 35673 |
| 32674 | 33674 | 34674 | 35674 |
| 32675 | 33675 | 34675 | 35675 |
| 32676 | 33676 | 34676 | 35676 |
| 32677 | 33677 | 34677 | 35677 |
| 32678 | 33678 | 34678 | 35678 |
| 32679 | 33679 | 34679 | 35679 |
| 32680 | 33680 | 34680 | 35680 |
| 32681 | 33681 | 34681 | 35681 |
| 32682 | 33682 | 34682 | 35682 |
| 32683 | 33683 | 34683 | 35683 |
| 32684 | 33684 | 34684 | 35684 |
| 32685 | 33685 | 34685 | 35685 |
| 32686 | 33686 | 34686 | 35686 |
| 32687 | 33687 | 34687 | 35687 |
| 32688 | 33688 | 34688 | 35688 |
| 32689 | 33689 | 34689 | 35689 |
| 32690 | 33690 | 34690 | 35690 |
| 32691 | 33691 | 34691 | 35691 |
| 32692 | 33692 | 34692 | 35692 |
| 32693 | 33693 | 34693 | 35693 |
| 32694 | 33694 | 34694 | 35694 |
| 32695 | 33695 | 34695 | 35695 |
| 32696 | 33696 | 34696 | 35696 |
| 32697 | 33697 | 34697 | 35697 |
| 32698 | 33698 | 34698 | 35698 |
| 32699 | 33699 | 34699 | 35699 |
| 32700 | 33700 | 34700 | 35700 |
| 32701 | 33701 | 34701 | 35701 |
| 32702 | 33702 | 34702 | 35702 |
| 32703 | 33703 | 34703 | 35703 |
| 32704 | 33704 | 34704 | 35704 |
| 32705 | 33705 | 34705 | 35705 |
| 32706 | 33706 | 34706 | 35706 |
| 32707 | 33707 | 34707 | 35707 |
| 32708 | 33708 | 34708 | 35708 |
| 32709 | 33709 | 34709 | 35709 |
| 32710 | 33710 | 34710 | 35710 |
| 32711 | 33711 | 34711 | 35711 |
| 32712 | 33712 | 34712 | 35712 |
| 32713 | 33713 | 34713 | 35713 |
| 32714 | 33714 | 34714 | 35714 |
| 32715 | 33715 | 34715 | 35715 |
| 32716 | 33716 | 34716 | 35716 |
| 32717 | 33717 | 34717 | 35717 |
| 32718 | 33718 | 34718 | 35718 |
| 32719 | 33719 | 34719 | 35719 |
| 32720 | 33720 | 34720 | 35720 |
| 32721 | 33721 | 34721 | 35721 |
| 32722 | 33722 | 34722 | 35722 |
| 32723 | 33723 | 34723 | 35723 |
| 32724 | 33724 | 34724 | 35724 |
| 32725 | 33725 | 34725 | 35725 |
| 32726 | 33726 | 34726 | 35726 |
| 32727 | 33727 | 34727 | 35727 |
| 32728 | 33728 | 34728 | 35728 |
| 32729 | 33729 | 34729 | 35729 |
| 32730 | 33730 | 34730 | 35730 |
| 32731 | 33731 | 34731 | 35731 |
| 32732 | 33732 | 34732 | 35732 |
| 32733 | 33733 | 34733 | 35733 |
| 32734 | 33734 | 34734 | 35734 |
| 32735 | 33735 | 34735 | 35735 |
| 32736 | 33736 | 34736 | 35736 |
| 32737 | 33737 | 34737 | 35737 |
| 32738 | 33738 | 34738 | 35738 |
| 32739 | 33739 | 34739 | 35739 |
| 32740 | 33740 | 34740 | 35740 |
| 32741 | 33741 | 34741 | 35741 |
| 32742 | 33742 | 34742 | 35742 |
| 32743 | 33743 | 34743 | 35743 |
| 32744 | 33744 | 34744 | 35744 |
| 32745 | 33745 | 34745 | 35745 |
| 32746 | 33746 | 34746 | 35746 |
| 32747 | 33747 | 34747 | 35747 |
| 32748 | 33748 | 34748 | 35748 |
| 32749 | 33749 | 34749 | 35749 |
| 32750 | 33750 | 34750 | 35750 |
| 32751 | 33751 | 34751 | 35751 |
| 32752 | 33752 | 34752 | 35752 |
| 32753 | 33753 | 34753 | 35753 |
| 32754 | 33754 | 34754 | 35754 |
| 32755 | 33755 | 34755 | 35755 |
| 32756 | 33756 | 34756 | 35756 |
| 32757 | 33757 | 34757 | 35757 |
| 32758 | 33758 | 34758 | 35758 |
| 32759 | 33759 | 34759 | 35759 |
| 32760 | 33760 | 34760 | 35760 |
| 32761 | 33761 | 34761 | 35761 |
| 32762 | 33762 | 34762 | 35762 |
| 32763 | 33763 | 34763 | 35763 |
| 32764 | 33764 | 34764 | 35764 |
| 32765 | 33765 | 34765 | 35765 |
| 32766 | 33766 | 34766 | 35766 |
| 32767 | 33767 | 34767 | 35767 |
| 32768 | 33768 | 34768 | 35768 |
| 32769 | 33769 | 34769 | 35769 |
| 32770 | 33770 | 34770 | 35770 |
| 32771 | 33771 | 34771 | 35771 |
| 32772 | 33772 | 34772 | 35772 |
| 32773 | 33773 | 34773 | 35773 |
| 32774 | 33774 | 34774 | 35774 |
| 32775 | 33775 | 34775 | 35775 |
| 32776 | 33776 | 34776 | 35776 |
| 32777 | 33777 | 34777 | 35777 |
| 32778 | 33778 | 34778 | 35778 |
| 32779 | 33779 | 34779 | 35779 |
| 32780 | 33780 | 34780 | 35780 |
| 32781 | 33781 | 34781 | 35781 |
| 32782 | 33782 | 34782 | 35782 |
| 32783 | 33783 | 34783 | 35783 |
| 32784 | 33784 | 34784 | 35784 |
| 32785 | 33785 | 34785 | 35785 |
| 32786 | 33786 | 34786 | 35786 |
| 32787 | 33787 | 34787 | 35787 |
| 32788 | 33788 | 34788 | 35788 |
| 32789 | 33789 | 34789 | 35789 |
| 32790 | 33790 | 34790 | 35790 |
| 32791 | 33791 | 34791 | 35791 |
| 32792 | 33792 | 34792 | 35792 |
| 32793 | 33793 | 34793 | 35793 |
| 32794 | 33794 | 34794 | 35794 |
| 32795 | 33795 | 34795 | 35795 |
| 32796 | 33796 | 34796 | 35796 |
| 32797 | 33797 | 34797 | 35797 |
| 32798 | 33798 | 34798 | 35798 |
| 32799 | 33799 | 34799 | 35799 |
| 32800 | 33800 | 34800 | 35800 |
| 32801 | 33801 | 34801 | 35801 |
| 32802 | 33802 | 34802 | 35802 |
| 32803 | 33803 | 34803 | 35803 |
| 32804 | 33804 | 34804 | 35804 |
| 32805 | 33805 | 34805 | 35805 |
| 32806 | 33806 | 34806 | 35806 |
| 32807 | 33807 | 34807 | 35807 |
| 32808 | 33808 | 34808 | 35808 |
| 32809 | 33809 | 34809 | 35809 |
| 32810 | 33810 | 34810 | 35810 |
| 32811 | 33811 | 34811 | 35811 |
| 32812 | 33812 | 34812 | 35812 |
| 32813 | 33813 | 34813 | 35813 |
| 32814 | 33814 | 34814 | 35814 |
| 32815 | 33815 | 34815 | 35815 |
| 32816 | 33816 | 34816 | 35816 |
| 32817 | 33817 | 34817 | 35817 |
| 32818 | 33818 | 34818 | 35818 |
| 32819 | 33819 | 34819 | 35819 |
| 32820 | 33820 | 34820 | 35820 |
| 32821 | 33821 | 34821 | 35821 |
| 32822 | 33822 | 34822 | 35822 |
| 32823 | 33823 | 34823 | 35823 |
| 32824 | 33824 | 34824 | 35824 |
| 32825 | 33825 | 34825 | 35825 |
| 32826 | 33826 | 34826 | 35826 |
| 32827 | 33827 | 34827 | 35827 |
| 32828 | 33828 | 34828 | 35828 |
| 32829 | 33829 | 34829 | 35829 |
| 32830 | 33830 | 34830 | 35830 |
| 32831 | 33831 | 34831 | 35831 |
| 32832 | 33832 | 34832 | 35832 |
| 32833 | 33833 | 34833 | 35833 |
| 32834 | 33834 | 34834 | 35834 |
| 32835 | 33835 | 34835 | 35835 |
| 32836 | 33836 | 34836 | 35836 |
| 32837 | 33837 | 34837 | 35837 |
| 32838 | 33838 | 34838 | 35838 |
| 32839 | 33839 | 34839 | 35839 |
| 32840 | 33840 | 34840 | 35840 |
| 32841 | 33841 | 34841 | 35841 |
| 32842 | 33842 | 34842 | 35842 |
| 32843 | 33843 | 34843 | 35843 |
| 32844 | 33844 | 34844 | 35844 |
| 32845 | 33845 | 34845 | 35845 |
| 32846 | 33846 | 34846 | 35846 |
| 32847 | 33847 | 34847 | 35847 |
| 32848 | 33848 | 34848 | 35848 |
| 32849 | 33849 | 34849 | 35849 |
| 32850 | 33850 | 34850 | 35850 |
| 32851 | 33851 | 34851 | 35851 |
| 32852 | 33852 | 34852 | 35852 |
| 32853 | 33853 | 34853 | 35853 |
| 32854 | 33854 | 34854 | 35854 |
| 32855 | 33855 | 34855 | 35855 |
| 32856 | 33856 | 34856 | 35856 |
| 32857 | 33857 | 34857 | 35857 |
| 32858 | 33858 | 34858 | 35858 |
| 32859 | 33859 | 34859 | 35859 |
| 32860 | 33860 | 34860 | 35860 |
| 32861 | 33861 | 34861 | 35861 |
| 32862 | 33862 | 34862 | 35862 |
| 32863 | 33863 | 34863 | 35863 |
| 32864 | 33864 | 34864 | 35864 |
| 32865 | 33865 | 34865 | 35865 |
| 32866 | 33866 | 34866 | 35866 |
| 32867 | 33867 | 34867 | 35867 |
| 32868 | 33868 | 34868 | 35868 |
| 32869 | 33869 | 34869 | 35869 |
| 32870 | 33870 | 34870 | 35870 |
| 32871 | 33871 | 34871 | 35871 |
| 32872 | 33872 | 34872 | 35872 |
| 32873 | 33873 | 34873 | 35873 |
| 32874 | 33874 | 34874 | 35874 |
| 32875 | 33875 | 34875 | 35875 |
| 32876 | 33876 | 34876 | 35876 |
| 32877 | 33877 | 34877 | 35877 |
| 32878 | 33878 | 34878 | 35878 |
| 32879 | 33879 | 34879 | 35879 |
| 32880 | 33880 | 34880 | 35880 |
| 32881 | 33881 | 34881 | 35881 |
| 32882 | 33882 | 34882 | 35882 |
| 32883 | 33883 | 34883 | 35883 |
| 32884 | 33884 | 34884 | 35884 |
| 32885 | 33885 | 34885 | 35885 |
| 32886 | 33886 | 34886 | 35886 |
| 32887 | 33887 | 34887 | 35887 |
| 32888 | 33888 | 34888 | 35888 |
| 32889 | 33889 | 34889 | 35889 |
| 32890 | 33890 | 34890 | 35890 |
| 32891 | 33891 | 34891 | 35891 |
| 32892 | 33892 | 34892 | 35892 |
| 32893 | 33893 | 34893 | 35893 |
| 32894 | 33894 | 34894 | 35894 |
| 32895 | 33895 | 34895 | 35895 |
| 32896 | 33896 | 34896 | 35896 |
| 32897 | 33897 | 34897 | 35897 |
| 32898 | 33898 | 34898 | 35898 |
| 32899 | 33899 | 34899 | 35899 |
| 32900 | 33900 | 34900 | 35900 |
| 32901 | 33901 | 34901 | 35901 |
| 32902 | 33902 | 34902 | 35902 |
| 32903 | 33903 | 34903 | 35903 |
| 32904 | 33904 | 34904 | 35904 |
| 32905 | 33905 | 34905 | 35905 |
| 32906 | 33906 | 34906 | 35906 |
| 32907 | 33907 | 34907 | 35907 |
| 32908 | 33908 | 34908 | 35908 |
| 32909 | 33909 | 34909 | 35909 |
| 32910 | 33910 | 34910 | 35910 |
| 32911 | 33911 | 34911 | 35911 |
| 32912 | 33912 | 34912 | 35912 |
| 32913 | 33913 | 34913 | 35913 |
| 32914 | 33914 | 34914 | 35914 |
| 32915 | 33915 | 34915 | 35915 |
| 32916 | 33916 | 34916 | 35916 |
| 32917 | 33917 | 34917 | 35917 |
| 32918 | 33918 | 34918 | 35918 |
| 32919 | 33919 | 34919 | 35919 |
| 32920 | 33920 | 34920 | 35920 |
| 32921 | 33921 | 34921 | 35921 |
| 32922 | 33922 | 34922 | 35922 |
| 32923 | 33923 | 34923 | 35923 |
| 32924 | 33924 | 34924 | 35924 |
| 32925 | 33925 | 34925 | 35925 |
| 32926 | 33926 | 34926 | 35926 |
| 32927 | 33927 | 34927 | 35927 |
| 32928 | 33928 | 34928 | 35928 |
| 32929 | 33929 | 34929 | 35929 |
| 32930 | 33930 | 34930 | 35930 |
| 32931 | 33931 | 34931 | 35931 |
| 32932 | 33932 | 34932 | 35932 |
| 32933 | 33933 | 34933 | 35933 |
| 32934 | 33934 | 34934 | 35934 |
| 32935 | 33935 | 34935 | 35935 |
| 32936 | 33936 | 34936 | 35936 |
| 32937 | 33937 | 34937 | 35937 |
| 32938 | 33938 | 34938 | 35938 |
| 32939 | 33939 | 34939 | 35939 |
| 32940 | 33940 | 34940 | 35940 |
| 32941 | 33941 | 34941 | 35941 |
| 32942 | 33942 | 34942 | 35942 |
| 32943 | 33943 | 34943 | 35943 |
| 32944 | 33944 | 34944 | 35944 |
| 32945 | 33945 | 34945 | 35945 |
| 32946 | 33946 | 34946 | 35946 |
| 32947 | 33947 | 34947 | 35947 |
| 32948 | 33948 | 34948 | 35948 |
| 32949 | 33949 | 34949 | 35949 |
| 32950 | 33950 | 34950 | 35950 |
| 32951 | 33951 | 34951 | 35951 |
| 32952 | 33952 | 34952 | 35952 |
| 32953 | 33953 | 34953 | 35953 |
| 32954 | 33954 | 34954 | 35954 |
| 32955 | 33955 | 34955 | 35955 |
| 32956 | 33956 | 34956 | 35956 |
| 32957 | 33957 | 34957 | 35957 |
| 32958 | 33958 | 34958 | 35958 |
| 32959 | 33959 | 34959 | 35959 |
| 32960 | 33960 | 34960 | 35960 |
| 32961 | 33961 | 34961 | 35961 |
| 32962 | 33962 | 34962 | 35962 |
| 32963 | 33963 | 34963 | 35963 |
| 32964 | 33964 | 34964 | 35964 |
| 32965 | 33965 | 34965 | 35965 |
| 32966 | 33966 | 34966 | 35966 |
| 32967 | 33967 | 34967 | 35967 |
| 32968 | 33968 | 34968 | 35968 |
| 32969 | 33969 | 34969 | 35969 |
| 32970 | 33970 | 34970 | 35970 |
| 32971 | 33971 | 34971 | 35971 |
| 32972 | 33972 | 34972 | 35972 |
| 32973 | 33973 | 34973 | 35973 |
| 32974 | 33974 | 34974 | 35974 |
| 32975 | 33975 | 34975 | 35975 |
| 32976 | 33976 | 34976 | 35976 |
| 32977 | 33977 | 34977 | 35977 |
| 32978 | 33978 | 34978 | 35978 |
| 32979 | 33979 | 34979 | 35979 |
| 32980 | 33980 | 34980 | 35980 |
| 32981 | 33981 | 34981 | 35981 |
| 32982 | 33982 | 34982 | 35982 |
| 32983 | 33983 | 34983 | 35983 |
| 32984 | 33984 | 34984 | 35984 |
| 32985 | 33985 | 34985 | 35985 |
| 32986 | 33986 | 34986 | 35986 |
| 32987 | 33987 | 34987 | 35987 |
| 32988 | 33988 | 34988 | 35988 |
| 32989 | 33989 | 34989 | 35989 |
| 32990 | 33990 | 34990 | 35990 |
| 32991 | 33991 | 34991 | 35991 |
| 32992 | 33992 | 34992 | 35992 |
| 32993 | 33993 | 34993 | 35993 |
| 32994 | 33994 | 34994 | 35994 |
| 32995 | 33995 | 34995 | 35995 |
| 32996 | 33996 | 34996 | 35996 |
| 32997 | 33997 | 34997 | 35997 |
| 32998 | 33998 | 34998 | 35998 |
| 32999 | 33999 | 34999 | 35999 |
| 33000 | 34000 | 35000 | 36000 |
| 33001 | 34001 | 35001 | 36001 |
| 33002 | 34002 | 35002 | 36002 |

In some embodiments, the sequences that can bind to control materials during detection assay can be selected through the present disclosure. As a non-limiting sequence, the sequences that bind to peanut control material are selected by the present method, which comprise an inner sequence selected from the group consisting of the nucleic acid sequences of SEQ ID NOs. 36003 to 37002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to peanut control material may comprise a nucleic acid sequence selected from the group consisting of SEQ ID NOs. 37003 to 38002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to peanut control material may comprise a nucleic acid sequence selected from the group consisting of SEQ ID NOs. 38003 to 39002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to peanut control material may comprise a nucleic acid sequence selected from the group consisting of SEQ ID NOs. 39003 to 40002. In one embodiment, the aptamer of the present disclosure that can be used to detect peanut control material may comprise a nucleic acid sequence selected from the group consisting of SEQ ID NOs. 36003 to 40002 listed in Table 11, or variant thereof.

TABLE 11
Aptamer peanut control sequences
Aptamer sequence

| | | | 5′-sequence |
|---|---|---|---|
| | 5′-sequence | Inner | and inner |
| Inner | and inner | sequence and | sequence and |
| Sequence | sequence | 3′-sequence | 3′-sequence |
| (SEQ ID NO.) | (SEQ ID NO.) | (SEQ ID NO.) | (SEQ ID NO.) |
| 36003 | 37003 | 38003 | 39003 |
| 36004 | 37004 | 38004 | 39004 |
| 36005 | 37005 | 38005 | 39005 |
| 36006 | 37006 | 38006 | 39006 |
| 36007 | 37007 | 38007 | 39007 |
| 36008 | 37008 | 38008 | 39008 |
| 36009 | 37009 | 38009 | 39009 |
| 36010 | 37010 | 38010 | 39010 |
| 36011 | 37011 | 38011 | 39011 |
| 36012 | 37012 | 38012 | 39012 |
| 36013 | 37013 | 38013 | 39013 |
| 36014 | 37014 | 38014 | 39014 |
| 36015 | 37015 | 38015 | 39015 |
| 36016 | 37016 | 38016 | 39016 |
| 36017 | 37017 | 38017 | 39017 |
| 36018 | 37018 | 38018 | 39018 |
| 36019 | 37019 | 38019 | 39019 |
| 36020 | 37020 | 38020 | 39020 |
| 36021 | 37021 | 38021 | 39021 |
| 36022 | 37022 | 38022 | 39022 |
| 36023 | 37023 | 38023 | 39023 |
| 36024 | 37024 | 38024 | 39024 |
| 36025 | 37025 | 38025 | 39025 |
| 36026 | 37026 | 38026 | 39026 |
| 36027 | 37027 | 38027 | 39027 |
| 36028 | 37028 | 38028 | 39028 |
| 36029 | 37029 | 38029 | 39029 |
| 36030 | 37030 | 38030 | 39030 |
| 36031 | 37031 | 38031 | 39031 |
| 36032 | 37032 | 38032 | 39032 |
| 36033 | 37033 | 38033 | 39033 |
| 36034 | 37034 | 38034 | 39034 |
| 36035 | 37035 | 38035 | 39035 |
| 36036 | 37036 | 38036 | 39036 |
| 36037 | 37037 | 38037 | 39037 |
| 36038 | 37038 | 38038 | 39038 |
| 36039 | 37039 | 38039 | 39039 |
| 36040 | 37040 | 38040 | 39040 |
| 36041 | 37041 | 38041 | 39041 |
| 36042 | 37042 | 38042 | 39042 |
| 36043 | 37043 | 38043 | 39043 |
| 36044 | 37044 | 38044 | 39044 |
| 36045 | 37045 | 38045 | 39045 |
| 36046 | 37046 | 38046 | 39046 |
| 36047 | 37047 | 38047 | 39047 |
| 36048 | 37048 | 38048 | 39048 |
| 36049 | 37049 | 38049 | 39049 |
| 36050 | 37050 | 38050 | 39050 |
| 36051 | 37051 | 38051 | 39051 |
| 36052 | 37052 | 38052 | 39052 |
| 36053 | 37053 | 38053 | 39053 |
| 36054 | 37054 | 38054 | 39054 |
| 36055 | 37055 | 38055 | 39055 |
| 36056 | 37056 | 38056 | 39056 |
| 36057 | 37057 | 38057 | 39057 |
| 36058 | 37058 | 38058 | 39058 |
| 36059 | 37059 | 38059 | 39059 |
| 36060 | 37060 | 38060 | 39060 |
| 36061 | 37061 | 38061 | 39061 |
| 36062 | 37062 | 38062 | 39062 |
| 36063 | 37063 | 38063 | 39063 |
| 36064 | 37064 | 38064 | 39064 |
| 36065 | 37065 | 38065 | 39065 |
| 36066 | 37066 | 38066 | 39066 |
| 36067 | 37067 | 38067 | 39067 |
| 36068 | 37068 | 38068 | 39068 |
| 36069 | 37069 | 38069 | 39069 |
| 36070 | 37070 | 38070 | 39070 |
| 36071 | 37071 | 38071 | 39071 |
| 36072 | 37072 | 38072 | 39072 |
| 36073 | 37073 | 38073 | 39073 |
| 36074 | 37074 | 38074 | 39074 |
| 36075 | 37075 | 38075 | 39075 |
| 36076 | 37076 | 38076 | 39076 |
| 36077 | 37077 | 38077 | 39077 |
| 36078 | 37078 | 38078 | 39078 |
| 36079 | 37079 | 38079 | 39079 |
| 36080 | 37080 | 38080 | 39080 |
| 36081 | 37081 | 38081 | 39081 |
| 36082 | 37082 | 38082 | 39082 |
| 36083 | 37083 | 38083 | 39083 |
| 36084 | 37084 | 38084 | 39084 |
| 36085 | 37085 | 38085 | 39085 |
| 36086 | 37086 | 38086 | 39086 |
| 36087 | 37087 | 38087 | 39087 |
| 36088 | 37088 | 38088 | 39088 |
| 36089 | 37089 | 38089 | 39089 |
| 36090 | 37090 | 38090 | 39090 |
| 36091 | 37091 | 38091 | 39091 |
| 36092 | 37092 | 38092 | 39092 |
| 36093 | 37093 | 38093 | 39093 |
| 36094 | 37094 | 38094 | 39094 |
| 36095 | 37095 | 38095 | 39095 |
| 36096 | 37096 | 38096 | 39096 |
| 36097 | 37097 | 38097 | 39097 |
| 36098 | 37098 | 38098 | 39098 |
| 36099 | 37099 | 38099 | 39099 |
| 36100 | 37100 | 38100 | 39100 |
| 36101 | 37101 | 38101 | 39101 |
| 36102 | 37102 | 38102 | 39102 |
| 36103 | 37103 | 38103 | 39103 |
| 36104 | 37104 | 38104 | 39104 |
| 36105 | 37105 | 38105 | 39105 |
| 36106 | 37106 | 38106 | 39106 |
| 36107 | 37107 | 38107 | 39107 |
| 36108 | 37108 | 38108 | 39108 |
| 36109 | 37109 | 38109 | 39109 |
| 36110 | 37110 | 38110 | 39110 |
| 36111 | 37111 | 38111 | 39111 |
| 36112 | 37112 | 38112 | 39112 |
| 36113 | 37113 | 38113 | 39113 |
| 36114 | 37114 | 38114 | 39114 |
| 36115 | 37115 | 38115 | 39115 |
| 36116 | 37116 | 38116 | 39116 |
| 36117 | 37117 | 38117 | 39117 |
| 36118 | 37118 | 38118 | 39118 |
| 36119 | 37119 | 38119 | 39119 |
| 36120 | 37120 | 38120 | 39120 |
| 36121 | 37121 | 38121 | 39121 |
| 36122 | 37122 | 38122 | 39122 |
| 36123 | 37123 | 38123 | 39123 |
| 36124 | 37124 | 38124 | 39124 |
| 36125 | 37125 | 38125 | 39125 |
| 36126 | 37126 | 38126 | 39126 |
| 36127 | 37127 | 38127 | 39127 |
| 36128 | 37128 | 38128 | 39128 |
| 36129 | 37129 | 38129 | 39129 |
| 36130 | 37130 | 38130 | 39130 |
| 36131 | 37131 | 38131 | 39131 |
| 36132 | 37132 | 38132 | 39132 |
| 36133 | 37133 | 38133 | 39133 |
| 36134 | 37134 | 38134 | 39134 |
| 36135 | 37135 | 38135 | 39135 |
| 36136 | 37136 | 38136 | 39136 |
| 36137 | 37137 | 38137 | 39137 |
| 36138 | 37138 | 38138 | 39138 |
| 36139 | 37139 | 38139 | 39139 |
| 36140 | 37140 | 38140 | 39140 |
| 36141 | 37141 | 38141 | 39141 |
| 36142 | 37142 | 38142 | 39142 |
| 36143 | 37143 | 38143 | 39143 |
| 36144 | 37144 | 38144 | 39144 |
| 36145 | 37145 | 38145 | 39145 |
| 36146 | 37146 | 38146 | 39146 |
| 36147 | 37147 | 38147 | 39147 |
| 36148 | 37148 | 38148 | 39148 |
| 36149 | 37149 | 38149 | 39149 |
| 36150 | 37150 | 38150 | 39150 |
| 36151 | 37151 | 38151 | 39151 |
| 36152 | 37152 | 38152 | 39152 |
| 36153 | 37153 | 38153 | 39153 |
| 36154 | 37154 | 38154 | 39154 |
| 36155 | 37155 | 38155 | 39155 |
| 36156 | 37156 | 38156 | 39156 |
| 36157 | 37157 | 38157 | 39157 |
| 36158 | 37158 | 38158 | 39158 |
| 36159 | 37159 | 38159 | 39159 |
| 36160 | 37160 | 38160 | 39160 |
| 36161 | 37161 | 38161 | 39161 |
| 36162 | 37162 | 38162 | 39162 |
| 36163 | 37163 | 38163 | 39163 |
| 36164 | 37164 | 38164 | 39164 |
| 36165 | 37165 | 38165 | 39165 |
| 36166 | 37166 | 38166 | 39166 |
| 36167 | 37167 | 38167 | 39167 |
| 36168 | 37168 | 38168 | 39168 |
| 36169 | 37169 | 38169 | 39169 |
| 36170 | 37170 | 38170 | 39170 |
| 36171 | 37171 | 38171 | 39171 |
| 36172 | 37172 | 38172 | 39172 |
| 36173 | 37173 | 38173 | 39173 |
| 36174 | 37174 | 38174 | 39174 |
| 36175 | 37175 | 38175 | 39175 |
| 36176 | 37176 | 38176 | 39176 |
| 36177 | 37177 | 38177 | 39177 |
| 36178 | 37178 | 38178 | 39178 |
| 36179 | 37179 | 38179 | 39179 |
| 36180 | 37180 | 38180 | 39180 |
| 36181 | 37181 | 38181 | 39181 |
| 36182 | 37182 | 38182 | 39182 |
| 36183 | 37183 | 38183 | 39183 |
| 36184 | 37184 | 38184 | 39184 |
| 36185 | 37185 | 38185 | 39185 |
| 36186 | 37186 | 38186 | 39186 |
| 36187 | 37187 | 38187 | 39187 |
| 36188 | 37188 | 38188 | 39188 |
| 36189 | 37189 | 38189 | 39189 |
| 36190 | 37190 | 38190 | 39190 |
| 36191 | 37191 | 38191 | 39191 |
| 36192 | 37192 | 38192 | 39192 |
| 36193 | 37193 | 38193 | 39193 |
| 36194 | 37194 | 38194 | 39194 |
| 36195 | 37195 | 38195 | 39195 |
| 36196 | 37196 | 38196 | 39196 |
| 36197 | 37197 | 38197 | 39197 |
| 36198 | 37198 | 38198 | 39198 |
| 36199 | 37199 | 38199 | 39199 |
| 36200 | 37200 | 38200 | 39200 |
| 36201 | 37201 | 38201 | 39201 |
| 36202 | 37202 | 38202 | 39202 |
| 36203 | 37203 | 38203 | 39203 |
| 36204 | 37204 | 38204 | 39204 |
| 36205 | 37205 | 38205 | 39205 |
| 36206 | 37206 | 38206 | 39206 |
| 36207 | 37207 | 38207 | 39207 |
| 36208 | 37208 | 38208 | 39208 |
| 36209 | 37209 | 38209 | 39209 |
| 36210 | 37210 | 38210 | 39210 |
| 36211 | 37211 | 38211 | 39211 |
| 36212 | 37212 | 38212 | 39212 |
| 36213 | 37213 | 38213 | 39213 |
| 36214 | 37214 | 38214 | 39214 |
| 36215 | 37215 | 38215 | 39215 |
| 36216 | 37216 | 38216 | 39216 |
| 36217 | 37217 | 38217 | 39217 |
| 36218 | 37218 | 38218 | 39218 |
| 36219 | 37219 | 38219 | 39219 |
| 36220 | 37220 | 38220 | 39220 |
| 36221 | 37221 | 38221 | 39221 |
| 36222 | 37222 | 38222 | 39222 |
| 36223 | 37223 | 38223 | 39223 |
| 36224 | 37224 | 38224 | 39224 |
| 36225 | 37225 | 38225 | 39225 |
| 36226 | 37226 | 38226 | 39226 |
| 36227 | 37227 | 38227 | 39227 |
| 36228 | 37228 | 38228 | 39228 |
| 36229 | 37229 | 38229 | 39229 |
| 36230 | 37230 | 38230 | 39230 |
| 36231 | 37231 | 38231 | 39231 |
| 36232 | 37232 | 38232 | 39232 |
| 36233 | 37233 | 38233 | 39233 |
| 36234 | 37234 | 38234 | 39234 |
| 36235 | 37235 | 38235 | 39235 |
| 36236 | 37236 | 38236 | 39236 |
| 36237 | 37237 | 38237 | 39237 |
| 36238 | 37238 | 38238 | 39238 |
| 36239 | 37239 | 38239 | 39239 |
| 36240 | 37240 | 38240 | 39240 |
| 36241 | 37241 | 38241 | 39241 |
| 36242 | 37242 | 38242 | 39242 |
| 36243 | 37243 | 38243 | 39243 |
| 36244 | 37244 | 38244 | 39244 |
| 36245 | 37245 | 38245 | 39245 |
| 36246 | 37246 | 38246 | 39246 |
| 36247 | 37247 | 38247 | 39247 |
| 36248 | 37248 | 38248 | 39248 |
| 36249 | 37249 | 38249 | 39249 |
| 36250 | 37250 | 38250 | 39250 |
| 36251 | 37251 | 38251 | 39251 |
| 36252 | 37252 | 38252 | 39252 |
| 36253 | 37253 | 38253 | 39253 |
| 36254 | 37254 | 38254 | 39254 |
| 36255 | 37255 | 38255 | 39255 |
| 36256 | 37256 | 38256 | 39256 |
| 36257 | 37257 | 38257 | 39257 |
| 36258 | 37258 | 38258 | 39258 |
| 36259 | 37259 | 38259 | 39259 |
| 36260 | 37260 | 38260 | 39260 |
| 36261 | 37261 | 38261 | 39261 |
| 36262 | 37262 | 38262 | 39262 |
| 36263 | 37263 | 38263 | 39263 |
| 36264 | 37264 | 38264 | 39264 |
| 36265 | 37265 | 38265 | 39265 |
| 36266 | 37266 | 38266 | 39266 |
| 36267 | 37267 | 38267 | 39267 |
| 36268 | 37268 | 38268 | 39268 |
| 36269 | 37269 | 38269 | 39269 |
| 36270 | 37270 | 38270 | 39270 |
| 36271 | 37271 | 38271 | 39271 |
| 36272 | 37272 | 38272 | 39272 |
| 36273 | 37273 | 38273 | 39273 |
| 36274 | 37274 | 38274 | 39274 |
| 36275 | 37275 | 38275 | 39275 |
| 36276 | 37276 | 38276 | 39276 |
| 36277 | 37277 | 38277 | 39277 |
| 36278 | 37278 | 38278 | 39278 |
| 36279 | 37279 | 38279 | 39279 |
| 36280 | 37280 | 38280 | 39280 |
| 36281 | 37281 | 38281 | 39281 |
| 36282 | 37282 | 38282 | 39282 |
| 36283 | 37283 | 38283 | 39283 |
| 36284 | 37284 | 38284 | 39284 |
| 36285 | 37285 | 38285 | 39285 |
| 36286 | 37286 | 38286 | 39286 |
| 36287 | 37287 | 38287 | 39287 |
| 36288 | 37288 | 38288 | 39288 |
| 36289 | 37289 | 38289 | 39289 |
| 36290 | 37290 | 38290 | 39290 |
| 36291 | 37291 | 38291 | 39291 |
| 36292 | 37292 | 38292 | 39292 |
| 36293 | 37293 | 38293 | 39293 |
| 36294 | 37294 | 38294 | 39294 |
| 36295 | 37295 | 38295 | 39295 |
| 36296 | 37296 | 38296 | 39296 |
| 36297 | 37297 | 38297 | 39297 |
| 36298 | 37298 | 38298 | 39298 |
| 36299 | 37299 | 38299 | 39299 |
| 36300 | 37300 | 38300 | 39300 |
| 36301 | 37301 | 38301 | 39301 |
| 36302 | 37302 | 38302 | 39302 |
| 36303 | 37303 | 38303 | 39303 |
| 36304 | 37304 | 38304 | 39304 |
| 36305 | 37305 | 38305 | 39305 |
| 36306 | 37306 | 38306 | 39306 |
| 36307 | 37307 | 38307 | 39307 |
| 36308 | 37308 | 38308 | 39308 |
| 36309 | 37309 | 38309 | 39309 |
| 36310 | 37310 | 38310 | 39310 |
| 36311 | 37311 | 38311 | 39311 |
| 36312 | 37312 | 38312 | 39312 |
| 36313 | 37313 | 38313 | 39313 |
| 36314 | 37314 | 38314 | 39314 |
| 36315 | 37315 | 38315 | 39315 |
| 36316 | 37316 | 38316 | 39316 |
| 36317 | 37317 | 38317 | 39317 |
| 36318 | 37318 | 38318 | 39318 |
| 36319 | 37319 | 38319 | 39319 |
| 36320 | 37320 | 38320 | 39320 |
| 36321 | 37321 | 38321 | 39321 |
| 36322 | 37322 | 38322 | 39322 |
| 36323 | 37323 | 38323 | 39323 |
| 36324 | 37324 | 38324 | 39324 |
| 36325 | 37325 | 38325 | 39325 |
| 36326 | 37326 | 38326 | 39326 |
| 36327 | 37327 | 38327 | 39327 |
| 36328 | 37328 | 38328 | 39328 |
| 36329 | 37329 | 38329 | 39329 |
| 36330 | 37330 | 38330 | 39330 |
| 36331 | 37331 | 38331 | 39331 |
| 36332 | 37332 | 38332 | 39332 |
| 36333 | 37333 | 38333 | 39333 |
| 36334 | 37334 | 38334 | 39334 |
| 36335 | 37335 | 38335 | 39335 |
| 36336 | 37336 | 38336 | 39336 |
| 36337 | 37337 | 38337 | 39337 |
| 36338 | 37338 | 38338 | 39338 |
| 36339 | 37339 | 38339 | 39339 |
| 36340 | 37340 | 38340 | 39340 |
| 36341 | 37341 | 38341 | 39341 |
| 36342 | 37342 | 38342 | 39342 |
| 36343 | 37343 | 38343 | 39343 |
| 36344 | 37344 | 38344 | 39344 |
| 36345 | 37345 | 38345 | 39345 |
| 36346 | 37346 | 38346 | 39346 |
| 36347 | 37347 | 38347 | 39347 |
| 36348 | 37348 | 38348 | 39348 |
| 36349 | 37349 | 38349 | 39349 |
| 36350 | 37350 | 38350 | 39350 |
| 36351 | 37351 | 38351 | 39351 |
| 36352 | 37352 | 38352 | 39352 |
| 36353 | 37353 | 38353 | 39353 |
| 36354 | 37354 | 38354 | 39354 |
| 36355 | 37355 | 38355 | 39355 |
| 36356 | 37356 | 38356 | 39356 |
| 36357 | 37357 | 38357 | 39357 |
| 36358 | 37358 | 38358 | 39358 |
| 36359 | 37359 | 38359 | 39359 |
| 36360 | 37360 | 38360 | 39360 |
| 36361 | 37361 | 38361 | 39361 |
| 36362 | 37362 | 38362 | 39362 |
| 36363 | 37363 | 38363 | 39363 |
| 36364 | 37364 | 38364 | 39364 |
| 36365 | 37365 | 38365 | 39365 |
| 36366 | 37366 | 38366 | 39366 |
| 36367 | 37367 | 38367 | 39367 |
| 36368 | 37368 | 38368 | 39368 |
| 36369 | 37369 | 38369 | 39369 |
| 36370 | 37370 | 38370 | 39370 |
| 36371 | 37371 | 38371 | 39371 |
| 36372 | 37372 | 38372 | 39372 |
| 36373 | 37373 | 38373 | 39373 |
| 36374 | 37374 | 38374 | 39374 |
| 36375 | 37375 | 38375 | 39375 |
| 36376 | 37376 | 38376 | 39376 |
| 36377 | 37377 | 38377 | 39377 |
| 36378 | 37378 | 38378 | 39378 |
| 36379 | 37379 | 38379 | 39379 |
| 36380 | 37380 | 38380 | 39380 |
| 36381 | 37381 | 38381 | 39381 |
| 36382 | 37382 | 38382 | 39382 |
| 36383 | 37383 | 38383 | 39383 |
| 36384 | 37384 | 38384 | 39384 |
| 36385 | 37385 | 38385 | 39385 |
| 36386 | 37386 | 38386 | 39386 |
| 36387 | 37387 | 38387 | 39387 |
| 36388 | 37388 | 38388 | 39388 |
| 36389 | 37389 | 38389 | 39389 |
| 36390 | 37390 | 38390 | 39390 |
| 36391 | 37391 | 38391 | 39391 |
| 36392 | 37392 | 38392 | 39392 |
| 36393 | 37393 | 38393 | 39393 |
| 36394 | 37394 | 38394 | 39394 |
| 36395 | 37395 | 38395 | 39395 |
| 36396 | 37396 | 38396 | 39396 |
| 36397 | 37397 | 38397 | 39397 |
| 36398 | 37398 | 38398 | 39398 |
| 36399 | 37399 | 38399 | 39399 |
| 36400 | 37400 | 38400 | 39400 |
| 36401 | 37401 | 38401 | 39401 |
| 36402 | 37402 | 38402 | 39402 |
| 36403 | 37403 | 38403 | 39403 |
| 36404 | 37404 | 38404 | 39404 |
| 36405 | 37405 | 38405 | 39405 |
| 36406 | 37406 | 38406 | 39406 |
| 36407 | 37407 | 38407 | 39407 |
| 36408 | 37408 | 38408 | 39408 |
| 36409 | 37409 | 38409 | 39409 |
| 36410 | 37410 | 38410 | 39410 |
| 36411 | 37411 | 38411 | 39411 |
| 36412 | 37412 | 38412 | 39412 |
| 36413 | 37413 | 38413 | 39413 |
| 36414 | 37414 | 38414 | 39414 |
| 36415 | 37415 | 38415 | 39415 |
| 36416 | 37416 | 38416 | 39416 |
| 36417 | 37417 | 38417 | 39417 |
| 36418 | 37418 | 38418 | 39418 |
| 36419 | 37419 | 38419 | 39419 |
| 36420 | 37420 | 38420 | 39420 |
| 36421 | 37421 | 38421 | 39421 |
| 36422 | 37422 | 38422 | 39422 |
| 36423 | 37423 | 38423 | 39423 |
| 36424 | 37424 | 38424 | 39424 |
| 36425 | 37425 | 38425 | 39425 |
| 36426 | 37426 | 38426 | 39426 |
| 36427 | 37427 | 38427 | 39427 |
| 36428 | 37428 | 38428 | 39428 |
| 36429 | 37429 | 38429 | 39429 |
| 36430 | 37430 | 38430 | 39430 |
| 36431 | 37431 | 38431 | 39431 |
| 36432 | 37432 | 38432 | 39432 |
| 36433 | 37433 | 38433 | 39433 |
| 36434 | 37434 | 38434 | 39434 |
| 36435 | 37435 | 38435 | 39435 |
| 36436 | 37436 | 38436 | 39436 |
| 36437 | 37437 | 38437 | 39437 |
| 36438 | 37438 | 38438 | 39438 |
| 36439 | 37439 | 38439 | 39439 |
| 36440 | 37440 | 38440 | 39440 |
| 36441 | 37441 | 38441 | 39441 |
| 36442 | 37442 | 38442 | 39442 |
| 36443 | 37443 | 38443 | 39443 |
| 36444 | 37444 | 38444 | 39444 |
| 36445 | 37445 | 38445 | 39445 |
| 36446 | 37446 | 38446 | 39446 |
| 36447 | 37447 | 38447 | 39447 |
| 36448 | 37448 | 38448 | 39448 |
| 36449 | 37449 | 38449 | 39449 |
| 36450 | 37450 | 38450 | 39450 |
| 36451 | 37451 | 38451 | 39451 |
| 36452 | 37452 | 38452 | 39452 |
| 36453 | 37453 | 38453 | 39453 |
| 36454 | 37454 | 38454 | 39454 |
| 36455 | 37455 | 38455 | 39455 |
| 36456 | 37456 | 38456 | 39456 |
| 36457 | 37457 | 38457 | 39457 |
| 36458 | 37458 | 38458 | 39458 |
| 36459 | 37459 | 38459 | 39459 |
| 36460 | 37460 | 38460 | 39460 |
| 36461 | 37461 | 38461 | 39461 |
| 36462 | 37462 | 38462 | 39462 |
| 36463 | 37463 | 38463 | 39463 |
| 36464 | 37464 | 38464 | 39464 |
| 36465 | 37465 | 38465 | 39465 |
| 36466 | 37466 | 38466 | 39466 |
| 36467 | 37467 | 38467 | 39467 |
| 36468 | 37468 | 38468 | 39468 |
| 36469 | 37469 | 38469 | 39469 |
| 36470 | 37470 | 38470 | 39470 |
| 36471 | 37471 | 38471 | 39471 |
| 36472 | 37472 | 38472 | 39472 |
| 36473 | 37473 | 38473 | 39473 |
| 36474 | 37474 | 38474 | 39474 |
| 36475 | 37475 | 38475 | 39475 |
| 36476 | 37476 | 38476 | 39476 |
| 36477 | 37477 | 38477 | 39477 |
| 36478 | 37478 | 38478 | 39478 |
| 36479 | 37479 | 38479 | 39479 |
| 36480 | 37480 | 38480 | 39480 |
| 36481 | 37481 | 38481 | 39481 |
| 36482 | 37482 | 38482 | 39482 |
| 36483 | 37483 | 38483 | 39483 |
| 36484 | 37484 | 38484 | 39484 |
| 36485 | 37485 | 38485 | 39485 |
| 36486 | 37486 | 38486 | 39486 |
| 36487 | 37487 | 38487 | 39487 |
| 36488 | 37488 | 38488 | 39488 |
| 36489 | 37489 | 38489 | 39489 |
| 36490 | 37490 | 38490 | 39490 |
| 36491 | 37491 | 38491 | 39491 |
| 36492 | 37492 | 38492 | 39492 |
| 36493 | 37493 | 38493 | 39493 |
| 36494 | 37494 | 38494 | 39494 |
| 36495 | 37495 | 38495 | 39495 |
| 36496 | 37496 | 38496 | 39496 |
| 36497 | 37497 | 38497 | 39497 |
| 36498 | 37498 | 38498 | 39498 |
| 36499 | 37499 | 38499 | 39499 |
| 36500 | 37500 | 38500 | 39500 |
| 36501 | 37501 | 38501 | 39501 |
| 36502 | 37502 | 38502 | 39502 |
| 36503 | 37503 | 38503 | 39503 |
| 36504 | 37504 | 38504 | 39504 |
| 36505 | 37505 | 38505 | 39505 |
| 36506 | 37506 | 38506 | 39506 |
| 36507 | 37507 | 38507 | 39507 |
| 36508 | 37508 | 38508 | 39508 |
| 36509 | 37509 | 38509 | 39509 |
| 36510 | 37510 | 38510 | 39510 |
| 36511 | 37511 | 38511 | 39511 |
| 36512 | 37512 | 38512 | 39512 |
| 36513 | 37513 | 38513 | 39513 |
| 36514 | 37514 | 38514 | 39514 |
| 36515 | 37515 | 38515 | 39515 |
| 36516 | 37516 | 38516 | 39516 |
| 36517 | 37517 | 38517 | 39517 |
| 36518 | 37518 | 38518 | 39518 |
| 36519 | 37519 | 38519 | 39519 |
| 36520 | 37520 | 38520 | 39520 |
| 36521 | 37521 | 38521 | 39521 |
| 36522 | 37522 | 38522 | 39522 |
| 36523 | 37523 | 38523 | 39523 |
| 36524 | 37524 | 38524 | 39524 |
| 36525 | 37525 | 38525 | 39525 |
| 36526 | 37526 | 38526 | 39526 |
| 36527 | 37527 | 38527 | 39527 |
| 36528 | 37528 | 38528 | 39528 |
| 36529 | 37529 | 38529 | 39529 |
| 36530 | 37530 | 38530 | 39530 |
| 36531 | 37531 | 38531 | 39531 |
| 36532 | 37532 | 38532 | 39532 |
| 36533 | 37533 | 38533 | 39533 |
| 36534 | 37534 | 38534 | 39534 |
| 36535 | 37535 | 38535 | 39535 |
| 36536 | 37536 | 38536 | 39536 |
| 36537 | 37537 | 38537 | 39537 |
| 36538 | 37538 | 38538 | 39538 |
| 36539 | 37539 | 38539 | 39539 |
| 36540 | 37540 | 38540 | 39540 |
| 36541 | 37541 | 38541 | 39541 |
| 36542 | 37542 | 38542 | 39542 |
| 36543 | 37543 | 38543 | 39543 |
| 36544 | 37544 | 38544 | 39544 |
| 36545 | 37545 | 38545 | 39545 |
| 36546 | 37546 | 38546 | 39546 |
| 36547 | 37547 | 38547 | 39547 |
| 36548 | 37548 | 38548 | 39548 |
| 36549 | 37549 | 38549 | 39549 |
| 36550 | 37550 | 38550 | 39550 |
| 36551 | 37551 | 38551 | 39551 |
| 36552 | 37552 | 38552 | 39552 |
| 36553 | 37553 | 38553 | 39553 |
| 36554 | 37554 | 38554 | 39554 |
| 36555 | 37555 | 38555 | 39555 |
| 36556 | 37556 | 38556 | 39556 |
| 36557 | 37557 | 38557 | 39557 |
| 36558 | 37558 | 38558 | 39558 |
| 36559 | 37559 | 38559 | 39559 |
| 36560 | 37560 | 38560 | 39560 |
| 36561 | 37561 | 38561 | 39561 |
| 36562 | 37562 | 38562 | 39562 |
| 36563 | 37563 | 38563 | 39563 |
| 36564 | 37564 | 38564 | 39564 |
| 36565 | 37565 | 38565 | 39565 |
| 36566 | 37566 | 38566 | 39566 |
| 36567 | 37567 | 38567 | 39567 |
| 36568 | 37568 | 38568 | 39568 |
| 36569 | 37569 | 38569 | 39569 |
| 36570 | 37570 | 38570 | 39570 |
| 36571 | 37571 | 38571 | 39571 |
| 36572 | 37572 | 38572 | 39572 |
| 36573 | 37573 | 38573 | 39573 |
| 36574 | 37574 | 38574 | 39574 |
| 36575 | 37575 | 38575 | 39575 |
| 36576 | 37576 | 38576 | 39576 |
| 36577 | 37577 | 38577 | 39577 |
| 36578 | 37578 | 38578 | 39578 |
| 36579 | 37579 | 38579 | 39579 |
| 36580 | 37580 | 38580 | 39580 |
| 36581 | 37581 | 38581 | 39581 |
| 36582 | 37582 | 38582 | 39582 |
| 36583 | 37583 | 38583 | 39583 |
| 36584 | 37584 | 38584 | 39584 |
| 36585 | 37585 | 38585 | 39585 |
| 36586 | 37586 | 38586 | 39586 |
| 36587 | 37587 | 38587 | 39587 |
| 36588 | 37588 | 38588 | 39588 |
| 36589 | 37589 | 38589 | 39589 |
| 36590 | 37590 | 38590 | 39590 |
| 36591 | 37591 | 38591 | 39591 |
| 36592 | 37592 | 38592 | 39592 |
| 36593 | 37593 | 38593 | 39593 |
| 36594 | 37594 | 38594 | 39594 |
| 36595 | 37595 | 38595 | 39595 |
| 36596 | 37596 | 38596 | 39596 |
| 36597 | 37597 | 38597 | 39597 |
| 36598 | 37598 | 38598 | 39598 |
| 36599 | 37599 | 38599 | 39599 |
| 36600 | 37600 | 38600 | 39600 |
| 36601 | 37601 | 38601 | 39601 |
| 36602 | 37602 | 38602 | 39602 |
| 36603 | 37603 | 38603 | 39603 |
| 36604 | 37604 | 38604 | 39604 |
| 36605 | 37605 | 38605 | 39605 |
| 36606 | 37606 | 38606 | 39606 |
| 36607 | 37607 | 38607 | 39607 |
| 36608 | 37608 | 38608 | 39608 |
| 36609 | 37609 | 38609 | 39609 |
| 36610 | 37610 | 38610 | 39610 |
| 36611 | 37611 | 38611 | 39611 |
| 36612 | 37612 | 38612 | 39612 |
| 36613 | 37613 | 38613 | 39613 |
| 36614 | 37614 | 38614 | 39614 |
| 36615 | 37615 | 38615 | 39615 |
| 36616 | 37616 | 38616 | 39616 |
| 36617 | 37617 | 38617 | 39617 |
| 36618 | 37618 | 38618 | 39618 |
| 36619 | 37619 | 38619 | 39619 |
| 36620 | 37620 | 38620 | 39620 |
| 36621 | 37621 | 38621 | 39621 |
| 36622 | 37622 | 38622 | 39622 |
| 36623 | 37623 | 38623 | 39623 |
| 36624 | 37624 | 38624 | 39624 |
| 36625 | 37625 | 38625 | 39625 |
| 36626 | 37626 | 38626 | 39626 |
| 36627 | 37627 | 38627 | 39627 |
| 36628 | 37628 | 38628 | 39628 |
| 36629 | 37629 | 38629 | 39629 |
| 36630 | 37630 | 38630 | 39630 |
| 36631 | 37631 | 38631 | 39631 |
| 36632 | 37632 | 38632 | 39632 |
| 36633 | 37633 | 38633 | 39633 |
| 36634 | 37634 | 38634 | 39634 |
| 36635 | 37635 | 38635 | 39635 |
| 36636 | 37636 | 38636 | 39636 |
| 36637 | 37637 | 38637 | 39637 |
| 36638 | 37638 | 38638 | 39638 |
| 36639 | 37639 | 38639 | 39639 |
| 36640 | 37640 | 38640 | 39640 |
| 36641 | 37641 | 38641 | 39641 |
| 36642 | 37642 | 38642 | 39642 |
| 36643 | 37643 | 38643 | 39643 |
| 36644 | 37644 | 38644 | 39644 |
| 36645 | 37645 | 38645 | 39645 |
| 36646 | 37646 | 38646 | 39646 |
| 36647 | 37647 | 38647 | 39647 |
| 36648 | 37648 | 38648 | 39648 |
| 36649 | 37649 | 38649 | 39649 |
| 36650 | 37650 | 38650 | 39650 |
| 36651 | 37651 | 38651 | 39651 |
| 36652 | 37652 | 38652 | 39652 |
| 36653 | 37653 | 38653 | 39653 |
| 36654 | 37654 | 38654 | 39654 |
| 36655 | 37655 | 38655 | 39655 |
| 36656 | 37656 | 38656 | 39656 |
| 36657 | 37657 | 38657 | 39657 |
| 36658 | 37658 | 38658 | 39658 |
| 36659 | 37659 | 38659 | 39659 |
| 36660 | 37660 | 38660 | 39660 |
| 36661 | 37661 | 38661 | 39661 |
| 36662 | 37662 | 38662 | 39662 |
| 36663 | 37663 | 38663 | 39663 |
| 36664 | 37664 | 38664 | 39664 |
| 36665 | 37665 | 38665 | 39665 |
| 36666 | 37666 | 38666 | 39666 |
| 36667 | 37667 | 38667 | 39667 |
| 36668 | 37668 | 38668 | 39668 |
| 36669 | 37669 | 38669 | 39669 |
| 36670 | 37670 | 38670 | 39670 |
| 36671 | 37671 | 38671 | 39671 |
| 36672 | 37672 | 38672 | 39672 |
| 36673 | 37673 | 38673 | 39673 |
| 36674 | 37674 | 38674 | 39674 |
| 36675 | 37675 | 38675 | 39675 |
| 36676 | 37676 | 38676 | 39676 |
| 36677 | 37677 | 38677 | 39677 |
| 36678 | 37678 | 38678 | 39678 |
| 36679 | 37679 | 38679 | 39679 |
| 36680 | 37680 | 38680 | 39680 |
| 36681 | 37681 | 38681 | 39681 |
| 36682 | 37682 | 38682 | 39682 |
| 36683 | 37683 | 38683 | 39683 |
| 36684 | 37684 | 38684 | 39684 |
| 36685 | 37685 | 38685 | 39685 |
| 36686 | 37686 | 38686 | 39686 |
| 36687 | 37687 | 38687 | 39687 |
| 36688 | 37688 | 38688 | 39688 |
| 36689 | 37689 | 38689 | 39689 |
| 36690 | 37690 | 38690 | 39690 |
| 36691 | 37691 | 38691 | 39691 |
| 36692 | 37692 | 38692 | 39692 |
| 36693 | 37693 | 38693 | 39693 |
| 36694 | 37694 | 38694 | 39694 |
| 36695 | 37695 | 38695 | 39695 |
| 36696 | 37696 | 38696 | 39696 |
| 36697 | 37697 | 38697 | 39697 |
| 36698 | 37698 | 38698 | 39698 |
| 36699 | 37699 | 38699 | 39699 |
| 36700 | 37700 | 38700 | 39700 |
| 36701 | 37701 | 38701 | 39701 |
| 36702 | 37702 | 38702 | 39702 |
| 36703 | 37703 | 38703 | 39703 |
| 36704 | 37704 | 38704 | 39704 |
| 36705 | 37705 | 38705 | 39705 |
| 36706 | 37706 | 38706 | 39706 |
| 36707 | 37707 | 38707 | 39707 |
| 36708 | 37708 | 38708 | 39708 |
| 36709 | 37709 | 38709 | 39709 |
| 36710 | 37710 | 38710 | 39710 |
| 36711 | 37711 | 38711 | 39711 |
| 36712 | 37712 | 38712 | 39712 |
| 36713 | 37713 | 38713 | 39713 |
| 36714 | 37714 | 38714 | 39714 |
| 36715 | 37715 | 38715 | 39715 |
| 36716 | 37716 | 38716 | 39716 |
| 36717 | 37717 | 38717 | 39717 |
| 36718 | 37718 | 38718 | 39718 |
| 36719 | 37719 | 38719 | 39719 |
| 36720 | 37720 | 38720 | 39720 |
| 36721 | 37721 | 38721 | 39721 |
| 36722 | 37722 | 38722 | 39722 |
| 36723 | 37723 | 38723 | 39723 |
| 36724 | 37724 | 38724 | 39724 |
| 36725 | 37725 | 38725 | 39725 |
| 36726 | 37726 | 38726 | 39726 |
| 36727 | 37727 | 38727 | 39727 |
| 36728 | 37728 | 38728 | 39728 |
| 36729 | 37729 | 38729 | 39729 |
| 36730 | 37730 | 38730 | 39730 |
| 36731 | 37731 | 38731 | 39731 |
| 36732 | 37732 | 38732 | 39732 |
| 36733 | 37733 | 38733 | 39733 |
| 36734 | 37734 | 38734 | 39734 |
| 36735 | 37735 | 38735 | 39735 |
| 36736 | 37736 | 38736 | 39736 |
| 36737 | 37737 | 38737 | 39737 |
| 36738 | 37738 | 38738 | 39738 |
| 36739 | 37739 | 38739 | 39739 |
| 36740 | 37740 | 38740 | 39740 |
| 36741 | 37741 | 38741 | 39741 |
| 36742 | 37742 | 38742 | 39742 |
| 36743 | 37743 | 38743 | 39743 |
| 36744 | 37744 | 38744 | 39744 |
| 36745 | 37745 | 38745 | 39745 |
| 36746 | 37746 | 38746 | 39746 |
| 36747 | 37747 | 38747 | 39747 |
| 36748 | 37748 | 38748 | 39748 |
| 36749 | 37749 | 38749 | 39749 |
| 36750 | 37750 | 38750 | 39750 |
| 36751 | 37751 | 38751 | 39751 |
| 36752 | 37752 | 38752 | 39752 |
| 36753 | 37753 | 38753 | 39753 |
| 36754 | 37754 | 38754 | 39754 |
| 36755 | 37755 | 38755 | 39755 |
| 36756 | 37756 | 38756 | 39756 |
| 36757 | 37757 | 38757 | 39757 |
| 36758 | 37758 | 38758 | 39758 |
| 36759 | 37759 | 38759 | 39759 |
| 36760 | 37760 | 38760 | 39760 |
| 36761 | 37761 | 38761 | 39761 |
| 36762 | 37762 | 38762 | 39762 |
| 36763 | 37763 | 38763 | 39763 |
| 36764 | 37764 | 38764 | 39764 |
| 36765 | 37765 | 38765 | 39765 |
| 36766 | 37766 | 38766 | 39766 |
| 36767 | 37767 | 38767 | 39767 |
| 36768 | 37768 | 38768 | 39768 |
| 36769 | 37769 | 38769 | 39769 |
| 36770 | 37770 | 38770 | 39770 |
| 36771 | 37771 | 38771 | 39771 |
| 36772 | 37772 | 38772 | 39772 |
| 36773 | 37773 | 38773 | 39773 |
| 36774 | 37774 | 38774 | 39774 |
| 36775 | 37775 | 38775 | 39775 |
| 36776 | 37776 | 38776 | 39776 |
| 36777 | 37777 | 38777 | 39777 |
| 36778 | 37778 | 38778 | 39778 |
| 36779 | 37779 | 38779 | 39779 |
| 36780 | 37780 | 38780 | 39780 |
| 36781 | 37781 | 38781 | 39781 |
| 36782 | 37782 | 38782 | 39782 |
| 36783 | 37783 | 38783 | 39783 |
| 36784 | 37784 | 38784 | 39784 |
| 36785 | 37785 | 38785 | 39785 |
| 36786 | 37786 | 38786 | 39786 |
| 36787 | 37787 | 38787 | 39787 |
| 36788 | 37788 | 38788 | 39788 |
| 36789 | 37789 | 38789 | 39789 |
| 36790 | 37790 | 38790 | 39790 |
| 36791 | 37791 | 38791 | 39791 |
| 36792 | 37792 | 38792 | 39792 |
| 36793 | 37793 | 38793 | 39793 |
| 36794 | 37794 | 38794 | 39794 |
| 36795 | 37795 | 38795 | 39795 |
| 36796 | 37796 | 38796 | 39796 |
| 36797 | 37797 | 38797 | 39797 |
| 36798 | 37798 | 38798 | 39798 |
| 36799 | 37799 | 38799 | 39799 |
| 36800 | 37800 | 38800 | 39800 |
| 36801 | 37801 | 38801 | 39801 |
| 36802 | 37802 | 38802 | 39802 |
| 36803 | 37803 | 38803 | 39803 |
| 36804 | 37804 | 38804 | 39804 |
| 36805 | 37805 | 38805 | 39805 |
| 36806 | 37806 | 38806 | 39806 |
| 36807 | 37807 | 38807 | 39807 |
| 36808 | 37808 | 38808 | 39808 |
| 36809 | 37809 | 38809 | 39809 |
| 36810 | 37810 | 38810 | 39810 |
| 36811 | 37811 | 38811 | 39811 |
| 36812 | 37812 | 38812 | 39812 |
| 36813 | 37813 | 38813 | 39813 |
| 36814 | 37814 | 38814 | 39814 |
| 36815 | 37815 | 38815 | 39815 |
| 36816 | 37816 | 38816 | 39816 |
| 36817 | 37817 | 38817 | 39817 |
| 36818 | 37818 | 38818 | 39818 |
| 36819 | 37819 | 38819 | 39819 |
| 36820 | 37820 | 38820 | 39820 |
| 36821 | 37821 | 38821 | 39821 |
| 36822 | 37822 | 38822 | 39822 |
| 36823 | 37823 | 38823 | 39823 |
| 36824 | 37824 | 38824 | 39824 |
| 36825 | 37825 | 38825 | 39825 |
| 36826 | 37826 | 38826 | 39826 |
| 36827 | 37827 | 38827 | 39827 |
| 36828 | 37828 | 38828 | 39828 |
| 36829 | 37829 | 38829 | 39829 |
| 36830 | 37830 | 38830 | 39830 |
| 36831 | 37831 | 38831 | 39831 |
| 36832 | 37832 | 38832 | 39832 |
| 36833 | 37833 | 38833 | 39833 |
| 36834 | 37834 | 38834 | 39834 |
| 36835 | 37835 | 38835 | 39835 |
| 36836 | 37836 | 38836 | 39836 |
| 36837 | 37837 | 38837 | 39837 |
| 36838 | 37838 | 38838 | 39838 |
| 36839 | 37839 | 38839 | 39839 |
| 36840 | 37840 | 38840 | 39840 |
| 36841 | 37841 | 38841 | 39841 |
| 36842 | 37842 | 38842 | 39842 |
| 36843 | 37843 | 38843 | 39843 |
| 36844 | 37844 | 38844 | 39844 |
| 36845 | 37845 | 38845 | 39845 |
| 36846 | 37846 | 38846 | 39846 |
| 36847 | 37847 | 38847 | 39847 |
| 36848 | 37848 | 38848 | 39848 |
| 36849 | 37849 | 38849 | 39849 |
| 36850 | 37850 | 38850 | 39850 |
| 36851 | 37851 | 38851 | 39851 |
| 36852 | 37852 | 38852 | 39852 |
| 36853 | 37853 | 38853 | 39853 |
| 36854 | 37854 | 38854 | 39854 |
| 36855 | 37855 | 38855 | 39855 |
| 36856 | 37856 | 38856 | 39856 |
| 36857 | 37857 | 38857 | 39857 |
| 36858 | 37858 | 38858 | 39858 |
| 36859 | 37859 | 38859 | 39859 |
| 36860 | 37860 | 38860 | 39860 |
| 36861 | 37861 | 38861 | 39861 |
| 36862 | 37862 | 38862 | 39862 |
| 36863 | 37863 | 38863 | 39863 |
| 36864 | 37864 | 38864 | 39864 |
| 36865 | 37865 | 38865 | 39865 |
| 36866 | 37866 | 38866 | 39866 |
| 36867 | 37867 | 38867 | 39867 |
| 36868 | 37868 | 38868 | 39868 |
| 36869 | 37869 | 38869 | 39869 |
| 36870 | 37870 | 38870 | 39870 |
| 36871 | 37871 | 38871 | 39871 |
| 36872 | 37872 | 38872 | 39872 |
| 36873 | 37873 | 38873 | 39873 |
| 36874 | 37874 | 38874 | 39874 |
| 36875 | 37875 | 38875 | 39875 |
| 36876 | 37876 | 38876 | 39876 |
| 36877 | 37877 | 38877 | 39877 |
| 36878 | 37878 | 38878 | 39878 |
| 36879 | 37879 | 38879 | 39879 |
| 36880 | 37880 | 38880 | 39880 |
| 36881 | 37881 | 38881 | 39881 |
| 36882 | 37882 | 38882 | 39882 |
| 36883 | 37883 | 38883 | 39883 |
| 36884 | 37884 | 38884 | 39884 |
| 36885 | 37885 | 38885 | 39885 |
| 36886 | 37886 | 38886 | 39886 |
| 36887 | 37887 | 38887 | 39887 |
| 36888 | 37888 | 38888 | 39888 |
| 36889 | 37889 | 38889 | 39889 |
| 36890 | 37890 | 38890 | 39890 |
| 36891 | 37891 | 38891 | 39891 |
| 36892 | 37892 | 38892 | 39892 |
| 36893 | 37893 | 38893 | 39893 |
| 36894 | 37894 | 38894 | 39894 |
| 36895 | 37895 | 38895 | 39895 |
| 36896 | 37896 | 38896 | 39896 |
| 36897 | 37897 | 38897 | 39897 |
| 36898 | 37898 | 38898 | 39898 |
| 36899 | 37899 | 38899 | 39899 |
| 36900 | 37900 | 38900 | 39900 |
| 36901 | 37901 | 38901 | 39901 |
| 36902 | 37902 | 38902 | 39902 |
| 36903 | 37903 | 38903 | 39903 |
| 36904 | 37904 | 38904 | 39904 |
| 36905 | 37905 | 38905 | 39905 |
| 36906 | 37906 | 38906 | 39906 |
| 36907 | 37907 | 38907 | 39907 |
| 36908 | 37908 | 38908 | 39908 |
| 36909 | 37909 | 38909 | 39909 |
| 36910 | 37910 | 38910 | 39910 |
| 36911 | 37911 | 38911 | 39911 |
| 36912 | 37912 | 38912 | 39912 |
| 36913 | 37913 | 38913 | 39913 |
| 36914 | 37914 | 38914 | 39914 |
| 36915 | 37915 | 38915 | 39915 |
| 36916 | 37916 | 38916 | 39916 |
| 36917 | 37917 | 38917 | 39917 |
| 36918 | 37918 | 38918 | 39918 |
| 36919 | 37919 | 38919 | 39919 |
| 36920 | 37920 | 38920 | 39920 |
| 36921 | 37921 | 38921 | 39921 |
| 36922 | 37922 | 38922 | 39922 |
| 36923 | 37923 | 38923 | 39923 |
| 36924 | 37924 | 38924 | 39924 |
| 36925 | 37925 | 38925 | 39925 |
| 36926 | 37926 | 38926 | 39926 |
| 36927 | 37927 | 38927 | 39927 |
| 36928 | 37928 | 38928 | 39928 |
| 36929 | 37929 | 38929 | 39929 |
| 36930 | 37930 | 38930 | 39930 |
| 36931 | 37931 | 38931 | 39931 |
| 36932 | 37932 | 38932 | 39932 |
| 36933 | 37933 | 38933 | 39933 |
| 36934 | 37934 | 38934 | 39934 |
| 36935 | 37935 | 38935 | 39935 |
| 36936 | 37936 | 38936 | 39936 |
| 36937 | 37937 | 38937 | 39937 |
| 36938 | 37938 | 38938 | 39938 |
| 36939 | 37939 | 38939 | 39939 |
| 36940 | 37940 | 38940 | 39940 |
| 36941 | 37941 | 38941 | 39941 |
| 36942 | 37942 | 38942 | 39942 |
| 36943 | 37943 | 38943 | 39943 |
| 36944 | 37944 | 38944 | 39944 |
| 36945 | 37945 | 38945 | 39945 |
| 36946 | 37946 | 38946 | 39946 |
| 36947 | 37947 | 38947 | 39947 |
| 36948 | 37948 | 38948 | 39948 |
| 36949 | 37949 | 38949 | 39949 |
| 36950 | 37950 | 38950 | 39950 |
| 36951 | 37951 | 38951 | 39951 |
| 36952 | 37952 | 38952 | 39952 |
| 36953 | 37953 | 38953 | 39953 |
| 36954 | 37954 | 38954 | 39954 |
| 36955 | 37955 | 38955 | 39955 |
| 36956 | 37956 | 38956 | 39956 |
| 36957 | 37957 | 38957 | 39957 |
| 36958 | 37958 | 38958 | 39958 |
| 36959 | 37959 | 38959 | 39959 |
| 36960 | 37960 | 38960 | 39960 |
| 36961 | 37961 | 38961 | 39961 |
| 36962 | 37962 | 38962 | 39962 |
| 36963 | 37963 | 38963 | 39963 |
| 36964 | 37964 | 38964 | 39964 |
| 36965 | 37965 | 38965 | 39965 |
| 36966 | 37966 | 38966 | 39966 |
| 36967 | 37967 | 38967 | 39967 |
| 36968 | 37968 | 38968 | 39968 |
| 36969 | 37969 | 38969 | 39969 |
| 36970 | 37970 | 38970 | 39970 |
| 36971 | 37971 | 38971 | 39971 |
| 36972 | 37972 | 38972 | 39972 |
| 36973 | 37973 | 38973 | 39973 |
| 36974 | 37974 | 38974 | 39974 |
| 36975 | 37975 | 38975 | 39975 |
| 36976 | 37976 | 38976 | 39976 |
| 36977 | 37977 | 38977 | 39977 |
| 36978 | 37978 | 38978 | 39978 |
| 36979 | 37979 | 38979 | 39979 |
| 36980 | 37980 | 38980 | 39980 |
| 36981 | 37981 | 38981 | 39981 |
| 36982 | 37982 | 38982 | 39982 |
| 36983 | 37983 | 38983 | 39983 |
| 36984 | 37984 | 38984 | 39984 |
| 36985 | 37985 | 38985 | 39985 |
| 36986 | 37986 | 38986 | 39986 |
| 36987 | 37987 | 38987 | 39987 |
| 36988 | 37988 | 38988 | 39988 |
| 36989 | 37989 | 38989 | 39989 |
| 36990 | 37990 | 38990 | 39990 |
| 36991 | 37991 | 38991 | 39991 |
| 36992 | 37992 | 38992 | 39992 |
| 36993 | 37993 | 38993 | 39993 |
| 36994 | 37994 | 38994 | 39994 |
| 36995 | 37995 | 38995 | 39995 |
| 36996 | 37996 | 38996 | 39996 |
| 36997 | 37997 | 38997 | 39997 |
| 36998 | 37998 | 38998 | 39998 |
| 36999 | 37999 | 38999 | 39999 |
| 37000 | 38000 | 39000 | 40000 |
| 37001 | 38001 | 39001 | 40001 |
| 37002 | 38002 | 39002 | 40002 |

In some embodiments, the sequences that specifically bind to gluten comprise an inner sequence selected from the group consisting of the nucleic acid sequences of SEQ ID NOs. 40003 to 41002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to gluten may comprise a nucleic acid sequence selected from the group consisting of SEQ ID NOs. 41003 to 42002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to gluten may comprise a nucleic acid sequence selected from the group consisting of SEQ ID NOs. 42003 to 43002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to gluten may comprise a nucleic acid sequence selected from the group consisting of SEQ ID NOs. 43003 to 44002. In one embodiment, the aptamer of the present disclosure that specifically binds to gluten may comprise a nucleic acid sequence selected from the group consisting of SEQ ID NOs. 40003 to 44002 listed in Table 12, or variant thereof.

TABLE 12
Aptamer sequences against gluten
Aptamer sequence

| | | | 5′-sequence |
|---|---|---|---|
| | 5′-sequence | Inner | and inner |
| Inner | and inner | sequence and | sequence and |
| Sequence | sequence | 3′-sequence | 3′-sequence |
| (SEQ ID NO.) | (SEQ ID NO.) | (SEQ ID NO.) | (SEQ ID NO.) |
| 40003 | 41003 | 42003 | 43003 |
| 40004 | 41004 | 42004 | 43004 |
| 40005 | 41005 | 42005 | 43005 |
| 40006 | 41006 | 42006 | 43006 |
| 40007 | 41007 | 42007 | 43007 |
| 40008 | 41008 | 42008 | 43008 |
| 40009 | 41009 | 42009 | 43009 |
| 40010 | 41010 | 42010 | 43010 |
| 40011 | 41011 | 42011 | 43011 |
| 40012 | 41012 | 42012 | 43012 |
| 40013 | 41013 | 42013 | 43013 |
| 40014 | 41014 | 42014 | 43014 |
| 40015 | 41015 | 42015 | 43015 |
| 40016 | 41016 | 42016 | 43016 |
| 40017 | 41017 | 42017 | 43017 |
| 40018 | 41018 | 42018 | 43018 |
| 40019 | 41019 | 42019 | 43019 |
| 40020 | 41020 | 42020 | 43020 |
| 40021 | 41021 | 42021 | 43021 |
| 40022 | 41022 | 42022 | 43022 |
| 40023 | 41023 | 42023 | 43023 |
| 40024 | 41024 | 42024 | 43024 |
| 40025 | 41025 | 42025 | 43025 |
| 40026 | 41026 | 42026 | 43026 |
| 40027 | 41027 | 42027 | 43027 |
| 40028 | 41028 | 42028 | 43028 |
| 40029 | 41029 | 42029 | 43029 |
| 40030 | 41030 | 42030 | 43030 |
| 40031 | 41031 | 42031 | 43031 |
| 40032 | 41032 | 42032 | 43032 |
| 40033 | 41033 | 42033 | 43033 |
| 40034 | 41034 | 42034 | 43034 |
| 40035 | 41035 | 42035 | 43035 |
| 40036 | 41036 | 42036 | 43036 |
| 40037 | 41037 | 42037 | 43037 |
| 40038 | 41038 | 42038 | 43038 |
| 40039 | 41039 | 42039 | 43039 |
| 40040 | 41040 | 42040 | 43040 |
| 40041 | 41041 | 42041 | 43041 |
| 40042 | 41042 | 42042 | 43042 |
| 40043 | 41043 | 42043 | 43043 |
| 40044 | 41044 | 42044 | 43044 |
| 40045 | 41045 | 42045 | 43045 |
| 40046 | 41046 | 42046 | 43046 |
| 40047 | 41047 | 42047 | 43047 |
| 40048 | 41048 | 42048 | 43048 |
| 40049 | 41049 | 42049 | 43049 |
| 40050 | 41050 | 42050 | 43050 |
| 40051 | 41051 | 42051 | 43051 |
| 40052 | 41052 | 42052 | 43052 |
| 40053 | 41053 | 42053 | 43053 |
| 40054 | 41054 | 42054 | 43054 |
| 40055 | 41055 | 42055 | 43055 |
| 40056 | 41056 | 42056 | 43056 |
| 40057 | 41057 | 42057 | 43057 |
| 40058 | 41058 | 42058 | 43058 |
| 40059 | 41059 | 42059 | 43059 |
| 40060 | 41060 | 42060 | 43060 |
| 40061 | 41061 | 42061 | 43061 |
| 40062 | 41062 | 42062 | 43062 |
| 40063 | 41063 | 42063 | 43063 |
| 40064 | 41064 | 42064 | 43064 |
| 40065 | 41065 | 42065 | 43065 |
| 40066 | 41066 | 42066 | 43066 |
| 40067 | 41067 | 42067 | 43067 |
| 40068 | 41068 | 42068 | 43068 |
| 40069 | 41069 | 42069 | 43069 |
| 40070 | 41070 | 42070 | 43070 |
| 40071 | 41071 | 42071 | 43071 |
| 40072 | 41072 | 42072 | 43072 |
| 40073 | 41073 | 42073 | 43073 |
| 40074 | 41074 | 42074 | 43074 |
| 40075 | 41075 | 42075 | 43075 |
| 40076 | 41076 | 42076 | 43076 |
| 40077 | 41077 | 42077 | 43077 |
| 40078 | 41078 | 42078 | 43078 |
| 40079 | 41079 | 42079 | 43079 |
| 40080 | 41080 | 42080 | 43080 |
| 40081 | 41081 | 42081 | 43081 |
| 40082 | 41082 | 42082 | 43082 |
| 40083 | 41083 | 42083 | 43083 |
| 40084 | 41084 | 42084 | 43084 |
| 40085 | 41085 | 42085 | 43085 |
| 40086 | 41086 | 42086 | 43086 |
| 40087 | 41087 | 42087 | 43087 |
| 40088 | 41088 | 42088 | 43088 |
| 40089 | 41089 | 42089 | 43089 |
| 40090 | 41090 | 42090 | 43090 |
| 40091 | 41091 | 42091 | 43091 |
| 40092 | 41092 | 42092 | 43092 |
| 40093 | 41093 | 42093 | 43093 |
| 40094 | 41094 | 42094 | 43094 |
| 40095 | 41095 | 42095 | 43095 |
| 40096 | 41096 | 42096 | 43096 |
| 40097 | 41097 | 42097 | 43097 |
| 40098 | 41098 | 42098 | 43098 |
| 40099 | 41099 | 42099 | 43099 |
| 40100 | 41100 | 42100 | 43100 |
| 40101 | 41101 | 42101 | 43101 |
| 40102 | 41102 | 42102 | 43102 |
| 40103 | 41103 | 42103 | 43103 |
| 40104 | 41104 | 42104 | 43104 |
| 40105 | 41105 | 42105 | 43105 |
| 40106 | 41106 | 42106 | 43106 |
| 40107 | 41107 | 42107 | 43107 |
| 40108 | 41108 | 42108 | 43108 |
| 40109 | 41109 | 42109 | 43109 |
| 40110 | 41110 | 42110 | 43110 |
| 40111 | 41111 | 42111 | 43111 |
| 40112 | 41112 | 42112 | 43112 |
| 40113 | 41113 | 42113 | 43113 |
| 40114 | 41114 | 42114 | 43114 |
| 40115 | 41115 | 42115 | 43115 |
| 40116 | 41116 | 42116 | 43116 |
| 40117 | 41117 | 42117 | 43117 |
| 40118 | 41118 | 42118 | 43118 |
| 40119 | 41119 | 42119 | 43119 |
| 40120 | 41120 | 42120 | 43120 |
| 40121 | 41121 | 42121 | 43121 |
| 40122 | 41122 | 42122 | 43122 |
| 40123 | 41123 | 42123 | 43123 |
| 40124 | 41124 | 42124 | 43124 |
| 40125 | 41125 | 42125 | 43125 |
| 40126 | 41126 | 42126 | 43126 |
| 40127 | 41127 | 42127 | 43127 |
| 40128 | 41128 | 42128 | 43128 |
| 40129 | 41129 | 42129 | 43129 |
| 40130 | 41130 | 42130 | 43130 |
| 40131 | 41131 | 42131 | 43131 |
| 40132 | 41132 | 42132 | 43132 |
| 40133 | 41133 | 42133 | 43133 |
| 40134 | 41134 | 42134 | 43134 |
| 40135 | 41135 | 42135 | 43135 |
| 40136 | 41136 | 42136 | 43136 |
| 40137 | 41137 | 42137 | 43137 |
| 40138 | 41138 | 42138 | 43138 |
| 40139 | 41139 | 42139 | 43139 |
| 40140 | 41140 | 42140 | 43140 |
| 40141 | 41141 | 42141 | 43141 |
| 40142 | 41142 | 42142 | 43142 |
| 40143 | 41143 | 42143 | 43143 |
| 40144 | 41144 | 42144 | 43144 |
| 40145 | 41145 | 42145 | 43145 |
| 40146 | 41146 | 42146 | 43146 |
| 40147 | 41147 | 42147 | 43147 |
| 40148 | 41148 | 42148 | 43148 |
| 40149 | 41149 | 42149 | 43149 |
| 40150 | 41150 | 42150 | 43150 |
| 40151 | 41151 | 42151 | 43151 |
| 40152 | 41152 | 42152 | 43152 |
| 40153 | 41153 | 42153 | 43153 |
| 40154 | 41154 | 42154 | 43154 |
| 40155 | 41155 | 42155 | 43155 |
| 40156 | 41156 | 42156 | 43156 |
| 40157 | 41157 | 42157 | 43157 |
| 40158 | 41158 | 42158 | 43158 |
| 40159 | 41159 | 42159 | 43159 |
| 40160 | 41160 | 42160 | 43160 |
| 40161 | 41161 | 42161 | 43161 |
| 40162 | 41162 | 42162 | 43162 |
| 40163 | 41163 | 42163 | 43163 |
| 40164 | 41164 | 42164 | 43164 |
| 40165 | 41165 | 42165 | 43165 |
| 40166 | 41166 | 42166 | 43166 |
| 40167 | 41167 | 42167 | 43167 |
| 40168 | 41168 | 42168 | 43168 |
| 40169 | 41169 | 42169 | 43169 |
| 40170 | 41170 | 42170 | 43170 |
| 40171 | 41171 | 42171 | 43171 |
| 40172 | 41172 | 42172 | 43172 |
| 40173 | 41173 | 42173 | 43173 |
| 40174 | 41174 | 42174 | 43174 |
| 40175 | 41175 | 42175 | 43175 |
| 40176 | 41176 | 42176 | 43176 |
| 40177 | 41177 | 42177 | 43177 |
| 40178 | 41178 | 42178 | 43178 |
| 40179 | 41179 | 42179 | 43179 |
| 40180 | 41180 | 42180 | 43180 |
| 40181 | 41181 | 42181 | 43181 |
| 40182 | 41182 | 42182 | 43182 |
| 40183 | 41183 | 42183 | 43183 |
| 40184 | 41184 | 42184 | 43184 |
| 40185 | 41185 | 42185 | 43185 |
| 40186 | 41186 | 42186 | 43186 |
| 40187 | 41187 | 42187 | 43187 |
| 40188 | 41188 | 42188 | 43188 |
| 40189 | 41189 | 42189 | 43189 |
| 40190 | 41190 | 42190 | 43190 |
| 40191 | 41191 | 42191 | 43191 |
| 40192 | 41192 | 42192 | 43192 |
| 40193 | 41193 | 42193 | 43193 |
| 40194 | 41194 | 42194 | 43194 |
| 40195 | 41195 | 42195 | 43195 |
| 40196 | 41196 | 42196 | 43196 |
| 40197 | 41197 | 42197 | 43197 |
| 40198 | 41198 | 42198 | 43198 |
| 40199 | 41199 | 42199 | 43199 |
| 40200 | 41200 | 42200 | 43200 |
| 40201 | 41201 | 42201 | 43201 |
| 40202 | 41202 | 42202 | 43202 |
| 40203 | 41203 | 42203 | 43203 |
| 40204 | 41204 | 42204 | 43204 |
| 40205 | 41205 | 42205 | 43205 |
| 40206 | 41206 | 42206 | 43206 |
| 40207 | 41207 | 42207 | 43207 |
| 40208 | 41208 | 42208 | 43208 |
| 40209 | 41209 | 42209 | 43209 |
| 40210 | 41210 | 42210 | 43210 |
| 40211 | 41211 | 42211 | 43211 |
| 40212 | 41212 | 42212 | 43212 |
| 40213 | 41213 | 42213 | 43213 |
| 40214 | 41214 | 42214 | 43214 |
| 40215 | 41215 | 42215 | 43215 |
| 40216 | 41216 | 42216 | 43216 |
| 40217 | 41217 | 42217 | 43217 |
| 40218 | 41218 | 42218 | 43218 |
| 40219 | 41219 | 42219 | 43219 |
| 40220 | 41220 | 42220 | 43220 |
| 40221 | 41221 | 42221 | 43221 |
| 40222 | 41222 | 42222 | 43222 |
| 40223 | 41223 | 42223 | 43223 |
| 40224 | 41224 | 42224 | 43224 |
| 40225 | 41225 | 42225 | 43225 |
| 40226 | 41226 | 42226 | 43226 |
| 40227 | 41227 | 42227 | 43227 |
| 40228 | 41228 | 42228 | 43228 |
| 40229 | 41229 | 42229 | 43229 |
| 40230 | 41230 | 42230 | 43230 |
| 40231 | 41231 | 42231 | 43231 |
| 40232 | 41232 | 42232 | 43232 |
| 40233 | 41233 | 42233 | 43233 |
| 40234 | 41234 | 42234 | 43234 |
| 40235 | 41235 | 42235 | 43235 |
| 40236 | 41236 | 42236 | 43236 |
| 40237 | 41237 | 42237 | 43237 |
| 40238 | 41238 | 42238 | 43238 |
| 40239 | 41239 | 42239 | 43239 |
| 40240 | 41240 | 42240 | 43240 |
| 40241 | 41241 | 42241 | 43241 |
| 40242 | 41242 | 42242 | 43242 |
| 40243 | 41243 | 42243 | 43243 |
| 40244 | 41244 | 42244 | 43244 |
| 40245 | 41245 | 42245 | 43245 |
| 40246 | 41246 | 42246 | 43246 |
| 40247 | 41247 | 42247 | 43247 |
| 40248 | 41248 | 42248 | 43248 |
| 40249 | 41249 | 42249 | 43249 |
| 40250 | 41250 | 42250 | 43250 |
| 40251 | 41251 | 42251 | 43251 |
| 40252 | 41252 | 42252 | 43252 |
| 40253 | 41253 | 42253 | 43253 |
| 40254 | 41254 | 42254 | 43254 |
| 40255 | 41255 | 42255 | 43255 |
| 40256 | 41256 | 42256 | 43256 |
| 40257 | 41257 | 42257 | 43257 |
| 40258 | 41258 | 42258 | 43258 |
| 40259 | 41259 | 42259 | 43259 |
| 40260 | 41260 | 42260 | 43260 |
| 40261 | 41261 | 42261 | 43261 |
| 40262 | 41262 | 42262 | 43262 |
| 40263 | 41263 | 42263 | 43263 |
| 40264 | 41264 | 42264 | 43264 |
| 40265 | 41265 | 42265 | 43265 |
| 40266 | 41266 | 42266 | 43266 |
| 40267 | 41267 | 42267 | 43267 |
| 40268 | 41268 | 42268 | 43268 |
| 40269 | 41269 | 42269 | 43269 |
| 40270 | 41270 | 42270 | 43270 |
| 40271 | 41271 | 42271 | 43271 |
| 40272 | 41272 | 42272 | 43272 |
| 40273 | 41273 | 42273 | 43273 |
| 40274 | 41274 | 42274 | 43274 |
| 40275 | 41275 | 42275 | 43275 |
| 40276 | 41276 | 42276 | 43276 |
| 40277 | 41277 | 42277 | 43277 |
| 40278 | 41278 | 42278 | 43278 |
| 40279 | 41279 | 42279 | 43279 |
| 40280 | 41280 | 42280 | 43280 |
| 40281 | 41281 | 42281 | 43281 |
| 40282 | 41282 | 42282 | 43282 |
| 40283 | 41283 | 42283 | 43283 |
| 40284 | 41284 | 42284 | 43284 |
| 40285 | 41285 | 42285 | 43285 |
| 40286 | 41286 | 42286 | 43286 |
| 40287 | 41287 | 42287 | 43287 |
| 40288 | 41288 | 42288 | 43288 |
| 40289 | 41289 | 42289 | 43289 |
| 40290 | 41290 | 42290 | 43290 |
| 40291 | 41291 | 42291 | 43291 |
| 40292 | 41292 | 42292 | 43292 |
| 40293 | 41293 | 42293 | 43293 |
| 40294 | 41294 | 42294 | 43294 |
| 40295 | 41295 | 42295 | 43295 |
| 40296 | 41296 | 42296 | 43296 |
| 40297 | 41297 | 42297 | 43297 |
| 40298 | 41298 | 42298 | 43298 |
| 40299 | 41299 | 42299 | 43299 |
| 40300 | 41300 | 42300 | 43300 |
| 40301 | 41301 | 42301 | 43301 |
| 40302 | 41302 | 42302 | 43302 |
| 40303 | 41303 | 42303 | 43303 |
| 40304 | 41304 | 42304 | 43304 |
| 40305 | 41305 | 42305 | 43305 |
| 40306 | 41306 | 42306 | 43306 |
| 40307 | 41307 | 42307 | 43307 |
| 40308 | 41308 | 42308 | 43308 |
| 40309 | 41309 | 42309 | 43309 |
| 40310 | 41310 | 42310 | 43310 |
| 40311 | 41311 | 42311 | 43311 |
| 40312 | 41312 | 42312 | 43312 |
| 40313 | 41313 | 42313 | 43313 |
| 40314 | 41314 | 42314 | 43314 |
| 40315 | 41315 | 42315 | 43315 |
| 40316 | 41316 | 42316 | 43316 |
| 40317 | 41317 | 42317 | 43317 |
| 40318 | 41318 | 42318 | 43318 |
| 40319 | 41319 | 42319 | 43319 |
| 40320 | 41320 | 42320 | 43320 |
| 40321 | 41321 | 42321 | 43321 |
| 40322 | 41322 | 42322 | 43322 |
| 40323 | 41323 | 42323 | 43323 |
| 40324 | 41324 | 42324 | 43324 |
| 40325 | 41325 | 42325 | 43325 |
| 40326 | 41326 | 42326 | 43326 |
| 40327 | 41327 | 42327 | 43327 |
| 40328 | 41328 | 42328 | 43328 |
| 40329 | 41329 | 42329 | 43329 |
| 40330 | 41330 | 42330 | 43330 |
| 40331 | 41331 | 42331 | 43331 |
| 40332 | 41332 | 42332 | 43332 |
| 40333 | 41333 | 42333 | 43333 |
| 40334 | 41334 | 42334 | 43334 |
| 40335 | 41335 | 42335 | 43335 |
| 40336 | 41336 | 42336 | 43336 |
| 40337 | 41337 | 42337 | 43337 |
| 40338 | 41338 | 42338 | 43338 |
| 40339 | 41339 | 42339 | 43339 |
| 40340 | 41340 | 42340 | 43340 |
| 40341 | 41341 | 42341 | 43341 |
| 40342 | 41342 | 42342 | 43342 |
| 40343 | 41343 | 42343 | 43343 |
| 40344 | 41344 | 42344 | 43344 |
| 40345 | 41345 | 42345 | 43345 |
| 40346 | 41346 | 42346 | 43346 |
| 40347 | 41347 | 42347 | 43347 |
| 40348 | 41348 | 42348 | 43348 |
| 40349 | 41349 | 42349 | 43349 |
| 40350 | 41350 | 42350 | 43350 |
| 40351 | 41351 | 42351 | 43351 |
| 40352 | 41352 | 42352 | 43352 |
| 40353 | 41353 | 42353 | 43353 |
| 40354 | 41354 | 42354 | 43354 |
| 40355 | 41355 | 42355 | 43355 |
| 40356 | 41356 | 42356 | 43356 |
| 40357 | 41357 | 42357 | 43357 |
| 40358 | 41358 | 42358 | 43358 |
| 40359 | 41359 | 42359 | 43359 |
| 40360 | 41360 | 42360 | 43360 |
| 40361 | 41361 | 42361 | 43361 |
| 40362 | 41362 | 42362 | 43362 |
| 40363 | 41363 | 42363 | 43363 |
| 40364 | 41364 | 42364 | 43364 |
| 40365 | 41365 | 42365 | 43365 |
| 40366 | 41366 | 42366 | 43366 |
| 40367 | 41367 | 42367 | 43367 |
| 40368 | 41368 | 42368 | 43368 |
| 40369 | 41369 | 42369 | 43369 |
| 40370 | 41370 | 42370 | 43370 |
| 40371 | 41371 | 42371 | 43371 |
| 40372 | 41372 | 42372 | 43372 |
| 40373 | 41373 | 42373 | 43373 |
| 40374 | 41374 | 42374 | 43374 |
| 40375 | 41375 | 42375 | 43375 |
| 40376 | 41376 | 42376 | 43376 |
| 40377 | 41377 | 42377 | 43377 |
| 40378 | 41378 | 42378 | 43378 |
| 40379 | 41379 | 42379 | 43379 |
| 40380 | 41380 | 42380 | 43380 |
| 40381 | 41381 | 42381 | 43381 |
| 40382 | 41382 | 42382 | 43382 |
| 40383 | 41383 | 42383 | 43383 |
| 40384 | 41384 | 42384 | 43384 |
| 40385 | 41385 | 42385 | 43385 |
| 40386 | 41386 | 42386 | 43386 |
| 40387 | 41387 | 42387 | 43387 |
| 40388 | 41388 | 42388 | 43388 |
| 40389 | 41389 | 42389 | 43389 |
| 40390 | 41390 | 42390 | 43390 |
| 40391 | 41391 | 42391 | 43391 |
| 40392 | 41392 | 42392 | 43392 |
| 40393 | 41393 | 42393 | 43393 |
| 40394 | 41394 | 42394 | 43394 |
| 40395 | 41395 | 42395 | 43395 |
| 40396 | 41396 | 42396 | 43396 |
| 40397 | 41397 | 42397 | 43397 |
| 40398 | 41398 | 42398 | 43398 |
| 40399 | 41399 | 42399 | 43399 |
| 40400 | 41400 | 42400 | 43400 |
| 40401 | 41401 | 42401 | 43401 |
| 40402 | 41402 | 42402 | 43402 |
| 40403 | 41403 | 42403 | 43403 |
| 40404 | 41404 | 42404 | 43404 |
| 40405 | 41405 | 42405 | 43405 |
| 40406 | 41406 | 42406 | 43406 |
| 40407 | 41407 | 42407 | 43407 |
| 40408 | 41408 | 42408 | 43408 |
| 40409 | 41409 | 42409 | 43409 |
| 40410 | 41410 | 42410 | 43410 |
| 40411 | 41411 | 42411 | 43411 |
| 40412 | 41412 | 42412 | 43412 |
| 40413 | 41413 | 42413 | 43413 |
| 40414 | 41414 | 42414 | 43414 |
| 40415 | 41415 | 42415 | 43415 |
| 40416 | 41416 | 42416 | 43416 |
| 40417 | 41417 | 42417 | 43417 |
| 40418 | 41418 | 42418 | 43418 |
| 40419 | 41419 | 42419 | 43419 |
| 40420 | 41420 | 42420 | 43420 |
| 40421 | 41421 | 42421 | 43421 |
| 40422 | 41422 | 42422 | 43422 |
| 40423 | 41423 | 42423 | 43423 |
| 40424 | 41424 | 42424 | 43424 |
| 40425 | 41425 | 42425 | 43425 |
| 40426 | 41426 | 42426 | 43426 |
| 40427 | 41427 | 42427 | 43427 |
| 40428 | 41428 | 42428 | 43428 |
| 40429 | 41429 | 42429 | 43429 |
| 40430 | 41430 | 42430 | 43430 |
| 40431 | 41431 | 42431 | 43431 |
| 40432 | 41432 | 42432 | 43432 |
| 40433 | 41433 | 42433 | 43433 |
| 40434 | 41434 | 42434 | 43434 |
| 40435 | 41435 | 42435 | 43435 |
| 40436 | 41436 | 42436 | 43436 |
| 40437 | 41437 | 42437 | 43437 |
| 40438 | 41438 | 42438 | 43438 |
| 40439 | 41439 | 42439 | 43439 |
| 40440 | 41440 | 42440 | 43440 |
| 40441 | 41441 | 42441 | 43441 |
| 40442 | 41442 | 42442 | 43442 |
| 40443 | 41443 | 42443 | 43443 |
| 40444 | 41444 | 42444 | 43444 |
| 40445 | 41445 | 42445 | 43445 |
| 40446 | 41446 | 42446 | 43446 |
| 40447 | 41447 | 42447 | 43447 |
| 40448 | 41448 | 42448 | 43448 |
| 40449 | 41449 | 42449 | 43449 |
| 40450 | 41450 | 42450 | 43450 |
| 40451 | 41451 | 42451 | 43451 |
| 40452 | 41452 | 42452 | 43452 |
| 40453 | 41453 | 42453 | 43453 |
| 40454 | 41454 | 42454 | 43454 |
| 40455 | 41455 | 42455 | 43455 |
| 40456 | 41456 | 42456 | 43456 |
| 40457 | 41457 | 42457 | 43457 |
| 40458 | 41458 | 42458 | 43458 |
| 40459 | 41459 | 42459 | 43459 |
| 40460 | 41460 | 42460 | 43460 |
| 40461 | 41461 | 42461 | 43461 |
| 40462 | 41462 | 42462 | 43462 |
| 40463 | 41463 | 42463 | 43463 |
| 40464 | 41464 | 42464 | 43464 |
| 40465 | 41465 | 42465 | 43465 |
| 40466 | 41466 | 42466 | 43466 |
| 40467 | 41467 | 42467 | 43467 |
| 40468 | 41468 | 42468 | 43468 |
| 40469 | 41469 | 42469 | 43469 |
| 40470 | 41470 | 42470 | 43470 |
| 40471 | 41471 | 42471 | 43471 |
| 40472 | 41472 | 42472 | 43472 |
| 40473 | 41473 | 42473 | 43473 |
| 40474 | 41474 | 42474 | 43474 |
| 40475 | 41475 | 42475 | 43475 |
| 40476 | 41476 | 42476 | 43476 |
| 40477 | 41477 | 42477 | 43477 |
| 40478 | 41478 | 42478 | 43478 |
| 40479 | 41479 | 42479 | 43479 |
| 40480 | 41480 | 42480 | 43480 |
| 40481 | 41481 | 42481 | 43481 |
| 40482 | 41482 | 42482 | 43482 |
| 40483 | 41483 | 42483 | 43483 |
| 40484 | 41484 | 42484 | 43484 |
| 40485 | 41485 | 42485 | 43485 |
| 40486 | 41486 | 42486 | 43486 |
| 40487 | 41487 | 42487 | 43487 |
| 40488 | 41488 | 42488 | 43488 |
| 40489 | 41489 | 42489 | 43489 |
| 40490 | 41490 | 42490 | 43490 |
| 40491 | 41491 | 42491 | 43491 |
| 40492 | 41492 | 42492 | 43492 |
| 40493 | 41493 | 42493 | 43493 |
| 40494 | 41494 | 42494 | 43494 |
| 40495 | 41495 | 42495 | 43495 |
| 40496 | 41496 | 42496 | 43496 |
| 40497 | 41497 | 42497 | 43497 |
| 40498 | 41498 | 42498 | 43498 |
| 40499 | 41499 | 42499 | 43499 |
| 40500 | 41500 | 42500 | 43500 |
| 40501 | 41501 | 42501 | 43501 |
| 40502 | 41502 | 42502 | 43502 |
| 40503 | 41503 | 42503 | 43503 |
| 40504 | 41504 | 42504 | 43504 |
| 40505 | 41505 | 42505 | 43505 |
| 40506 | 41506 | 42506 | 43506 |
| 40507 | 41507 | 42507 | 43507 |
| 40508 | 41508 | 42508 | 43508 |
| 40509 | 41509 | 42509 | 43509 |
| 40510 | 41510 | 42510 | 43510 |
| 40511 | 41511 | 42511 | 43511 |
| 40512 | 41512 | 42512 | 43512 |
| 40513 | 41513 | 42513 | 43513 |
| 40514 | 41514 | 42514 | 43514 |
| 40515 | 41515 | 42515 | 43515 |
| 40516 | 41516 | 42516 | 43516 |
| 40517 | 41517 | 42517 | 43517 |
| 40518 | 41518 | 42518 | 43518 |
| 40519 | 41519 | 42519 | 43519 |
| 40520 | 41520 | 42520 | 43520 |
| 40521 | 41521 | 42521 | 43521 |
| 40522 | 41522 | 42522 | 43522 |
| 40523 | 41523 | 42523 | 43523 |
| 40524 | 41524 | 42524 | 43524 |
| 40525 | 41525 | 42525 | 43525 |
| 40526 | 41526 | 42526 | 43526 |
| 40527 | 41527 | 42527 | 43527 |
| 40528 | 41528 | 42528 | 43528 |
| 40529 | 41529 | 42529 | 43529 |
| 40530 | 41530 | 42530 | 43530 |
| 40531 | 41531 | 42531 | 43531 |
| 40532 | 41532 | 42532 | 43532 |
| 40533 | 41533 | 42533 | 43533 |
| 40534 | 41534 | 42534 | 43534 |
| 40535 | 41535 | 42535 | 43535 |
| 40536 | 41536 | 42536 | 43536 |
| 40537 | 41537 | 42537 | 43537 |
| 40538 | 41538 | 42538 | 43538 |
| 40539 | 41539 | 42539 | 43539 |
| 40540 | 41540 | 42540 | 43540 |
| 40541 | 41541 | 42541 | 43541 |
| 40542 | 41542 | 42542 | 43542 |
| 40543 | 41543 | 42543 | 43543 |
| 40544 | 41544 | 42544 | 43544 |
| 40545 | 41545 | 42545 | 43545 |
| 40546 | 41546 | 42546 | 43546 |
| 40547 | 41547 | 42547 | 43547 |
| 40548 | 41548 | 42548 | 43548 |
| 40549 | 41549 | 42549 | 43549 |
| 40550 | 41550 | 42550 | 43550 |
| 40551 | 41551 | 42551 | 43551 |
| 40552 | 41552 | 42552 | 43552 |
| 40553 | 41553 | 42553 | 43553 |
| 40554 | 41554 | 42554 | 43554 |
| 40555 | 41555 | 42555 | 43555 |
| 40556 | 41556 | 42556 | 43556 |
| 40557 | 41557 | 42557 | 43557 |
| 40558 | 41558 | 42558 | 43558 |
| 40559 | 41559 | 42559 | 43559 |
| 40560 | 41560 | 42560 | 43560 |
| 40561 | 41561 | 42561 | 43561 |
| 40562 | 41562 | 42562 | 43562 |
| 40563 | 41563 | 42563 | 43563 |
| 40564 | 41564 | 42564 | 43564 |
| 40565 | 41565 | 42565 | 43565 |
| 40566 | 41566 | 42566 | 43566 |
| 40567 | 41567 | 42567 | 43567 |
| 40568 | 41568 | 42568 | 43568 |
| 40569 | 41569 | 42569 | 43569 |
| 40570 | 41570 | 42570 | 43570 |
| 40571 | 41571 | 42571 | 43571 |
| 40572 | 41572 | 42572 | 43572 |
| 40573 | 41573 | 42573 | 43573 |
| 40574 | 41574 | 42574 | 43574 |
| 40575 | 41575 | 42575 | 43575 |
| 40576 | 41576 | 42576 | 43576 |
| 40577 | 41577 | 42577 | 43577 |
| 40578 | 41578 | 42578 | 43578 |
| 40579 | 41579 | 42579 | 43579 |
| 40580 | 41580 | 42580 | 43580 |
| 40581 | 41581 | 42581 | 43581 |
| 40582 | 41582 | 42582 | 43582 |
| 40583 | 41583 | 42583 | 43583 |
| 40584 | 41584 | 42584 | 43584 |
| 40585 | 41585 | 42585 | 43585 |
| 40586 | 41586 | 42586 | 43586 |
| 40587 | 41587 | 42587 | 43587 |
| 40588 | 41588 | 42588 | 43588 |
| 40589 | 41589 | 42589 | 43589 |
| 40590 | 41590 | 42590 | 43590 |
| 40591 | 41591 | 42591 | 43591 |
| 40592 | 41592 | 42592 | 43592 |
| 40593 | 41593 | 42593 | 43593 |
| 40594 | 41594 | 42594 | 43594 |
| 40595 | 41595 | 42595 | 43595 |
| 40596 | 41596 | 42596 | 43596 |
| 40597 | 41597 | 42597 | 43597 |
| 40598 | 41598 | 42598 | 43598 |
| 40599 | 41599 | 42599 | 43599 |
| 40600 | 41600 | 42600 | 43600 |
| 40601 | 41601 | 42601 | 43601 |
| 40602 | 41602 | 42602 | 43602 |
| 40603 | 41603 | 42603 | 43603 |
| 40604 | 41604 | 42604 | 43604 |
| 40605 | 41605 | 42605 | 43605 |
| 40606 | 41606 | 42606 | 43606 |
| 40607 | 41607 | 42607 | 43607 |
| 40608 | 41608 | 42608 | 43608 |
| 40609 | 41609 | 42609 | 43609 |
| 40610 | 41610 | 42610 | 43610 |
| 40611 | 41611 | 42611 | 43611 |
| 40612 | 41612 | 42612 | 43612 |
| 40613 | 41613 | 42613 | 43613 |
| 40614 | 41614 | 42614 | 43614 |
| 40615 | 41615 | 42615 | 43615 |
| 40616 | 41616 | 42616 | 43616 |
| 40617 | 41617 | 42617 | 43617 |
| 40618 | 41618 | 42618 | 43618 |
| 40619 | 41619 | 42619 | 43619 |
| 40620 | 41620 | 42620 | 43620 |
| 40621 | 41621 | 42621 | 43621 |
| 40622 | 41622 | 42622 | 43622 |
| 40623 | 41623 | 42623 | 43623 |
| 40624 | 41624 | 42624 | 43624 |
| 40625 | 41625 | 42625 | 43625 |
| 40626 | 41626 | 42626 | 43626 |
| 40627 | 41627 | 42627 | 43627 |
| 40628 | 41628 | 42628 | 43628 |
| 40629 | 41629 | 42629 | 43629 |
| 40630 | 41630 | 42630 | 43630 |
| 40631 | 41631 | 42631 | 43631 |
| 40632 | 41632 | 42632 | 43632 |
| 40633 | 41633 | 42633 | 43633 |
| 40634 | 41634 | 42634 | 43634 |
| 40635 | 41635 | 42635 | 43635 |
| 40636 | 41636 | 42636 | 43636 |
| 40637 | 41637 | 42637 | 43637 |
| 40638 | 41638 | 42638 | 43638 |
| 40639 | 41639 | 42639 | 43639 |
| 40640 | 41640 | 42640 | 43640 |
| 40641 | 41641 | 42641 | 43641 |
| 40642 | 41642 | 42642 | 43642 |
| 40643 | 41643 | 42643 | 43643 |
| 40644 | 41644 | 42644 | 43644 |
| 40645 | 41645 | 42645 | 43645 |
| 40646 | 41646 | 42646 | 43646 |
| 40647 | 41647 | 42647 | 43647 |
| 40648 | 41648 | 42648 | 43648 |
| 40649 | 41649 | 42649 | 43649 |
| 40650 | 41650 | 42650 | 43650 |
| 40651 | 41651 | 42651 | 43651 |
| 40652 | 41652 | 42652 | 43652 |
| 40653 | 41653 | 42653 | 43653 |
| 40654 | 41654 | 42654 | 43654 |
| 40655 | 41655 | 42655 | 43655 |
| 40656 | 41656 | 42656 | 43656 |
| 40657 | 41657 | 42657 | 43657 |
| 40658 | 41658 | 42658 | 43658 |
| 40659 | 41659 | 42659 | 43659 |
| 40660 | 41660 | 42660 | 43660 |
| 40661 | 41661 | 42661 | 43661 |
| 40662 | 41662 | 42662 | 43662 |
| 40663 | 41663 | 42663 | 43663 |
| 40664 | 41664 | 42664 | 43664 |
| 40665 | 41665 | 42665 | 43665 |
| 40666 | 41666 | 42666 | 43666 |
| 40667 | 41667 | 42667 | 43667 |
| 40668 | 41668 | 42668 | 43668 |
| 40669 | 41669 | 42669 | 43669 |
| 40670 | 41670 | 42670 | 43670 |
| 40671 | 41671 | 42671 | 43671 |
| 40672 | 41672 | 42672 | 43672 |
| 40673 | 41673 | 42673 | 43673 |
| 40674 | 41674 | 42674 | 43674 |
| 40675 | 41675 | 42675 | 43675 |
| 40676 | 41676 | 42676 | 43676 |
| 40677 | 41677 | 42677 | 43677 |
| 40678 | 41678 | 42678 | 43678 |
| 40679 | 41679 | 42679 | 43679 |
| 40680 | 41680 | 42680 | 43680 |
| 40681 | 41681 | 42681 | 43681 |
| 40682 | 41682 | 42682 | 43682 |
| 40683 | 41683 | 42683 | 43683 |
| 40684 | 41684 | 42684 | 43684 |
| 40685 | 41685 | 42685 | 43685 |
| 40686 | 41686 | 42686 | 43686 |
| 40687 | 41687 | 42687 | 43687 |
| 40688 | 41688 | 42688 | 43688 |
| 40689 | 41689 | 42689 | 43689 |
| 40690 | 41690 | 42690 | 43690 |
| 40691 | 41691 | 42691 | 43691 |
| 40692 | 41692 | 42692 | 43692 |
| 40693 | 41693 | 42693 | 43693 |
| 40694 | 41694 | 42694 | 43694 |
| 40695 | 41695 | 42695 | 43695 |
| 40696 | 41696 | 42696 | 43696 |
| 40697 | 41697 | 42697 | 43697 |
| 40698 | 41698 | 42698 | 43698 |
| 40699 | 41699 | 42699 | 43699 |
| 40700 | 41700 | 42700 | 43700 |
| 40701 | 41701 | 42701 | 43701 |
| 40702 | 41702 | 42702 | 43702 |
| 40703 | 41703 | 42703 | 43703 |
| 40704 | 41704 | 42704 | 43704 |
| 40705 | 41705 | 42705 | 43705 |
| 40706 | 41706 | 42706 | 43706 |
| 40707 | 41707 | 42707 | 43707 |
| 40708 | 41708 | 42708 | 43708 |
| 40709 | 41709 | 42709 | 43709 |
| 40710 | 41710 | 42710 | 43710 |
| 40711 | 41711 | 42711 | 43711 |
| 40712 | 41712 | 42712 | 43712 |
| 40713 | 41713 | 42713 | 43713 |
| 40714 | 41714 | 42714 | 43714 |
| 40715 | 41715 | 42715 | 43715 |
| 40716 | 41716 | 42716 | 43716 |
| 40717 | 41717 | 42717 | 43717 |
| 40718 | 41718 | 42718 | 43718 |
| 40719 | 41719 | 42719 | 43719 |
| 40720 | 41720 | 42720 | 43720 |
| 40721 | 41721 | 42721 | 43721 |
| 40722 | 41722 | 42722 | 43722 |
| 40723 | 41723 | 42723 | 43723 |
| 40724 | 41724 | 42724 | 43724 |
| 40725 | 41725 | 42725 | 43725 |
| 40726 | 41726 | 42726 | 43726 |
| 40727 | 41727 | 42727 | 43727 |
| 40728 | 41728 | 42728 | 43728 |
| 40729 | 41729 | 42729 | 43729 |
| 40730 | 41730 | 42730 | 43730 |
| 40731 | 41731 | 42731 | 43731 |
| 40732 | 41732 | 42732 | 43732 |
| 40733 | 41733 | 42733 | 43733 |
| 40734 | 41734 | 42734 | 43734 |
| 40735 | 41735 | 42735 | 43735 |
| 40736 | 41736 | 42736 | 43736 |
| 40737 | 41737 | 42737 | 43737 |
| 40738 | 41738 | 42738 | 43738 |
| 40739 | 41739 | 42739 | 43739 |
| 40740 | 41740 | 42740 | 43740 |
| 40741 | 41741 | 42741 | 43741 |
| 40742 | 41742 | 42742 | 43742 |
| 40743 | 41743 | 42743 | 43743 |
| 40744 | 41744 | 42744 | 43744 |
| 40745 | 41745 | 42745 | 43745 |
| 40746 | 41746 | 42746 | 43746 |
| 40747 | 41747 | 42747 | 43747 |
| 40748 | 41748 | 42748 | 43748 |
| 40749 | 41749 | 42749 | 43749 |
| 40750 | 41750 | 42750 | 43750 |
| 40751 | 41751 | 42751 | 43751 |
| 40752 | 41752 | 42752 | 43752 |
| 40753 | 41753 | 42753 | 43753 |
| 40754 | 41754 | 42754 | 43754 |
| 40755 | 41755 | 42755 | 43755 |
| 40756 | 41756 | 42756 | 43756 |
| 40757 | 41757 | 42757 | 43757 |
| 40758 | 41758 | 42758 | 43758 |
| 40759 | 41759 | 42759 | 43759 |
| 40760 | 41760 | 42760 | 43760 |
| 40761 | 41761 | 42761 | 43761 |
| 40762 | 41762 | 42762 | 43762 |
| 40763 | 41763 | 42763 | 43763 |
| 40764 | 41764 | 42764 | 43764 |
| 40765 | 41765 | 42765 | 43765 |
| 40766 | 41766 | 42766 | 43766 |
| 40767 | 41767 | 42767 | 43767 |
| 40768 | 41768 | 42768 | 43768 |
| 40769 | 41769 | 42769 | 43769 |
| 40770 | 41770 | 42770 | 43770 |
| 40771 | 41771 | 42771 | 43771 |
| 40772 | 41772 | 42772 | 43772 |
| 40773 | 41773 | 42773 | 43773 |
| 40774 | 41774 | 42774 | 43774 |
| 40775 | 41775 | 42775 | 43775 |
| 40776 | 41776 | 42776 | 43776 |
| 40777 | 41777 | 42777 | 43777 |
| 40778 | 41778 | 42778 | 43778 |
| 40779 | 41779 | 42779 | 43779 |
| 40780 | 41780 | 42780 | 43780 |
| 40781 | 41781 | 42781 | 43781 |
| 40782 | 41782 | 42782 | 43782 |
| 40783 | 41783 | 42783 | 43783 |
| 40784 | 41784 | 42784 | 43784 |
| 40785 | 41785 | 42785 | 43785 |
| 40786 | 41786 | 42786 | 43786 |
| 40787 | 41787 | 42787 | 43787 |
| 40788 | 41788 | 42788 | 43788 |
| 40789 | 41789 | 42789 | 43789 |
| 40790 | 41790 | 42790 | 43790 |
| 40791 | 41791 | 42791 | 43791 |
| 40792 | 41792 | 42792 | 43792 |
| 40793 | 41793 | 42793 | 43793 |
| 40794 | 41794 | 42794 | 43794 |
| 40795 | 41795 | 42795 | 43795 |
| 40796 | 41796 | 42796 | 43796 |
| 40797 | 41797 | 42797 | 43797 |
| 40798 | 41798 | 42798 | 43798 |
| 40799 | 41799 | 42799 | 43799 |
| 40800 | 41800 | 42800 | 43800 |
| 40801 | 41801 | 42801 | 43801 |
| 40802 | 41802 | 42802 | 43802 |
| 40803 | 41803 | 42803 | 43803 |
| 40804 | 41804 | 42804 | 43804 |
| 40805 | 41805 | 42805 | 43805 |
| 40806 | 41806 | 42806 | 43806 |
| 40807 | 41807 | 42807 | 43807 |
| 40808 | 41808 | 42808 | 43808 |
| 40809 | 41809 | 42809 | 43809 |
| 40810 | 41810 | 42810 | 43810 |
| 40811 | 41811 | 42811 | 43811 |
| 40812 | 41812 | 42812 | 43812 |
| 40813 | 41813 | 42813 | 43813 |
| 40814 | 41814 | 42814 | 43814 |
| 40815 | 41815 | 42815 | 43815 |
| 40816 | 41816 | 42816 | 43816 |
| 40817 | 41817 | 42817 | 43817 |
| 40818 | 41818 | 42818 | 43818 |
| 40819 | 41819 | 42819 | 43819 |
| 40820 | 41820 | 42820 | 43820 |
| 40821 | 41821 | 42821 | 43821 |
| 40822 | 41822 | 42822 | 43822 |
| 40823 | 41823 | 42823 | 43823 |
| 40824 | 41824 | 42824 | 43824 |
| 40825 | 41825 | 42825 | 43825 |
| 40826 | 41826 | 42826 | 43826 |
| 40827 | 41827 | 42827 | 43827 |
| 40828 | 41828 | 42828 | 43828 |
| 40829 | 41829 | 42829 | 43829 |
| 40830 | 41830 | 42830 | 43830 |
| 40831 | 41831 | 42831 | 43831 |
| 40832 | 41832 | 42832 | 43832 |
| 40833 | 41833 | 42833 | 43833 |
| 40834 | 41834 | 42834 | 43834 |
| 40835 | 41835 | 42835 | 43835 |
| 40836 | 41836 | 42836 | 43836 |
| 40837 | 41837 | 42837 | 43837 |
| 40838 | 41838 | 42838 | 43838 |
| 40839 | 41839 | 42839 | 43839 |
| 40840 | 41840 | 42840 | 43840 |
| 40841 | 41841 | 42841 | 43841 |
| 40842 | 41842 | 42842 | 43842 |
| 40843 | 41843 | 42843 | 43843 |
| 40844 | 41844 | 42844 | 43844 |
| 40845 | 41845 | 42845 | 43845 |
| 40846 | 41846 | 42846 | 43846 |
| 40847 | 41847 | 42847 | 43847 |
| 40848 | 41848 | 42848 | 43848 |
| 40849 | 41849 | 42849 | 43849 |
| 40850 | 41850 | 42850 | 43850 |
| 40851 | 41851 | 42851 | 43851 |
| 40852 | 41852 | 42852 | 43852 |
| 40853 | 41853 | 42853 | 43853 |
| 40854 | 41854 | 42854 | 43854 |
| 40855 | 41855 | 42855 | 43855 |
| 40856 | 41856 | 42856 | 43856 |
| 40857 | 41857 | 42857 | 43857 |
| 40858 | 41858 | 42858 | 43858 |
| 40859 | 41859 | 42859 | 43859 |
| 40860 | 41860 | 42860 | 43860 |
| 40861 | 41861 | 42861 | 43861 |
| 40862 | 41862 | 42862 | 43862 |
| 40863 | 41863 | 42863 | 43863 |
| 40864 | 41864 | 42864 | 43864 |
| 40865 | 41865 | 42865 | 43865 |
| 40866 | 41866 | 42866 | 43866 |
| 40867 | 41867 | 42867 | 43867 |
| 40868 | 41868 | 42868 | 43868 |
| 40869 | 41869 | 42869 | 43869 |
| 40870 | 41870 | 42870 | 43870 |
| 40871 | 41871 | 42871 | 43871 |
| 40872 | 41872 | 42872 | 43872 |
| 40873 | 41873 | 42873 | 43873 |
| 40874 | 41874 | 42874 | 43874 |
| 40875 | 41875 | 42875 | 43875 |
| 40876 | 41876 | 42876 | 43876 |
| 40877 | 41877 | 42877 | 43877 |
| 40878 | 41878 | 42878 | 43878 |
| 40879 | 41879 | 42879 | 43879 |
| 40880 | 41880 | 42880 | 43880 |
| 40881 | 41881 | 42881 | 43881 |
| 40882 | 41882 | 42882 | 43882 |
| 40883 | 41883 | 42883 | 43883 |
| 40884 | 41884 | 42884 | 43884 |
| 40885 | 41885 | 42885 | 43885 |
| 40886 | 41886 | 42886 | 43886 |
| 40887 | 41887 | 42887 | 43887 |
| 40888 | 41888 | 42888 | 43888 |
| 40889 | 41889 | 42889 | 43889 |
| 40890 | 41890 | 42890 | 43890 |
| 40891 | 41891 | 42891 | 43891 |
| 40892 | 41892 | 42892 | 43892 |
| 40893 | 41893 | 42893 | 43893 |
| 40894 | 41894 | 42894 | 43894 |
| 40895 | 41895 | 42895 | 43895 |
| 40896 | 41896 | 42896 | 43896 |
| 40897 | 41897 | 42897 | 43897 |
| 40898 | 41898 | 42898 | 43898 |
| 40899 | 41899 | 42899 | 43899 |
| 40900 | 41900 | 42900 | 43900 |
| 40901 | 41901 | 42901 | 43901 |
| 40902 | 41902 | 42902 | 43902 |
| 40903 | 41903 | 42903 | 43903 |
| 40904 | 41904 | 42904 | 43904 |
| 40905 | 41905 | 42905 | 43905 |
| 40906 | 41906 | 42906 | 43906 |
| 40907 | 41907 | 42907 | 43907 |
| 40908 | 41908 | 42908 | 43908 |
| 40909 | 41909 | 42909 | 43909 |
| 40910 | 41910 | 42910 | 43910 |
| 40911 | 41911 | 42911 | 43911 |
| 40912 | 41912 | 42912 | 43912 |
| 40913 | 41913 | 42913 | 43913 |
| 40914 | 41914 | 42914 | 43914 |
| 40915 | 41915 | 42915 | 43915 |
| 40916 | 41916 | 42916 | 43916 |
| 40917 | 41917 | 42917 | 43917 |
| 40918 | 41918 | 42918 | 43918 |
| 40919 | 41919 | 42919 | 43919 |
| 40920 | 41920 | 42920 | 43920 |
| 40921 | 41921 | 42921 | 43921 |
| 40922 | 41922 | 42922 | 43922 |
| 40923 | 41923 | 42923 | 43923 |
| 40924 | 41924 | 42924 | 43924 |
| 40925 | 41925 | 42925 | 43925 |
| 40926 | 41926 | 42926 | 43926 |
| 40927 | 41927 | 42927 | 43927 |
| 40928 | 41928 | 42928 | 43928 |
| 40929 | 41929 | 42929 | 43929 |
| 40930 | 41930 | 42930 | 43930 |
| 40931 | 41931 | 42931 | 43931 |
| 40932 | 41932 | 42932 | 43932 |
| 40933 | 41933 | 42933 | 43933 |
| 40934 | 41934 | 42934 | 43934 |
| 40935 | 41935 | 42935 | 43935 |
| 40936 | 41936 | 42936 | 43936 |
| 40937 | 41937 | 42937 | 43937 |
| 40938 | 41938 | 42938 | 43938 |
| 40939 | 41939 | 42939 | 43939 |
| 40940 | 41940 | 42940 | 43940 |
| 40941 | 41941 | 42941 | 43941 |
| 40942 | 41942 | 42942 | 43942 |
| 40943 | 41943 | 42943 | 43943 |
| 40944 | 41944 | 42944 | 43944 |
| 40945 | 41945 | 42945 | 43945 |
| 40946 | 41946 | 42946 | 43946 |
| 40947 | 41947 | 42947 | 43947 |
| 40948 | 41948 | 42948 | 43948 |
| 40949 | 41949 | 42949 | 43949 |
| 40950 | 41950 | 42950 | 43950 |
| 40951 | 41951 | 42951 | 43951 |
| 40952 | 41952 | 42952 | 43952 |
| 40953 | 41953 | 42953 | 43953 |
| 40954 | 41954 | 42954 | 43954 |
| 40955 | 41955 | 42955 | 43955 |
| 40956 | 41956 | 42956 | 43956 |
| 40957 | 41957 | 42957 | 43957 |
| 40958 | 41958 | 42958 | 43958 |
| 40959 | 41959 | 42959 | 43959 |
| 40960 | 41960 | 42960 | 43960 |
| 40961 | 41961 | 42961 | 43961 |
| 40962 | 41962 | 42962 | 43962 |
| 40963 | 41963 | 42963 | 43963 |
| 40964 | 41964 | 42964 | 43964 |
| 40965 | 41965 | 42965 | 43965 |
| 40966 | 41966 | 42966 | 43966 |
| 40967 | 41967 | 42967 | 43967 |
| 40968 | 41968 | 42968 | 43968 |
| 40969 | 41969 | 42969 | 43969 |
| 40970 | 41970 | 42970 | 43970 |
| 40971 | 41971 | 42971 | 43971 |
| 40972 | 41972 | 42972 | 43972 |
| 40973 | 41973 | 42973 | 43973 |
| 40974 | 41974 | 42974 | 43974 |
| 40975 | 41975 | 42975 | 43975 |
| 40976 | 41976 | 42976 | 43976 |
| 40977 | 41977 | 42977 | 43977 |
| 40978 | 41978 | 42978 | 43978 |
| 40979 | 41979 | 42979 | 43979 |
| 40980 | 41980 | 42980 | 43980 |
| 40981 | 41981 | 42981 | 43981 |
| 40982 | 41982 | 42982 | 43982 |
| 40983 | 41983 | 42983 | 43983 |
| 40984 | 41984 | 42984 | 43984 |
| 40985 | 41985 | 42985 | 43985 |
| 40986 | 41986 | 42986 | 43986 |
| 40987 | 41987 | 42987 | 43987 |
| 40988 | 41988 | 42988 | 43988 |
| 40989 | 41989 | 42989 | 43989 |
| 40990 | 41990 | 42990 | 43990 |
| 40991 | 41991 | 42991 | 43991 |
| 40992 | 41992 | 42992 | 43992 |
| 40993 | 41993 | 42993 | 43993 |
| 40994 | 41994 | 42994 | 43994 |
| 40995 | 41995 | 42995 | 43995 |
| 40996 | 41996 | 42996 | 43996 |
| 40997 | 41997 | 42997 | 43997 |
| 40998 | 41998 | 42998 | 43998 |
| 40999 | 41999 | 42999 | 43999 |
| 41000 | 42000 | 43000 | 44000 |
| 41001 | 42001 | 43001 | 44001 |
| 41002 | 42002 | 43002 | 44002 |

In some embodiments, the sequences that specifically bind to whey comprise an inner sequence selected from the group consisting of the nucleic acid sequences of SEQ ID NOs. 44003 to 45002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to whey may comprise a nucleic acid sequence selected from the group consisting of SEQ ID NOs. 45003 to 46002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to whey may comprise a nucleic acid sequence selected from the group consisting of SEQ ID NOs. 46003 to 47002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to whey may comprise a nucleic acid sequence selected from the group consisting of SEQ ID NOs. 47003 to 48002. In one embodiment, the aptamer of the present disclosure that specifically binds to whey may comprise a nucleic acid sequence selected from the group consisting of SEQ ID NOs. 44003 to 48002 listed in Table 13, or variant thereof.

TABLE 13
Aptamer sequences against whey
Aptamer sequence

| | | | 5′-sequence |
|---|---|---|---|
| | 5′-sequence | Inner | and inner |
| Inner | and inner | sequence | sequence and |
| sequence | sequence | 3′-sequence | 3′-sequence |
| (SEQ ID NO.) | (SEQ ID NO.) | (SEQ ID NO.) | (SEQ ID NO.) |
| 44003 | 45003 | 46003 | 47003 |
| 44004 | 45004 | 46004 | 47004 |
| 44005 | 45005 | 46005 | 47005 |
| 44006 | 45006 | 46006 | 47006 |
| 44007 | 45007 | 46007 | 47007 |
| 44008 | 45008 | 46008 | 47008 |
| 44009 | 45009 | 46009 | 47009 |
| 44010 | 45010 | 46010 | 47010 |
| 44011 | 45011 | 46011 | 47011 |
| 44012 | 45012 | 46012 | 47012 |
| 44013 | 45013 | 46013 | 47013 |
| 44014 | 45014 | 46014 | 47014 |
| 44015 | 45015 | 46015 | 47015 |
| 44016 | 45016 | 46016 | 47016 |
| 44017 | 45017 | 46017 | 47017 |
| 44018 | 45018 | 46018 | 47018 |
| 44019 | 45019 | 46019 | 47019 |
| 44020 | 45020 | 46020 | 47020 |
| 44021 | 45021 | 46021 | 47021 |
| 44022 | 45022 | 46022 | 47022 |
| 44023 | 45023 | 46023 | 47023 |
| 44024 | 45024 | 46024 | 47024 |
| 44025 | 45025 | 46025 | 47025 |
| 44026 | 45026 | 46026 | 47026 |
| 44027 | 45027 | 46027 | 47027 |
| 44028 | 45028 | 46028 | 47028 |
| 44029 | 45029 | 46029 | 47029 |
| 44030 | 45030 | 46030 | 47030 |
| 44031 | 45031 | 46031 | 47031 |
| 44032 | 45032 | 46032 | 47032 |
| 44033 | 45033 | 46033 | 47033 |
| 44034 | 45034 | 46034 | 47034 |
| 44035 | 45035 | 46035 | 47035 |
| 44036 | 45036 | 46036 | 47036 |
| 44037 | 45037 | 46037 | 47037 |
| 44038 | 45038 | 46038 | 47038 |
| 44039 | 45039 | 46039 | 47039 |
| 44040 | 45040 | 46040 | 47040 |
| 44041 | 45041 | 46041 | 47041 |
| 44042 | 45042 | 46042 | 47042 |
| 44043 | 45043 | 46043 | 47043 |
| 44044 | 45044 | 46044 | 47044 |
| 44045 | 45045 | 46045 | 47045 |
| 44046 | 45046 | 46046 | 47046 |
| 44047 | 45047 | 46047 | 47047 |
| 44048 | 45048 | 46048 | 47048 |
| 44049 | 45049 | 46049 | 47049 |
| 44050 | 45050 | 46050 | 47050 |
| 44051 | 45051 | 46051 | 47051 |
| 44052 | 45052 | 46052 | 47052 |
| 44053 | 45053 | 46053 | 47053 |
| 44054 | 45054 | 46054 | 47054 |
| 44055 | 45055 | 46055 | 47055 |
| 44056 | 45056 | 46056 | 47056 |
| 44057 | 45057 | 46057 | 47057 |
| 44058 | 45058 | 46058 | 47058 |
| 44059 | 45059 | 46059 | 47059 |
| 44060 | 45060 | 46060 | 47060 |
| 44061 | 45061 | 46061 | 47061 |
| 44062 | 45062 | 46062 | 47062 |
| 44063 | 45063 | 46063 | 47063 |
| 44064 | 45064 | 46064 | 47064 |
| 44065 | 45065 | 46065 | 47065 |
| 44066 | 45066 | 46066 | 47066 |
| 44067 | 45067 | 46067 | 47067 |
| 44068 | 45068 | 46068 | 47068 |
| 44069 | 45069 | 46069 | 47069 |
| 44070 | 45070 | 46070 | 47070 |
| 44071 | 45071 | 46071 | 47071 |
| 44072 | 45072 | 46072 | 47072 |
| 44073 | 45073 | 46073 | 47073 |
| 44074 | 45074 | 46074 | 47074 |
| 44075 | 45075 | 46075 | 47075 |
| 44076 | 45076 | 46076 | 47076 |
| 44077 | 45077 | 46077 | 47077 |
| 44078 | 45078 | 46078 | 47078 |
| 44079 | 45079 | 46079 | 47079 |
| 44080 | 45080 | 46080 | 47080 |
| 44081 | 45081 | 46081 | 47081 |
| 44082 | 45082 | 46082 | 47082 |
| 44083 | 45083 | 46083 | 47083 |
| 44084 | 45084 | 46084 | 47084 |
| 44085 | 45085 | 46085 | 47085 |
| 44086 | 45086 | 46086 | 47086 |
| 44087 | 45087 | 46087 | 47087 |
| 44088 | 45088 | 46088 | 47088 |
| 44089 | 45089 | 46089 | 47089 |
| 44090 | 45090 | 46090 | 47090 |
| 44091 | 45091 | 46091 | 47091 |
| 44092 | 45092 | 46092 | 47092 |
| 44093 | 45093 | 46093 | 47093 |
| 44094 | 45094 | 46094 | 47094 |
| 44095 | 45095 | 46095 | 47095 |
| 44096 | 45096 | 46096 | 47096 |
| 44097 | 45097 | 46097 | 47097 |
| 44098 | 45098 | 46098 | 47098 |
| 44099 | 45099 | 46099 | 47099 |
| 44100 | 45100 | 46100 | 47100 |
| 44101 | 45101 | 46101 | 47101 |
| 44102 | 45102 | 46102 | 47102 |
| 44103 | 45103 | 46103 | 47103 |
| 44104 | 45104 | 46104 | 47104 |
| 44105 | 45105 | 46105 | 47105 |
| 44106 | 45106 | 46106 | 47106 |
| 44107 | 45107 | 46107 | 47107 |
| 44108 | 45108 | 46108 | 47108 |
| 44109 | 45109 | 46109 | 47109 |
| 44110 | 45110 | 46110 | 47110 |
| 44111 | 45111 | 46111 | 47111 |
| 44112 | 45112 | 46112 | 47112 |
| 44113 | 45113 | 46113 | 47113 |
| 44114 | 45114 | 46114 | 47114 |
| 44115 | 45115 | 46115 | 47115 |
| 44116 | 45116 | 46116 | 47116 |
| 44117 | 45117 | 46117 | 47117 |
| 44118 | 45118 | 46118 | 47118 |
| 44119 | 45119 | 46119 | 47119 |
| 44120 | 45120 | 46120 | 47120 |
| 44121 | 45121 | 46121 | 47121 |
| 44122 | 45122 | 46122 | 47122 |
| 44123 | 45123 | 46123 | 47123 |
| 44124 | 45124 | 46124 | 47124 |
| 44125 | 45125 | 46125 | 47125 |
| 44126 | 45126 | 46126 | 47126 |
| 44127 | 45127 | 46127 | 47127 |
| 44128 | 45128 | 46128 | 47128 |
| 44129 | 45129 | 46129 | 47129 |
| 44130 | 45130 | 46130 | 47130 |
| 44131 | 45131 | 46131 | 47131 |
| 44132 | 45132 | 46132 | 47132 |
| 44133 | 45133 | 46133 | 47133 |
| 44134 | 45134 | 46134 | 47134 |
| 44135 | 45135 | 46135 | 47135 |
| 44136 | 45136 | 46136 | 47136 |
| 44137 | 45137 | 46137 | 47137 |
| 44138 | 45138 | 46138 | 47138 |
| 44139 | 45139 | 46139 | 47139 |
| 44140 | 45140 | 46140 | 47140 |
| 44141 | 45141 | 46141 | 47141 |
| 44142 | 45142 | 46142 | 47142 |
| 44143 | 45143 | 46143 | 47143 |
| 44144 | 45144 | 46144 | 47144 |
| 44145 | 45145 | 46145 | 47145 |
| 44146 | 45146 | 46146 | 47146 |
| 44147 | 45147 | 46147 | 47147 |
| 44148 | 45148 | 46148 | 47148 |
| 44149 | 45149 | 46149 | 47149 |
| 44150 | 45150 | 46150 | 47150 |
| 44151 | 45151 | 46151 | 47151 |
| 44152 | 45152 | 46152 | 47152 |
| 44153 | 45153 | 46153 | 47153 |
| 44154 | 45154 | 46154 | 47154 |
| 44155 | 45155 | 46155 | 47155 |
| 44156 | 45156 | 46156 | 47156 |
| 44157 | 45157 | 46157 | 47157 |
| 44158 | 45158 | 46158 | 47158 |
| 44159 | 45159 | 46159 | 47159 |
| 44160 | 45160 | 46160 | 47160 |
| 44161 | 45161 | 46161 | 47161 |
| 44162 | 45162 | 46162 | 47162 |
| 44163 | 45163 | 46163 | 47163 |
| 44164 | 45164 | 46164 | 47164 |
| 44165 | 45165 | 46165 | 47165 |
| 44166 | 45166 | 46166 | 47166 |
| 44167 | 45167 | 46167 | 47167 |
| 44168 | 45168 | 46168 | 47168 |
| 44169 | 45169 | 46169 | 47169 |
| 44170 | 45170 | 46170 | 47170 |
| 44171 | 45171 | 46171 | 47171 |
| 44172 | 45172 | 46172 | 47172 |
| 44173 | 45173 | 46173 | 47173 |
| 44174 | 45174 | 46174 | 47174 |
| 44175 | 45175 | 46175 | 47175 |
| 44176 | 45176 | 46176 | 47176 |
| 44177 | 45177 | 46177 | 47177 |
| 44178 | 45178 | 46178 | 47178 |
| 44179 | 45179 | 46179 | 47179 |
| 44180 | 45180 | 46180 | 47180 |
| 44181 | 45181 | 46181 | 47181 |
| 44182 | 45182 | 46182 | 47182 |
| 44183 | 45183 | 46183 | 47183 |
| 44184 | 45184 | 46184 | 47184 |
| 44185 | 45185 | 46185 | 47185 |
| 44186 | 45186 | 46186 | 47186 |
| 44187 | 45187 | 46187 | 47187 |
| 44188 | 45188 | 46188 | 47188 |
| 44189 | 45189 | 46189 | 47189 |
| 44190 | 45190 | 46190 | 47190 |
| 44191 | 45191 | 46191 | 47191 |
| 44192 | 45192 | 46192 | 47192 |
| 44193 | 45193 | 46193 | 47193 |
| 44194 | 45194 | 46194 | 47194 |
| 44195 | 45195 | 46195 | 47195 |
| 44196 | 45196 | 46196 | 47196 |
| 44197 | 45197 | 46197 | 47197 |
| 44198 | 45198 | 46198 | 47198 |
| 44199 | 45199 | 46199 | 47199 |
| 44200 | 45200 | 46200 | 47200 |
| 44201 | 45201 | 46201 | 47201 |
| 44202 | 45202 | 46202 | 47202 |
| 44203 | 45203 | 46203 | 47203 |
| 44204 | 45204 | 46204 | 47204 |
| 44205 | 45205 | 46205 | 47205 |
| 44206 | 45206 | 46206 | 47206 |
| 44207 | 45207 | 46207 | 47207 |
| 44208 | 45208 | 46208 | 47208 |
| 44209 | 45209 | 46209 | 47209 |
| 44210 | 45210 | 46210 | 47210 |
| 44211 | 45211 | 46211 | 47211 |
| 44212 | 45212 | 46212 | 47212 |
| 44213 | 45213 | 46213 | 47213 |
| 44214 | 45214 | 46214 | 47214 |
| 44215 | 45215 | 46215 | 47215 |
| 44216 | 45216 | 46216 | 47216 |
| 44217 | 45217 | 46217 | 47217 |
| 44218 | 45218 | 46218 | 47218 |
| 44219 | 45219 | 46219 | 47219 |
| 44220 | 45220 | 46220 | 47220 |
| 44221 | 45221 | 46221 | 47221 |
| 44222 | 45222 | 46222 | 47222 |
| 44223 | 45223 | 46223 | 47223 |
| 44224 | 45224 | 46224 | 47224 |
| 44225 | 45225 | 46225 | 47225 |
| 44226 | 45226 | 46226 | 47226 |
| 44227 | 45227 | 46227 | 47227 |
| 44228 | 45228 | 46228 | 47228 |
| 44229 | 45229 | 46229 | 47229 |
| 44230 | 45230 | 46230 | 47230 |
| 44231 | 45231 | 46231 | 47231 |
| 44232 | 45232 | 46232 | 47232 |
| 44233 | 45233 | 46233 | 47233 |
| 44234 | 45234 | 46234 | 47234 |
| 44235 | 45235 | 46235 | 47235 |
| 44236 | 45236 | 46236 | 47236 |
| 44237 | 45237 | 46237 | 47237 |
| 44238 | 45238 | 46238 | 47238 |
| 44239 | 45239 | 46239 | 47239 |
| 44240 | 45240 | 46240 | 47240 |
| 44241 | 45241 | 46241 | 47241 |
| 44242 | 45242 | 46242 | 47242 |
| 44243 | 45243 | 46243 | 47243 |
| 44244 | 45244 | 46244 | 47244 |
| 44245 | 45245 | 46245 | 47245 |
| 44246 | 45246 | 46246 | 47246 |
| 44247 | 45247 | 46247 | 47247 |
| 44248 | 45248 | 46248 | 47248 |
| 44249 | 45249 | 46249 | 47249 |
| 44250 | 45250 | 46250 | 47250 |
| 44251 | 45251 | 46251 | 47251 |
| 44252 | 45252 | 46252 | 47252 |
| 44253 | 45253 | 46253 | 47253 |
| 44254 | 45254 | 46254 | 47254 |
| 44255 | 45255 | 46255 | 47255 |
| 44256 | 45256 | 46256 | 47256 |
| 44257 | 45257 | 46257 | 47257 |
| 44258 | 45258 | 46258 | 47258 |
| 44259 | 45259 | 46259 | 47259 |
| 44260 | 45260 | 46260 | 47260 |
| 44261 | 45261 | 46261 | 47261 |
| 44262 | 45262 | 46262 | 47262 |
| 44263 | 45263 | 46263 | 47263 |
| 44264 | 45264 | 46264 | 47264 |
| 44265 | 45265 | 46265 | 47265 |
| 44266 | 45266 | 46266 | 47266 |
| 44267 | 45267 | 46267 | 47267 |
| 44268 | 45268 | 46268 | 47268 |
| 44269 | 45269 | 46269 | 47269 |
| 44270 | 45270 | 46270 | 47270 |
| 44271 | 45271 | 46271 | 47271 |
| 44272 | 45272 | 46272 | 47272 |
| 44273 | 45273 | 46273 | 47273 |
| 44274 | 45274 | 46274 | 47274 |
| 44275 | 45275 | 46275 | 47275 |
| 44276 | 45276 | 46276 | 47276 |
| 44277 | 45277 | 46277 | 47277 |
| 44278 | 45278 | 46278 | 47278 |
| 44279 | 45279 | 46279 | 47279 |
| 44280 | 45280 | 46280 | 47280 |
| 44281 | 45281 | 46281 | 47281 |
| 44282 | 45282 | 46282 | 47282 |
| 44283 | 45283 | 46283 | 47283 |
| 44284 | 45284 | 46284 | 47284 |
| 44285 | 45285 | 46285 | 47285 |
| 44286 | 45286 | 46286 | 47286 |
| 44287 | 45287 | 46287 | 47287 |
| 44288 | 45288 | 46288 | 47288 |
| 44289 | 45289 | 46289 | 47289 |
| 44290 | 45290 | 46290 | 47290 |
| 44291 | 45291 | 46291 | 47291 |
| 44292 | 45292 | 46292 | 47292 |
| 44293 | 45293 | 46293 | 47293 |
| 44294 | 45294 | 46294 | 47294 |
| 44295 | 45295 | 46295 | 47295 |
| 44296 | 45296 | 46296 | 47296 |
| 44297 | 45297 | 46297 | 47297 |
| 44298 | 45298 | 46298 | 47298 |
| 44299 | 45299 | 46299 | 47299 |
| 44300 | 45300 | 46300 | 47300 |
| 44301 | 45301 | 46301 | 47301 |
| 44302 | 45302 | 46302 | 47302 |
| 44303 | 45303 | 46303 | 47303 |
| 44304 | 45304 | 46304 | 47304 |
| 44305 | 45305 | 46305 | 47305 |
| 44306 | 45306 | 46306 | 47306 |
| 44307 | 45307 | 46307 | 47307 |
| 44308 | 45308 | 46308 | 47308 |
| 44309 | 45309 | 46309 | 47309 |
| 44310 | 45310 | 46310 | 47310 |
| 44311 | 45311 | 46311 | 47311 |
| 44312 | 45312 | 46312 | 47312 |
| 44313 | 45313 | 46313 | 47313 |
| 44314 | 45314 | 46314 | 47314 |
| 44315 | 45315 | 46315 | 47315 |
| 44316 | 45316 | 46316 | 47316 |
| 44317 | 45317 | 46317 | 47317 |
| 44318 | 45318 | 46318 | 47318 |
| 44319 | 45319 | 46319 | 47319 |
| 44320 | 45320 | 46320 | 47320 |
| 44321 | 45321 | 46321 | 47321 |
| 44322 | 45322 | 46322 | 47322 |
| 44323 | 45323 | 46323 | 47323 |
| 44324 | 45324 | 46324 | 47324 |
| 44325 | 45325 | 46325 | 47325 |
| 44326 | 45326 | 46326 | 47326 |
| 44327 | 45327 | 46327 | 47327 |
| 44328 | 45328 | 46328 | 47328 |
| 44329 | 45329 | 46329 | 47329 |
| 44330 | 45330 | 46330 | 47330 |
| 44331 | 45331 | 46331 | 47331 |
| 44332 | 45332 | 46332 | 47332 |
| 44333 | 45333 | 46333 | 47333 |
| 44334 | 45334 | 46334 | 47334 |
| 44335 | 45335 | 46335 | 47335 |
| 44336 | 45336 | 46336 | 47336 |
| 44337 | 45337 | 46337 | 47337 |
| 44338 | 45338 | 46338 | 47338 |
| 44339 | 45339 | 46339 | 47339 |
| 44340 | 45340 | 46340 | 47340 |
| 44341 | 45341 | 46341 | 47341 |
| 44342 | 45342 | 46342 | 47342 |
| 44343 | 45343 | 46343 | 47343 |
| 44344 | 45344 | 46344 | 47344 |
| 44345 | 45345 | 46345 | 47345 |
| 44346 | 45346 | 46346 | 47346 |
| 44347 | 45347 | 46347 | 47347 |
| 44348 | 45348 | 46348 | 47348 |
| 44349 | 45349 | 46349 | 47349 |
| 44350 | 45350 | 46350 | 47350 |
| 44351 | 45351 | 46351 | 47351 |
| 44352 | 45352 | 46352 | 47352 |
| 44353 | 45353 | 46353 | 47353 |
| 44354 | 45354 | 46354 | 47354 |
| 44355 | 45355 | 46355 | 47355 |
| 44356 | 45356 | 46356 | 47356 |
| 44357 | 45357 | 46357 | 47357 |
| 44358 | 45358 | 46358 | 47358 |
| 44359 | 45359 | 46359 | 47359 |
| 44360 | 45360 | 46360 | 47360 |
| 44361 | 45361 | 46361 | 47361 |
| 44362 | 45362 | 46362 | 47362 |
| 44363 | 45363 | 46363 | 47363 |
| 44364 | 45364 | 46364 | 47364 |
| 44365 | 45365 | 46365 | 47365 |
| 44366 | 45366 | 46366 | 47366 |
| 44367 | 45367 | 46367 | 47367 |
| 44368 | 45368 | 46368 | 47368 |
| 44369 | 45369 | 46369 | 47369 |
| 44370 | 45370 | 46370 | 47370 |
| 44371 | 45371 | 46371 | 47371 |
| 44372 | 45372 | 46372 | 47372 |
| 44373 | 45373 | 46373 | 47373 |
| 44374 | 45374 | 46374 | 47374 |
| 44375 | 45375 | 46375 | 47375 |
| 44376 | 45376 | 46376 | 47376 |
| 44377 | 45377 | 46377 | 47377 |
| 44378 | 45378 | 46378 | 47378 |
| 44379 | 45379 | 46379 | 47379 |
| 44380 | 45380 | 46380 | 47380 |
| 44381 | 45381 | 46381 | 47381 |
| 44382 | 45382 | 46382 | 47382 |
| 44383 | 45383 | 46383 | 47383 |
| 44384 | 45384 | 46384 | 47384 |
| 44385 | 45385 | 46385 | 47385 |
| 44386 | 45386 | 46386 | 47386 |
| 44387 | 45387 | 46387 | 47387 |
| 44388 | 45388 | 46388 | 47388 |
| 44389 | 45389 | 46389 | 47389 |
| 44390 | 45390 | 46390 | 47390 |
| 44391 | 45391 | 46391 | 47391 |
| 44392 | 45392 | 46392 | 47392 |
| 44393 | 45393 | 46393 | 47393 |
| 44394 | 45394 | 46394 | 47394 |
| 44395 | 45395 | 46395 | 47395 |
| 44396 | 45396 | 46396 | 47396 |
| 44397 | 45397 | 46397 | 47397 |
| 44398 | 45398 | 46398 | 47398 |
| 44399 | 45399 | 46399 | 47399 |
| 44400 | 45400 | 46400 | 47400 |
| 44401 | 45401 | 46401 | 47401 |
| 44402 | 45402 | 46402 | 47402 |
| 44403 | 45403 | 46403 | 47403 |
| 44404 | 45404 | 46404 | 47404 |
| 44405 | 45405 | 46405 | 47405 |
| 44406 | 45406 | 46406 | 47406 |
| 44407 | 45407 | 46407 | 47407 |
| 44408 | 45408 | 46408 | 47408 |
| 44409 | 45409 | 46409 | 47409 |
| 44410 | 45410 | 46410 | 47410 |
| 44411 | 45411 | 46411 | 47411 |
| 44412 | 45412 | 46412 | 47412 |
| 44413 | 45413 | 46413 | 47413 |
| 44414 | 45414 | 46414 | 47414 |
| 44415 | 45415 | 46415 | 47415 |
| 44416 | 45416 | 46416 | 47416 |
| 44417 | 45417 | 46417 | 47417 |
| 44418 | 45418 | 46418 | 47418 |
| 44419 | 45419 | 46419 | 47419 |
| 44420 | 45420 | 46420 | 47420 |
| 44421 | 45421 | 46421 | 47421 |
| 44422 | 45422 | 46422 | 47422 |
| 44423 | 45423 | 46423 | 47423 |
| 44424 | 45424 | 46424 | 47424 |
| 44425 | 45425 | 46425 | 47425 |
| 44426 | 45426 | 46426 | 47426 |
| 44427 | 45427 | 46427 | 47427 |
| 44428 | 45428 | 46428 | 47428 |
| 44429 | 45429 | 46429 | 47429 |
| 44430 | 45430 | 46430 | 47430 |
| 44431 | 45431 | 46431 | 47431 |
| 44432 | 45432 | 46432 | 47432 |
| 44433 | 45433 | 46433 | 47433 |
| 44434 | 45434 | 46434 | 47434 |
| 44435 | 45435 | 46435 | 47435 |
| 44436 | 45436 | 46436 | 47436 |
| 44437 | 45437 | 46437 | 47437 |
| 44438 | 45438 | 46438 | 47438 |
| 44439 | 45439 | 46439 | 47439 |
| 44440 | 45440 | 46440 | 47440 |
| 44441 | 45441 | 46441 | 47441 |
| 44442 | 45442 | 46442 | 47442 |
| 44443 | 45443 | 46443 | 47443 |
| 44444 | 45444 | 46444 | 47444 |
| 44445 | 45445 | 46445 | 47445 |
| 44446 | 45446 | 46446 | 47446 |
| 44447 | 45447 | 46447 | 47447 |
| 44448 | 45448 | 46448 | 47448 |
| 44449 | 45449 | 46449 | 47449 |
| 44450 | 45450 | 46450 | 47450 |
| 44451 | 45451 | 46451 | 47451 |
| 44452 | 45452 | 46452 | 47452 |
| 44453 | 45453 | 46453 | 47453 |
| 44454 | 45454 | 46454 | 47454 |
| 44455 | 45455 | 46455 | 47455 |
| 44456 | 45456 | 46456 | 47456 |
| 44457 | 45457 | 46457 | 47457 |
| 44458 | 45458 | 46458 | 47458 |
| 44459 | 45459 | 46459 | 47459 |
| 44460 | 45460 | 46460 | 47460 |
| 44461 | 45461 | 46461 | 47461 |
| 44462 | 45462 | 46462 | 47462 |
| 44463 | 45463 | 46463 | 47463 |
| 44464 | 45464 | 46464 | 47464 |
| 44465 | 45465 | 46465 | 47465 |
| 44466 | 45466 | 46466 | 47466 |
| 44467 | 45467 | 46467 | 47467 |
| 44468 | 45468 | 46468 | 47468 |
| 44469 | 45469 | 46469 | 47469 |
| 44470 | 45470 | 46470 | 47470 |
| 44471 | 45471 | 46471 | 47471 |
| 44472 | 45472 | 46472 | 47472 |
| 44473 | 45473 | 46473 | 47473 |
| 44474 | 45474 | 46474 | 47474 |
| 44475 | 45475 | 46475 | 47475 |
| 44476 | 45476 | 46476 | 47476 |
| 44477 | 45477 | 46477 | 47477 |
| 44478 | 45478 | 46478 | 47478 |
| 44479 | 45479 | 46479 | 47479 |
| 44480 | 45480 | 46480 | 47480 |
| 44481 | 45481 | 46481 | 47481 |
| 44482 | 45482 | 46482 | 47482 |
| 44483 | 45483 | 46483 | 47483 |
| 44484 | 45484 | 46484 | 47484 |
| 44485 | 45485 | 46485 | 47485 |
| 44486 | 45486 | 46486 | 47486 |
| 44487 | 45487 | 46487 | 47487 |
| 44488 | 45488 | 46488 | 47488 |
| 44489 | 45489 | 46489 | 47489 |
| 44490 | 45490 | 46490 | 47490 |
| 44491 | 45491 | 46491 | 47491 |
| 44492 | 45492 | 46492 | 47492 |
| 44493 | 45493 | 46493 | 47493 |
| 44494 | 45494 | 46494 | 47494 |
| 44495 | 45495 | 46495 | 47495 |
| 44496 | 45496 | 46496 | 47496 |
| 44497 | 45497 | 46497 | 47497 |
| 44498 | 45498 | 46498 | 47498 |
| 44499 | 45499 | 46499 | 47499 |
| 44500 | 45500 | 46500 | 47500 |
| 44501 | 45501 | 46501 | 47501 |
| 44502 | 45502 | 46502 | 47502 |
| 44503 | 45503 | 46503 | 47503 |
| 44504 | 45504 | 46504 | 47504 |
| 44505 | 45505 | 46505 | 47505 |
| 44506 | 45506 | 46506 | 47506 |
| 44507 | 45507 | 46507 | 47507 |
| 44508 | 45508 | 46508 | 47508 |
| 44509 | 45509 | 46509 | 47509 |
| 44510 | 45510 | 46510 | 47510 |
| 44511 | 45511 | 46511 | 47511 |
| 44512 | 45512 | 46512 | 47512 |
| 44513 | 45513 | 46513 | 47513 |
| 44514 | 45514 | 46514 | 47514 |
| 44515 | 45515 | 46515 | 47515 |
| 44516 | 45516 | 46516 | 47516 |
| 44517 | 45517 | 46517 | 47517 |
| 44518 | 45518 | 46518 | 47518 |
| 44519 | 45519 | 46519 | 47519 |
| 44520 | 45520 | 46520 | 47520 |
| 44521 | 45521 | 46521 | 47521 |
| 44522 | 45522 | 46522 | 47522 |
| 44523 | 45523 | 46523 | 47523 |
| 44524 | 45524 | 46524 | 47524 |
| 44525 | 45525 | 46525 | 47525 |
| 44526 | 45526 | 46526 | 47526 |
| 44527 | 45527 | 46527 | 47527 |
| 44528 | 45528 | 46528 | 47528 |
| 44529 | 45529 | 46529 | 47529 |
| 44530 | 45530 | 46530 | 47530 |
| 44531 | 45531 | 46531 | 47531 |
| 44532 | 45532 | 46532 | 47532 |
| 44533 | 45533 | 46533 | 47533 |
| 44534 | 45534 | 46534 | 47534 |
| 44535 | 45535 | 46535 | 47535 |
| 44536 | 45536 | 46536 | 47536 |
| 44537 | 45537 | 46537 | 47537 |
| 44538 | 45538 | 46538 | 47538 |
| 44539 | 45539 | 46539 | 47539 |
| 44540 | 45540 | 46540 | 47540 |
| 44541 | 45541 | 46541 | 47541 |
| 44542 | 45542 | 46542 | 47542 |
| 44543 | 45543 | 46543 | 47543 |
| 44544 | 45544 | 46544 | 47544 |
| 44545 | 45545 | 46545 | 47545 |
| 44546 | 45546 | 46546 | 47546 |
| 44547 | 45547 | 46547 | 47547 |
| 44548 | 45548 | 46548 | 47548 |
| 44549 | 45549 | 46549 | 47549 |
| 44550 | 45550 | 46550 | 47550 |
| 44551 | 45551 | 46551 | 47551 |
| 44552 | 45552 | 46552 | 47552 |
| 44553 | 45553 | 46553 | 47553 |
| 44554 | 45554 | 46554 | 47554 |
| 44555 | 45555 | 46555 | 47555 |
| 44556 | 45556 | 46556 | 47556 |
| 44557 | 45557 | 46557 | 47557 |
| 44558 | 45558 | 46558 | 47558 |
| 44559 | 45559 | 46559 | 47559 |
| 44560 | 45560 | 46560 | 47560 |
| 44561 | 45561 | 46561 | 47561 |
| 44562 | 45562 | 46562 | 47562 |
| 44563 | 45563 | 46563 | 47563 |
| 44564 | 45564 | 46564 | 47564 |
| 44565 | 45565 | 46565 | 47565 |
| 44566 | 45566 | 46566 | 47566 |
| 44567 | 45567 | 46567 | 47567 |
| 44568 | 45568 | 46568 | 47568 |
| 44569 | 45569 | 46569 | 47569 |
| 44570 | 45570 | 46570 | 47570 |
| 44571 | 45571 | 46571 | 47571 |
| 44572 | 45572 | 46572 | 47572 |
| 44573 | 45573 | 46573 | 47573 |
| 44574 | 45574 | 46574 | 47574 |
| 44575 | 45575 | 46575 | 47575 |
| 44576 | 45576 | 46576 | 47576 |
| 44577 | 45577 | 46577 | 47577 |
| 44578 | 45578 | 46578 | 47578 |
| 44579 | 45579 | 46579 | 47579 |
| 44580 | 45580 | 46580 | 47580 |
| 44581 | 45581 | 46581 | 47581 |
| 44582 | 45582 | 46582 | 47582 |
| 44583 | 45583 | 46583 | 47583 |
| 44584 | 45584 | 46584 | 47584 |
| 44585 | 45585 | 46585 | 47585 |
| 44586 | 45586 | 46586 | 47586 |
| 44587 | 45587 | 46587 | 47587 |
| 44588 | 45588 | 46588 | 47588 |
| 44589 | 45589 | 46589 | 47589 |
| 44590 | 45590 | 46590 | 47590 |
| 44591 | 45591 | 46591 | 47591 |
| 44592 | 45592 | 46592 | 47592 |
| 44593 | 45593 | 46593 | 47593 |
| 44594 | 45594 | 46594 | 47594 |
| 44595 | 45595 | 46595 | 47595 |
| 44596 | 45596 | 46596 | 47596 |
| 44597 | 45597 | 46597 | 47597 |
| 44598 | 45598 | 46598 | 47598 |
| 44599 | 45599 | 46599 | 47599 |
| 44600 | 45600 | 46600 | 47600 |
| 44601 | 45601 | 46601 | 47601 |
| 44602 | 45602 | 46602 | 47602 |
| 44603 | 45603 | 46603 | 47603 |
| 44604 | 45604 | 46604 | 47604 |
| 44605 | 45605 | 46605 | 47605 |
| 44606 | 45606 | 46606 | 47606 |
| 44607 | 45607 | 46607 | 47607 |
| 44608 | 45608 | 46608 | 47608 |
| 44609 | 45609 | 46609 | 47609 |
| 44610 | 45610 | 46610 | 47610 |
| 44611 | 45611 | 46611 | 47611 |
| 44612 | 45612 | 46612 | 47612 |
| 44613 | 45613 | 46613 | 47613 |
| 44614 | 45614 | 46614 | 47614 |
| 44615 | 45615 | 46615 | 47615 |
| 44616 | 45616 | 46616 | 47616 |
| 44617 | 45617 | 46617 | 47617 |
| 44618 | 45618 | 46618 | 47618 |
| 44619 | 45619 | 46619 | 47619 |
| 44620 | 45620 | 46620 | 47620 |
| 44621 | 45621 | 46621 | 47621 |
| 44622 | 45622 | 46622 | 47622 |
| 44623 | 45623 | 46623 | 47623 |
| 44624 | 45624 | 46624 | 47624 |
| 44625 | 45625 | 46625 | 47625 |
| 44626 | 45626 | 46626 | 47626 |
| 44627 | 45627 | 46627 | 47627 |
| 44628 | 45628 | 46628 | 47628 |
| 44629 | 45629 | 46629 | 47629 |
| 44630 | 45630 | 46630 | 47630 |
| 44631 | 45631 | 46631 | 47631 |
| 44632 | 45632 | 46632 | 47632 |
| 44633 | 45633 | 46633 | 47633 |
| 44634 | 45634 | 46634 | 47634 |
| 44635 | 45635 | 46635 | 47635 |
| 44636 | 45636 | 46636 | 47636 |
| 44637 | 45637 | 46637 | 47637 |
| 44638 | 45638 | 46638 | 47638 |
| 44639 | 45639 | 46639 | 47639 |
| 44640 | 45640 | 46640 | 47640 |
| 44641 | 45641 | 46641 | 47641 |
| 44642 | 45642 | 46642 | 47642 |
| 44643 | 45643 | 46643 | 47643 |
| 44644 | 45644 | 46644 | 47644 |
| 44645 | 45645 | 46645 | 47645 |
| 44646 | 45646 | 46646 | 47646 |
| 44647 | 45647 | 46647 | 47647 |
| 44648 | 45648 | 46648 | 47648 |
| 44649 | 45649 | 46649 | 47649 |
| 44650 | 45650 | 46650 | 47650 |
| 44651 | 45651 | 46651 | 47651 |
| 44652 | 45652 | 46652 | 47652 |
| 44653 | 45653 | 46653 | 47653 |
| 44654 | 45654 | 46654 | 47654 |
| 44655 | 45655 | 46655 | 47655 |
| 44656 | 45656 | 46656 | 47656 |
| 44657 | 45657 | 46657 | 47657 |
| 44658 | 45658 | 46658 | 47658 |
| 44659 | 45659 | 46659 | 47659 |
| 44660 | 45660 | 46660 | 47660 |
| 44661 | 45661 | 46661 | 47661 |
| 44662 | 45662 | 46662 | 47662 |
| 44663 | 45663 | 46663 | 47663 |
| 44664 | 45664 | 46664 | 47664 |
| 44665 | 45665 | 46665 | 47665 |
| 44666 | 45666 | 46666 | 47666 |
| 44667 | 45667 | 46667 | 47667 |
| 44668 | 45668 | 46668 | 47668 |
| 44669 | 45669 | 46669 | 47669 |
| 44670 | 45670 | 46670 | 47670 |
| 44671 | 45671 | 46671 | 47671 |
| 44672 | 45672 | 46672 | 47672 |
| 44673 | 45673 | 46673 | 47673 |
| 44674 | 45674 | 46674 | 47674 |
| 44675 | 45675 | 46675 | 47675 |
| 44676 | 45676 | 46676 | 47676 |
| 44677 | 45677 | 46677 | 47677 |
| 44678 | 45678 | 46678 | 47678 |
| 44679 | 45679 | 46679 | 47679 |
| 44680 | 45680 | 46680 | 47680 |
| 44681 | 45681 | 46681 | 47681 |
| 44682 | 45682 | 46682 | 47682 |
| 44683 | 45683 | 46683 | 47683 |
| 44684 | 45684 | 46684 | 47684 |
| 44685 | 45685 | 46685 | 47685 |
| 44686 | 45686 | 46686 | 47686 |
| 44687 | 45687 | 46687 | 47687 |
| 44688 | 45688 | 46688 | 47688 |
| 44689 | 45689 | 46689 | 47689 |
| 44690 | 45690 | 46690 | 47690 |
| 44691 | 45691 | 46691 | 47691 |
| 44692 | 45692 | 46692 | 47692 |
| 44693 | 45693 | 46693 | 47693 |
| 44694 | 45694 | 46694 | 47694 |
| 44695 | 45695 | 46695 | 47695 |
| 44696 | 45696 | 46696 | 47696 |
| 44697 | 45697 | 46697 | 47697 |
| 44698 | 45698 | 46698 | 47698 |
| 44699 | 45699 | 46699 | 47699 |
| 44700 | 45700 | 46700 | 47700 |
| 44701 | 45701 | 46701 | 47701 |
| 44702 | 45702 | 46702 | 47702 |
| 44703 | 45703 | 46703 | 47703 |
| 44704 | 45704 | 46704 | 47704 |
| 44705 | 45705 | 46705 | 47705 |
| 44706 | 45706 | 46706 | 47706 |
| 44707 | 45707 | 46707 | 47707 |
| 44708 | 45708 | 46708 | 47708 |
| 44709 | 45709 | 46709 | 47709 |
| 44710 | 45710 | 46710 | 47710 |
| 44711 | 45711 | 46711 | 47711 |
| 44712 | 45712 | 46712 | 47712 |
| 44713 | 45713 | 46713 | 47713 |
| 44714 | 45714 | 46714 | 47714 |
| 44715 | 45715 | 46715 | 47715 |
| 44716 | 45716 | 46716 | 47716 |
| 44717 | 45717 | 46717 | 47717 |
| 44718 | 45718 | 46718 | 47718 |
| 44719 | 45719 | 46719 | 47719 |
| 44720 | 45720 | 46720 | 47720 |
| 44721 | 45721 | 46721 | 47721 |
| 44722 | 45722 | 46722 | 47722 |
| 44723 | 45723 | 46723 | 47723 |
| 44724 | 45724 | 46724 | 47724 |
| 44725 | 45725 | 46725 | 47725 |
| 44726 | 45726 | 46726 | 47726 |
| 44727 | 45727 | 46727 | 47727 |
| 44728 | 45728 | 46728 | 47728 |
| 44729 | 45729 | 46729 | 47729 |
| 44730 | 45730 | 46730 | 47730 |
| 44731 | 45731 | 46731 | 47731 |
| 44732 | 45732 | 46732 | 47732 |
| 44733 | 45733 | 46733 | 47733 |
| 44734 | 45734 | 46734 | 47734 |
| 44735 | 45735 | 46735 | 47735 |
| 44736 | 45736 | 46736 | 47736 |
| 44737 | 45737 | 46737 | 47737 |
| 44738 | 45738 | 46738 | 47738 |
| 44739 | 45739 | 46739 | 47739 |
| 44740 | 45740 | 46740 | 47740 |
| 44741 | 45741 | 46741 | 47741 |
| 44742 | 45742 | 46742 | 47742 |
| 44743 | 45743 | 46743 | 47743 |
| 44744 | 45744 | 46744 | 47744 |
| 44745 | 45745 | 46745 | 47745 |
| 44746 | 45746 | 46746 | 47746 |
| 44747 | 45747 | 46747 | 47747 |
| 44748 | 45748 | 46748 | 47748 |
| 44749 | 45749 | 46749 | 47749 |
| 44750 | 45750 | 46750 | 47750 |
| 44751 | 45751 | 46751 | 47751 |
| 44752 | 45752 | 46752 | 47752 |
| 44753 | 45753 | 46753 | 47753 |
| 44754 | 45754 | 46754 | 47754 |
| 44755 | 45755 | 46755 | 47755 |
| 44756 | 45756 | 46756 | 47756 |
| 44757 | 45757 | 46757 | 47757 |
| 44758 | 45758 | 46758 | 47758 |
| 44759 | 45759 | 46759 | 47759 |
| 44760 | 45760 | 46760 | 47760 |
| 44761 | 45761 | 46761 | 47761 |
| 44762 | 45762 | 46762 | 47762 |
| 44763 | 45763 | 46763 | 47763 |
| 44764 | 45764 | 46764 | 47764 |
| 44765 | 45765 | 46765 | 47765 |
| 44766 | 45766 | 46766 | 47766 |
| 44767 | 45767 | 46767 | 47767 |
| 44768 | 45768 | 46768 | 47768 |
| 44769 | 45769 | 46769 | 47769 |
| 44770 | 45770 | 46770 | 47770 |
| 44771 | 45771 | 46771 | 47771 |
| 44772 | 45772 | 46772 | 47772 |
| 44773 | 45773 | 46773 | 47773 |
| 44774 | 45774 | 46774 | 47774 |
| 44775 | 45775 | 46775 | 47775 |
| 44776 | 45776 | 46776 | 47776 |
| 44777 | 45777 | 46777 | 47777 |
| 44778 | 45778 | 46778 | 47778 |
| 44779 | 45779 | 46779 | 47779 |
| 44780 | 45780 | 46780 | 47780 |
| 44781 | 45781 | 46781 | 47781 |
| 44782 | 45782 | 46782 | 47782 |
| 44783 | 45783 | 46783 | 47783 |
| 44784 | 45784 | 46784 | 47784 |
| 44785 | 45785 | 46785 | 47785 |
| 44786 | 45786 | 46786 | 47786 |
| 44787 | 45787 | 46787 | 47787 |
| 44788 | 45788 | 46788 | 47788 |
| 44789 | 45789 | 46789 | 47789 |
| 44790 | 45790 | 46790 | 47790 |
| 44791 | 45791 | 46791 | 47791 |
| 44792 | 45792 | 46792 | 47792 |
| 44793 | 45793 | 46793 | 47793 |
| 44794 | 45794 | 46794 | 47794 |
| 44795 | 45795 | 46795 | 47795 |
| 44796 | 45796 | 46796 | 47796 |
| 44797 | 45797 | 46797 | 47797 |
| 44798 | 45798 | 46798 | 47798 |
| 44799 | 45799 | 46799 | 47799 |
| 44800 | 45800 | 46800 | 47800 |
| 44801 | 45801 | 46801 | 47801 |
| 44802 | 45802 | 46802 | 47802 |
| 44803 | 45803 | 46803 | 47803 |
| 44804 | 45804 | 46804 | 47804 |
| 44805 | 45805 | 46805 | 47805 |
| 44806 | 45806 | 46806 | 47806 |
| 44807 | 45807 | 46807 | 47807 |
| 44808 | 45808 | 46808 | 47808 |
| 44809 | 45809 | 46809 | 47809 |
| 44810 | 45810 | 46810 | 47810 |
| 44811 | 45811 | 46811 | 47811 |
| 44812 | 45812 | 46812 | 47812 |
| 44813 | 45813 | 46813 | 47813 |
| 44814 | 45814 | 46814 | 47814 |
| 44815 | 45815 | 46815 | 47815 |
| 44816 | 45816 | 46816 | 47816 |
| 44817 | 45817 | 46817 | 47817 |
| 44818 | 45818 | 46818 | 47818 |
| 44819 | 45819 | 46819 | 47819 |
| 44820 | 45820 | 46820 | 47820 |
| 44821 | 45821 | 46821 | 47821 |
| 44822 | 45822 | 46822 | 47822 |
| 44823 | 45823 | 46823 | 47823 |
| 44824 | 45824 | 46824 | 47824 |
| 44825 | 45825 | 46825 | 47825 |
| 44826 | 45826 | 46826 | 47826 |
| 44827 | 45827 | 46827 | 47827 |
| 44828 | 45828 | 46828 | 47828 |
| 44829 | 45829 | 46829 | 47829 |
| 44830 | 45830 | 46830 | 47830 |
| 44831 | 45831 | 46831 | 47831 |
| 44832 | 45832 | 46832 | 47832 |
| 44833 | 45833 | 46833 | 47833 |
| 44834 | 45834 | 46834 | 47834 |
| 44835 | 45835 | 46835 | 47835 |
| 44836 | 45836 | 46836 | 47836 |
| 44837 | 45837 | 46837 | 47837 |
| 44838 | 45838 | 46838 | 47838 |
| 44839 | 45839 | 46839 | 47839 |
| 44840 | 45840 | 46840 | 47840 |
| 44841 | 45841 | 46841 | 47841 |
| 44842 | 45842 | 46842 | 47842 |
| 44843 | 45843 | 46843 | 47843 |
| 44844 | 45844 | 46844 | 47844 |
| 44845 | 45845 | 46845 | 47845 |
| 44846 | 45846 | 46846 | 47846 |
| 44847 | 45847 | 46847 | 47847 |
| 44848 | 45848 | 46848 | 47848 |
| 44849 | 45849 | 46849 | 47849 |
| 44850 | 45850 | 46850 | 47850 |
| 44851 | 45851 | 46851 | 47851 |
| 44852 | 45852 | 46852 | 47852 |
| 44853 | 45853 | 46853 | 47853 |
| 44854 | 45854 | 46854 | 47854 |
| 44855 | 45855 | 46855 | 47855 |
| 44856 | 45856 | 46856 | 47856 |
| 44857 | 45857 | 46857 | 47857 |
| 44858 | 45858 | 46858 | 47858 |
| 44859 | 45859 | 46859 | 47859 |
| 44860 | 45860 | 46860 | 47860 |
| 44861 | 45861 | 46861 | 47861 |
| 44862 | 45862 | 46862 | 47862 |
| 44863 | 45863 | 46863 | 47863 |
| 44864 | 45864 | 46864 | 47864 |
| 44865 | 45865 | 46865 | 47865 |
| 44866 | 45866 | 46866 | 47866 |
| 44867 | 45867 | 46867 | 47867 |
| 44868 | 45868 | 46868 | 47868 |
| 44869 | 45869 | 46869 | 47869 |
| 44870 | 45870 | 46870 | 47870 |
| 44871 | 45871 | 46871 | 47871 |
| 44872 | 45872 | 46872 | 47872 |
| 44873 | 45873 | 46873 | 47873 |
| 44874 | 45874 | 46874 | 47874 |
| 44875 | 45875 | 46875 | 47875 |
| 44876 | 45876 | 46876 | 47876 |
| 44877 | 45877 | 46877 | 47877 |
| 44878 | 45878 | 46878 | 47878 |
| 44879 | 45879 | 46879 | 47879 |
| 44880 | 45880 | 46880 | 47880 |
| 44881 | 45881 | 46881 | 47881 |
| 44882 | 45882 | 46882 | 47882 |
| 44883 | 45883 | 46883 | 47883 |
| 44884 | 45884 | 46884 | 47884 |
| 44885 | 45885 | 46885 | 47885 |
| 44886 | 45886 | 46886 | 47886 |
| 44887 | 45887 | 46887 | 47887 |
| 44888 | 45888 | 46888 | 47888 |
| 44889 | 45889 | 46889 | 47889 |
| 44890 | 45890 | 46890 | 47890 |
| 44891 | 45891 | 46891 | 47891 |
| 44892 | 45892 | 46892 | 47892 |
| 44893 | 45893 | 46893 | 47893 |
| 44894 | 45894 | 46894 | 47894 |
| 44895 | 45895 | 46895 | 47895 |
| 44896 | 45896 | 46896 | 47896 |
| 44897 | 45897 | 46897 | 47897 |
| 44898 | 45898 | 46898 | 47898 |
| 44899 | 45899 | 46899 | 47899 |
| 44900 | 45900 | 46900 | 47900 |
| 44901 | 45901 | 46901 | 47901 |
| 44902 | 45902 | 46902 | 47902 |
| 44903 | 45903 | 46903 | 47903 |
| 44904 | 45904 | 46904 | 47904 |
| 44905 | 45905 | 46905 | 47905 |
| 44906 | 45906 | 46906 | 47906 |
| 44907 | 45907 | 46907 | 47907 |
| 44908 | 45908 | 46908 | 47908 |
| 44909 | 45909 | 46909 | 47909 |
| 44910 | 45910 | 46910 | 47910 |
| 44911 | 45911 | 46911 | 47911 |
| 44912 | 45912 | 46912 | 47912 |
| 44913 | 45913 | 46913 | 47913 |
| 44914 | 45914 | 46914 | 47914 |
| 44915 | 45915 | 46915 | 47915 |
| 44916 | 45916 | 46916 | 47916 |
| 44917 | 45917 | 46917 | 47917 |
| 44918 | 45918 | 46918 | 47918 |
| 44919 | 45919 | 46919 | 47919 |
| 44920 | 45920 | 46920 | 47920 |
| 44921 | 45921 | 46921 | 47921 |
| 44922 | 45922 | 46922 | 47922 |
| 44923 | 45923 | 46923 | 47923 |
| 44924 | 45924 | 46924 | 47924 |
| 44925 | 45925 | 46925 | 47925 |
| 44926 | 45926 | 46926 | 47926 |
| 44927 | 45927 | 46927 | 47927 |
| 44928 | 45928 | 46928 | 47928 |
| 44929 | 45929 | 46929 | 47929 |
| 44930 | 45930 | 46930 | 47930 |
| 44931 | 45931 | 46931 | 47931 |
| 44932 | 45932 | 46932 | 47932 |
| 44933 | 45933 | 46933 | 47933 |
| 44934 | 45934 | 46934 | 47934 |
| 44935 | 45935 | 46935 | 47935 |
| 44936 | 45936 | 46936 | 47936 |
| 44937 | 45937 | 46937 | 47937 |
| 44938 | 45938 | 46938 | 47938 |
| 44939 | 45939 | 46939 | 47939 |
| 44940 | 45940 | 46940 | 47940 |
| 44941 | 45941 | 46941 | 47941 |
| 44942 | 45942 | 46942 | 47942 |
| 44943 | 45943 | 46943 | 47943 |
| 44944 | 45944 | 46944 | 47944 |
| 44945 | 45945 | 46945 | 47945 |
| 44946 | 45946 | 46946 | 47946 |
| 44947 | 45947 | 46947 | 47947 |
| 44948 | 45948 | 46948 | 47948 |
| 44949 | 45949 | 46949 | 47949 |
| 44950 | 45950 | 46950 | 47950 |
| 44951 | 45951 | 46951 | 47951 |
| 44952 | 45952 | 46952 | 47952 |
| 44953 | 45953 | 46953 | 47953 |
| 44954 | 45954 | 46954 | 47954 |
| 44955 | 45955 | 46955 | 47955 |
| 44956 | 45956 | 46956 | 47956 |
| 44957 | 45957 | 46957 | 47957 |
| 44958 | 45958 | 46958 | 47958 |
| 44959 | 45959 | 46959 | 47959 |
| 44960 | 45960 | 46960 | 47960 |
| 44961 | 45961 | 46961 | 47961 |
| 44962 | 45962 | 46962 | 47962 |
| 44963 | 45963 | 46963 | 47963 |
| 44964 | 45964 | 46964 | 47964 |
| 44965 | 45965 | 46965 | 47965 |
| 44966 | 45966 | 46966 | 47966 |
| 44967 | 45967 | 46967 | 47967 |
| 44968 | 45968 | 46968 | 47968 |
| 44969 | 45969 | 46969 | 47969 |
| 44970 | 45970 | 46970 | 47970 |
| 44971 | 45971 | 46971 | 47971 |
| 44972 | 45972 | 46972 | 47972 |
| 44973 | 45973 | 46973 | 47973 |
| 44974 | 45974 | 46974 | 47974 |
| 44975 | 45975 | 46975 | 47975 |
| 44976 | 45976 | 46976 | 47976 |
| 44977 | 45977 | 46977 | 47977 |
| 44978 | 45978 | 46978 | 47978 |
| 44979 | 45979 | 46979 | 47979 |
| 44980 | 45980 | 46980 | 47980 |
| 44981 | 45981 | 46981 | 47981 |
| 44982 | 45982 | 46982 | 47982 |
| 44983 | 45983 | 46983 | 47983 |
| 44984 | 45984 | 46984 | 47984 |
| 44985 | 45985 | 46985 | 47985 |
| 44986 | 45986 | 46986 | 47986 |
| 44987 | 45987 | 46987 | 47987 |
| 44988 | 45988 | 46988 | 47988 |
| 44989 | 45989 | 46989 | 47989 |
| 44990 | 45990 | 46990 | 47990 |
| 44991 | 45991 | 46991 | 47991 |
| 44992 | 45992 | 46992 | 47992 |
| 44993 | 45993 | 46993 | 47993 |
| 44994 | 45994 | 46994 | 47994 |
| 44995 | 45995 | 46995 | 47995 |
| 44996 | 45996 | 46996 | 47996 |
| 44997 | 45997 | 46997 | 47997 |
| 44998 | 45998 | 46998 | 47998 |
| 44999 | 45999 | 46999 | 47999 |
| 45000 | 46000 | 47000 | 48000 |
| 45001 | 46001 | 47001 | 48001 |
| 45002 | 46002 | 47002 | 48002 |

In some embodiments, the sequences that specifically bind to casein comprise an inner sequence selected from the group consisting of the nucleic acid sequences of SEQ ID NOs. 48003 to 49002. In some examples, a short nucleic acid sequence may be affixed to the 5-end of the inner sequence. The short nucleic acid sequence may be the 5′ primer sequence used in the random ssDNA library, i.e. the nucleic acid sequence of SEQ ID NO. 1. Accordingly, the aptamer that specifically binds to casein may comprise a nucleic acid sequence selected from the group consisting of SEQ ID NOs. 49003 to 50002. In other examples, a short nucleic acid sequence may be affixed to the 3-end of the inner sequence. The short nucleic acid sequence may be the 3′ primer sequence used in the random ssDNA library, i.e. the nucleic acid sequence of SEQ ID NO. 2. Accordingly, the aptamer that specifically binds to casein may comprise a nucleic acid sequence selected from the group consisting of SEQ ID NOs. 50003 to 51002. In other examples, the inner sequence may comprise a 5′ end short sequence (i.e., SEQ ID NO. 1) and a 3′ end short sequence (i.e., SEQ ID NO. 2). Accordingly, the aptamer that specifically binds to casein may comprise a nucleic acid sequence selected from the group consisting of SEQ ID NOs. 51003 to 52002. In one embodiment, the aptamer of the present disclosure that specifically binds to casein may comprise a nucleic acid sequence selected from the group consisting of SEQ ID NOs. 48003 to 52002 listed in Table 14, or variant thereof.

TABLE 14
Aptamer sequences against casein
Aptamer sequence

| | | | 5′-sequence |
|---|---|---|---|
| | 5′-sequence | Inner | and inner |
| Inner | and inner | sequence and | sequence and |
| sequence | sequence | 3′-sequence | 3′-sequence |
| (SEQ ID NO.) | (SEQ ID NO.) | (SEQ ID NO.) | (SEQ ID NO.) |
| 48003 | 49003 | 50003 | 51003 |
| 48004 | 49004 | 50004 | 51004 |
| 48005 | 49005 | 50005 | 51005 |
| 48006 | 49006 | 50006 | 51006 |
| 48007 | 49007 | 50007 | 51007 |
| 48008 | 49008 | 50008 | 51008 |
| 48009 | 49009 | 50009 | 51009 |
| 48010 | 49010 | 50010 | 51010 |
| 48011 | 49011 | 50011 | 51011 |
| 48012 | 49012 | 50012 | 51012 |
| 48013 | 49013 | 50013 | 51013 |
| 48014 | 49014 | 50014 | 51014 |
| 48015 | 49015 | 50015 | 51015 |
| 48016 | 49016 | 50016 | 51016 |
| 48017 | 49017 | 50017 | 51017 |
| 48018 | 49018 | 50018 | 51018 |
| 48019 | 49019 | 50019 | 51019 |
| 48020 | 49020 | 50020 | 51020 |
| 48021 | 49021 | 50021 | 51021 |
| 48022 | 49022 | 50022 | 51022 |
| 48023 | 49023 | 50023 | 51023 |
| 48024 | 49024 | 50024 | 51024 |
| 48025 | 49025 | 50025 | 51025 |
| 48026 | 49026 | 50026 | 51026 |
| 48027 | 49027 | 50027 | 51027 |
| 48028 | 49028 | 50028 | 51028 |
| 48029 | 49029 | 50029 | 51029 |
| 48030 | 49030 | 50030 | 51030 |
| 48031 | 49031 | 50031 | 51031 |
| 48032 | 49032 | 50032 | 51032 |
| 48033 | 49033 | 50033 | 51033 |
| 48034 | 49034 | 50034 | 51034 |
| 48035 | 49035 | 50035 | 51035 |
| 48036 | 49036 | 50036 | 51036 |
| 48037 | 49037 | 50037 | 51037 |
| 48038 | 49038 | 50038 | 51038 |
| 48039 | 49039 | 50039 | 51039 |
| 48040 | 49040 | 50040 | 51040 |
| 48041 | 49041 | 50041 | 51041 |
| 48042 | 49042 | 50042 | 51042 |
| 48043 | 49043 | 50043 | 51043 |
| 48044 | 49044 | 50044 | 51044 |
| 48045 | 49045 | 50045 | 51045 |
| 48046 | 49046 | 50046 | 51046 |
| 48047 | 49047 | 50047 | 51047 |
| 48048 | 49048 | 50048 | 51048 |
| 48049 | 49049 | 50049 | 51049 |
| 48050 | 49050 | 50050 | 51050 |
| 48051 | 49051 | 50051 | 51051 |
| 48052 | 49052 | 50052 | 51052 |
| 48053 | 49053 | 50053 | 51053 |
| 48054 | 49054 | 50054 | 51054 |
| 48055 | 49055 | 50055 | 51055 |
| 48056 | 49056 | 50056 | 51056 |
| 48057 | 49057 | 50057 | 51057 |
| 48058 | 49058 | 50058 | 51058 |
| 48059 | 49059 | 50059 | 51059 |
| 48060 | 49060 | 50060 | 51060 |
| 48061 | 49061 | 50061 | 51061 |
| 48062 | 49062 | 50062 | 51062 |
| 48063 | 49063 | 50063 | 51063 |
| 48064 | 49064 | 50064 | 51064 |
| 48065 | 49065 | 50065 | 51065 |
| 48066 | 49066 | 50066 | 51066 |
| 48067 | 49067 | 50067 | 51067 |
| 48068 | 49068 | 50068 | 51068 |
| 48069 | 49069 | 50069 | 51069 |
| 48070 | 49070 | 50070 | 51070 |
| 48071 | 49071 | 50071 | 51071 |
| 48072 | 49072 | 50072 | 51072 |
| 48073 | 49073 | 50073 | 51073 |
| 48074 | 49074 | 50074 | 51074 |
| 48075 | 49075 | 50075 | 51075 |
| 48076 | 49076 | 50076 | 51076 |
| 48077 | 49077 | 50077 | 51077 |
| 48078 | 49078 | 50078 | 51078 |
| 48079 | 49079 | 50079 | 51079 |
| 48080 | 49080 | 50080 | 51080 |
| 48081 | 49081 | 50081 | 51081 |
| 48082 | 49082 | 50082 | 51082 |
| 48083 | 49083 | 50083 | 51083 |
| 48084 | 49084 | 50084 | 51084 |
| 48085 | 49085 | 50085 | 51085 |
| 48086 | 49086 | 50086 | 51086 |
| 48087 | 49087 | 50087 | 51087 |
| 48088 | 49088 | 50088 | 51088 |
| 48089 | 49089 | 50089 | 51089 |
| 48090 | 49090 | 50090 | 51090 |
| 48091 | 49091 | 50091 | 51091 |
| 48092 | 49092 | 50092 | 51092 |
| 48093 | 49093 | 50093 | 51093 |
| 48094 | 49094 | 50094 | 51094 |
| 48095 | 49095 | 50095 | 51095 |
| 48096 | 49096 | 50096 | 51096 |
| 48097 | 49097 | 50097 | 51097 |
| 48098 | 49098 | 50098 | 51098 |
| 48099 | 49099 | 50099 | 51099 |
| 48100 | 49100 | 50100 | 51100 |
| 48101 | 49101 | 50101 | 51101 |
| 48102 | 49102 | 50102 | 51102 |
| 48103 | 49103 | 50103 | 51103 |
| 48104 | 49104 | 50104 | 51104 |
| 48105 | 49105 | 50105 | 51105 |
| 48106 | 49106 | 50106 | 51106 |
| 48107 | 49107 | 50107 | 51107 |
| 48108 | 49108 | 50108 | 51108 |
| 48109 | 49109 | 50109 | 51109 |
| 48110 | 49110 | 50110 | 51110 |
| 48111 | 49111 | 50111 | 51111 |
| 48112 | 49112 | 50112 | 51112 |
| 48113 | 49113 | 50113 | 51113 |
| 48114 | 49114 | 50114 | 51114 |
| 48115 | 49115 | 50115 | 51115 |
| 48116 | 49116 | 50116 | 51116 |
| 48117 | 49117 | 50117 | 51117 |
| 48118 | 49118 | 50118 | 51118 |
| 48119 | 49119 | 50119 | 51119 |
| 48120 | 49120 | 50120 | 51120 |
| 48121 | 49121 | 50121 | 51121 |
| 48122 | 49122 | 50122 | 51122 |
| 48123 | 49123 | 50123 | 51123 |
| 48124 | 49124 | 50124 | 51124 |
| 48125 | 49125 | 50125 | 51125 |
| 48126 | 49126 | 50126 | 51126 |
| 48127 | 49127 | 50127 | 51127 |
| 48128 | 49128 | 50128 | 51128 |
| 48129 | 49129 | 50129 | 51129 |
| 48130 | 49130 | 50130 | 51130 |
| 48131 | 49131 | 50131 | 51131 |
| 48132 | 49132 | 50132 | 51132 |
| 48133 | 49133 | 50133 | 51133 |
| 48134 | 49134 | 50134 | 51134 |
| 48135 | 49135 | 50135 | 51135 |
| 48136 | 49136 | 50136 | 51136 |
| 48137 | 49137 | 50137 | 51137 |
| 48138 | 49138 | 50138 | 51138 |
| 48139 | 49139 | 50139 | 51139 |
| 48140 | 49140 | 50140 | 51140 |
| 48141 | 49141 | 50141 | 51141 |
| 48142 | 49142 | 50142 | 51142 |
| 48143 | 49143 | 50143 | 51143 |
| 48144 | 49144 | 50144 | 51144 |
| 48145 | 49145 | 50145 | 51145 |
| 48146 | 49146 | 50146 | 51146 |
| 48147 | 49147 | 50147 | 51147 |
| 48148 | 49148 | 50148 | 51148 |
| 48149 | 49149 | 50149 | 51149 |
| 48150 | 49150 | 50150 | 51150 |
| 48151 | 49151 | 50151 | 51151 |
| 48152 | 49152 | 50152 | 51152 |
| 48153 | 49153 | 50153 | 51153 |
| 48154 | 49154 | 50154 | 51154 |
| 48155 | 49155 | 50155 | 51155 |
| 48156 | 49156 | 50156 | 51156 |
| 48157 | 49157 | 50157 | 51157 |
| 48158 | 49158 | 50158 | 51158 |
| 48159 | 49159 | 50159 | 51159 |
| 48160 | 49160 | 50160 | 51160 |
| 48161 | 49161 | 50161 | 51161 |
| 48162 | 49162 | 50162 | 51162 |
| 48163 | 49163 | 50163 | 51163 |
| 48164 | 49164 | 50164 | 51164 |
| 48165 | 49165 | 50165 | 51165 |
| 48166 | 49166 | 50166 | 51166 |
| 48167 | 49167 | 50167 | 51167 |
| 48168 | 49168 | 50168 | 51168 |
| 48169 | 49169 | 50169 | 51169 |
| 48170 | 49170 | 50170 | 51170 |
| 48171 | 49171 | 50171 | 51171 |
| 48172 | 49172 | 50172 | 51172 |
| 48173 | 49173 | 50173 | 51173 |
| 48174 | 49174 | 50174 | 51174 |
| 48175 | 49175 | 50175 | 51175 |
| 48176 | 49176 | 50176 | 51176 |
| 48177 | 49177 | 50177 | 51177 |
| 48178 | 49178 | 50178 | 51178 |
| 48179 | 49179 | 50179 | 51179 |
| 48180 | 49180 | 50180 | 51180 |
| 48181 | 49181 | 50181 | 51181 |
| 48182 | 49182 | 50182 | 51182 |
| 48183 | 49183 | 50183 | 51183 |
| 48184 | 49184 | 50184 | 51184 |
| 48185 | 49185 | 50185 | 51185 |
| 48186 | 49186 | 50186 | 51186 |
| 48187 | 49187 | 50187 | 51187 |
| 48188 | 49188 | 50188 | 51188 |
| 48189 | 49189 | 50189 | 51189 |
| 48190 | 49190 | 50190 | 51190 |
| 48191 | 49191 | 50191 | 51191 |
| 48192 | 49192 | 50192 | 51192 |
| 48193 | 49193 | 50193 | 51193 |
| 48194 | 49194 | 50194 | 51194 |
| 48195 | 49195 | 50195 | 51195 |
| 48196 | 49196 | 50196 | 51196 |
| 48197 | 49197 | 50197 | 51197 |
| 48198 | 49198 | 50198 | 51198 |
| 48199 | 49199 | 50199 | 51199 |
| 48200 | 49200 | 50200 | 51200 |
| 48201 | 49201 | 50201 | 51201 |
| 48202 | 49202 | 50202 | 51202 |
| 48203 | 49203 | 50203 | 51203 |
| 48204 | 49204 | 50204 | 51204 |
| 48205 | 49205 | 50205 | 51205 |
| 48206 | 49206 | 50206 | 51206 |
| 48207 | 49207 | 50207 | 51207 |
| 48208 | 49208 | 50208 | 51208 |
| 48209 | 49209 | 50209 | 51209 |
| 48210 | 49210 | 50210 | 51210 |
| 48211 | 49211 | 50211 | 51211 |
| 48212 | 49212 | 50212 | 51212 |
| 48213 | 49213 | 50213 | 51213 |
| 48214 | 49214 | 50214 | 51214 |
| 48215 | 49215 | 50215 | 51215 |
| 48216 | 49216 | 50216 | 51216 |
| 48217 | 49217 | 50217 | 51217 |
| 48218 | 49218 | 50218 | 51218 |
| 48219 | 49219 | 50219 | 51219 |
| 48220 | 49220 | 50220 | 51220 |
| 48221 | 49221 | 50221 | 51221 |
| 48222 | 49222 | 50222 | 51222 |
| 48223 | 49223 | 50223 | 51223 |
| 48224 | 49224 | 50224 | 51224 |
| 48225 | 49225 | 50225 | 51225 |
| 48226 | 49226 | 50226 | 51226 |
| 48227 | 49227 | 50227 | 51227 |
| 48228 | 49228 | 50228 | 51228 |
| 48229 | 49229 | 50229 | 51229 |
| 48230 | 49230 | 50230 | 51230 |
| 48231 | 49231 | 50231 | 51231 |
| 48232 | 49232 | 50232 | 51232 |
| 48233 | 49233 | 50233 | 51233 |
| 48234 | 49234 | 50234 | 51234 |
| 48235 | 49235 | 50235 | 51235 |
| 48236 | 49236 | 50236 | 51236 |
| 48237 | 49237 | 50237 | 51237 |
| 48238 | 49238 | 50238 | 51238 |
| 48239 | 49239 | 50239 | 51239 |
| 48240 | 49240 | 50240 | 51240 |
| 48241 | 49241 | 50241 | 51241 |
| 48242 | 49242 | 50242 | 51242 |
| 48243 | 49243 | 50243 | 51243 |
| 48244 | 49244 | 50244 | 51244 |
| 48245 | 49245 | 50245 | 51245 |
| 48246 | 49246 | 50246 | 51246 |
| 48247 | 49247 | 50247 | 51247 |
| 48248 | 49248 | 50248 | 51248 |
| 48249 | 49249 | 50249 | 51249 |
| 48250 | 49250 | 50250 | 51250 |
| 48251 | 49251 | 50251 | 51251 |
| 48252 | 49252 | 50252 | 51252 |
| 48253 | 49253 | 50253 | 51253 |
| 48254 | 49254 | 50254 | 51254 |
| 48255 | 49255 | 50255 | 51255 |
| 48256 | 49256 | 50256 | 51256 |
| 48257 | 49257 | 50257 | 51257 |
| 48258 | 49258 | 50258 | 51258 |
| 48259 | 49259 | 50259 | 51259 |
| 48260 | 49260 | 50260 | 51260 |
| 48261 | 49261 | 50261 | 51261 |
| 48262 | 49262 | 50262 | 51262 |
| 48263 | 49263 | 50263 | 51263 |
| 48264 | 49264 | 50264 | 51264 |
| 48265 | 49265 | 50265 | 51265 |
| 48266 | 49266 | 50266 | 51266 |
| 48267 | 49267 | 50267 | 51267 |
| 48268 | 49268 | 50268 | 51268 |
| 48269 | 49269 | 50269 | 51269 |
| 48270 | 49270 | 50270 | 51270 |
| 48271 | 49271 | 50271 | 51271 |
| 48272 | 49272 | 50272 | 51272 |
| 48273 | 49273 | 50273 | 51273 |
| 48274 | 49274 | 50274 | 51274 |
| 48275 | 49275 | 50275 | 51275 |
| 48276 | 49276 | 50276 | 51276 |
| 48277 | 49277 | 50277 | 51277 |
| 48278 | 49278 | 50278 | 51278 |
| 48279 | 49279 | 50279 | 51279 |
| 48280 | 49280 | 50280 | 51280 |
| 48281 | 49281 | 50281 | 51281 |
| 48282 | 49282 | 50282 | 51282 |
| 48283 | 49283 | 50283 | 51283 |
| 48284 | 49284 | 50284 | 51284 |
| 48285 | 49285 | 50285 | 51285 |
| 48286 | 49286 | 50286 | 51286 |
| 48287 | 49287 | 50287 | 51287 |
| 48288 | 49288 | 50288 | 51288 |
| 48289 | 49289 | 50289 | 51289 |
| 48290 | 49290 | 50290 | 51290 |
| 48291 | 49291 | 50291 | 51291 |
| 48292 | 49292 | 50292 | 51292 |
| 48293 | 49293 | 50293 | 51293 |
| 48294 | 49294 | 50294 | 51294 |
| 48295 | 49295 | 50295 | 51295 |
| 48296 | 49296 | 50296 | 51296 |
| 48297 | 49297 | 50297 | 51297 |
| 48298 | 49298 | 50298 | 51298 |
| 48299 | 49299 | 50299 | 51299 |
| 48300 | 49300 | 50300 | 51300 |
| 48301 | 49301 | 50301 | 51301 |
| 48302 | 49302 | 50302 | 51302 |
| 48303 | 49303 | 50303 | 51303 |
| 48304 | 49304 | 50304 | 51304 |
| 48305 | 49305 | 50305 | 51305 |
| 48306 | 49306 | 50306 | 51306 |
| 48307 | 49307 | 50307 | 51307 |
| 48308 | 49308 | 50308 | 51308 |
| 48309 | 49309 | 50309 | 51309 |
| 48310 | 49310 | 50310 | 51310 |
| 48311 | 49311 | 50311 | 51311 |
| 48312 | 49312 | 50312 | 51312 |
| 48313 | 49313 | 50313 | 51313 |
| 48314 | 49314 | 50314 | 51314 |
| 48315 | 49315 | 50315 | 51315 |
| 48316 | 49316 | 50316 | 51316 |
| 48317 | 49317 | 50317 | 51317 |
| 48318 | 49318 | 50318 | 51318 |
| 48319 | 49319 | 50319 | 51319 |
| 48320 | 49320 | 50320 | 51320 |
| 48321 | 49321 | 50321 | 51321 |
| 48322 | 49322 | 50322 | 51322 |
| 48323 | 49323 | 50323 | 51323 |
| 48324 | 49324 | 50324 | 51324 |
| 48325 | 49325 | 50325 | 51325 |
| 48326 | 49326 | 50326 | 51326 |
| 48327 | 49327 | 50327 | 51327 |
| 48328 | 49328 | 50328 | 51328 |
| 48329 | 49329 | 50329 | 51329 |
| 48330 | 49330 | 50330 | 51330 |
| 48331 | 49331 | 50331 | 51331 |
| 48332 | 49332 | 50332 | 51332 |
| 48333 | 49333 | 50333 | 51333 |
| 48334 | 49334 | 50334 | 51334 |
| 48335 | 49335 | 50335 | 51335 |
| 48336 | 49336 | 50336 | 51336 |
| 48337 | 49337 | 50337 | 51337 |
| 48338 | 49338 | 50338 | 51338 |
| 48339 | 49339 | 50339 | 51339 |
| 48340 | 49340 | 50340 | 51340 |
| 48341 | 49341 | 50341 | 51341 |
| 48342 | 49342 | 50342 | 51342 |
| 48343 | 49343 | 50343 | 51343 |
| 48344 | 49344 | 50344 | 51344 |
| 48345 | 49345 | 50345 | 51345 |
| 48346 | 49346 | 50346 | 51346 |
| 48347 | 49347 | 50347 | 51347 |
| 48348 | 49348 | 50348 | 51348 |
| 48349 | 49349 | 50349 | 51349 |
| 48350 | 49350 | 50350 | 51350 |
| 48351 | 49351 | 50351 | 51351 |
| 48352 | 49352 | 50352 | 51352 |
| 48353 | 49353 | 50353 | 51353 |
| 48354 | 49354 | 50354 | 51354 |
| 48355 | 49355 | 50355 | 51355 |
| 48356 | 49356 | 50356 | 51356 |
| 48357 | 49357 | 50357 | 51357 |
| 48358 | 49358 | 50358 | 51358 |
| 48359 | 49359 | 50359 | 51359 |
| 48360 | 49360 | 50360 | 51360 |
| 48361 | 49361 | 50361 | 51361 |
| 48362 | 49362 | 50362 | 51362 |
| 48363 | 49363 | 50363 | 51363 |
| 48364 | 49364 | 50364 | 51364 |
| 48365 | 49365 | 50365 | 51365 |
| 48366 | 49366 | 50366 | 51366 |
| 48367 | 49367 | 50367 | 51367 |
| 48368 | 49368 | 50368 | 51368 |
| 48369 | 49369 | 50369 | 51369 |
| 48370 | 49370 | 50370 | 51370 |
| 48371 | 49371 | 50371 | 51371 |
| 48372 | 49372 | 50372 | 51372 |
| 48373 | 49373 | 50373 | 51373 |
| 48374 | 49374 | 50374 | 51374 |
| 48375 | 49375 | 50375 | 51375 |
| 48376 | 49376 | 50376 | 51376 |
| 48377 | 49377 | 50377 | 51377 |
| 48378 | 49378 | 50378 | 51378 |
| 48379 | 49379 | 50379 | 51379 |
| 48380 | 49380 | 50380 | 51380 |
| 48381 | 49381 | 50381 | 51381 |
| 48382 | 49382 | 50382 | 51382 |
| 48383 | 49383 | 50383 | 51383 |
| 48384 | 49384 | 50384 | 51384 |
| 48385 | 49385 | 50385 | 51385 |
| 48386 | 49386 | 50386 | 51386 |
| 48387 | 49387 | 50387 | 51387 |
| 48388 | 49388 | 50388 | 51388 |
| 48389 | 49389 | 50389 | 51389 |
| 48390 | 49390 | 50390 | 51390 |
| 48391 | 49391 | 50391 | 51391 |
| 48392 | 49392 | 50392 | 51392 |
| 48393 | 49393 | 50393 | 51393 |
| 48394 | 49394 | 50394 | 51394 |
| 48395 | 49395 | 50395 | 51395 |
| 48396 | 49396 | 50396 | 51396 |
| 48397 | 49397 | 50397 | 51397 |
| 48398 | 49398 | 50398 | 51398 |
| 48399 | 49399 | 50399 | 51399 |
| 48400 | 49400 | 50400 | 51400 |
| 48401 | 49401 | 50401 | 51401 |
| 48402 | 49402 | 50402 | 51402 |
| 48403 | 49403 | 50403 | 51403 |
| 48404 | 49404 | 50404 | 51404 |
| 48405 | 49405 | 50405 | 51405 |
| 48406 | 49406 | 50406 | 51406 |
| 48407 | 49407 | 50407 | 51407 |
| 48408 | 49408 | 50408 | 51408 |
| 48409 | 49409 | 50409 | 51409 |
| 48410 | 49410 | 50410 | 51410 |
| 48411 | 49411 | 50411 | 51411 |
| 48412 | 49412 | 50412 | 51412 |
| 48413 | 49413 | 50413 | 51413 |
| 48414 | 49414 | 50414 | 51414 |
| 48415 | 49415 | 50415 | 51415 |
| 48416 | 49416 | 50416 | 51416 |
| 48417 | 49417 | 50417 | 51417 |
| 48418 | 49418 | 50418 | 51418 |
| 48419 | 49419 | 50419 | 51419 |
| 48420 | 49420 | 50420 | 51420 |
| 48421 | 49421 | 50421 | 51421 |
| 48422 | 49422 | 50422 | 51422 |
| 48423 | 49423 | 50423 | 51423 |
| 48424 | 49424 | 50424 | 51424 |
| 48425 | 49425 | 50425 | 51425 |
| 48426 | 49426 | 50426 | 51426 |
| 48427 | 49427 | 50427 | 51427 |
| 48428 | 49428 | 50428 | 51428 |
| 48429 | 49429 | 50429 | 51429 |
| 48430 | 49430 | 50430 | 51430 |
| 48431 | 49431 | 50431 | 51431 |
| 48432 | 49432 | 50432 | 51432 |
| 48433 | 49433 | 50433 | 51433 |
| 48434 | 49434 | 50434 | 51434 |
| 48435 | 49435 | 50435 | 51435 |
| 48436 | 49436 | 50436 | 51436 |
| 48437 | 49437 | 50437 | 51437 |
| 48438 | 49438 | 50438 | 51438 |
| 48439 | 49439 | 50439 | 51439 |
| 48440 | 49440 | 50440 | 51440 |
| 48441 | 49441 | 50441 | 51441 |
| 48442 | 49442 | 50442 | 51442 |
| 48443 | 49443 | 50443 | 51443 |
| 48444 | 49444 | 50444 | 51444 |
| 48445 | 49445 | 50445 | 51445 |
| 48446 | 49446 | 50446 | 51446 |
| 48447 | 49447 | 50447 | 51447 |
| 48448 | 49448 | 50448 | 51448 |
| 48449 | 49449 | 50449 | 51449 |
| 48450 | 49450 | 50450 | 51450 |
| 48451 | 49451 | 50451 | 51451 |
| 48452 | 49452 | 50452 | 51452 |
| 48453 | 49453 | 50453 | 51453 |
| 48454 | 49454 | 50454 | 51454 |
| 48455 | 49455 | 50455 | 51455 |
| 48456 | 49456 | 50456 | 51456 |
| 48457 | 49457 | 50457 | 51457 |
| 48458 | 49458 | 50458 | 51458 |
| 48459 | 49459 | 50459 | 51459 |
| 48460 | 49460 | 50460 | 51460 |
| 48461 | 49461 | 50461 | 51461 |
| 48462 | 49462 | 50462 | 51462 |
| 48463 | 49463 | 50463 | 51463 |
| 48464 | 49464 | 50464 | 51464 |
| 48465 | 49465 | 50465 | 51465 |
| 48466 | 49466 | 50466 | 51466 |
| 48467 | 49467 | 50467 | 51467 |
| 48468 | 49468 | 50468 | 51468 |
| 48469 | 49469 | 50469 | 51469 |
| 48470 | 49470 | 50470 | 51470 |
| 48471 | 49471 | 50471 | 51471 |
| 48472 | 49472 | 50472 | 51472 |
| 48473 | 49473 | 50473 | 51473 |
| 48474 | 49474 | 50474 | 51474 |
| 48475 | 49475 | 50475 | 51475 |
| 48476 | 49476 | 50476 | 51476 |
| 48477 | 49477 | 50477 | 51477 |
| 48478 | 49478 | 50478 | 51478 |
| 48479 | 49479 | 50479 | 51479 |
| 48480 | 49480 | 50480 | 51480 |
| 48481 | 49481 | 50481 | 51481 |
| 48482 | 49482 | 50482 | 51482 |
| 48483 | 49483 | 50483 | 51483 |
| 48484 | 49484 | 50484 | 51484 |
| 48485 | 49485 | 50485 | 51485 |
| 48486 | 49486 | 50486 | 51486 |
| 48487 | 49487 | 50487 | 51487 |
| 48488 | 49488 | 50488 | 51488 |
| 48489 | 49489 | 50489 | 51489 |
| 48490 | 49490 | 50490 | 51490 |
| 48491 | 49491 | 50491 | 51491 |
| 48492 | 49492 | 50492 | 51492 |
| 48493 | 49493 | 50493 | 51493 |
| 48494 | 49494 | 50494 | 51494 |
| 48495 | 49495 | 50495 | 51495 |
| 48496 | 49496 | 50496 | 51496 |
| 48497 | 49497 | 50497 | 51497 |
| 48498 | 49498 | 50498 | 51498 |
| 48499 | 49499 | 50499 | 51499 |
| 48500 | 49500 | 50500 | 51500 |
| 48501 | 49501 | 50501 | 51501 |
| 48502 | 49502 | 50502 | 51502 |
| 48503 | 49503 | 50503 | 51503 |
| 48504 | 49504 | 50504 | 51504 |
| 48505 | 49505 | 50505 | 51505 |
| 48506 | 49506 | 50506 | 51506 |
| 48507 | 49507 | 50507 | 51507 |
| 48508 | 49508 | 50508 | 51508 |
| 48509 | 49509 | 50509 | 51509 |
| 48510 | 49510 | 50510 | 51510 |
| 48511 | 49511 | 50511 | 51511 |
| 48512 | 49512 | 50512 | 51512 |
| 48513 | 49513 | 50513 | 51513 |
| 48514 | 49514 | 50514 | 51514 |
| 48515 | 49515 | 50515 | 51515 |
| 48516 | 49516 | 50516 | 51516 |
| 48517 | 49517 | 50517 | 51517 |
| 48518 | 49518 | 50518 | 51518 |
| 48519 | 49519 | 50519 | 51519 |
| 48520 | 49520 | 50520 | 51520 |
| 48521 | 49521 | 50521 | 51521 |
| 48522 | 49522 | 50522 | 51522 |
| 48523 | 49523 | 50523 | 51523 |
| 48524 | 49524 | 50524 | 51524 |
| 48525 | 49525 | 50525 | 51525 |
| 48526 | 49526 | 50526 | 51526 |
| 48527 | 49527 | 50527 | 51527 |
| 48528 | 49528 | 50528 | 51528 |
| 48529 | 49529 | 50529 | 51529 |
| 48530 | 49530 | 50530 | 51530 |
| 48531 | 49531 | 50531 | 51531 |
| 48532 | 49532 | 50532 | 51532 |
| 48533 | 49533 | 50533 | 51533 |
| 48534 | 49534 | 50534 | 51534 |
| 48535 | 49535 | 50535 | 51535 |
| 48536 | 49536 | 50536 | 51536 |
| 48537 | 49537 | 50537 | 51537 |
| 48538 | 49538 | 50538 | 51538 |
| 48539 | 49539 | 50539 | 51539 |
| 48540 | 49540 | 50540 | 51540 |
| 48541 | 49541 | 50541 | 51541 |
| 48542 | 49542 | 50542 | 51542 |
| 48543 | 49543 | 50543 | 51543 |
| 48544 | 49544 | 50544 | 51544 |
| 48545 | 49545 | 50545 | 51545 |
| 48546 | 49546 | 50546 | 51546 |
| 48547 | 49547 | 50547 | 51547 |
| 48548 | 49548 | 50548 | 51548 |
| 48549 | 49549 | 50549 | 51549 |
| 48550 | 49550 | 50550 | 51550 |
| 48551 | 49551 | 50551 | 51551 |
| 48552 | 49552 | 50552 | 51552 |
| 48553 | 49553 | 50553 | 51553 |
| 48554 | 49554 | 50554 | 51554 |
| 48555 | 49555 | 50555 | 51555 |
| 48556 | 49556 | 50556 | 51556 |
| 48557 | 49557 | 50557 | 51557 |
| 48558 | 49558 | 50558 | 51558 |
| 48559 | 49559 | 50559 | 51559 |
| 48560 | 49560 | 50560 | 51560 |
| 48561 | 49561 | 50561 | 51561 |
| 48562 | 49562 | 50562 | 51562 |
| 48563 | 49563 | 50563 | 51563 |
| 48564 | 49564 | 50564 | 51564 |
| 48565 | 49565 | 50565 | 51565 |
| 48566 | 49566 | 50566 | 51566 |
| 48567 | 49567 | 50567 | 51567 |
| 48568 | 49568 | 50568 | 51568 |
| 48569 | 49569 | 50569 | 51569 |
| 48570 | 49570 | 50570 | 51570 |
| 48571 | 49571 | 50571 | 51571 |
| 48572 | 49572 | 50572 | 51572 |
| 48573 | 49573 | 50573 | 51573 |
| 48574 | 49574 | 50574 | 51574 |
| 48575 | 49575 | 50575 | 51575 |
| 48576 | 49576 | 50576 | 51576 |
| 48577 | 49577 | 50577 | 51577 |
| 48578 | 49578 | 50578 | 51578 |
| 48579 | 49579 | 50579 | 51579 |
| 48580 | 49580 | 50580 | 51580 |
| 48581 | 49581 | 50581 | 51581 |
| 48582 | 49582 | 50582 | 51582 |
| 48583 | 49583 | 50583 | 51583 |
| 48584 | 49584 | 50584 | 51584 |
| 48585 | 49585 | 50585 | 51585 |
| 48586 | 49586 | 50586 | 51586 |
| 48587 | 49587 | 50587 | 51587 |
| 48588 | 49588 | 50588 | 51588 |
| 48589 | 49589 | 50589 | 51589 |
| 48590 | 49590 | 50590 | 51590 |
| 48591 | 49591 | 50591 | 51591 |
| 48592 | 49592 | 50592 | 51592 |
| 48593 | 49593 | 50593 | 51593 |
| 48594 | 49594 | 50594 | 51594 |
| 48595 | 49595 | 50595 | 51595 |
| 48596 | 49596 | 50596 | 51596 |
| 48597 | 49597 | 50597 | 51597 |
| 48598 | 49598 | 50598 | 51598 |
| 48599 | 49599 | 50599 | 51599 |
| 48600 | 49600 | 50600 | 51600 |
| 48601 | 49601 | 50601 | 51601 |
| 48602 | 49602 | 50602 | 51602 |
| 48603 | 49603 | 50603 | 51603 |
| 48604 | 49604 | 50604 | 51604 |
| 48605 | 49605 | 50605 | 51605 |
| 48606 | 49606 | 50606 | 51606 |
| 48607 | 49607 | 50607 | 51607 |
| 48608 | 49608 | 50608 | 51608 |
| 48609 | 49609 | 50609 | 51609 |
| 48610 | 49610 | 50610 | 51610 |
| 48611 | 49611 | 50611 | 51611 |
| 48612 | 49612 | 50612 | 51612 |
| 48613 | 49613 | 50613 | 51613 |
| 48614 | 49614 | 50614 | 51614 |
| 48615 | 49615 | 50615 | 51615 |
| 48616 | 49616 | 50616 | 51616 |
| 48617 | 49617 | 50617 | 51617 |
| 48618 | 49618 | 50618 | 51618 |
| 48619 | 49619 | 50619 | 51619 |
| 48620 | 49620 | 50620 | 51620 |
| 48621 | 49621 | 50621 | 51621 |
| 48622 | 49622 | 50622 | 51622 |
| 48623 | 49623 | 50623 | 51623 |
| 48624 | 49624 | 50624 | 51624 |
| 48625 | 49625 | 50625 | 51625 |
| 48626 | 49626 | 50626 | 51626 |
| 48627 | 49627 | 50627 | 51627 |
| 48628 | 49628 | 50628 | 51628 |
| 48629 | 49629 | 50629 | 51629 |
| 48630 | 49630 | 50630 | 51630 |
| 48631 | 49631 | 50631 | 51631 |
| 48632 | 49632 | 50632 | 51632 |
| 48633 | 49633 | 50633 | 51633 |
| 48634 | 49634 | 50634 | 51634 |
| 48635 | 49635 | 50635 | 51635 |
| 48636 | 49636 | 50636 | 51636 |
| 48637 | 49637 | 50637 | 51637 |
| 48638 | 49638 | 50638 | 51638 |
| 48639 | 49639 | 50639 | 51639 |
| 48640 | 49640 | 50640 | 51640 |
| 48641 | 49641 | 50641 | 51641 |
| 48642 | 49642 | 50642 | 51642 |
| 48643 | 49643 | 50643 | 51643 |
| 48644 | 49644 | 50644 | 51644 |
| 48645 | 49645 | 50645 | 51645 |
| 48646 | 49646 | 50646 | 51646 |
| 48647 | 49647 | 50647 | 51647 |
| 48648 | 49648 | 50648 | 51648 |
| 48649 | 49649 | 50649 | 51649 |
| 48650 | 49650 | 50650 | 51650 |
| 48651 | 49651 | 50651 | 51651 |
| 48652 | 49652 | 50652 | 51652 |
| 48653 | 49653 | 50653 | 51653 |
| 48654 | 49654 | 50654 | 51654 |
| 48655 | 49655 | 50655 | 51655 |
| 48656 | 49656 | 50656 | 51656 |
| 48657 | 49657 | 50657 | 51657 |
| 48658 | 49658 | 50658 | 51658 |
| 48659 | 49659 | 50659 | 51659 |
| 48660 | 49660 | 50660 | 51660 |
| 48661 | 49661 | 50661 | 51661 |
| 48662 | 49662 | 50662 | 51662 |
| 48663 | 49663 | 50663 | 51663 |
| 48664 | 49664 | 50664 | 51664 |
| 48665 | 49665 | 50665 | 51665 |
| 48666 | 49666 | 50666 | 51666 |
| 48667 | 49667 | 50667 | 51667 |
| 48668 | 49668 | 50668 | 51668 |
| 48669 | 49669 | 50669 | 51669 |
| 48670 | 49670 | 50670 | 51670 |
| 48671 | 49671 | 50671 | 51671 |
| 48672 | 49672 | 50672 | 51672 |
| 48673 | 49673 | 50673 | 51673 |
| 48674 | 49674 | 50674 | 51674 |
| 48675 | 49675 | 50675 | 51675 |
| 48676 | 49676 | 50676 | 51676 |
| 48677 | 49677 | 50677 | 51677 |
| 48678 | 49678 | 50678 | 51678 |
| 48679 | 49679 | 50679 | 51679 |
| 48680 | 49680 | 50680 | 51680 |
| 48681 | 49681 | 50681 | 51681 |
| 48682 | 49682 | 50682 | 51682 |
| 48683 | 49683 | 50683 | 51683 |
| 48684 | 49684 | 50684 | 51684 |
| 48685 | 49685 | 50685 | 51685 |
| 48686 | 49686 | 50686 | 51686 |
| 48687 | 49687 | 50687 | 51687 |
| 48688 | 49688 | 50688 | 51688 |
| 48689 | 49689 | 50689 | 51689 |
| 48690 | 49690 | 50690 | 51690 |
| 48691 | 49691 | 50691 | 51691 |
| 48692 | 49692 | 50692 | 51692 |
| 48693 | 49693 | 50693 | 51693 |
| 48694 | 49694 | 50694 | 51694 |
| 48695 | 49695 | 50695 | 51695 |
| 48696 | 49696 | 50696 | 51696 |
| 48697 | 49697 | 50697 | 51697 |
| 48698 | 49698 | 50698 | 51698 |
| 48699 | 49699 | 50699 | 51699 |
| 48700 | 49700 | 50700 | 51700 |
| 48701 | 49701 | 50701 | 51701 |
| 48702 | 49702 | 50702 | 51702 |
| 48703 | 49703 | 50703 | 51703 |
| 48704 | 49704 | 50704 | 51704 |
| 48705 | 49705 | 50705 | 51705 |
| 48706 | 49706 | 50706 | 51706 |
| 48707 | 49707 | 50707 | 51707 |
| 48708 | 49708 | 50708 | 51708 |
| 48709 | 49709 | 50709 | 51709 |
| 48710 | 49710 | 50710 | 51710 |
| 48711 | 49711 | 50711 | 51711 |
| 48712 | 49712 | 50712 | 51712 |
| 48713 | 49713 | 50713 | 51713 |
| 48714 | 49714 | 50714 | 51714 |
| 48715 | 49715 | 50715 | 51715 |
| 48716 | 49716 | 50716 | 51716 |
| 48717 | 49717 | 50717 | 51717 |
| 48718 | 49718 | 50718 | 51718 |
| 48719 | 49719 | 50719 | 51719 |
| 48720 | 49720 | 50720 | 51720 |
| 48721 | 49721 | 50721 | 51721 |
| 48722 | 49722 | 50722 | 51722 |
| 48723 | 49723 | 50723 | 51723 |
| 48724 | 49724 | 50724 | 51724 |
| 48725 | 49725 | 50725 | 51725 |
| 48726 | 49726 | 50726 | 51726 |
| 48727 | 49727 | 50727 | 51727 |
| 48728 | 49728 | 50728 | 51728 |
| 48729 | 49729 | 50729 | 51729 |
| 48730 | 49730 | 50730 | 51730 |
| 48731 | 49731 | 50731 | 51731 |
| 48732 | 49732 | 50732 | 51732 |
| 48733 | 49733 | 50733 | 51733 |
| 48734 | 49734 | 50734 | 51734 |
| 48735 | 49735 | 50735 | 51735 |
| 48736 | 49736 | 50736 | 51736 |
| 48737 | 49737 | 50737 | 51737 |
| 48738 | 49738 | 50738 | 51738 |
| 48739 | 49739 | 50739 | 51739 |
| 48740 | 49740 | 50740 | 51740 |
| 48741 | 49741 | 50741 | 51741 |
| 48742 | 49742 | 50742 | 51742 |
| 48743 | 49743 | 50743 | 51743 |
| 48744 | 49744 | 50744 | 51744 |
| 48745 | 49745 | 50745 | 51745 |
| 48746 | 49746 | 50746 | 51746 |
| 48747 | 49747 | 50747 | 51747 |
| 48748 | 49748 | 50748 | 51748 |
| 48749 | 49749 | 50749 | 51749 |
| 48750 | 49750 | 50750 | 51750 |
| 48751 | 49751 | 50751 | 51751 |
| 48752 | 49752 | 50752 | 51752 |
| 48753 | 49753 | 50753 | 51753 |
| 48754 | 49754 | 50754 | 51754 |
| 48755 | 49755 | 50755 | 51755 |
| 48756 | 49756 | 50756 | 51756 |
| 48757 | 49757 | 50757 | 51757 |
| 48758 | 49758 | 50758 | 51758 |
| 48759 | 49759 | 50759 | 51759 |
| 48760 | 49760 | 50760 | 51760 |
| 48761 | 49761 | 50761 | 51761 |
| 48762 | 49762 | 50762 | 51762 |
| 48763 | 49763 | 50763 | 51763 |
| 48764 | 49764 | 50764 | 51764 |
| 48765 | 49765 | 50765 | 51765 |
| 48766 | 49766 | 50766 | 51766 |
| 48767 | 49767 | 50767 | 51767 |
| 48768 | 49768 | 50768 | 51768 |
| 48769 | 49769 | 50769 | 51769 |
| 48770 | 49770 | 50770 | 51770 |
| 48771 | 49771 | 50771 | 51771 |
| 48772 | 49772 | 50772 | 51772 |
| 48773 | 49773 | 50773 | 51773 |
| 48774 | 49774 | 50774 | 51774 |
| 48775 | 49775 | 50775 | 51775 |
| 48776 | 49776 | 50776 | 51776 |
| 48777 | 49777 | 50777 | 51777 |
| 48778 | 49778 | 50778 | 51778 |
| 48779 | 49779 | 50779 | 51779 |
| 48780 | 49780 | 50780 | 51780 |
| 48781 | 49781 | 50781 | 51781 |
| 48782 | 49782 | 50782 | 51782 |
| 48783 | 49783 | 50783 | 51783 |
| 48784 | 49784 | 50784 | 51784 |
| 48785 | 49785 | 50785 | 51785 |
| 48786 | 49786 | 50786 | 51786 |
| 48787 | 49787 | 50787 | 51787 |
| 48788 | 49788 | 50788 | 51788 |
| 48789 | 49789 | 50789 | 51789 |
| 48790 | 49790 | 50790 | 51790 |
| 48791 | 49791 | 50791 | 51791 |
| 48792 | 49792 | 50792 | 51792 |
| 48793 | 49793 | 50793 | 51793 |
| 48794 | 49794 | 50794 | 51794 |
| 48795 | 49795 | 50795 | 51795 |
| 48796 | 49796 | 50796 | 51796 |
| 48797 | 49797 | 50797 | 51797 |
| 48798 | 49798 | 50798 | 51798 |
| 48799 | 49799 | 50799 | 51799 |
| 48800 | 49800 | 50800 | 51800 |
| 48801 | 49801 | 50801 | 51801 |
| 48802 | 49802 | 50802 | 51802 |
| 48803 | 49803 | 50803 | 51803 |
| 48804 | 49804 | 50804 | 51804 |
| 48805 | 49805 | 50805 | 51805 |
| 48806 | 49806 | 50806 | 51806 |
| 48807 | 49807 | 50807 | 51807 |
| 48808 | 49808 | 50808 | 51808 |
| 48809 | 49809 | 50809 | 51809 |
| 48810 | 49810 | 50810 | 51810 |
| 48811 | 49811 | 50811 | 51811 |
| 48812 | 49812 | 50812 | 51812 |
| 48813 | 49813 | 50813 | 51813 |
| 48814 | 49814 | 50814 | 51814 |
| 48815 | 49815 | 50815 | 51815 |
| 48816 | 49816 | 50816 | 51816 |
| 48817 | 49817 | 50817 | 51817 |
| 48818 | 49818 | 50818 | 51818 |
| 48819 | 49819 | 50819 | 51819 |
| 48820 | 49820 | 50820 | 51820 |
| 48821 | 49821 | 50821 | 51821 |
| 48822 | 49822 | 50822 | 51822 |
| 48823 | 49823 | 50823 | 51823 |
| 48824 | 49824 | 50824 | 51824 |
| 48825 | 49825 | 50825 | 51825 |
| 48826 | 49826 | 50826 | 51826 |
| 48827 | 49827 | 50827 | 51827 |
| 48828 | 49828 | 50828 | 51828 |
| 48829 | 49829 | 50829 | 51829 |
| 48830 | 49830 | 50830 | 51830 |
| 48831 | 49831 | 50831 | 51831 |
| 48832 | 49832 | 50832 | 51832 |
| 48833 | 49833 | 50833 | 51833 |
| 48834 | 49834 | 50834 | 51834 |
| 48835 | 49835 | 50835 | 51835 |
| 48836 | 49836 | 50836 | 51836 |
| 48837 | 49837 | 50837 | 51837 |
| 48838 | 49838 | 50838 | 51838 |
| 48839 | 49839 | 50839 | 51839 |
| 48840 | 49840 | 50840 | 51840 |
| 48841 | 49841 | 50841 | 51841 |
| 48842 | 49842 | 50842 | 51842 |
| 48843 | 49843 | 50843 | 51843 |
| 48844 | 49844 | 50844 | 51844 |
| 48845 | 49845 | 50845 | 51845 |
| 48846 | 49846 | 50846 | 51846 |
| 48847 | 49847 | 50847 | 51847 |
| 48848 | 49848 | 50848 | 51848 |
| 48849 | 49849 | 50849 | 51849 |
| 48850 | 49850 | 50850 | 51850 |
| 48851 | 49851 | 50851 | 51851 |
| 48852 | 49852 | 50852 | 51852 |
| 48853 | 49853 | 50853 | 51853 |
| 48854 | 49854 | 50854 | 51854 |
| 48855 | 49855 | 50855 | 51855 |
| 48856 | 49856 | 50856 | 51856 |
| 48857 | 49857 | 50857 | 51857 |
| 48858 | 49858 | 50858 | 51858 |
| 48859 | 49859 | 50859 | 51859 |
| 48860 | 49860 | 50860 | 51860 |
| 48861 | 49861 | 50861 | 51861 |
| 48862 | 49862 | 50862 | 51862 |
| 48863 | 49863 | 50863 | 51863 |
| 48864 | 49864 | 50864 | 51864 |
| 48865 | 49865 | 50865 | 51865 |
| 48866 | 49866 | 50866 | 51866 |
| 48867 | 49867 | 50867 | 51867 |
| 48868 | 49868 | 50868 | 51868 |
| 48869 | 49869 | 50869 | 51869 |
| 48870 | 49870 | 50870 | 51870 |
| 48871 | 49871 | 50871 | 51871 |
| 48872 | 49872 | 50872 | 51872 |
| 48873 | 49873 | 50873 | 51873 |
| 48874 | 49874 | 50874 | 51874 |
| 48875 | 49875 | 50875 | 51875 |
| 48876 | 49876 | 50876 | 51876 |
| 48877 | 49877 | 50877 | 51877 |
| 48878 | 49878 | 50878 | 51878 |
| 48879 | 49879 | 50879 | 51879 |
| 48880 | 49880 | 50880 | 51880 |
| 48881 | 49881 | 50881 | 51881 |
| 48882 | 49882 | 50882 | 51882 |
| 48883 | 49883 | 50883 | 51883 |
| 48884 | 49884 | 50884 | 51884 |
| 48885 | 49885 | 50885 | 51885 |
| 48886 | 49886 | 50886 | 51886 |
| 48887 | 49887 | 50887 | 51887 |
| 48888 | 49888 | 50888 | 51888 |
| 48889 | 49889 | 50889 | 51889 |
| 48890 | 49890 | 50890 | 51890 |
| 48891 | 49891 | 50891 | 51891 |
| 48892 | 49892 | 50892 | 51892 |
| 48893 | 49893 | 50893 | 51893 |
| 48894 | 49894 | 50894 | 51894 |
| 48895 | 49895 | 50895 | 51895 |
| 48896 | 49896 | 50896 | 51896 |
| 48897 | 49897 | 50897 | 51897 |
| 48898 | 49898 | 50898 | 51898 |
| 48899 | 49899 | 50899 | 51899 |
| 48900 | 49900 | 50900 | 51900 |
| 48901 | 49901 | 50901 | 51901 |
| 48902 | 49902 | 50902 | 51902 |
| 48903 | 49903 | 50903 | 51903 |
| 48904 | 49904 | 50904 | 51904 |
| 48905 | 49905 | 50905 | 51905 |
| 48906 | 49906 | 50906 | 51906 |
| 48907 | 49907 | 50907 | 51907 |
| 48908 | 49908 | 50908 | 51908 |
| 48909 | 49909 | 50909 | 51909 |
| 48910 | 49910 | 50910 | 51910 |
| 48911 | 49911 | 50911 | 51911 |
| 48912 | 49912 | 50912 | 51912 |
| 48913 | 49913 | 50913 | 51913 |
| 48914 | 49914 | 50914 | 51914 |
| 48915 | 49915 | 50915 | 51915 |
| 48916 | 49916 | 50916 | 51916 |
| 48917 | 49917 | 50917 | 51917 |
| 48918 | 49918 | 50918 | 51918 |
| 48919 | 49919 | 50919 | 51919 |
| 48920 | 49920 | 50920 | 51920 |
| 48921 | 49921 | 50921 | 51921 |
| 48922 | 49922 | 50922 | 51922 |
| 48923 | 49923 | 50923 | 51923 |
| 48924 | 49924 | 50924 | 51924 |
| 48925 | 49925 | 50925 | 51925 |
| 48926 | 49926 | 50926 | 51926 |
| 48927 | 49927 | 50927 | 51927 |
| 48928 | 49928 | 50928 | 51928 |
| 48929 | 49929 | 50929 | 51929 |
| 48930 | 49930 | 50930 | 51930 |
| 48931 | 49931 | 50931 | 51931 |
| 48932 | 49932 | 50932 | 51932 |
| 48933 | 49933 | 50933 | 51933 |
| 48934 | 49934 | 50934 | 51934 |
| 48935 | 49935 | 50935 | 51935 |
| 48936 | 49936 | 50936 | 51936 |
| 48937 | 49937 | 50937 | 51937 |
| 48938 | 49938 | 50938 | 51938 |
| 48939 | 49939 | 50939 | 51939 |
| 48940 | 49940 | 50940 | 51940 |
| 48941 | 49941 | 50941 | 51941 |
| 48942 | 49942 | 50942 | 51942 |
| 48943 | 49943 | 50943 | 51943 |
| 48944 | 49944 | 50944 | 51944 |
| 48945 | 49945 | 50945 | 51945 |
| 48946 | 49946 | 50946 | 51946 |
| 48947 | 49947 | 50947 | 51947 |
| 48948 | 49948 | 50948 | 51948 |
| 48949 | 49949 | 50949 | 51949 |
| 48950 | 49950 | 50950 | 51950 |
| 48951 | 49951 | 50951 | 51951 |
| 48952 | 49952 | 50952 | 51952 |
| 48953 | 49953 | 50953 | 51953 |
| 48954 | 49954 | 50954 | 51954 |
| 48955 | 49955 | 50955 | 51955 |
| 48956 | 49956 | 50956 | 51956 |
| 48957 | 49957 | 50957 | 51957 |
| 48958 | 49958 | 50958 | 51958 |
| 48959 | 49959 | 50959 | 51959 |
| 48960 | 49960 | 50960 | 51960 |
| 48961 | 49961 | 50961 | 51961 |
| 48962 | 49962 | 50962 | 51962 |
| 48963 | 49963 | 50963 | 51963 |
| 48964 | 49964 | 50964 | 51964 |
| 48965 | 49965 | 50965 | 51965 |
| 48966 | 49966 | 50966 | 51966 |
| 48967 | 49967 | 50967 | 51967 |
| 48968 | 49968 | 50968 | 51968 |
| 48969 | 49969 | 50969 | 51969 |
| 48970 | 49970 | 50970 | 51970 |
| 48971 | 49971 | 50971 | 51971 |
| 48972 | 49972 | 50972 | 51972 |
| 48973 | 49973 | 50973 | 51973 |
| 48974 | 49974 | 50974 | 51974 |
| 48975 | 49975 | 50975 | 51975 |
| 48976 | 49976 | 50976 | 51976 |
| 48977 | 49977 | 50977 | 51977 |
| 48978 | 49978 | 50978 | 51978 |
| 48979 | 49979 | 50979 | 51979 |
| 48980 | 49980 | 50980 | 51980 |
| 48981 | 49981 | 50981 | 51981 |
| 48982 | 49982 | 50982 | 51982 |
| 48983 | 49983 | 50983 | 51983 |
| 48984 | 49984 | 50984 | 51984 |
| 48985 | 49985 | 50985 | 51985 |
| 48986 | 49986 | 50986 | 51986 |
| 48987 | 49987 | 50987 | 51987 |
| 48988 | 49988 | 50988 | 51988 |
| 48989 | 49989 | 50989 | 51989 |
| 48990 | 49990 | 50990 | 51990 |
| 48991 | 49991 | 50991 | 51991 |
| 48992 | 49992 | 50992 | 51992 |
| 48993 | 49993 | 50993 | 51993 |
| 48994 | 49994 | 50994 | 51994 |
| 48995 | 49995 | 50995 | 51995 |
| 48996 | 49996 | 50996 | 51996 |
| 48997 | 49997 | 50997 | 51997 |
| 48998 | 49998 | 50998 | 51998 |
| 48999 | 49999 | 50999 | 51999 |
| 49000 | 50000 | 51000 | 52000 |
| 49001 | 50001 | 51001 | 52001 |
| 49002 | 50002 | 51002 | 52002 |

In some embodiments, the SPN of the present disclosure comprises an aptamer selected by the present method and a short oligonucleotide anchor sequence that may be coated to a solid support. The short anchor oligonucleotide comprises a nucleic acid sequence complementary to a portion of the same aptamer sequence. In some embodiments, a SPN for detecting peanut allergen comprises an aptamer sequence selected from the group consisting of SEQ ID Nos. 3 to 4002 and one or more short anchor sequences that are complementary to the aptamer sequence. As a non-limiting example, the complementary sequence may comprise a nucleic acid sequence selected from SEQ ID NOs. 52003 to 52042 (as shown in Table 15). In some embodiments, the anchor oligonucleotide may be modified to contain a spacer at one end of the sequence. As a non-limiting example, the anchor sequence is modified to contain either a 12-Carbon atom spacer or a 6-Carbon atom spacer at the 5′ end of the sequence (Table 15), or a polyA tail at one end of the sequence. The short complementary sequences may be covalently attached to the solid support (e.g., a glass or plastic chip) directly or through a linker. Accordingly, the length of the linker (carbon atoms or polyA tail) from the solid surface can prevent steric hindrance and reduce the probability of interference due to auto-fluorescence of matrices.

TABLE 15
Short complementary anchor sequences

| SEQ ID NO. | Sequence (5′-3′) |
|---|---|
| 52003 | 6C-AAAAATCAAGTGGTC |
| 52004 | 12C-AAAAATCAAGTGGTC |
| 52005 | 6C-AAAAATCAAGTG |
| 52006 | 12C-AAAAATCAAGTG |
| 52007 | 6C-AAAAAAGTGGTC |
| 52008 | 12C-AAAAAAGTGGTC |
| 52009 | 6C-AAAAATCAAGAGGTC |
| 52010 | 12C-AAAAATCAAGAGGTC |
| 52011 | 6C-AAAAATCAACAGGTC |
| 52012 | 12C-AAAAATCAACAGGTC |
| 52013 | 6C-AAAAAAGTGGTCATG |
| 52014 | 12C-AAAAAAGTGGTCATG |
| 52015 | 6C-AAAAATGGTCATGTA |
| 52016 | 12C-AAAAATGGTCATGTA |
| 52017 | 6C-AAAAATGGTCAT |
| 52018 | 12C-AAAAATGGTCAT |
| 52019 | 6C-AAAAATCATGTA |
| 52020 | 12C-AAAAATCATGTA |
| 52021 | 6C-AAAAATGGTCTTGTA |
| 52022 | 12C-AAAAATGGTCTTGTA |
| 52023 | 6C-AAAAATGGTGTTGTA |
| 52024 | 12C-AAAAATGGTGTTGTA |
| 52025 | 6C-AAAAATCATGTACTA |
| 52026 | 12C-AAAAATCATGTACTA |
| 52027 | 6C-AAAAACTCTTCCCTA |
| 52028 | 12C-AAAAACTCTTCCCTA |
| 52029 | 6C-AAAAACTCTTCC |
| 52030 | 12C-AAAAACTCTTCC |
| 52031 | 6C-AAAAATTCCCTA |
| 52032 | 12C-AAAAATTCCCTA |
| 52033 | 6C-AAAAACTCTTGCCTA |
| 52034 | 12C-AAAAACTCTTGCCTA |
| 52035 | 6C-AAAAACTCTAGCCTA |
| 52036 | 12C-AAAAACTCTAGCCTA |
| 52037 | 6C-AAAAAGATCAGGCCA |
| 52038 | 6C-AAAAACACTTGCGGT |
| 52039 | 6C-AAAAACACGGACACG |
| 52040 | 6C-AAAAAGGCCATGCTT |
| 52041 | 6C-AAAAAGCTGCTCATC |
| 52042 | 6C-AAAAAGAAGACACAC |

### Detection Kits

In some embodiments, the present disclosure provides a detection kit for allergen detection. The kit comprises (a) a SPN comprising an aptamer sequence that specifically binds to a target of interest, wherein the aptamer does not bind to its complementary sequence in the presence of the target of interest; and (b) a solid support of which the surface is coated with short nucleic acid sequences that are complementary to the sequence of the aptamer. The detection kit may further comprise one or more buffer solutions and other reagents. The buffers are suitable for preparing sample solutions, SPN solutions, and/or other solutions necessary for running a detection assay (e.g., wash buffers). One or more of these kit components may be separated into individual containers, or they may be provided in their aggregated state. In some embodiments, the kit may comprise multiple SPNs specific to multiple allergen targets. For example, the kit may comprise a panel of SPNs specific to peanut and common tree nuts including almond, brazil nut, cashew, hazel nut, pecan, pistachio and walnut.

In some embodiments, the detection kit may further comprise one or more control aptamer sequences; the control sequences may be used to measure total protein and normalize the baseline. For example, a detection kit comprising SPNs specific to peanut for peanut detection may comprise peanut control sequences that can measure total protein and normalize the baseline during peanut detection. As a non-limiting example, a peanut detection kit may comprise one or more peanut specific aptamers comprising nucleic acid sequences selected from SEQ ID NOs. 3-4002 and one or more peanut control aptamers comprising nucleic acid sequences selected from SEQ ID NOs. 36003 to 40002.

### Detection Assays

In some embodiments, the present disclosure provides a method for detecting the presence and/or absence of an allergen in a food sample, the method comprising the steps of (i) preparing a sample to be tested solution and a SPN solution; (ii) mixing the sample and SPN solutions and incubating the mixture to induce the binding of the target to the SPN; (iii) contacting the mixture to a solid support that is coated with short oligonucleotides comprising sequences complementary to the SPN; and (iv) measuring a signal and detecting the presence and/or absence of the allergen of interest. The SPN may be labeled with a fluorophore at one end of the sequence, e.g., Cy5 and Alexa Fluor 647.

In some embodiments, the solid support is a glass chip (e.g., a borosilicate glass chip) wherein the surface of the glass chip is divided into several panels including at least one reactive panel and at least two control panels. The reactive panel of the glass chip are covalently coated with short oligonucleotides comprising sequences complementary to the SPN to which the SPN can hybridize to form a double stranded nucleic acid when the SPN is free from the binding of the target of interest. The reactive panel may be flanked by two control panels at each side. The control panels may be coated with random sequences that do not bind to the SPN nor the target.

The chip can be any size suitable for the use in a detection device/system, e.g., 10×10 mm. In some embodiments, the detection chip may be a plastic chip.

In some embodiments, the food sample may be processed with a homogenization buffer that contains a SPN specific to an allergen of interest (e.g., peanut). The food slurry passes over a reactive panel on a glass chip, embedded in a cartridge designed to position the chip to face a laser and an optical sensor. Wash buffer is flowed over the reactive panel, thereby removing any non-specific binding interactions from the panel. Multiple steps of the assay are read by the optical sensor and analyzed by an algorithm to provide an “allergen detected” or “allergen not detected” response. In the absence of the target allergen, the SPN is free to bind to the complementary oligonucleotides on the reactive panel, resulting in a high fluorescence signal. In the presence of the target allergen, the SPN:complement binding interface is occluded, thereby resulting in a decrease in fluorescence signal on the reactive panel.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

### Example 1: Positive Graphene Oxide (GO)-SELEX Selection

As illustrated in FIG. 1, to begin a round of SELEX, the ssDNA molecules from either a random DNA library (round 1) or from the previous round (enriched library) is diluted at a concentration in water (e.g., 20 ng/μL). A target protein solution is prepared in the appropriate extraction buffer and diluted to a desired concentration depending on the round. A volume of the diluted ssDNA molecules solution (e.g., 100 μL) and a volume of the target protein solution (e.g., 300 μL) is mixed and the resulting mixture is incubated at room temperature with shaking for a set of time depending on the round. A graphene oxide (GO) solution diluted to a defined amount in the extraction buffer (e.g., 600 μL) is added to the ssDNA molecules and target mixture. Graphene oxide (GO) can adsorb the unbound sequences and let the sequences bound to the target free. The unbound sequences and GO are then removed by centrifugation. The ssDNA/target/GO mixture is incubated for 20 minutes at room temperature with shaking, during which any ssDNA that is not bound to the target material will be adsorbed onto the GO surface. After 20 minutes, the mixture is centrifuged at 10,000 g for 3 minutes, and the supernatant, containing ssDNA bound to the target protein and excess target protein, is collected. The pellet containing the GO and ssDNA adsorbed onto the GO surface is discarded.

To separate the bound ssDNAs from the target, 10% Strataclean resin is added to the collected supernatant which contains target protein and ssDNA complexes. The resulting mixture is heated to 80° C. for 3 minutes, followed by centrifugation at 10,000 g for 3 minutes. The pellet containing the resin bound target proteins is discarded and the supernatant is collected. The strataclean step is repeated for at least one more round. The concentration of ssDNAs in the final supernatant is measured and compared to the initial concentration prior to addition of target proteins and GO. The ssDNA ratio after each round of selection is used to determine if further round selection is necessary. For example, if the ratio is below 50%, the same conditions are repeated in the next round until recovery improves.

The collected final ssDNA pool is then amplified by PCR using a biotinylated reverse primer and a Cy5-tagged forward primer. The PCR amplified DNAs are cleaned for removal of any residual reagent (e.g., PCR Clean Up Kit) and measured for the concentration of DNA molecules. The clean PCR product is added to streptavidin coated magnetic beads. The biotinylated complimentary strand binds the streptavidin coated beads, then base is added to denature the dsDNA molecules. Using a magnet, the beads, with the biotinylated complimentary strand still bound, are pulled out of solution and the desired ssDNA strands with the Cy5-tag are collected. The isolated ssDNA pool is concentrated, measured and prepared for next selection round.

### Example 2: Positive On-Glass Selection

The ssDNA pool from Example 1 is diluted to 0.2 ng/μL in the extraction buffer. The same target solution is prepared and diluted to stringent conditions. 50 μL of the ssDNA solution is mixed with 50 μL of target protein and incubated for 1 minute at room temperature with shaking. This ssDNA/protein mixture is then added to two wells of a 16-well slide containing short complimentary anchors to the primer regions of the ssDNA molecules. After incubation for 1 minute at room temperature with shaking, the ssDNA/protein mixture is transferred to the next two wells of the same slide. This process is repeated for a total of eight incubations. Following the final incubation, the ssDNA/protein mixture is collected. The cleaning, amplification, and strand separation steps are the same as in the positive GO-SELEX selection (See Example 1). This on-glass selection can be repeated multiple times until the recovery ratio is acceptable.

### Example 3: Non-Binding On-Glass Counter Selection

The ssDNA molecules from the final round of positive on-glass SELEX (Example 2) are diluted to a concentration of 0.1 ng/μL in extraction buffer. 50 μL of the ssDNA solution is added to two wells of a new 16-well slide as described above. Without any protein present, all sequences in the pool that are capable of binding the complimentary sequences should bind. After incubation for 1 minute at room temperature with shaking, the ssDNA solution is transferred to the next two wells of the same slide and incubated for another 1 minute. This process is repeated for a total of eight incubations. Following the final incubation, the ssDNA is collected and saved for sequencing.

### Example 4: Binding On-Glass Counter Selection

The ssDNA molecules from the final round of positive on-glass SELEX (Example 2) are diluted to a concentration of 0.1 ng/μL in extraction buffer. The counter proteins of interest are dissociated in extraction buffer and diluted to 10000 ppm. 50 uL of the ssDNA solution is mixed with 50 μL of the counter target mixture and the mixture is incubated for 1 minute at room temperature with shaking. This ssDNA/protein mixture is then added to two wells of a fresh 16-well slide as previously described. Any ssDNA in the mixture that also has the capability to bind these undesired counter targets will bind the proteins and not bind the short complimentary sequences on the glass. After incubation for 1 minute at room temperature with shaking, the ssDNA/protein mixture is transferred to the next two wells of the same slide. This process is repeated for a total of eight incubations. Following the final incubation, the ssDNA is collected, cleaned as previously described, and saved for sequencing.

### Example 5: Selection of Aptamers that Bind to Gluten

Gluten is found in wheat, buckwheat, barley, and rye, which is composed of two primary fractions, the water and alcohol soluble gliadins, and the insoluble glutenins (Journal of AOAC International 2013; 96, 1-8). Due to these solubility differences, aptamers that recognize the gliadin fraction are selected using the combined SELEX methods.

Gluten Extraction from Food

Several different extraction methods are used and compared (Fallahbaghery et al., J. Agric. Food Chem. 2017; 65, 2857-2866; and Ito et al., Anal Bioanal Chem. 2016; 408, 5973-5984). Surfactants, salts, and reducing agents are tested. The surfactants tested include 0.1% Tween 20 or 1% SDS. The salts tested include 25 mM NaCl and 5 mM MgCl₂ or 2 mM guanidinium HCl. The only reducing agent tested is 100 mM sodium sulfite, as other reducing agents present a serious health hazard for a consumer device.

In order to select the ideal extraction buffer, 24 different buffers were tested, and their extraction efficiency was measured by ELISA. The first round of testing was performed simply on wheat, while further rounds of testing used four different wheat-incurred foods: oatmeal, wine, ground pork, and ice cream. From this testing, the best gluten extraction buffer comprises 20 mM HEPES, 30% EtOH, 0.1% Tween20, 2 mM guanidinium HCl, 25 mM NaCl, and 5 mM MgCl₂.

Determining the Ratio of GO and ssDNA Molecules

The optimal ratio of GO to ssDNA molecules in the extraction buffer is determined to achieve the maximal recovery of ssDNA molecules during the selection process. The optimal ratio is 10-fold excess of GO to ssDNA, but the affinity of ssDNAs to GO varies depending on salt content of the buffer. A dilution curve of GO using the same amount of ssDNA revealed that a 2000:1 mass ratio of GO to ssDNA is needed in the gluten extraction buffer.

Every target protein has distinct cross-reactivity concerns. For gluten, several counter proteins classes are tested, including tree nuts, commonly used wheat replacements (arrowroot, rice flour, buckwheat), and the other major allergens (egg, milk, soy).

### Example 6: Selection of Sequences as Peanut Control

Control sequences can be used in a detection assay, for example in an allergen detection assay to measure the total protein. Signals from control sequences may be incorporated into the assay algorithm in place of, or in addition to the fiducials. In this example, control sequences for peanut detection (i.e. peanut control sequences) were selected from the ssDNA library using SELEX methods as described herein. The criteria for control sequences include: 1) having similar response to a corresponding matrix, e.g., food type, as the target such as AraH1 (peanut allergen); 2) having no or litter response to the target material, e.g., peanut; 3) having no binding to either the aptamer against the target (AraH1) or its anchor sequences.

To select peanut control sequences, repeated selections with different binding materials were performed. Before each round of selection, a counter selection against 10,000 ppm peanut was performed and the collected sequences from each round of the counter selection were used. Table 16 lists the repeated selections with different binding materials.

TABLE 16
Materials and selections for peanut control sequences

| Round | Selection Materials | SELEX step |
|---|---|---|
| 1 | 1000 ppm Actin | GO-SELEX |
| 2 | 1000 ppm BSA | GO-SELEX |
| 3 | 1000 ppm Soy flour | GO-SELEX |
| 4 | 1000 ppm Tannin | GO-SELEX |

| | |
|---|---|
| 5-12 | Repeat rounds 1-4 twice |

| | | |
|---|---|---|
| 13 | 100 ppm Actin | Glass-SELEX |
| 14 | 100 ppm BSA | Glass-SELEX |
| 15 | 100 ppm Soy flour | Glass-SELEX |
| 16 | 100 ppm Tannin | Glass-SELEX |
| 17 | 100 ppm Peanut | Glass-SELEX |
| 18 | No protein | Glass-SELEX |

Sequences collected from Rounds 16, 17 and 18 were sequenced and tested. The heat maps, predicted binding to an aptamer specific to the peanut allergen protein AraH1 (AraH1 probe) and the anchor sequences, and folded structures of each sequence were analyzed. Table 11 lists the top 1000 hits from the selection. 13 control sequences (Table 17) were picked and further characterized,

TABLE 17
Control aptamers for peanut control materials

| Control | | SEQ |
|---|---|---|
| sequence | Sequence (5′-3′) | ID NO |
| PC14 | TAGGGAAGAGAAGGACATATGATGTCGTGACTG | 39016 |
| | GCTAGCTGGACATGCACTGCTTGACTAGTACAT | |
| | GACCACTTGA | |
| PC36 | TAGGGAAGAGAAGGACATATGATGCACTGGCTG | 39038 |
| | ACCTACACGTGGACGATGTGTTGACTAGTACAT | |
| | GACCACTTGA | |
| PC41 | TAGGGAAGAGAAGGACATATGATGCACGCCGAT | 39043 |
| | GCCCTCATGTGGCCGTGGATTGACTAGTACATG | |
| | ACCACTTGA | |
| PC48 | TAGGGAAGAGAAGGACATATGATGACGACACGA | 39050 |
| | CCTTCAAGCATGGCCTAGCGTTGACTAGTACAT | |
| | GACCACTTGA | |
| PC54 | TAGGGAAGAGAAGGACATATGATGGACGCAACG | 39056 |
| | TACCGTATCGTGGCCATGTGTTGACTAGTACAT | |
| | GACCACTTGA | |
| PC55 | TAGGGAAGAGAAGGACATATGATGCACGTACGC | 39057 |
| | CTTGCCTATCTGTGCTCATGTTGACTAGTACAT | |
| | GACCACTTGA | |
| PC58 | TAGGGAAGAGAAGGACATATGATGGCATGCGCT | 39060 |
| | GGGTAGTGATCACGTACGGTTTGACTAGTACAT | |
| | GACCACTTGA | |
| PC60 | TAGGGAAGAGAAGGACATATGATCGTACCGCAA | 39062 |
| | GTGACGTGTCCGTGCCGTGATTGACTAGTACAT | |
| | GACCACTTGA | |
| PC66 | TAGGGAAGAGAAGGACATATGATGTCATGCGCG | 39068 |
| | TACCATCGAGGGGGCGTGGATTGACTAGTACAT | |
| | GACCACTTGA | |
| PC77 | TAGGGAAGAGAAGGACATATGATGGACTGAACG | 39079 |
| | TACTGCCAGGTGAGCATGCATTGACTAGTACAT | |
| | GACCACTTGA | |
| PC85 | TAGGGAAGAGAAGGACATATGATCGATGGTACG | 39087 |
| | AACGCCACGTCATGCGGTCATTGACTAGTACAT | |
| | GACCACTTGA | |
| PC87 | TAGGGAAGAGAAGGACATATGATGCGTGTCAGC | 39089 |
| | AATACGTCCTCATCTGCCCGTTGACTAGTACAT | |
| | GACCACTTGA | |
| PC96 | TAGGGAAGAGAAGGACATATGATGGACAACGTG | 39098 |
| | GCTGGTAGGTATCGTGGGCATTGACTAGTACAT | |
| | GACCACTTGA | |

None of these peanut control sequences bind to the aptamer specific to AraH1 (AraH1 probe) up to the concentration of 100 nM.

The binding of the control sequences to an anchor sequence (AAAAATCAAGTGGTC; SEQ ID NO. 52003), the interference of each sequence with the binding of the AraH1 probe to peanut, and the affinity of each sequence to peanut, were evaluated. The data showed that three sequences PC36, PC60 and PC87 have minimal response to 5000 ppm peanut when tested at a concentration of 100 nM.

Two food types including sugar free wafer and strawberry poptart were compared for response to AraH1 aptamer and peanut control sequences PC36, PC60 and PC87 (each at a concentration of 100 nM). The foods were spiked with either 0 ppm or 5000 ppm peanut. The data indicate the AraH1 aptamer creates high signal for wafer but a lower signal for poptart.

TABLE 18
signals of AraH1 aptamer and peanut control sequences

| | poptart | | | | wafer | | | | Ration |
|---|---|---|---|---|---|---|---|---|---|
| | 0 | 5000 | % of | T- | 0 | 5000 | % of | T- | (Wafer:poptart) |
| sequence | ppm | ppm | change | test | ppm | ppm | change | test | 0 ppm |
| AraH1 | 1.9 | 1.8 | 93% | 0.032 | 6.8 | 1.7 | 26% | 0.003 | 3.5 |
| aptamer | | | | | | | | | |
| PC36 | 5.9 | 6.5 | 109% | 0.612 | 23.3 | 15.8 | 68% | 0.069 | 3.9 |
| PC60 | 5.4 | 5.3 | 100% | 0.981 | 9.5 | 7.6 | 80% | 0.981 | 1.8 |
| PC87 | 103.9 | 102.3 | 99% | 0.920 | 127.6 | 132.5 | 104% | 0.791 | 1.2 |

1. A method for identifying an aptamer that specifically binds to a target of interest comprising:
(a) preparing an input DNA library comprising a plurality of single stranded DNA (ssDNA) molecules, each of which comprises a central randomized nucleic acid sequence flanked by a constant sequence at the 5′ end and a constant sequence at the 3′ end, the constant 5′ end and the constant 3′ end functioning as primers;
(b) selecting, from the input DNA library, a first pool of ssDNA molecules that substantially bind to the target;
(c) selecting a second pool of ssDNA molecules, from the first target binding pool of ssDNA molecules, that do not substantially hybridize to their complementary sequences in the presence of the target;
(d) counter-selecting a third pool of ssDNA molecules, from the second positive binding pool of ssDNA molecules obtained, that do not hybridize to the complementary sequences in the absence of the target, and a fourth pool of sequences that substantially bind to counter targets; and
(e) subtracting the ssDNA molecules in the third and fourth pools from the second positive binding pool of ssDNA molecules, and identifying ssDNA molecules that specifically bind to the target of interest.

2. The method of claim 1, wherein the first pool of ssDNA molecules that substantially bind to the target material is selected through the steps of:
(i) contacting the input ssDNA library with a target material wherein complexes are formed between the target and a plurality of ssDNA molecules present in the input library;
(ii) partitioning the ssDNA:target complexes formed in step (i) from unbound ssDNA molecules using a Graphene Oxide (GO) solution, and isolating the ssDNA molecules in the complexes to produce a subset of ssDNA molecules for the target;
(iii) contacting the subset of ssDNA molecules in (ii) with the same target wherein complexes are formed between the target and a second plurality of ssDNA molecules present in the subset of ssDNA molecules to generate a second subset group of ssDNA molecules for the target; and
(iv) optionally repeating steps (ii) to (iii), one, two, three, four or more rounds to produce a respective third, fourth, fifth, sixth or more subset group of ssDNA molecules, thereby producing the enriched pool of ssDNA molecules that substantially bind to the target after the final round.

3. The method of claim 2, wherein the second positive binding pool of ssDNA molecules is selected through an on-chip positive selection process using the first target binding pool of ssDNA molecules, the same target and a solid support that is coated with short oligonucleotides that are complementary to the constant sequence of the ssDNA molecules, the on-chip positive selection process comprising the steps of:
(i) mixing the first target binding pool of ssDNA molecules with the same target in a buffer solution and inducing them to bind to each other;
(ii) contacting the mixture of step (i) with the solid support of which the surface is covalently coated with short oligonucleotides that are complementary to the constant sequence of the ssDNA molecules;
(iii) collecting a flow-through containing ssDNA:target complexes that are not bound to the solid support;
(iv) optionally contacting the collected flow-through again with the solid support coated with the complementary oligonucleotides for two, three, four, five, six, seven, eight, or more times; and
(v) removing the target and collecting an enriched subset of ssDNA molecules after the final incubation.

4. (canceled)

5. The method of claim 3, wherein the third pool of ssDNA molecules is selected by an on-chip non-binding counter process using the second positive binding pool of ssDNA molecules and a solid support that is coated with short oligonucleotides that are complementary to the constant sequence of the ssDNA molecules.

6. The method of claim 3, wherein the fourth pool of ssDNA molecules is selected through an on-chip counter binding process using the second positive binding pool of ssDNA molecules, one or more counter targets and a solid support that is coated with short oligonucleotides that are complementary to the constant sequence of the ssDNA molecules.

7. The method of claim 1, wherein the method further comprises:
(f) amplifying and sequencing the ssDNA molecules in each pool obtained in steps (b) to (d); and
(g) generating a sequence map for each pool of ssDNA molecules and analyzing the sequence information and selecting a final pool of ssDNA molecules that specifically and preferentially bind to the target of interest in the presence of the complementary sequences.

8. The method of claim 7, wherein the step (g) comprises:
(i) amplifying all the ssDNA molecules in the first, second, third and fourth pools, and barcoding each sequence from each pool;
(ii) pooling together the sequences from each pool and running sequencing together;
(iii) analyzing the data from (ii) and separating data for each sequence into the original pool according to the barcode information;
(iv) generating heat maps for each individual pool that represent the frequency of each sequence in the pool; and
(v) subtracting the sequences in the heat maps of the third pool of ssDNA molecules and the sequences in the fourth pool of ssDNA molecules, from the heat maps of the second positive binding pool of ssDNA molecules,
wherein the final pool of the sequences after step (v) represents aptamer candidates that specifically bind to the target of interest and preferentially bind to the target of the interest in competing the binding of various short complementary sequences.

9. The method of claim 8, wherein the method further comprises subtracting any sequences from an artifact pool of ssDNA molecules from the first target binding pool of ssDNA molecules, wherein the artifact pool of ssDNA molecules is generated by PCR amplification and strand separation of the input DNA library, representing the sequences that are over-amplified by PCR amplification.

10. The method of claim 9, wherein the sequences of the final pool are analyzed for sequence similarities and secondary structures.

11. The method of claim 7, wherein the sequences of ssDNA molecules in each pool are amplified by PCR using a pair of Cy5 labeled primers and biotinylated primers.

12. The method of claim 1, wherein the target is an allergen.

13. An aptamer that binds to an allergen with high specificity and affinity, wherein the aptamer does not hybridize to its complementary sequences in the presence of the target allergen.

14. (canceled)

15. The aptamer of claim 13, wherein the allergen is peanut and the aptamer specific to peanut comprises a nucleic acid sequence selected from the group consisting of SEQ ID NOs.3 to 4002.

16. (canceled)

17. The aptamer of claim 13, wherein the allergen is almond and the aptamer specific to almond comprises a nucleic acid sequence selected from the group consisting of SEQ ID NOs. 4003 to 8002.

18. (canceled)

19. The aptamer of claim 13, wherein the allergen is brazil nut and the aptamer specific to brazil nut comprises a nucleic acid sequence selected from the group consisting of SEQ ID NOs. 8003 to 12002.

20. (canceled)

21. The aptamer of claim 13, wherein the allergen is cashew and the aptamer specific to cashew comprises a nucleic acid sequence selected from the group consisting of SEQ ID NOs. 12003 to 16002.

22. (canceled)

23. The aptamer of claim 13, wherein the allergen is hazelnut and the aptamer specific to hazelnut comprises a nucleic acid sequence selected from the group consisting of SEQ ID NOs. 16003 to 20002.

24. (canceled)

25. The aptamer of claim 13, wherein the allergen is pecan and the aptamer specific to pecan comprises a nucleic acid sequence selected from the group consisting of SEQ ID NOs. 20003 to 24002.

26. (canceled)

27. The aptamer of claim 13, wherein the allergen is pistachio and the aptamer specific to pistachio comprises a nucleic acid sequence selected from the group consisting of SEQ ID NOs. 24003 to 28002.

28. (canceled)

29. The aptamer of claim 13, wherein the allergen is walnut and the aptamer specific to walnut comprises a nucleic acid sequence selected from the group consisting of SEQ ID NOs. 28003 to 32002.

30. (canceled)

31. The aptamer of claim 13, wherein the allergen is a mix of nuts and the aptamer specific to the mixed nuts comprises a nucleic acid sequence selected from the group consisting of SEQ ID NOs. 32003 to 36002.

32. (canceled)

33. The aptamer of claim 31, wherein the mixed nuts comprise peanut, almond, brazil nut, cashew, hazelnut, pistachio, pecan and walnut.

34. The aptamer of claim 13, wherein the allergen is gluten and the aptamer specific to gluten comprises a nucleic acid sequence selected from the group consisting of SEQ ID NOs. 40003 to 44002.

35. (canceled)

36. The aptamer of claim 13, wherein the allergen is whey and the aptamer specific to whey comprises a nucleic acid sequence selected from the group consisting of SEQ ID NOs. 44003 to 48002.

37. (canceled)

38. The aptamer of claim 13, wherein the allergen is casein and the aptamer specific to casein comprises a nucleic acid sequence selected from the group consisting of SEQ ID NOs. 48003 to 52002.

39. (canceled)

40. (canceled)

41. A control aptamer that recognizes a panel of control materials that are used for peanut allergen comprising a nucleic acid sequence selected from the group consisting of SEQ ID NOs. 36003 to 40002.

42. (canceled)

43. A signaling polynucleotide (SPN) comprising an aptamer sequence that binds to an allergen with high specificity and affinity, wherein the aptamer sequence does not hybridize to its complementary sequences in the presence of the target allergen, and a fluorophore conjugated to one end of the aptamer sequence, and a short oligonucleotide sequence that is complementary to the aptamer sequence or a portion of the aptamer sequence.

44. The SPN of claim 43, wherein the fluorophore is Cy5, Texas red or Alexa Fluor 647.

45. (canceled)

46. The SPN of claim 44,
wherein the allergen is peanut and the aptamer specific to peanut comprises a nucleic acid sequence selected from the group consisting of SEQ ID NOs. 3 to 4002;
wherein the allergen is almond and the aptamer specific to almond comprises a nucleic acid sequence selected from the group consisting of SEQ ID NOs. 4003 to 8002;
wherein the allergen is brazil nut and the aptamer specific to brazil nut comprises a nucleic acid sequence selected from the group consisting of SEQ ID NOs. 8003 to 12002;
wherein the allergen is cashew and the aptamer specific to cashew comprises a nucleic acid sequence selected from the group consisting of SEQ ID NOs. 12003 to 16002;
wherein the allergen is hazelnut and the aptamer specific to hazelnut comprises a nucleic acid sequence selected from the group consisting of SEQ ID NOs. 16003 to 20002;
wherein the allergen is pecan and the aptamer specific to pecan comprises a nucleic acid sequence selected from the group consisting of SEQ ID NOs. 20003 to 24002;
wherein the allergen is pistachio and the aptamer specific to pistachio comprises a nucleic acid sequence selected from the group consisting of SEQ ID NOs. 24003 to 28002;
wherein the allergen is walnut and the aptamer specific to walnut comprises a nucleic acid sequence selected from the group consisting of SEQ ID NOs. 28003 to 32002;
wherein the allergen is a mix of nuts and the aptamer against the nuts comprises a nucleic acid sequence selected from the group consisting of SEQ ID NOs. 32003 to 36002;
wherein the allergen is gluten and the aptamer specific to gluten comprises a nucleic acid sequence selected from the group consisting of SEQ ID NOs. 40003 to 44002;
wherein the allergen is whey and the aptamer specific to whey comprises a nucleic acid sequence selected from the group consisting of SEQ ID NOs. 44003 to 48002; or
wherein the allergen is casein and the aptamer specific to casein comprises a nucleic acid sequence selected from the group consisting of SEQ ID NOs. 48003 to 52002.

47. (canceled)

48. (canceled)

49. (canceled)

50. (canceled)

51. (canceled)

52. (canceled)

53. (canceled)

54. (canceled)

55. The SPN of claim 46, wherein the mixed nuts comprise peanut, almond, brazil nut, cashew, hazelnut, pistachio, pecan and walnut.

56. (canceled)

57. (canceled)

58. (canceled)

59. (canceled)

60. The SPN of claim 43, wherein the short complementary sequence comprises a nucleic acid sequence selected from the group consisting of SEQ ID NOs. 52003 to 52042.

61. A detection sensor comprising:
(i) signaling polynucleotide (SPN), wherein the SPN comprises an aptamer sequence that binds to an allergen with high specificity and affinity and that does not hybridize to its complementary sequences in the presence of the target allergen, and
(ii) a short nucleic acid sequence that is printed on a solid surface, wherein the short nucleic acid sequence is complementary to the SPN.

62. (canceled)

63. (canceled)

64. The detection sensor of claim 61, wherein the allergen is peanut, almond, brazil nut, cashew, hazelnut, pecan, pistachio, gluten, whey or casein.

65. The detection sensor of claim 61 further comprising a control nucleic acid sequence, wherein the control sequence has the features including: i) no binding affinity to the target of interest; ii) no binding affinity to the target specific aptamer and iii) no binding affinity to the short anchor sequences on the solid surface.

66. A detection kit comprising,
(a) a signaling polynucleotide (SPN) comprising an aptamer sequence that binds to a target of interest with high specificity and affinity and that does not hybridize to its complementary sequences in the presence of the target of interest;
(b) a solid support of which the surface is coated with short nucleic acid sequences that are complementary to the sequence of the aptamer; and
(c) one or more buffer solutions.

67. (canceled)

68. The detection kit of claim 66 further comprising a SPN comprising an aptamer sequence that binds to a control material.

69. A method for detecting the presence, and/or absence of an allergen in a food sample comprising:
(a) preparing a food sample solution wherein the solution comprising a SPN comprising an aptamer that specifically binds to said allergen an allergen and that is labeled with a fluorophore;
(b) contacting the mixture of the sample and SPN to a solid support that is coated with short nucleic acid sequences that are complementary to the aptamer sequence; and
(c) measuring fluorescence signals and detecting the presence and/or absence of the allergen of interest in the food sample.

70. The method of claim 69 further comprising a step of
(d) measuring the total protein from the food sample using a SPN comprising an aptamer that bind to the allergen control material.

71. The method of claim 69, wherein the allergen is peanut.

72. The method of claim 71, wherein the SPN that specifically binds to peanut comprising a nucleic acid sequence selected the group consisting of SEQ ID NOs. 3 to 4002.

73. The method of claim 72, wherein the SPN that binds to peanut control material comprising a nucleic acid sequence selected the group consisting of SEQ ID NOs. 36003 to 40002.
